(19) United States
(12) Patent Application Publication
(10) Pub. No.: US 2026/0137795 A1
(43) Pub. Date: May 21, 2026

(54) EXATECAN IMMUNOCONJUGATES

(71) Applicant: Immunome, Inc., Bothell, WA (US)

(72) Inventors: Xiaojun Han, San Diego, CA (US); Kevin Duane Bunker, Escondido, CA (US); Kimberlee Fischer, San Diego, CA (US)

(21) Appl. No.: 19/151,187

(22) PCT Filed: Jan. 25, 2024
(86) PCT No.: PCT/US2024/012923

§ 371 Date: Jul. 25, 2025

Related U.S. Application Data
(60) Provisional application No. 63/481,567, filed on Jan. 25, 2023.

(51) Int. Cl. A61K 47/68; A61P 35/00

(52) U.S. Cl. CPC A61K 47/68037; A61P 35/00

(57) ABSTRACT

Immunoconjugates of the Formula (I), or a pharmaceutically acceptable salt thereof, include a linking group for linking an antibody targeting ligand (Ab) to a drug (D). Embodiments of such immunoconjugates are useful for delivering the drug to selected cells or tissues, e.g., for the treatment, inhibition, or amelioration of a cancer.

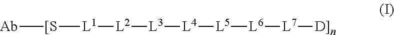

### CROSS-REFERENCE TO RELATED APPLICATIONS

The application claims the benefit of, and priority to, U.S. Patent Application No. 63/481,567, filed Jan. 25, 2023, the contents of which are incorporated by reference herein in their entirety.

### SEQUENCE STATEMENT

This application contains a Sequence Listing, which has been submitted electronically and is hereby incorporated by reference in its entirety. The sequence listing, was created on Jan. 23, 2023, is named ZENO_161PR and is 46 kb in size.

### BACKGROUND

### Field

The application relates to conjugates that include a linking group for linking an antibody targeting ligand to a cell-killing moiety (such as a drug), methods of making such conjugates, and methods of using such conjugates to deliver the cell-killing moiety to selected cells or tissues, e.g., for the treatment or inhibition of a cancer.

### Description

A number of antibody-drug conjugates (ADC) have been developed for medical uses. See, e.g., Nejadmoghaddam, M, et al., “Antibody-Drug Conjugates: Possibilities and Challenges”, Avicenna J Med Biotech 11(1), 3-23 (2019). The antibody in the ADC functions as a targeting agent to deliver the drug to a selected cell or tissue such as a cancer cell or tumor. In the United States, the U.S. Food and Drug Administration (FDA) has approved several ADC formulations, including inotuzumab ozogamicin (tradename BESPONSA), gemtuzumab ozogamicin (tradename MYLOTARG), brentuximab vedotin (tradename ADCETRIS), ado-trastuzumab emtansine (tradename KADCYLA), mirvetuximab-soravtansine-gynx (Elahere™), tisotumab vedotin-tftv (Tivdak™), loncastuximab tesirine-lpyl (Zynlonta®), sacituzumab govitecan (Trodelvy®), trastuzumab deruxtecan (Enhertu®), enfortumab vedotin (Padcev®), polatuzumab vedotin-piiq (Polivy®), moxetumomab pasudotox (Lumoxiti®) and inotuzumab ozogamicin (Besponsa®).

U.S. Pat. No. 10,155,821 discloses ADCs in which an antitumor compound is conjugated to an anti-HER2 antibody via a linker. See also U.S. Patent Publication Nos. 2020/0385486 and 2019/0077880. Trastuzumab deruxtecan is an example of an ADC in which an anti-HER2 antibody (trastuzumab) is attached via a cleavable maleimide tetrapeptide linker to an antitumor compound (Dxd). The FDA has approved a formulation known as fam-trastuzumab deruxtecan-nxki (tradename ENHERTU) for the treatment of adult patients with unresectable or metastatic HER2-positive breast cancer who have received two or more prior anti-HER2-based regimens in the metastatic setting. FIG. 1 illustrates the manner in which it is believed the linker connects the antibody (mAb) to the drug moiety.

The FDA approvals represent milestones in the ongoing development of therapeutic ADCs. However, there remains a need for improved ADCs to help address the long-felt need for additional options to treat cancer and/or deliver therapeutic payloads to selected cells or tissues.

### SUMMARY

Some embodiments provide an immunoconjugate of Formula (I) that comprises an antibody or antigen-binding fragment (Ab), and drug moiety (D) and a linker connecting Ab to D. In an embodiment, the immunoconjugate of Formula (I) comprises a drug moiety of the Formula (II).

An embodiment provides an immunoconjugate having Formula (I),

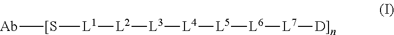

or a pharmaceutically acceptable salt thereof, wherein:
Ab is an antibody or an antigen-binding fragment;
L¹ is

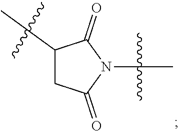 [SMILES: *C1CC(=O)N(*)C1=O]

L² is absent,

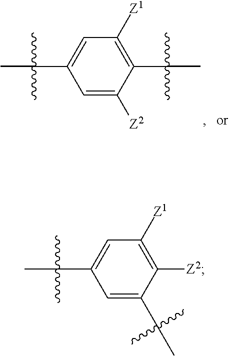

Z¹ and Z² are each individually hydrogen, halogen, —NO₂, —O—(C₁-C₆ alkyl), or C₁-C₆ alkyl;
L³ is —(CH₂)n¹-C(═O)— or —(CH₂CH₂O)n¹-(CH₂)n¹C(═O)—;
n¹ are independently integers of 0 to 12;
L⁴ is a tetrapeptide residue;
L⁵ is absent or —[NH(CH₂)n²]n³-;
n² is an integer of 0 to 6;
n³ is an integer of 0 to 2;
L⁶ is absent or

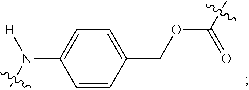

L⁷ is absent,

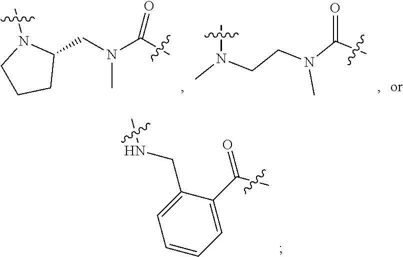

D is a drug moiety; and
n is an integer from 1 to 10.

In an embodiment, D in Formula (I) is a drug moiety of Formula (II), or a pharmaceutically acceptable salt thereof, having the structure:

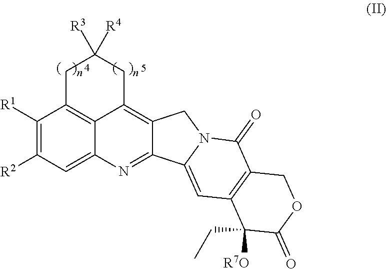

wherein:
R¹ and R² are each individually selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, or a substituted or an unsubstituted —O—(CR⁵R⁶)ₘ—O— such that R¹ and R² taken together form a ring;
R³ and R⁴ are each individually selected from hydrogen, —OH, —N₃, —NH₂, —NH(C═O)—CH₂—R^{3D}, a substituted or an unsubstituted C₁-C₆ alkyl, a substituted or an unsubstituted C₂-C₆ alkenyl and [(CY₂)ₚO(CY₂)_{q}]ₜOH, with the proviso that R³ and R⁴ are not both hydrogen, wherein when the C₁-C₆ alkyl or C₂-C₆ alkenyl is substituted, the C₁-C₆ alkyl or C₂-C₆ alkenyl is substituted with one or more R^{3A} groups selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl); or one of R³ and R⁴ is a substituted or an unsubstituted (C₁-C₆ alkyl)-X² or a substituted or an unsubstituted —(C₂-C₆ alkenyl)-X², wherein when-(C₁-C₆ alkyl)-X² or —(C₁-C₆ alkenyl)-X² is substituted, the —(C₁-C₆ alkyl)-X² or the —(C₁-C₆ alkenyl)-X² is substituted with one or more R^{3A} groups selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl);
R^{3D} is selected from H, —CH₃, —OH and —CH₂Y¹, wherein Y¹ is halogen;
X² is —OR⁹, —SR⁹, or —NHR⁹;
R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl;
n⁴ and n⁵ are each individually 0, 1 or 2, with the proviso that n⁴ and n⁵ are not both ( );
each Y is individually H or halogen;
each m is individually 1 or 2;
each p is individually 1, 2, 3, 4, 5, or 6;
each q is individually 0, 1, 2, 3, 4, 5, or 6;
each t is individually 1, 2, 3, 4, 5, or 6;
R⁷ is H, —COR⁸, —CO₂R⁸, —(CO)—NHR⁸, L⁴, L⁵, L⁶, or L⁷;
R⁸ is a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³;
R⁹ is H, —COR⁸, —CO₂R⁸, —(CO)—NHR⁸, L⁴, L⁵, L⁶, or L⁷, with the proviso that exactly one of R¹ and R⁹ is L⁴, L⁵, L⁶, or L⁷; and
each X³ is individually —H, —OH, —SH, or —NH₂.

An embodiment provides a compound of Formula (IV), or a pharmaceutically acceptable salt thereof, having the structure:

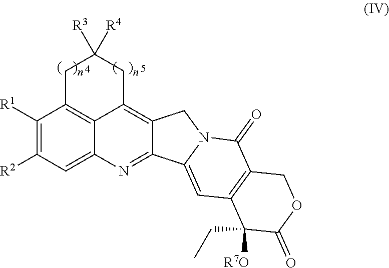

wherein:
R¹ and R² are each individually selected from hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, or a substituted or an unsubstituted —O—(CR⁵R⁶)ₘ—O— such that R¹ and R² taken together form a ring:
R³ and R⁴ are each individually selected from hydrogen, —OH, —N₃, —NH₂, —NH(C═O)—CH₂—R^{3D}, a substituted or an unsubstituted C₁-C₆ alkyl, a substituted or an unsubstituted C₂-C₆ alkenyl and [(CY₂)ₚO(CY₂)_{q}]ₜOH, with the proviso that R³ and R⁴ are not both hydrogen; wherein when the C₁-C₆ alkyl or C₂-C₆ alkenyl is substituted, the C₁-C₆ alkyl or C₂-C₆ alkenyl is substituted with one or more R^{3A} groups individually selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl):
R^{3D} is selected from H, —CH₃, —OH and —CH₂Y¹, wherein Y¹ is halogen;
R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl;
n⁴ and n⁵ are each individually 0, 1 or 2, with the proviso that n⁴ and n⁵ are not both 0;
each Y is individually H or halogen;
each m is individually 1 or 2;
each p is individually 1, 2, 3, 4, 5, or 6;
each q is individually 0, 1, 2, 3, 4, 5, or 6; and
each t is individually 1, 2, 3, 4, 5, or 6;
R⁷ is H, —COR⁸, —CO₂R⁸, or —(CO)—NHR⁸; and
R⁸ is a substituted or an unsubstituted C₁-C₆ alkyl, a substituted or an unsubstituted C₁-C₆ haloalkyl, or —[(CY₂)ₚO(CY₂)_{q}]ₜCY₃.

An embodiment provides a pharmaceutical composition comprising an immunoconjugate as described herein, a drug compound as described herein, or a pharmaceutically active salt thereof, and a pharmaceutically acceptable carrier, diluent, excipient or combination thereof.

An embodiment provides a method for treating a cancer or a tumor comprising administering an effective amount of an immunoconjugate as described herein, a drug compound as described herein, or a pharmaceutically active salt thereof, or a pharmaceutical composition as described herein, to a subject having the cancer or the tumor.

An embodiment provides a use of an effective amount of an immunoconjugate as described herein, a drug compound as described herein, or a pharmaceutically active salt thereof, or a pharmaceutical composition as described herein, in the manufacture of a medicament for treating a cancer or a tumor.

Some embodiments provide a conjugate of Formula (III) that comprises a functional group M1, a drug moiety (D) and a linker connecting Mi to D. In an embodiment, the conjugate of Formula (III) comprises a drug moiety of the Formula (II).

An embodiment provides a conjugate having Formula (III),

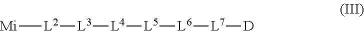

or a pharmaceutically acceptable salt thereof, wherein:
Mi is

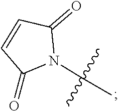 [SMILES: *N1C(=O)C=CC1=O]

L² is absent,

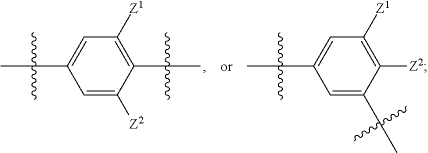

Z¹ and Z² are each individually hydrogen, halogen, —NO₂, —O—(C₁-C₆ alkyl), or C₁-C₆ alkyl;
L³ is —(CH₂)n¹-C(═O)— or —(CH₂CH₂O)n¹-(CH₂)n¹C(═O)—;
n¹ are independently integers of 0 to 12;
L⁴ is a tetrapeptide residue;
L⁵ is absent or —[NH(CH₂)n²]n³-;
n² is an integer of 0 to 6;
n³ is an integer of 0 to 2;
L⁶ is absent or

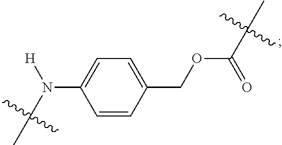 [SMILES: *Nc1ccc(COC(*)=O)cc1]

L⁷ is absent,

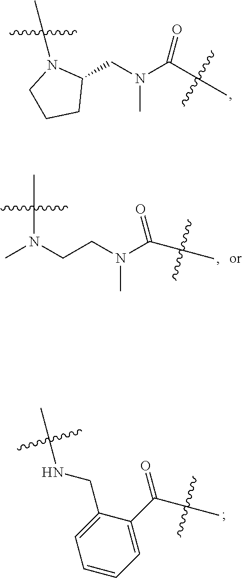

and
D is a drug moiety.

An embodiment provides a process of producing an immunoconjugate, comprising: reacting an effective amount of a thiol-functionalized antibody or antigen-binding fragment thereof with a conjugate as described herein under reaction conditions effective to form an immunoconjugate as described herein.

An embodiment provides an immunoconjugate, pharmaceutical composition, method of treatment, inhibition, or amelioration, use, or process of making as described herein, wherein Ab is an antibody or antigen-binding fragment thereof comprising:
a) a heavy chain comprising:
VHCDR 1 comprising an amino acid sequence having at least 95% sequence identity to the amino acid sequence of SEQ ID NO:1;
VHCDR 2 comprising an amino acid sequence having at least 95% sequence identity to the amino acid sequence of SEQ ID NO:2; and
VHCDR 3 comprising an amino acid sequence having at least 95% sequence identity to the amino acid sequence of SEQ ID NO:3; and
b) a light chain comprising:
VLCDR 1 comprising an amino acid sequence having at least 95% sequence identity to the amino acid sequence of SEQ ID NO:8;
VLCDR 2 comprising an amino acid sequence having at least 95% sequence identity to the amino acid sequence of AAS; and
VLCDR 3 comprising an amino acid sequence having at least 95% sequence identity to the amino acid sequence of SEQ ID NO: 10;
wherein the antibody or antigen-binding fragment thereof specifically binds to the extracellular domain of human receptor tyrosine kinase like orphan receptor 1 (ROR1).

An embodiment provides an immunoconjugate, pharmaceutical composition, method of treatment, inhibition, amelioration, use, or process of making as described herein, wherein Ab is an antibody or antigen-binding fragment thereof comprising:
a) a heavy chain comprising:
VHCDR 1 comprising an amino acid sequence having at least 95% sequence identity to the amino acid sequence of SEQ ID NO: 15;
VHCDR 2 comprising an amino acid sequence having at least 95% sequence identity to the amino acid sequence of SEQ ID NO: 16; and
VHCDR 3 comprising an amino acid sequence having at least 95% sequence identity to the amino acid sequence of SEQ ID NO: 17; and
b) a light chain comprising:
VLCDR 1 comprising an amino acid sequence having at least 95% sequence identity to the amino acid sequence of SEQ ID NO:22:
VLCDR 2 comprising an amino acid sequence having at least 95% sequence identity to the amino acid sequence of DAY; and
VLCDR 3 comprising an amino acid sequence having at least 95% sequence identity to the amino acid sequence of SEQ ID NO:24;
wherein the antibody or antigen-binding fragment thereof specifically binds to the extracellular domain of human receptor tyrosine kinase like orphan receptor 1 (ROR1).

An embodiment provides an immunoconjugate, pharmaceutical composition, method of treatment, inhibition, amelioration, use, or process of making as described herein, wherein Ab is an antibody or antigen-binding fragment thereof comprising:
a) a heavy chain comprising:
VHCDR 1 comprising an amino acid sequence having at least 95% sequence identity to the amino acid sequence of SEQ ID NO:29:
VHCDR 2 comprising an amino acid sequence having at least 95% sequence identity to the amino acid sequence of SEQ ID NO:30; and
VHCDR 3 comprising an amino acid sequence having at least 95% sequence identity to the amino acid sequence of SEQ ID NO:31; and
b) a light chain comprising:
VLCDR 1 comprising an amino acid sequence having at least 95% sequence identity to the amino acid sequence of SEQ ID NO:36;
VLCDR 2 comprising an amino acid sequence having at least 95% sequence identity to the amino acid sequence of DAS; and
VLCDR 3 comprising an amino acid sequence having at least 95% sequence identity to the amino acid sequence of SEQ ID NO:38:
wherein the antibody or antigen-binding fragment specifically binds to the extracellular domain of human receptor tyrosine kinase like orphan receptor 1 (ROR1).

These and other embodiments are described in greater detail below.

### BRIEF DESCRIPTION OF THE DRAWINGS

FIG. 1 illustrates a trastuzumab deruxtecan antibody-drug conjugate.

FIG. 2 illustrates a reaction scheme for making a compound of the Formula (IV) with n⁴=2 and n⁵=0.

FIG. 3A illustrates a reaction scheme for making an immunoconjugate of the Formula (I).

FIG. 3B illustrates a reaction scheme for a conjugate of Formula (III).

FIG. 4 illustrates a reaction scheme for making compounds 1-11a, 1-11b, 1-11c and 1-11d.

FIG. 5 illustrates a reaction scheme for making compounds 2-15a, 2-15b, 2-15c and 2-15d.

FIG. 6 illustrates a reaction scheme for making compounds 3-21a and 3-21b.

FIG. 7 illustrates a reaction scheme for making compounds 4-27a and 4-27b.

FIG. 8 illustrates a reaction scheme for making compounds 5-34a and 5-34b.

FIG. 9 illustrates a reaction scheme for making compounds 6-40a and 6-40b.

FIG. 10 illustrates a reaction scheme for making compounds 7-42a, 7-42b, 7-42c and 7-42d.

FIG. 11 illustrates a reaction scheme for making compounds 8-47a, 8-47b, 8-47c and 8-47d.

FIG. 12 illustrates a reaction scheme for making compounds 9-52a, 9-52b, 9-52c and 9-52d.

FIG. 13 illustrates a reaction scheme for making an exemplary conjugate of Formula (III).

FIG. 14 illustrates a reaction scheme for making compound 10-60, which is an exemplary conjugate of Formula (III).

FIG. 15 illustrates a reaction scheme for making compound 10-59, which is an exemplary intermediate in the preparation of an exemplary conjugate of Formula (III).

FIG. 16 illustrates a measurement of cell binding saturation data for the anti-ROR-1 antibodies generated by the methods described herein. A ROR-1 positive cell line JeKo-1 was incubated in a titration series with the anti-ROR-1 antibodies ATX-P-875. ATX-P-885, and ATX-P-890 in comparison to the positive control antibody UC961. Cells were washed, stained with secondary antibody and cell binding saturation was detected by flow cytometry and reported as mean fluorescent intensity (MFI).

FIG. 17 illustrates ROR-1 receptor internalization data for the anti-ROR-1 antibodies ATX-875. ATX-P-885. ATX-P-890. ROR-1 positive cell lines JeKo-1 and MDA-MB-468 were incubated with the anti-ROR-1 antibodies ATX-P-875, ATX-P-885, and ATX-P-890 and positive control antibody UC961 at super saturating conditions so as to bind all available ROR-1 receptors. Cells were washed and incubated at 4 different timepoints (30 min. 1 hour, 2 hours and 4 hours) at 37° C., before internalization was halted by placing the cells in ice. Receptor internalization was determined by flow cytometry and reported as percent receptor internalization relative to zero hours.

FIG. 18-18D illustrates cellular binning data for the anti-ROR-1 antibodies ATX-P-875, ATX-P-885, and ATX-P-890. A cellular binning assay was performed to assess if ATX-P-875, ATX-P-885, and ATX-P-890 bound the same epitopes on the ROR-1 receptor as control antibodies UC961 and 4A5. FIG. 18A depicts a staining profile for antibodies that bind the same epitope. FIG. 18B depicts the staining profile for antibodies that bind different epitopes. ATX-P-875, ATX-P-885, and ATX-P-890 were separately incubated with ROR-1_+MDA-MB-468 at various amounts. Next, the anti-ROR-1 antibodies were fluorescently labeled with a secondary antibody. Finally, MDA-MB-468 cells coated with the anti-ROR-1 antibodies were incubated with a saturating dose of a fluorescently labeled UC961 (FIG. 18C) or 4A5 (FIG. 18D) and analyzed by flow cytometry and the ATX-P-875. ATX-P-885, and ATX-P-890 antibody signal were compared with the UC961 or 4A5 signal.

FIG. 19 illustrates AC-SINS data for the anti-ROR-1 antibodies ATX-P-875, ATX-P-885, and ATX-P-890. Antibody developability was assessed by performing an AC-SINS assay and evaluating the potential for self-interaction. Rituximab and Infliximab were used as controls to demonstrate a low and high shift, respectively. Assay results for ATX-P-875, ATX-P-885, and ATX-P-890 fell within the range determined by the control antibodies.

FIG. 20 illustrates biochemical binning data by SPR for the anti-ROR1 antibodies ATX-P-875, ATX-P-885, ATX-P-890 as compared against control anti-ROR-1 antibodies UC961 (ATX-P-453) and 4a5.

FIG. 21 illustrates nucleotide and amino acid sequences for anti-ROR-1 antibodies ATX-P-875, ATX-P-885, and ATX-P-890.

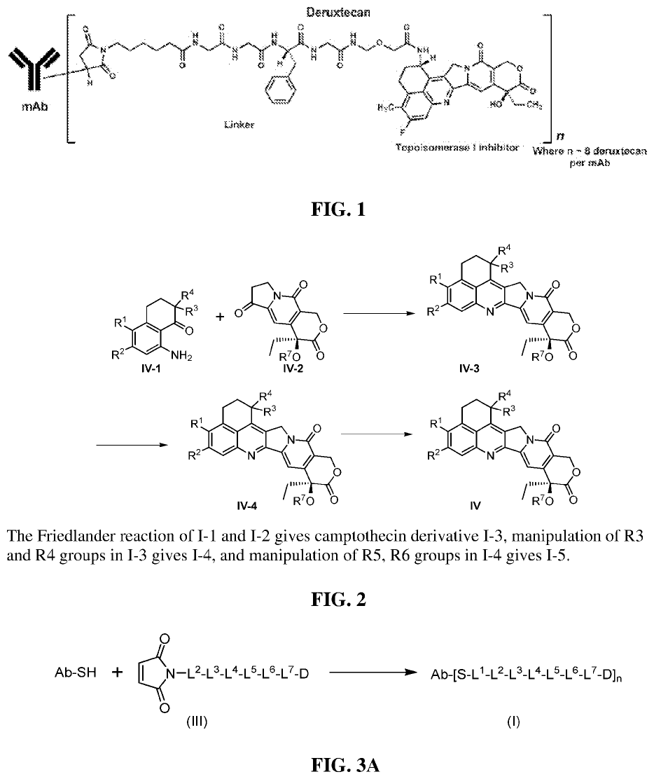

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, a “conjugate” is a compound that comprises two or more substances (such as an antibody, a linker moiety and/or a drug moiety) joined together by chemical bonds. Examples of conjugates include antibody-drug conjugates (which may optionally include a linker moiety), drug-linker conjugates, and antibody-linker conjugates. An “immunoconjugate” is a conjugate that comprise an immunological substance such as an antibody.

As used herein, an “antibody” (Ab) is a protein made by the immune system, or a synthetic variant thereof, that binds to specific sites on cells or tissues. An “antigen-binding fragment” (Fab) is a portion of an antibody that binds to a specific antigen. Monoclonal antibodies are a type of synthetic antibody. In cancer treatment, monoclonal antibodies may kill cancer cells directly, they may block development of tumor blood vessels, or they may help the immune system kill cancer cells.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group, a di-substituted amine group, a mono-substituted amine (alkyl) and a di-substituted amine (alkyl).

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is. CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if ortho R¹ and R² substituents on a phenyl ring are indicated to be —O—(CR⁵R⁶)ₘ—O— such that R¹ and R² “taken together” form a ring, it means that the —O—(CR⁵R⁶)ₘ—O— is covalently bonded to the phenyl ring at the R¹ and R² positions to form a heterocyclic ring:

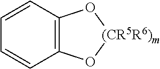

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted. An alkyl group is typically monovalent unless the context indicates otherwise. For example, those skilled in the art recognize that C₁-C₆ alkyl is bivalent in the following formula: —(C₁-C₆ alkyl)-X².

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

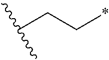 [SMILES: *CC*]

to represent ethylene. The alkylene group may have 1 to 30 carbon atoms (whenever it appears herein, a numerical range such as “1 to 30” refers to each integer in the given range: e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 4 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group

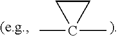

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

The term “halogen atom” or “halogen” as used herein, means any one of the radio-stable atoms of column 7 of the Periodic Table of the Elements, such as, fluorine, chlorine, bromine and iodine.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkenyl” refers to an alkenyl group in which one or more of the hydrogen atoms are replaced by a halogen (e.g., mono-haloalkenyl, di-haloalkenyl, tri-haloalkenyl and polyhaloalkenyl).

As used herein, “haloalkynyl” refers to an alkynyl group in which one or more of the hydrogen atoms are replaced by a halogen (e.g., mono-haloalkynyl, di-haloalkynyl, tri-haloalkynyl and polyhaloalkynyl).

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isooxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

Where the number of substituents is not specified (e.g., haloalkyl, haloalkenyl, haloalkynyl), there may be one or more substituents present. For example, “haloalkyl” may include one or more of the same or different halogens. As another example, “C₁-C₃ alkoxy phenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For compounds of Formula (I), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl).

It Is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included. It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘ containing,’ or ‘characterized by,’ and is inclusive or open-ended and does not exclude additional, unrecited elements or method steps; the term ‘having’ should be interpreted as ‘having at least;’ the term “includes” should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired.’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

It should be understood that the labeling of compounds herein may include similar numbers but have a letter associated therewith such that the identified compounds can be different (and unrelated). For example, compound 2-15a is a different compound from compound 2-15 despite having a similar ring structure.

### Compounds

Various embodiments disclosed herein relate to a compound of Formula (IV), or a pharmaceutically acceptable salt thereof, having the structure:

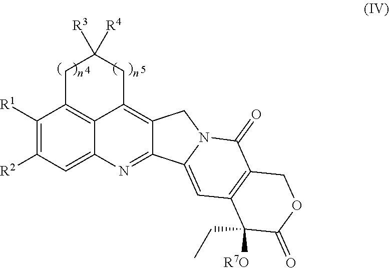

In various embodiments, R¹ and R² in Formula (IV) are each individually selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, or a substituted or an unsubstituted —O—(CR⁵R⁶)ₘ—O-such that R¹ and R² taken together form a ring. In an embodiment, at least one of R¹ and R² is hydrogen. In an embodiment, at least one of R¹ and R² is halogen. For example, in an embodiment, at least one of R¹ and R² is fluoro. In an embodiment, at least one of R¹ and R² is —CN. In an embodiment, at least one of R¹ and R² is —OR⁵, wherein R⁵ is a substituted or an unsubstituted C₁-C₆ alkyl. For example, in an embodiment, at least one of R¹ and R² is methoxy.

In an embodiment, at least one of R¹ and R² in Formula (IV) is —NR⁵R⁶, wherein R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl.

In an embodiment, at least one of R¹ and R² in Formula (IV) is a substituted or an unsubstituted C₁-C₆ alkyl. In an embodiment, at least one of R¹ and R² is C₁-C₃ alkyl. For example, in an embodiment, at least one of R¹ and R² is methyl. In an embodiment, at least one of R¹ and R² is C₁-C₃ alkyl and the other is a halogen. For example, in an embodiment, at least one of R¹ and R² is methyl and the other is fluoro.

In an embodiment, at least one of R¹ and R² in Formula (IV) is a substituted or an unsubstituted C₁-C₆ haloalkyl. For example, in an embodiment, at least one of R¹ and R² is difluoromethyl. In an embodiment, at least one of R¹ and R² is a substituted or an unsubstituted —O—(C₁-C₆ alkyl). For example, in an embodiment, at least one of R¹ and R² is methoxy. In an embodiment, at least one of R¹ and R² is —[(CY₂)ₚO(CY₂)_{q}]ₜCY₃. In an embodiment. R¹ and R² are a substituted or an unsubstituted —O—(CR⁵R⁶)ₘ—O— such that R¹ and R² taken together form a ring in which the ends of the —O—(CR⁵R⁶)ₘ—O— are covalently bonded to the phenyl ring at the R¹ and R² positions of Formula (IV) to form a heterocyclic ring.

In an embodiment, one of R¹ and R² in Formula (IV) is hydrogen and the other of R¹ and R² is halogen. In an embodiment, one of R¹ and R² is hydrogen and the other of R¹ and R² is a substituted or an unsubstituted C₁-C₆ alkyl. In an embodiment, one of R¹ and R² is hydrogen and the other of R¹ and R² is a substituted or an unsubstituted C₁-C₆ haloalkyl. In an embodiment, one of R¹ and R² is hydrogen and the other of R¹ and R² is a substituted or an unsubstituted —O—(C₁-C₆ alkyl). In an embodiment, both R¹ and R² are hydrogen. In an embodiment, neither R¹ nor R² is hydrogen.

In an embodiment, one of R¹ and R² in Formula (IV) is halogen and the other of R¹ and R² is a substituted or an unsubstituted C₁-C₆ alkyl. In an embodiment, one of R¹ and R² is halogen and the other of R¹ and R² is a substituted or an unsubstituted C₁-C₆ haloalkyl. In an embodiment, one of R¹ and R² is halogen and the other of R¹ and R² is a substituted or an unsubstituted —O—(C₁-C₆ alkyl). In an embodiment, both R¹ and R² are independently halogen. In an embodiment, neither R¹ nor R² is halogen.

In an embodiment, one of R¹ and R² in Formula (IV) is a substituted or an unsubstituted C₁-C₆ alkyl and the other of R¹ and R² is a substituted or an unsubstituted C₁-C₆ haloalkyl. In an embodiment, one of R¹ and R² is a substituted or an unsubstituted C₁-C₆ alkyl and the other of R¹ and R² is a substituted or an unsubstituted —O—(C₁-C₆ alkyl). In an embodiment, both R¹ and R² are independently a substituted or an unsubstituted C₁-C₆ alkyl. In an embodiment, neither R¹ nor R² is a substituted or an unsubstituted C₁-C₆ alkyl.

In an embodiment, one of R¹ and R² in Formula (IV) is a substituted or an unsubstituted C₁-C₆ haloalkyl and the other of R¹ and R² is a substituted or an unsubstituted —O—(C₁-C₆ alkyl). In an embodiment, both R¹ and R² are independently a substituted or an unsubstituted C₁-C₆ haloalkyl. In an embodiment, neither R¹ nor R² is a substituted or an unsubstituted C₁-C₆ haloalkyl.

In an embodiment, one of R¹ and R² in Formula (IV) is a substituted or an unsubstituted —O—(C₁-C₆ alkyl). In an embodiment, both R¹ and R² are independently a substituted or an unsubstituted —O—(C₁-C₆ alkyl). In an embodiment, neither R¹ nor R² is a substituted or an unsubstituted —O—(C₁-C₆ alkyl). In an embodiment, R¹ and R² are a substituted or an unsubstituted —O—(CR⁵R⁶)ₘ—O— such that R¹ and R² taken together form a ring. In various embodiments, R¹ and R² are each individually selected from the group consisting of hydrogen, fluoro, methoxy, methyl, difluoromethyl, and —O—(CH₂)—O-such that R¹ and R² taken together form a ring.

In various embodiments, R³ in Formula (IV) is hydrogen, —OH, —N₃, —NH₂, —NH(C═O)CH₃, a substituted or an unsubstituted C₁-C₆ alkyl, a substituted or an unsubstituted C₂-C₆ alkenyl or [(CY₂)ₚO(CY₂)_{q}]ₜOH, with the proviso that R³ and R⁴ are not both hydrogen. In an embodiment, R³ is —OH. In an embodiment, R³ is —N₃. In an embodiment, R³ is —NH₂. In an embodiment, R³ is —NH(C═O)—CH₂—R^{3D}, wherein R^{3D} can be selected from H, —CH₃, —OH and —CH₂Y¹, wherein Y¹ is halogen. For example, R³ can be —NH(C═O)—CH₃, —NH(C═O)—CH₂CH₃, —NH(C═O)—CH₂OH, —NH(C═O)—CH₂CH₂—Y¹. In some embodiments, Y¹ can be F or Cl. In an embodiment, R³ is an unsubstituted C₁-C₆ alkyl. In an embodiment, R³ is a substituted C₁-C₆ alkyl. Examples of C₁-C₆ alkyls include methyl, ethyl, n-propyl, iso-propyl, n-butyl, iso-butyl, sec-butyl, tert-butyl, pentyl (straight-chained or branched) and hexyl (straight-chained or branched). In an embodiment. R³ is a substituted C₂-C₆ alkenyl. In an embodiment, R³ is an unsubstituted C₂-C₆ alkenyl. When R³ is a substituted C₁-C₆ alkyl or a substituted C₂-C₆ alkenyl, the C₁-C₆ alkyl and/or C₂-C₆ alkenyl can be substituted with one or more R^{3A} groups (such as 1, 2 or 3 R^{3A} groups) individually selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl). In some embodiments, R³ is a substituted C₁-C₆ alkyl substituted by —OH and —NR^{3B}R^{3C}. For example, in an embodiment. R³ is methyl, —CH₂OH, —CH₂CH₂OH, —CH₂CH(OH)CH₂OH, —CH(NH₂))(CH₂OH), —CH(NH₂)(CH₂CH₂OH), —CH(NH(CH₃))(CH₂OH), —CH(NH(CH₃))(CH₂CH₂OH), —CH(N(CH₃)₂)(CH₂OH), —CH(N(CH₃)₂)(CH₂CH₂OH), —CH(NH(isopropyl))(CH₂OH), —CH(NH(isopropyl))(CH₂CH₂OH), —CH₂CH═CH₂, —CH(NH—(C(═O)CH₃)(CH₂OH), —CH(NH—(C(═O)CH₃))(CH₂CH₂OH) and —CH(NH—(C(═O)CH₃))(CH₂CH═CH₂). In some embodiments, R³ is a substituted C₁-C₆ alkyl substituted by —OH and —NR^{3B}R^{3C}. In some embodiments, R³ is a substituted C₁-C₆ alkyl substituted —C(═O) (an unsubstituted C₁-C₆ alkyl). Examples of suitable C₁-C₆ alkyls are described herein. In some embodiments. R³ is a substituted C₁-C₆ alkyl substituted by one or more OH groups (such as 1, 2, 3 or 4 OH groups). In an embodiment. R³ is —[(CY₂)ₚO(CY₂)_{q}]ₜOH. Exemplary-[(CY₂)ₚO(CY₂)_{q}]ₜOH groups for R³ include-CH₂OCH₂CH₂OH.

In various embodiments, R⁴ in Formula (IV) is hydrogen, —OH, —N₃, —NH₂, —NH(C—O)CH₃, a substituted or an unsubstituted C₁-C₆ alkyl, a substituted or an unsubstituted C₂-C₆ alkenyl or [(CY₂)ₚO(CY₂)_{q}]ₜOH, with the proviso that R³ and R⁴ are not both hydrogen. In an embodiment. R⁴ is hydrogen. In an embodiment. R⁴ is —OH. In an embodiment, R⁴ is —N₃. In an embodiment, R⁴ is —NH₂. In an embodiment, R⁴ is —NH(C═O)—CH₂—R^{3D}, wherein R^{3D} can be selected from H, —CH₃, —OH and —CH₂Y¹, wherein Y¹ is halogen. For example, R⁴ can be —NH(C═O)—CH₃, —NH(C═O)—CH₂CH₃, —NH(C═O)—CH₂OH, —NH(C═O)—CH₂CH₂—Y¹. In some embodiments, Y¹ can be F or Cl. In an embodiment, R⁴ is an unsubstituted C₁-C₆ alkyl. In an embodiment, R⁴ is a substituted C₁-C₆ alkyl. Examples of C₁-C₆ alkyls include methyl, ethyl, n-propyl, iso-propyl, n-butyl, iso-butyl, sec-butyl, tert-butyl, pentyl (straight-chained or branched) and hexyl (straight-chained or branched). In an embodiment. R⁴ is a substituted C₂-C₆ alkenyl. In an embodiment, R⁴ is an unsubstituted C₂-C₆ alkenyl. When R⁴ is a substituted C₁-C₆ alkyl or a substituted C₂-C₆ alkenyl, the C₁-C₆ alkyl and/or C₂-C₆ alkenyl can be substituted with one or more R^{3A} groups (such as 1, 2 or 3 R^{3A} groups) individually selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl). In some embodiments, R⁴ is a substituted C₁-C₆ alkyl substituted by —OH and —NR^{3B}R^{3C}. For example, in an embodiment. R⁴ is methyl, —CH₂OH, —CH₂CH₂OH, —CH₂CH(OH)CH₂OH, —CH(NH₂))(CH₂OH), —CH(NH₂)(CH₂CH₂OH), —CH(NH(CH₃))(CH₂OH), —CH(NH(CH₃))(CH₂CH₂OH), —CH(N(CH₃)₂)(CH₂OH), —CH(N(CH₃)₂)(CH₂CH₂OH), —CH(NH(isopropyl))(CH₂OH), —CH(NH(isopropyl))(CH₂CH₂OH), —CH₂CH═CH₂, —CH(NH—(C(═O)CH₃))(CH₂OH), —CH(NH—(C(═O)CH₃))(CH₂CH₂OH) and —CH(NH—(C(═O)CH₃))(CH₂CH═CH₂). In some embodiments. R⁴ is a substituted C₁-C₆ alkyl substituted by —OH and —NR^{3B}R^{3C}. In some embodiments, R⁴ is a substituted C₁-C₆ alkyl substituted C(═O) (an unsubstituted C₁-C₆ alkyl). Examples of suitable C₁-C₆ alkyls are described herein. In some embodiments. R⁴ is a substituted C₁-C₆ alkyl substituted by one or more OH groups (such as 1, 2, 3 or 4 OH groups). In an embodiment. R⁴ is —[(CY₂)ₚO(CY₂)_{q}]ₜOH. Exemplary —[(CY₂)ₚO(CY₂)_{q}]ₜOH groups for R⁴ include-CH₂OCH₂CH₂OH.

In some embodiments, one of R³ and R⁴ in Formula (IV) is hydrogen, and the other of R³ and R⁴ is a substituted or an unsubstituted C₁-C₆ alkyl. In some embodiments, one of R³ and R⁴ in Formula (IV) is hydrogen, and the other of R³ and R⁴ is a substituted C₁-C₆ alkyl. In some embodiments, one of R³ and R⁴ in Formula (IV) is hydrogen, and the other of the other of R³ and R⁴ is a substituted C₁-C₆ alkyl substituted by —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl). In some embodiments, one of R³ and R⁴ in Formula (IV) is hydrogen, and the other of R³ and R⁴ is a substituted or an unsubstituted C₂₋₆ alkenyl. In other embodiments, one of R³ and R⁴ in Formula (IV) is —N₃, and the other of R³ and R⁴ is a substituted or an unsubstituted C₂₋₆ alkenyl. In still other embodiments, one of R³ and R⁴ in Formula (IV) is —OH, and the other of R³ and R⁴ is a substituted or an unsubstituted C₂₋₆ alkenyl. In yet still other embodiments, one of R³ and R⁴ in Formula (IV) is —NH₂, and the other of R³ and R⁴ is a substituted or an unsubstituted C₂₋₆ alkenyl. For example, one of R³ and R⁴ in Formula (IV) is —OH—N₃, —NH₂ or —NH(C═O)—CH₂—R^{3D}, and the other of R³ and R⁴ is —CH₂CH═CH₂. In some embodiments, one of R³ and R⁴ in Formula (IV) is hydrogen, and the other of the other of R³ and R⁴ is —CH(NH₂))(CH₂OH), —CH(NH₂)(CH₂CH₂OH), —CH(NH(CH₃)(CH₂OH), —CH(NH(CH₃)(CH₂CH₂OH), —CH(N(CH₃)₂)(CH₂OH), —CH(N(CH₃)₂)(CH₂CH₂OH), —CH(NH(isopropyl))(CH₂OH), —CH(NH(isopropyl))(CH₂CH₂OH), —CH(NH—(C(═O)CH₃))(CH₂OH), —CH(NH—(C(═O)CH₃))(CH₂CH₂OH) or —CH(NH—(C(═O)CH₃))(CH₂CH═CH₂). In some embodiments, one of R³ and R⁴ in Formula (IV) is —OH, and the other of R³ and R⁴ is a substituted or an unsubstituted C₁-C₆ alkyl. In other embodiments, one of R³ and R⁴ in Formula (IV) is —NH₂, and the other of R³ and R⁴ is a substituted or an unsubstituted C₁-C₆ alkyl. In still other embodiments, one of R³ and R⁴ in Formula (IV) is —NH(C═O)—CH₂—R^{3D}, and the other of R³ and R⁴ is a substituted or an unsubstituted C₁-C₆ alkyl. In some embodiments, one of R³ and R⁴ in Formula (IV) is —OH, and the other of R³ and R⁴ is a hydroxy-substituted C₁-C₆ alkyl, such as —CH₂OH, —CH₂CH₂OH and —CH₂CH(OH)CH₂OH. In other embodiments, one of R³ and R⁴ in Formula (IV) is —NH₂, and the other of R³ and R⁴ is a hydroxy-substituted C₁-C₆ alkyl, such as —CH₂OH, —CH₂CH₂OH and —CH₂CH(OH)CH₂OH. As provided herein, in some embodiments, one or more hydroxy groups can be present on a hydroxy-substituted C₁-C₆ alkyl, such as 1, 2, 3 or 4 OH groups. In some embodiments, when R³ is —OH, then R⁴ cannot be an unsubstituted C₁₋₆ alkyl, such as methyl; and when R⁴ is —OH, then R³ cannot be an unsubstituted C₁₋₆ alkyl, such as methyl. In some embodiments, when R³ is —NH₂, then R⁴ cannot be an unsubstituted C₁₆ alkyl, such as methyl, and R³ is —NH₂, then R⁴ cannot be an unsubstituted C₁₋₆ alkyl, such as methyl. In some embodiments, one of R³ and R⁴ is CH₃, with the proviso that R³ and R⁴ are not both-CH₃. In some embodiments, R³ and R⁴ are not each an unsubstituted C₁₋₆ alkyl, for example, CH₃. In some embodiments of this paragraph, R¹ can be a substituted or an unsubstituted C₁-C₆ alkyl (for example, methyl, ethyl, n-propyl, iso-propyl, n-butyl, iso-butyl, sec-butyl, tert-butyl, pentyl (straight-chained or branched) and hexyl (straight-chained or branched)); and R² can be halogen. In some embodiments of this paragraph, R¹ can be an unsubstituted C₁-C₆ alkyl; and R² can be halogen (such as F or Cl). In some embodiments of this paragraph, R⁷ can be H.

In some embodiments, a compound of Formula (IV), or a pharmaceutically acceptable salt thereof, can have the structure of Formula (IV-a), or a pharmaceutically acceptable salt thereof:

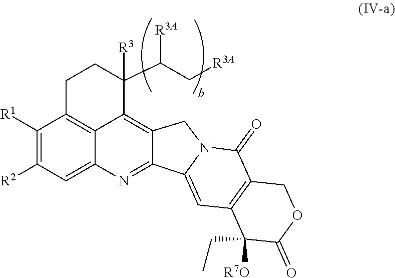

wherein:
R¹, R², R^{3A} and R⁷ are as provided herein for Formula (IV);
R³ is —OH, —CH₃, —NH₂ or —NH(C═O)CH₃; and
b is individually 1, 2, or 3.

In some embodiments, each R^{3A} can independently be OH. In some embodiments, each R^{3A} can independently be H. In some embodiments, each R^{3A} can independently be —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl). For example, —NR^{3B}R^{3C} can be —NH₂, —NHAc, —NHCH(CH₃)₂, or —N(CH₃)₂. In some embodiments of this paragraph, b can be 1. In other embodiments of this paragraph, b can be 2. In still other embodiments of this paragraph, b can be 3.

In some embodiments, a compound of Formula (IV) can be where R¹ and R² are each individually selected from hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, or a substituted or an unsubstituted —O—(CR⁵R⁶)ₘ—O-such that R¹ and R² taken together form a ring; R³ and R⁴ are each individually selected from hydrogen, —OH, —N₃, —NH₂, —NH(C═O)CH₂R^{3D}, a substituted or an unsubstituted C₁-C₆ alkyl, a substituted or an unsubstituted C₂-C₆ alkenyl and [(CY₂)ₚO(CY₂)_{q}]ₜOH, with the proviso that R³ and R⁴ are not both hydrogen, wherein when the C₁-C₆ alkyl or C₂-C₆ alkenyl is substituted, the C₁-C₆ alkyl or C₂-C₆ alkenyl is substituted with one or more R^{3A} groups selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl); R^{3D} is selected from H, —CH₃, —OH and —CH₂Y¹, wherein Y¹ is halogen; R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl; n⁴ and n⁵ are each individually 0, 1 or 2, with the proviso that n⁴ and n⁵ are not both 0; each Y is individually H or halogen; each m is individually 1 or 2; each p is individually 1, 2, 3, 4, 5, or 6; each q is individually 0, 1, 2, 3, 4, 5, or 6; and each t is individually 1, 2, 3, 4, 5, or 6; R⁷ is H, —COR⁸, —CO₂R⁸, or —(CO)—NHR⁸; and R⁸ is a substituted or an unsubstituted C₁-C₆ alkyl, a substituted or an unsubstituted C₁-C₆ haloalkyl, or —[(CY₂)ₚO(CY₂)_{q}]ₜCY₃.

In some embodiments, a compound of Formula (IV), or a pharmaceutically acceptable salt thereof, can have a structure selected from the following:

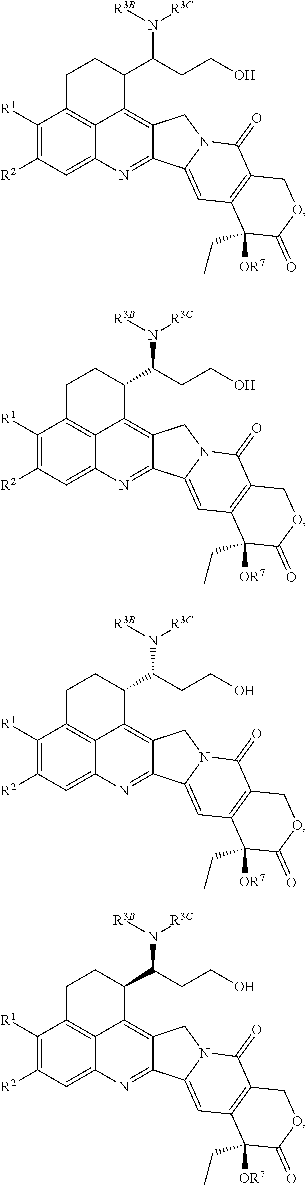

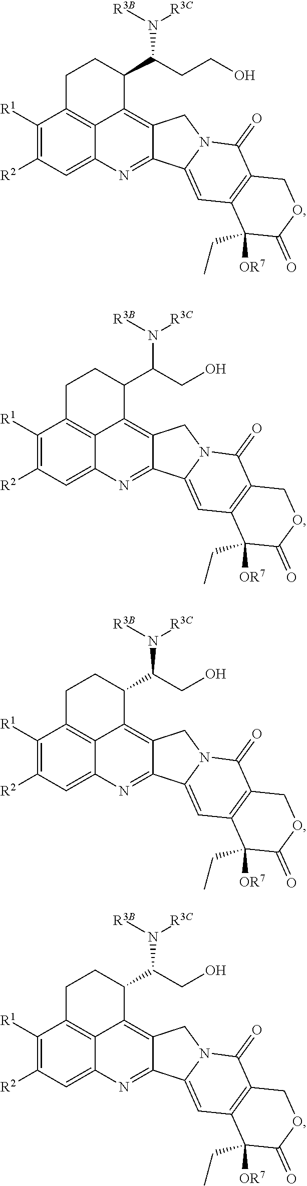

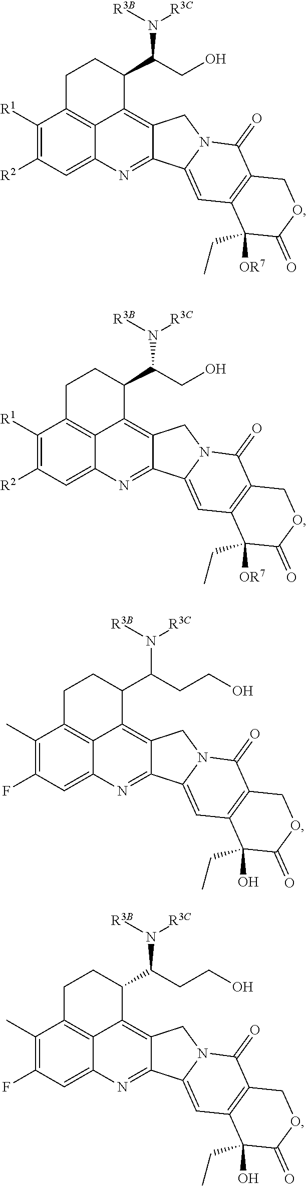

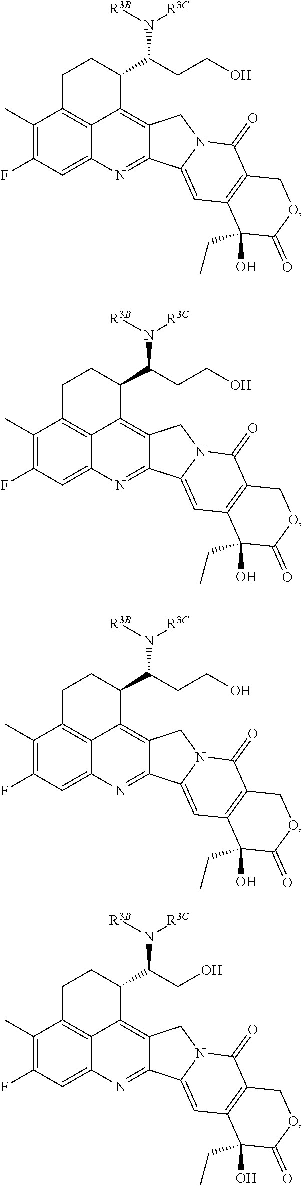

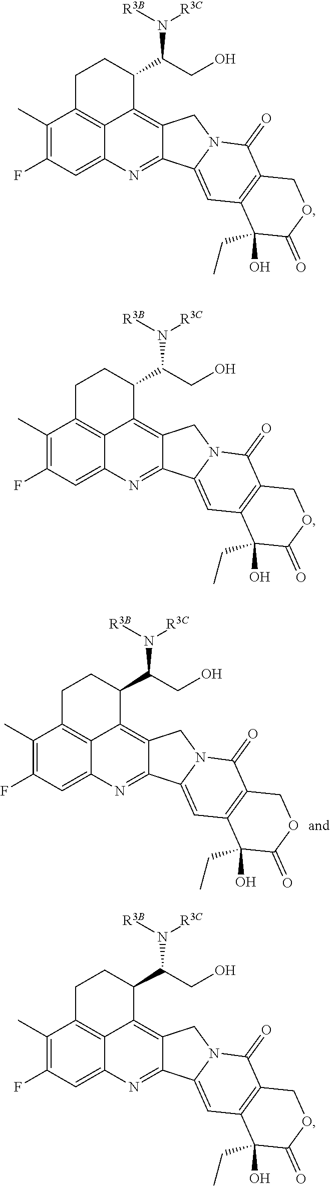

wherein R¹, R², R^{3B}, R^{3B} and R^{3C} are as provided herein for Formula (IV). In some embodiments of this paragraph, R^{3B} and R^{3C} can be each hydrogen. In other embodiments of this paragraph, one of R^{3B} and R^{3C} can be hydrogen, and the other of R^{3B} and R^{3C} can be a substituted C₁-C₆ alkyl. In still other embodiments of this paragraph, one of R^{3B} and R^{3C} can be hydrogen, and the other of R^{3B} and R^{3C} can be an unsubstituted C₁-C₆ alkyl. In other embodiments of this paragraph, one of R^{3B} and R^{3C} can be hydrogen, and the other of R^{3B} and R^{3C} can be —C(═O) (an unsubstituted C₁-C₆ alkyl). In still other embodiments of this paragraph, one of R^{3B} and R^{3C} can be an unsubstituted C₁-C₆ alkyl, and the other of R^{3B} and R^{3C} can be —C(═O) (an unsubstituted C₁-C₆ alkyl). Suitable C₁-C₆ alkyls include methyl, ethyl, n-propyl, iso-propyl, n-butyl, iso-butyl, sec-butyl, tert-butyl, pentyl (straight-chained or branched) and hexyl (straight-chained or branched).

In various embodiments, R⁷ in Formula (IV) is H, —COR⁸, —CO₂R⁸, or —(CO)—NHR⁸, wherein R⁸ is described elsewhere herein. In an embodiment. R⁷ is H. In an embodiment, R⁷ is —COR⁸. In an embodiment, R⁷ is —CO₂R⁸. In an embodiment, R⁷ is —(CO)—NHR⁸.

In various embodiments, R⁸ in Formula (IV) is a substituted or an unsubstituted C₁-C₆ alkyl, a substituted or an unsubstituted C₁-C₆ haloalkyl, or —[(CY₂)ₚO(CY₂)_{q}]ₜCY₃, where the variables p, q, t and Y are described elsewhere herein. In an embodiment, R⁸ is a substituted or an unsubstituted C₁-C₆ alkyl. In an embodiment, R⁸ is a substituted or an unsubstituted C₁-C₆ haloalkyl. In an embodiment, R⁸ is a-[(CY₂)ₚO(CY₂)_{q}]ₜCY₃.

In various embodiments, m in Formula (IV) is 1 or 2. In an embodiment, m is 1. In another embodiment, m is 2.

In various embodiments, n⁴ and n⁵ in Formula (IV) are each individually 0, 1 or 2, with the proviso that n⁴ and n⁵ are not both 0. In an embodiment, n⁴ and n⁵ are both 1. In an embodiment, n⁴ is 0 and n⁵ is 1. In an embodiment, n⁴ is 0 and n⁵ is 2. In an embodiment, n⁴ is 1 and n⁵ is 0. In an embodiment, n⁴ is 2 and n⁵ is 0).

In various embodiments, each Y in Formula (IV) is individually H or halogen. In an embodiment, each Y is hydrogen. In an embodiment, —CY₂ is —CH₂. In an embodiment, —CY₃ is —CH₃. In an embodiment, —CY₃ is —CHF₂. In an embodiment, —CY₃ is —CH₂F. In an embodiment, —CY₃ is CF₃.

In various embodiments, each p in Formula (IV) is individually 1, 2, 3, 4, 5, or 6. In an embodiment, p is 1. In an embodiment, p is 2.

In various embodiments, each q in Formula (IV) is individually 0, 1, 2, 3, 4, 5, or 6. In an embodiment, q is 1. In an embodiment, q is 2.

In various embodiments, each t in Formula (IV) is individually 1, 2, 3, 4, 5, or 6. In an embodiment, t is 1. In an embodiment, p is t.

In various embodiments, a compound of Formula (IV) can be represented by a structure selected from the following, or a pharmaceutically acceptable salt thereof:

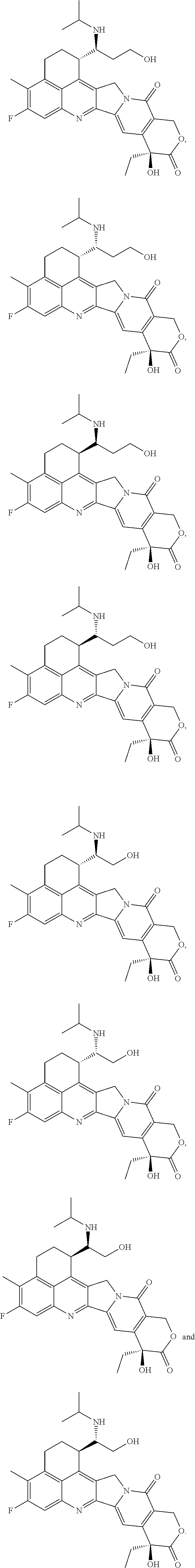

In various embodiments, a compound of Formula (IV) can be represented by a structure selected from the following, or a pharmaceutically acceptable salt thereof:

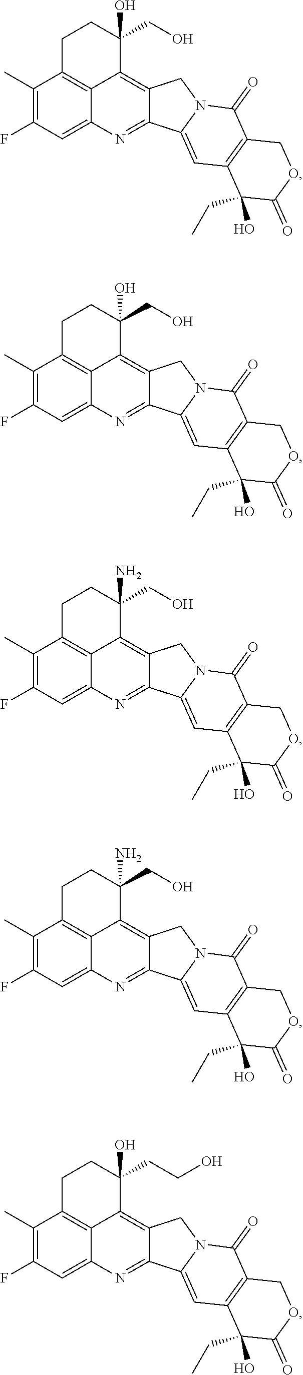 [SMILES: CC[C@@]1(O)C(=O)OCc2c1cc1n(c2=O)Cc2c-1nc1cc(F)c(C)c3c1c2[C@@](O)(CO)CC3 ; CC[C@@]1(O)C(=O)OCc2c1cc1n(c2=O)Cc2c-1nc1cc(F)c(C)c3c1c2[C@](O)(CO)CC3 ; CC[C@@]1(O)C(=O)OCc2c1cc1n(c2=O)Cc2c-1nc1cc(F)c(C)c3c1c2[C@@](N)(CO)CC3 ; CC[C@@]1(O)C(=O)OCc2c1cc1n(c2=O)Cc2c-1nc1cc(F)c(C)c3c1c2[C@](N)(CO)CC3 ; CC[C@@]1(O)C(=O)OCc2c1cc1n(c2=O)Cc2c-1nc1cc(F)c(C)c3c1c2[C@@](O)(CCO)CC3]

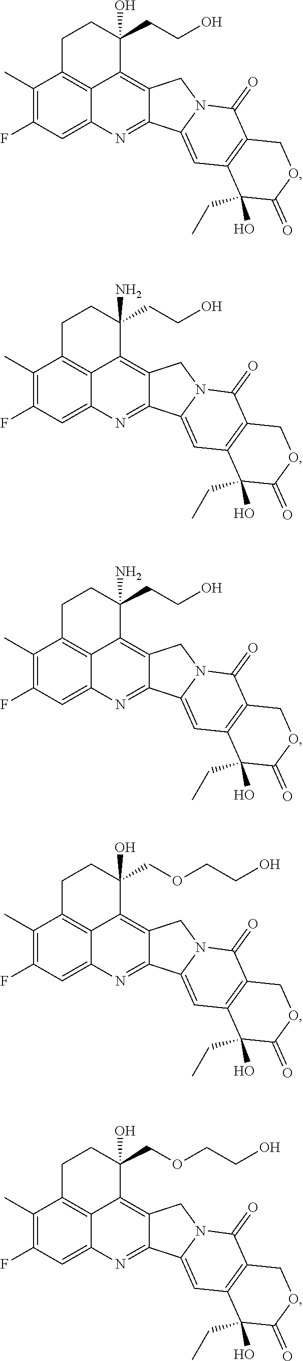 [SMILES: CC[C@@]1(O)C(=O)OCc2c1cc1n(c2=O)Cc2c-1nc1cc(F)c(C)c3c1c2[C@](O)(CCO)CC3 ; CC[C@@]1(O)C(=O)OCc2c1cc1n(c2=O)Cc2c-1nc1cc(F)c(C)c3c1c2[C@@](N)(CCO)CC3 ; CC[C@@]1(O)C(=O)OCc2c1cc1n(c2=O)Cc2c-1nc1cc(F)c(C)c3c1c2[C@](N)(CCO)CC3 ; CC[C@@]1(O)C(=O)OCc2c1cc1n(c2=O)Cc2c-1nc1cc(F)c(C)c3c1c2[C@@](O)(COCCO)CC3 ; CC[C@@]1(O)C(=O)OCc2c1cc1n(c2=O)Cc2c-1nc1cc(F)c(C)c3c1c2[C@](O)(COCCO)CC3]

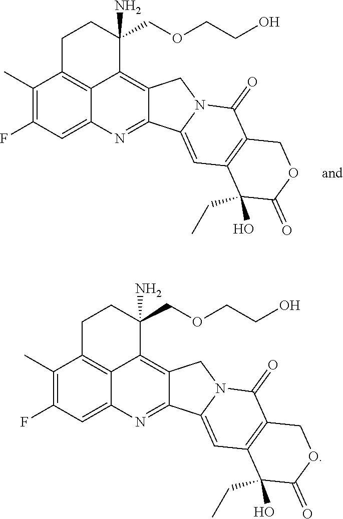 [SMILES: CC[C@@]1(O)C(=O)OCc2c1cc1n(c2=O)Cc2c-1nc1cc(F)c(C)c3c1c2[C@@](N)(COCCO)CC3 ; "and" ; CC[C@@]1(O)C(=O)OCc2c1cc1n(c2=O)Cc2c-1nc1cc(F)c(C)c3c1c2[C@](N)(COCCO)CC3]

In various embodiments, a compound of Formula (IV) can be represented by a structure selected from the following, or a pharmaceutically acceptable salt thereof:

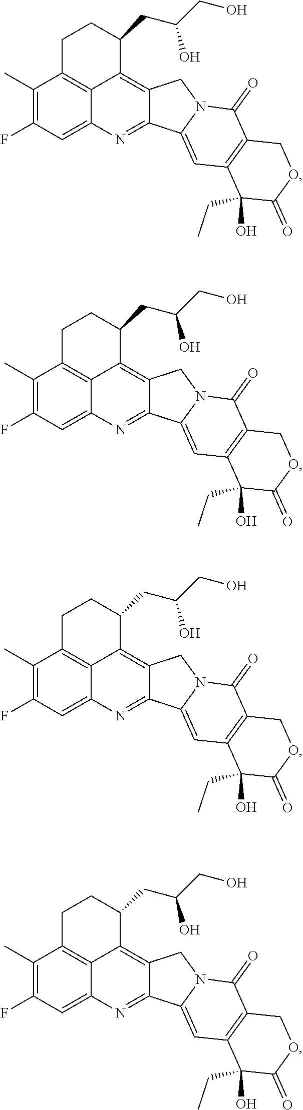 [SMILES: CC[C@@]1(O)C(=O)OCc2c1cc1n(c2=O)Cc2c-1nc1cc(F)c(C)c3c1c2[C@H](C[C@@H](O)CO)CC3 ; CC[C@@]1(O)C(=O)OCc2c1cc1n(c2=O)Cc2c-1nc1cc(F)c(C)c3c1c2[C@H](C[C@H](O)CO)CC3 ; CC[C@@]1(O)C(=O)OCc2c1cc1n(c2=O)Cc2c-1nc1cc(F)c(C)c3c1c2[C@@H](C[C@@H](O)CO)CC3 ; CC[C@@]1(O)C(=O)OCc2c1cc1n(c2=O)Cc2c-1nc1cc(F)c(C)c3c1c2[C@@H](C[C@H](O)CO)CC3]

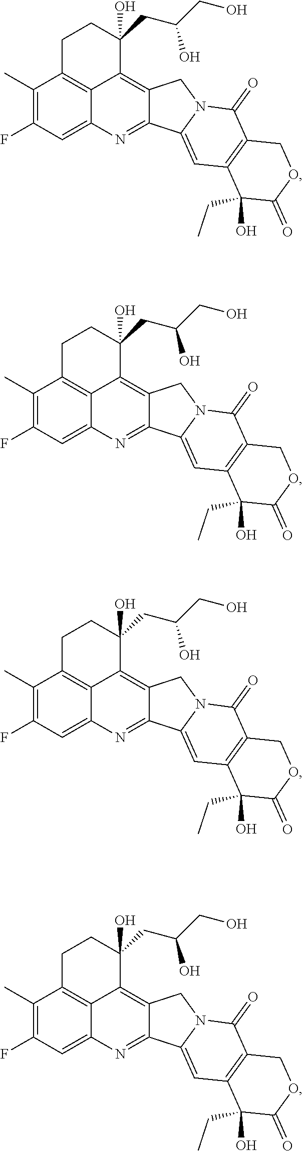 [SMILES: CC[C@@]1(O)C(=O)OCc2c1cc1n(c2=O)Cc2c-1nc1cc(F)c(C)c3c1c2[C@](O)(C[C@@H](O)CO)CC3 ; CC[C@@]1(O)C(=O)OCc2c1cc1n(c2=O)Cc2c-1nc1cc(F)c(C)c3c1c2[C@](O)(C[C@H](O)CO)CC3 ; CC[C@@]1(O)C(=O)OCc2c1cc1n(c2=O)Cc2c-1nc1cc(F)c(C)c3c1c2[C@@](O)(C[C@@H](O)CO)CC3 ; CC[C@@]1(O)C(=O)OCc2c1cc1n(c2=O)Cc2c-1nc1cc(F)c(C)c3c1c2[C@@](O)(C[C@H](O)CO)CC3]

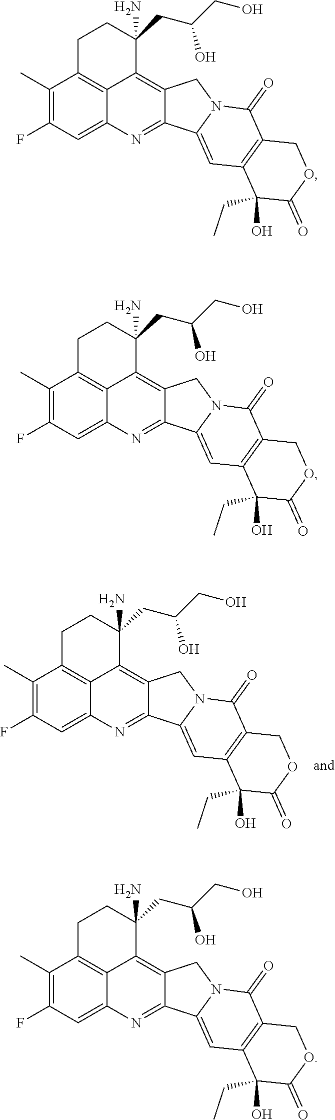 [SMILES: CC[C@@]1(O)C(=O)OCc2c1cc1n(c2=O)Cc2c-1nc1cc(F)c(C)c3c1c2[C@](N)(C[C@@H](O)CO)CC3 ; CC[C@@]1(O)C(=O)OCc2c1cc1n(c2=O)Cc2c-1nc1cc(F)c(C)c3c1c2[C@](N)(C[C@H](O)CO)CC3 ; CC[C@@]1(O)C(=O)OCc2c1cc1n(c2=O)Cc2c-1nc1cc(F)c(C)c3c1c2[C@@](N)(C[C@@H](O)CO)CC3 ; CC[C@@]1(O)C(=O)OCc2c1cc1n(c2=O)Cc2c-1nc1cc(F)c(C)c3c1c2[C@@](N)(C[C@H](O)CO)CC3]

In various embodiments, a compound of Formula (IV) can be represented by a structure selected from the following, or a pharmaceutically acceptable salt thereof:

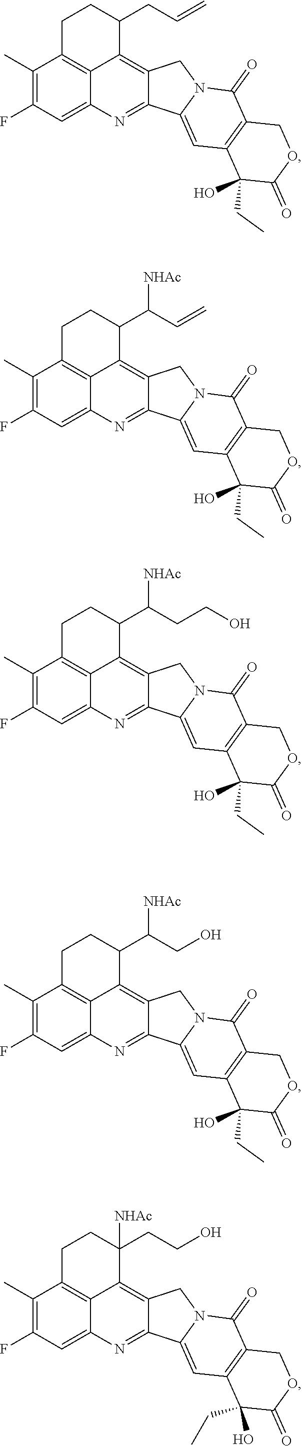 [SMILES: C=CCC1CCc2c(C)c(F)cc3nc4c(c1c23)Cn1c-4cc2c(c1=O)COC(=O)[C@]2(O)CC ; C=CC(NC(C)=O)C1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2C(C(CCO)NC(C)=O)CC3 ; CC[C@@]1(O)C(=O)OCc2c1cc1n(c2=O)Cc2c-1nc1cc(F)c(C)c3c1c2C(C(CO)NC(C)=O)CC3 ; CC[C@@]1(O)C(=O)OCc2c1cc1n(c2=O)Cc2c-1nc1cc(F)c(C)c3c1c2C(CCO)(NC(C)=O)CC3]

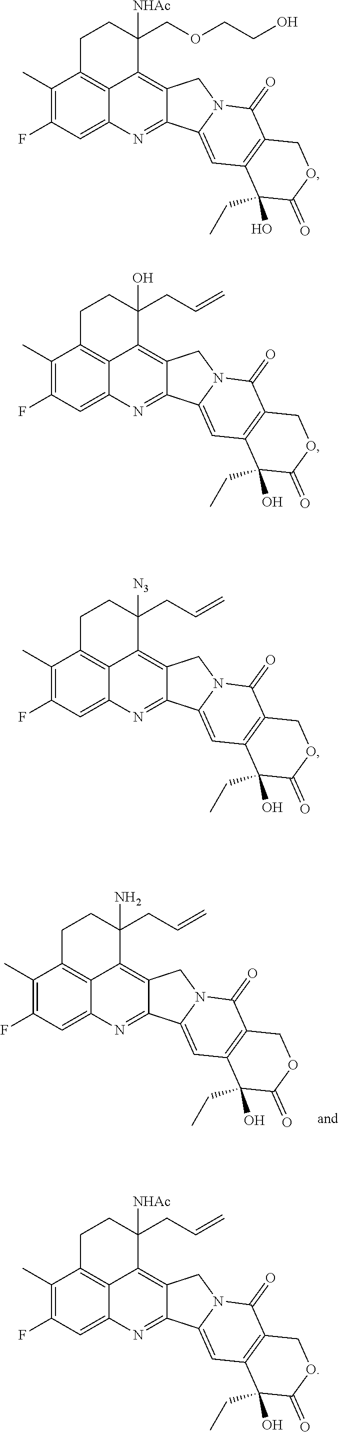 [SMILES: CC[C@@]1(O)C(=O)OCc2c1cc1n(c2=O)Cc2c-1nc1cc(F)c(C)c3c1c2C(COCCO)(NC(C)=O)CC3 ; C=CCC1(O)CCc2c(C)c(F)cc3nc4c(c1c23)Cn1c-4cc2c(c1=O)COC(=O)[C@]2(O)CC ; C=CCC1(N=[N+]=[N-])CCc2c(C)c(F)cc3nc4c(c1c23)Cn1c-4cc2c(c1=O)COC(=O)[C@]2(O)CC ; C=CCC1(N)CCc2c(C)c(F)cc3nc4c(c1c23)Cn1c-4cc2c(c1=O)COC(=O)[C@]2(O)CC ; C=CCC1(NC(C)=O)CCc2c(C)c(F)cc3nc4c(c1c23)Cn1c-4cc2c(c1=O)COC(=O)[C@]2(O)CC]

In some embodiments, a compound of Formula (IV), or a pharmaceutically acceptable salt thereof, cannot be selected from:

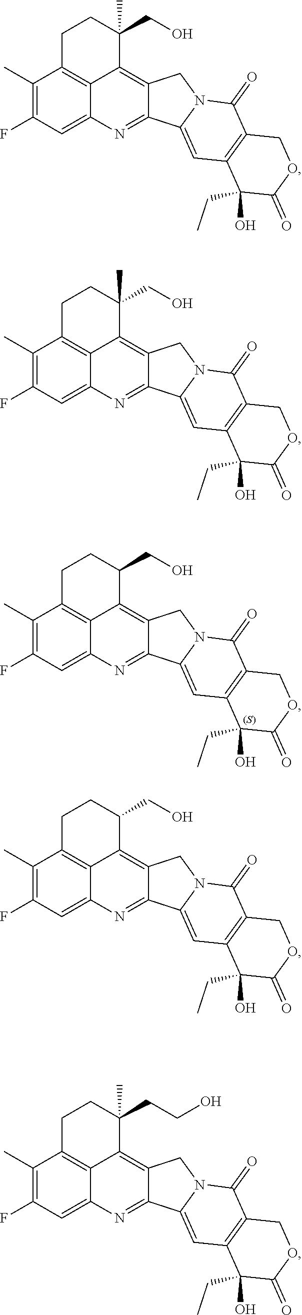

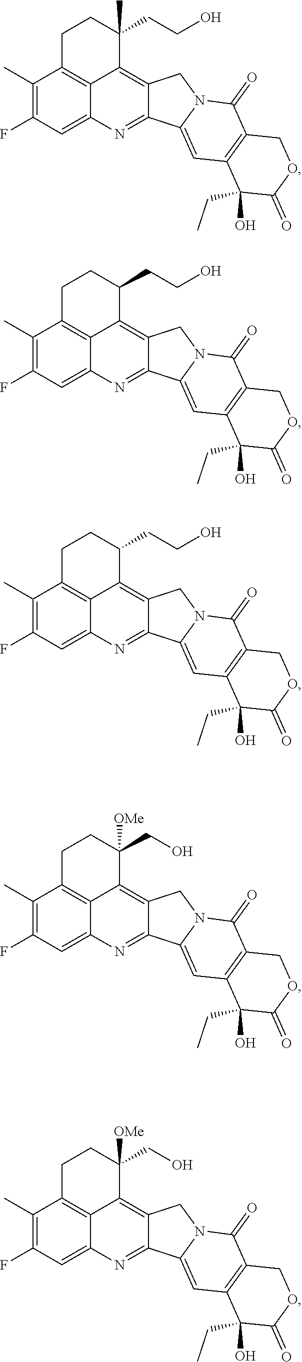

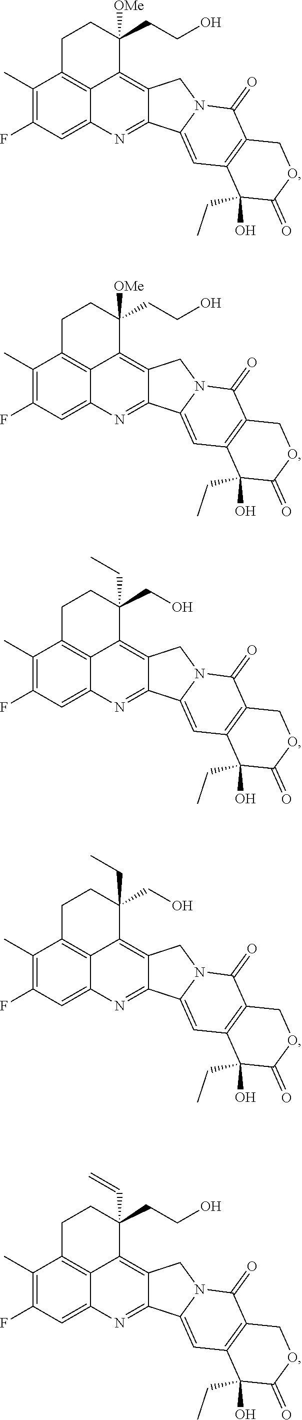 [SMILES: CC[C@@]1(O)C(=O)OCc2c1cc1n(c2=O)Cc2c-1nc1cc(F)c(C)c3c1c2[C@@](CCO)(OC)CC3 ; CC[C@@]1(O)C(=O)OCc2c1cc1n(c2=O)Cc2c-1nc1cc(F)c(C)c3c1c2[C@](CCO)(OC)CC3 ; CC[C@@]1(CO)CCc2c(C)c(F)cc3nc4c(c1c23)Cn1c-4cc2c(c1=O)COC(=O)[C@]2(O)CC ; CC[C@]1(CO)CCc2c(C)c(F)cc3nc4c(c1c23)Cn1c-4cc2c(c1=O)COC(=O)[C@]2(O)CC ; C=C[C@@]1(CCO)CCc2c(C)c(F)cc3nc4c(c1c23)Cn1c-4cc2c(c1=O)COC(=O)[C@]2(O)CC]

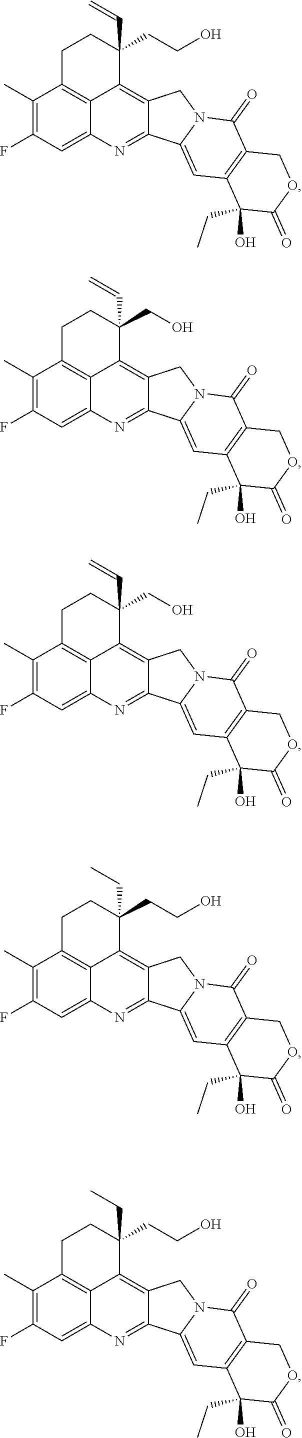 [SMILES: C=C[C@]1(CCO)CCc2c(C)c(F)cc3nc4c(c1c23)Cn1c-4cc2c(c1=O)COC(=O)[C@]2(O)CC ; C=C[C@@]1(CO)CCc2c(C)c(F)cc3nc4c(c1c23)Cn1c-4cc2c(c1=O)COC(=O)[C@]2(O)CC ; C=C[C@]1(CO)CCc2c(C)c(F)cc3nc4c(c1c23)Cn1c-4cc2c(c1=O)COC(=O)[C@]2(O)CC ; CC[C@@]1(CCO)CCc2c(C)c(F)cc3nc4c(c1c23)Cn1c-4cc2c(c1=O)COC(=O)[C@]2(O)CC ; CC[C@]1(CCO)CCc2c(C)c(F)cc3nc4c(c1c23)Cn1c-4cc2c(c1=O)COC(=O)[C@]2(O)CC]

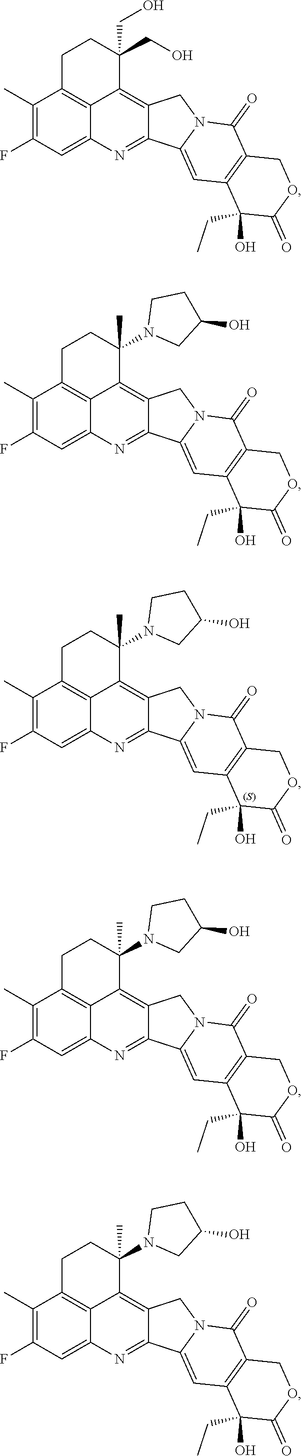

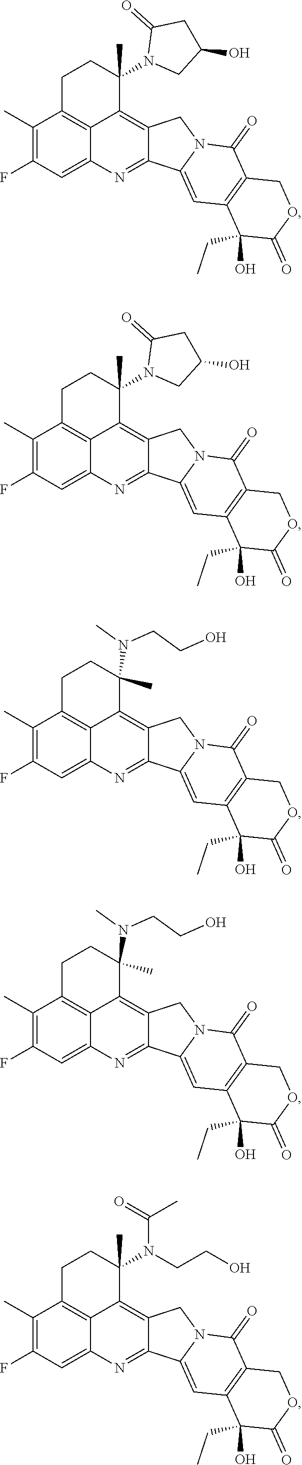

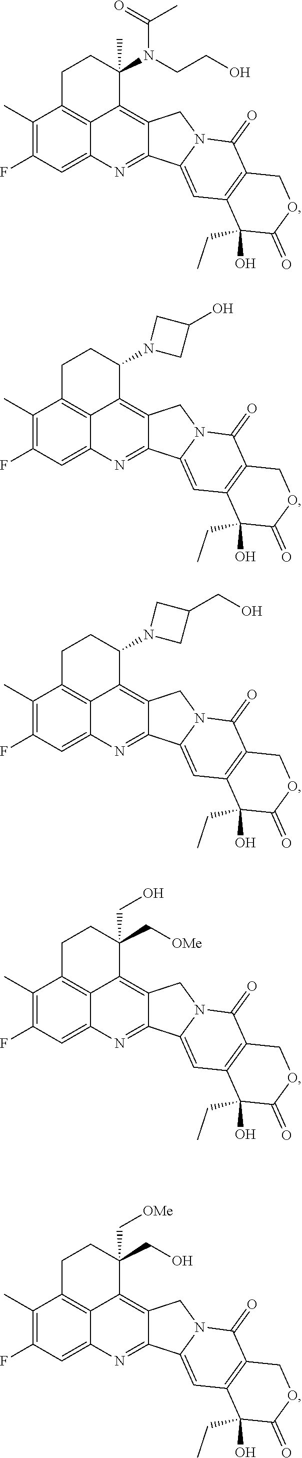

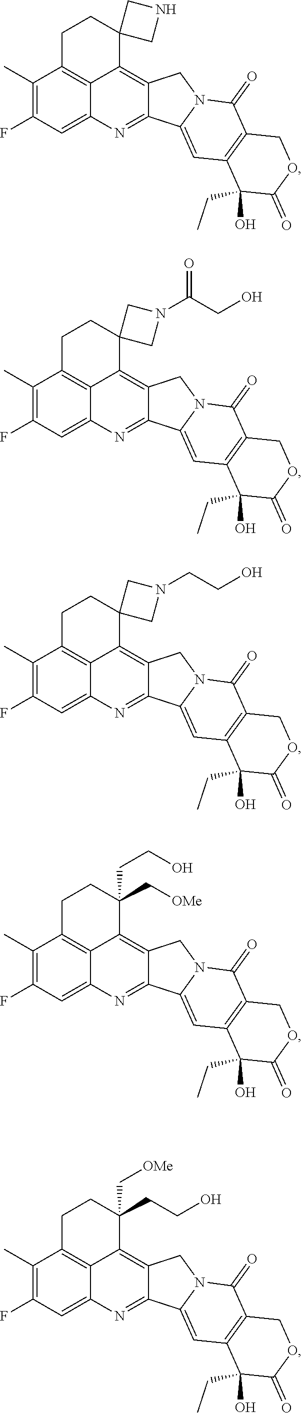

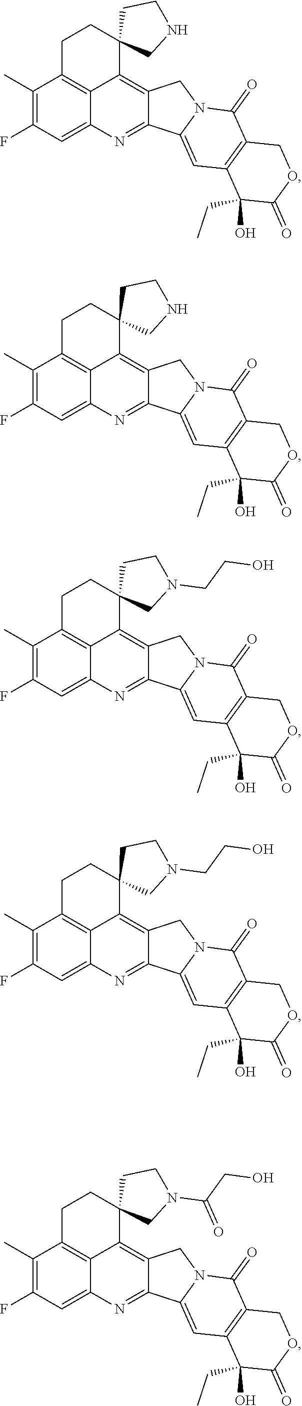 [SMILES: CC[C@@]1(O)C(=O)OCc2c1cc1n(c2=O)Cc2c-1nc1cc(F)c(C)c3c1c2[C@@]1(CCNC1)CC3 ; CC[C@@]1(O)C(=O)OCc2c1cc1n(c2=O)Cc2c-1nc1cc(F)c(C)c3c1c2[C@]1(CCNC1)CC3 ; CC[C@@]1(O)C(=O)OCc2c1cc1n(c2=O)Cc2c-1nc1cc(F)c(C)c3c1c2[C@]1(CC3)CCN(CCO)C1 ; CC[C@@]1(O)C(=O)OCc2c1cc1n(c2=O)Cc2c-1nc1cc(F)c(C)c3c1c2[C@@]1(CC3)CCN(CCO)C1 ; CC[C@@]1(O)C(=O)OCc2c1cc1n(c2=O)Cc2c-1nc1cc(F)c(C)c3c1c2[C@]1(CC3)CCN(C(=O)CO)C1]

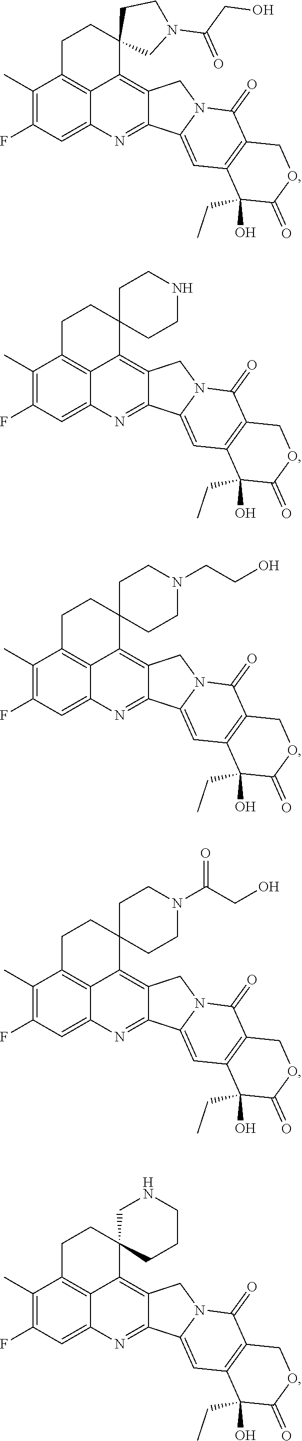

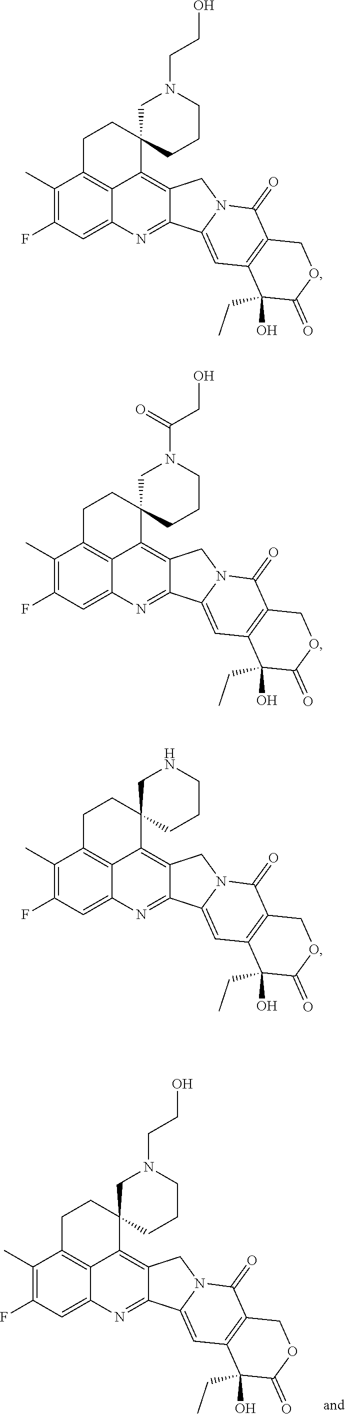

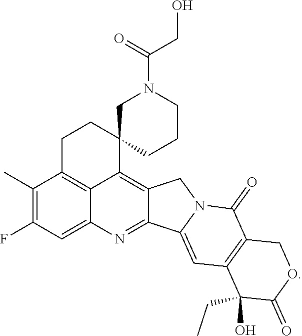 [SMILES: CC[C@@]1(O)C(=O)OCc2c1cc1n(c2=O)Cc2c-1nc1cc(F)c(C)c3c1c2[C@@]1(CCCN(C(=O)CO)C1)CC3]

In some embodiments, a compound of Formula (IV), or a pharmaceutically acceptable salt thereof, cannot be selected from:

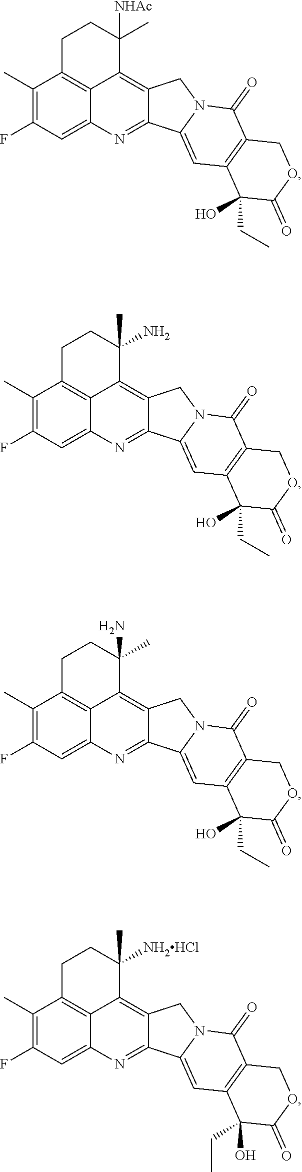

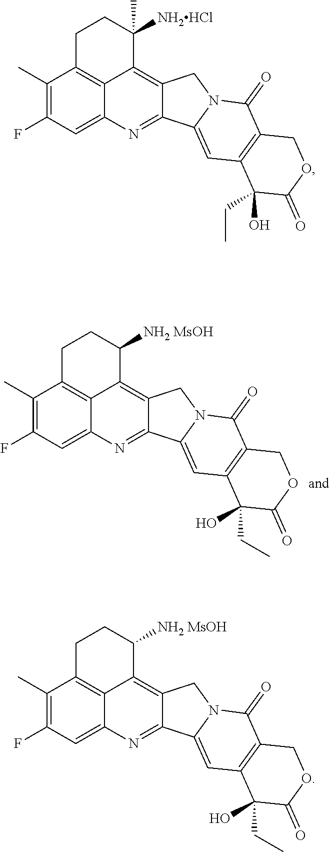

In some embodiments, a compound of Formula (IV), or a pharmaceutically acceptable salt thereof, cannot be selected from:

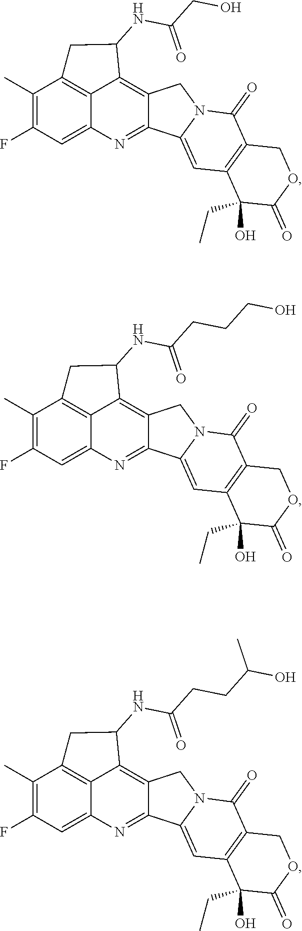 [SMILES: CC[C@@]1(O)C(=O)OCc2c1cc1n(c2=O)Cc2c-1nc1cc(F)c(C)c3c1c2C(NC(=O)CO)C3 ; CC[C@@]1(O)C(=O)OCc2c1cc1n(c2=O)Cc2c-1nc1cc(F)c(C)c3c1c2C(NC(=O)CCCO)C3 ; CC[C@@]1(O)C(=O)OCc2c1cc1n(c2=O)Cc2c-1nc1cc(F)c(C)c3c1c2C(NC(=O)CCC(C)O)C3]

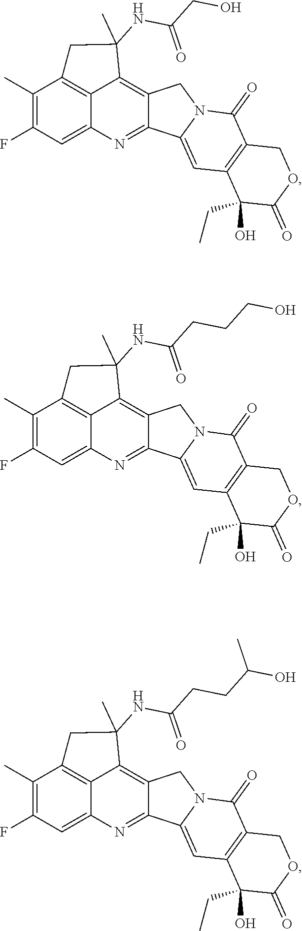 [SMILES: CC[C@@]1(O)C(=O)OCc2c1cc1n(c2=O)Cc2c-1nc1cc(F)c(C)c3c1c2C(C)(NC(=O)CO)C3 ; CC[C@@]1(O)C(=O)OCc2c1cc1n(c2=O)Cc2c-1nc1cc(F)c(C)c3c1c2C(C)(NC(=O)CCCO)C3 ; CC[C@@]1(O)C(=O)OCc2c1cc1n(c2=O)Cc2c-1nc1cc(F)c(C)c3c1c2C(C)(NC(=O)CCC(C)O)C3]

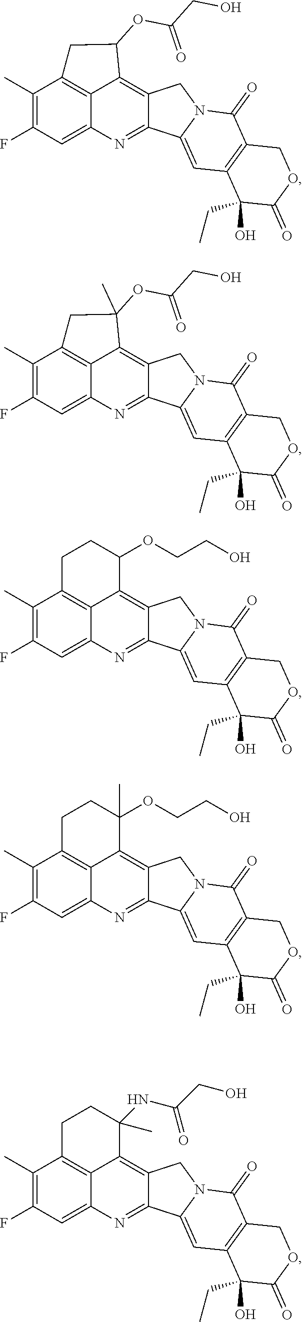

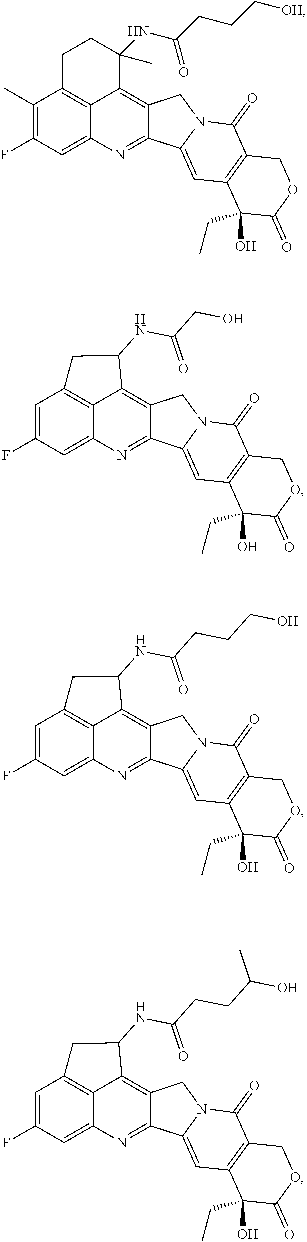 [SMILES: CC[C@@]1(O)C(=O)OCc2c1cc1n(c2=O)Cc2c-1nc1cc(F)c(C)c3c1c2C(C)(NC(=O)CCCO)CC3 ; CC[C@@]1(O)C(=O)OCc2c1cc1n(c2=O)Cc2c-1nc1cc(F)cc3c1c2C(NC(=O)CO)C3 ; CC[C@@]1(O)C(=O)OCc2c1cc1n(c2=O)Cc2c-1nc1cc(F)cc3c1c2C(NC(=O)CCCO)C3 ; CC[C@@]1(O)C(=O)OCc2c1cc1n(c2=O)Cc2c-1nc1cc(F)cc3c1c2C(NC(=O)CCC(C)O)C3]

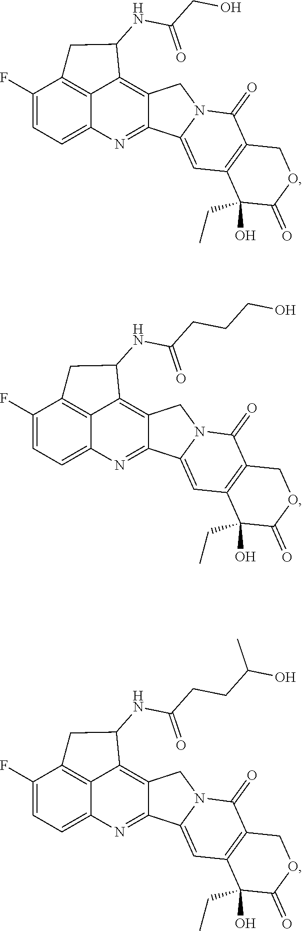 [SMILES: CC[C@@]1(O)C(=O)OCc2c1cc1n(c2=O)Cc2c-1nc1ccc(F)c3c1c2C(NC(=O)CO)C3 ; CC[C@@]1(O)C(=O)OCc2c1cc1n(c2=O)Cc2c-1nc1ccc(F)c3c1c2C(NC(=O)CCCO)C3 ; CC[C@@]1(O)C(=O)OCc2c1cc1n(c2=O)Cc2c-1nc1ccc(F)c3c1c2C(NC(=O)CCC(C)O)C3]

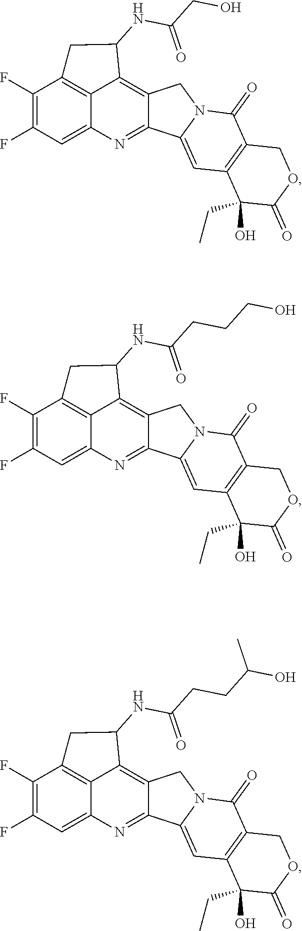 [SMILES: CC[C@@]1(O)C(=O)OCc2c1cc1n(c2=O)Cc2c-1nc1cc(F)c(F)c3c1c2C(NC(=O)CO)C3 ; CC[C@@]1(O)C(=O)OCc2c1cc1n(c2=O)Cc2c-1nc1cc(F)c(F)c3c1c2C(NC(=O)CCCO)C3 ; CC[C@@]1(O)C(=O)OCc2c1cc1n(c2=O)Cc2c-1nc1cc(F)c(F)c3c1c2C(NC(=O)CCC(C)O)C3]

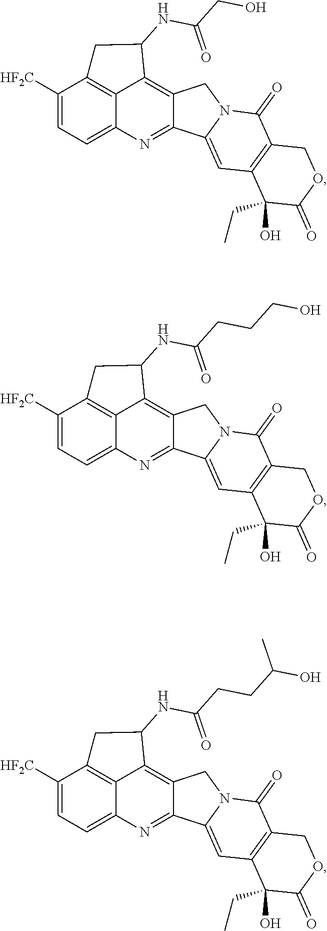

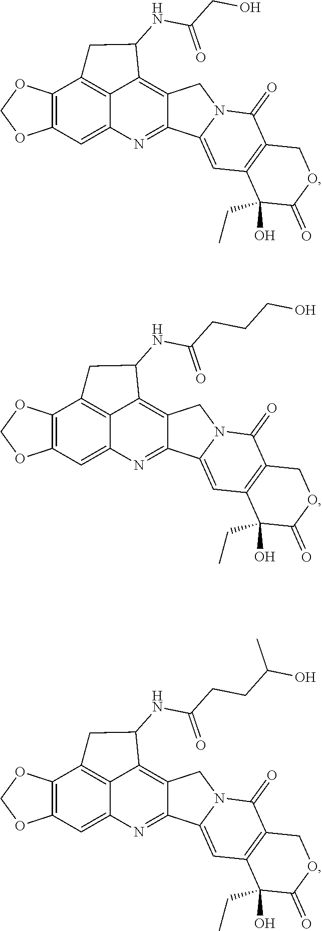 [SMILES: CC[C@@]1(O)C(=O)OCc2c1cc1n(c2=O)Cc2c-1nc1cc3c(c4c1c2C(NC(=O)CO)C4)OCO3 ; CC[C@@]1(O)C(=O)OCc2c1cc1n(c2=O)Cc2c-1nc1cc3c(c4c1c2C(NC(=O)CCCO)C4)OCO3 ; CC[C@@]1(O)C(=O)OCc2c1cc1n(c2=O)Cc2c-1nc1cc3c(c4c1c2C(NC(=O)CCC(C)O)C4)OCO3]

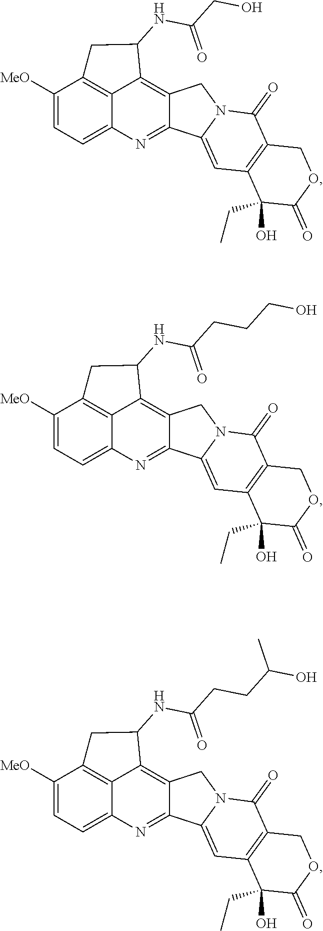 [SMILES: CC[C@@]1(O)C(=O)OCc2c1cc1n(c2=O)Cc2c-1nc1ccc(OC)c3c1c2C(NC(=O)CO)C3 ; CC[C@@]1(O)C(=O)OCc2c1cc1n(c2=O)Cc2c-1nc1ccc(OC)c3c1c2C(NC(=O)CCCO)C3 ; CC[C@@]1(O)C(=O)OCc2c1cc1n(c2=O)Cc2c-1nc1ccc(OC)c3c1c2C(NC(=O)CCC(C)O)C3]

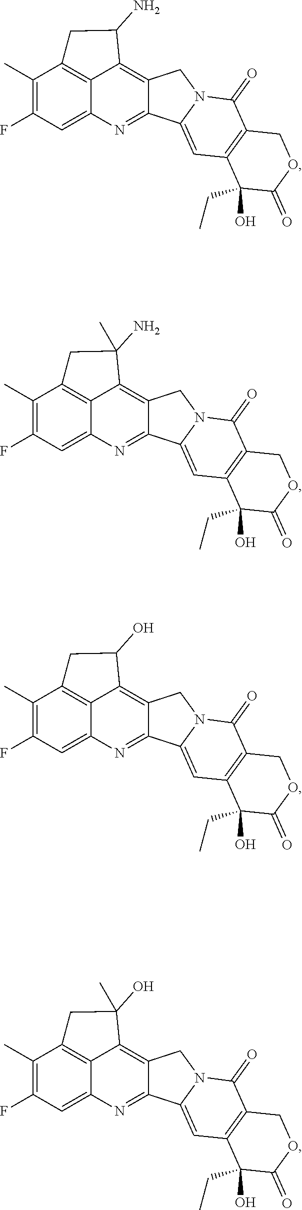 [SMILES: CC[C@@]1(O)C(=O)OCc2c1cc1n(c2=O)Cc2c-1nc1cc(F)c(C)c3c1c2C(N)C3 ; CC[C@@]1(O)C(=O)OCc2c1cc1n(c2=O)Cc2c-1nc1cc(F)c(C)c3c1c2C(C)(N)C3 ; CC[C@@]1(O)C(=O)OCc2c1cc1n(c2=O)Cc2c-1nc1cc(F)c(C)c3c1c2C(O)C3 ; CC[C@@]1(O)C(=O)OCc2c1cc1n(c2=O)Cc2c-1nc1cc(F)c(C)c3c1c2C(C)(O)C3]

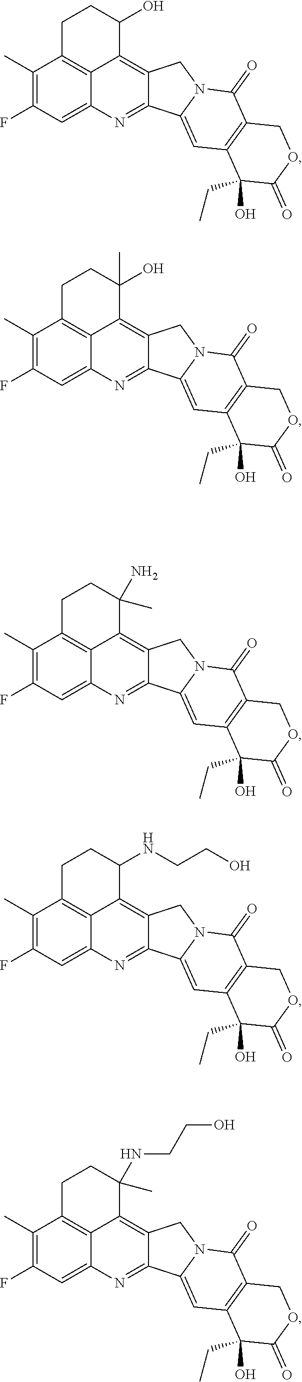 [SMILES: CC[C@@]1(O)C(=O)OCc2c1cc1n(c2=O)Cc2c-1nc1cc(F)c(C)c3c1c2C(O)CC3 ; CC[C@@]1(O)C(=O)OCc2c1cc1n(c2=O)Cc2c-1nc1cc(F)c(C)c3c1c2C(C)(O)CC3 ; CC[C@@]1(O)C(=O)OCc2c1cc1n(c2=O)Cc2c-1nc1cc(F)c(C)c3c1c2C(C)(N)CC3 ; CC[C@@]1(O)C(=O)OCc2c1cc1n(c2=O)Cc2c-1nc1cc(F)c(C)c3c1c2C(NCCO)CC3 ; CC[C@@]1(O)C(=O)OCc2c1cc1n(c2=O)Cc2c-1nc1cc(F)c(C)c3c1c2C(C)(NCCO)CC3]

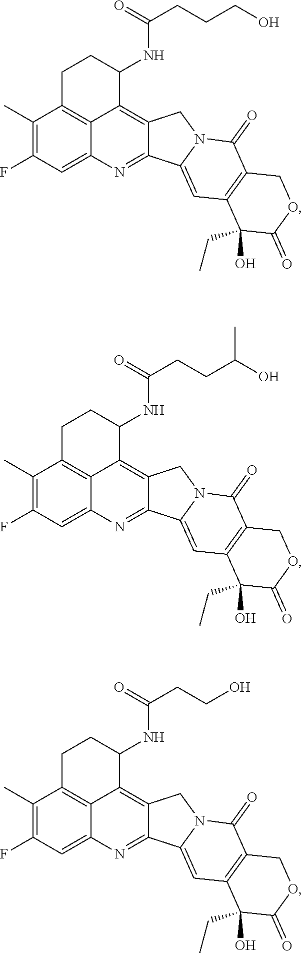 [SMILES: CC[C@@]1(O)C(=O)OCc2c1cc1n(c2=O)Cc2c-1nc1cc(F)c(C)c3c1c2C(NC(=O)CCCO)CC3 ; CC[C@@]1(O)C(=O)OCc2c1cc1n(c2=O)Cc2c-1nc1cc(F)c(C)c3c1c2C(NC(=O)CCC(C)O)CC3 ; CC[C@@]1(O)C(=O)OCc2c1cc1n(c2=O)Cc2c-1nc1cc(F)c(C)c3c1c2C(NC(=O)CCO)CC3]

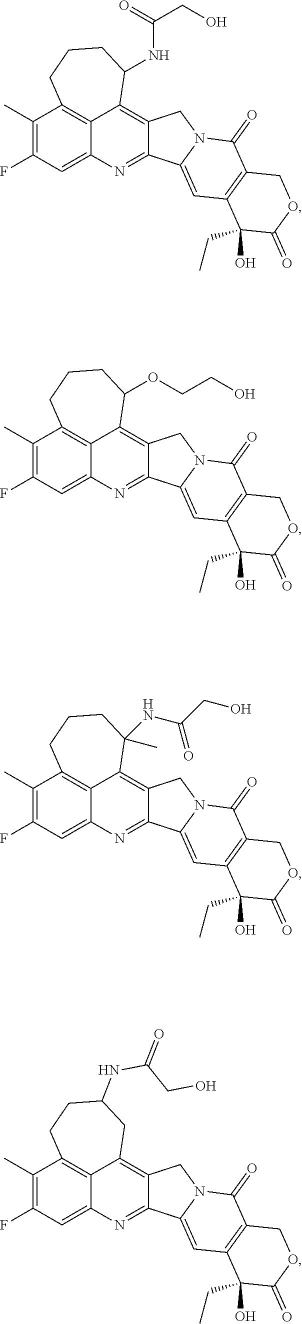 [SMILES: CC[C@@]1(O)C(=O)OCc2c1cc1n(c2=O)Cc2c-1nc1cc(F)c(C)c3c1c2C(NC(=O)CO)CCC3 ; CC[C@@]1(O)C(=O)OCc2c1cc1n(c2=O)Cc2c-1nc1cc(F)c(C)c3c1c2C(OCCO)CCC3 ; CC[C@@]1(O)C(=O)OCc2c1cc1n(c2=O)Cc2c-1nc1cc(F)c(C)c3c1c2C(C)(NC(=O)CO)CCC3 ; CC[C@@]1(O)C(=O)OCc2c1cc1n(c2=O)Cc2c-1nc1cc(F)c(C)c3c1c2CC(NC(=O)CO)CC3]

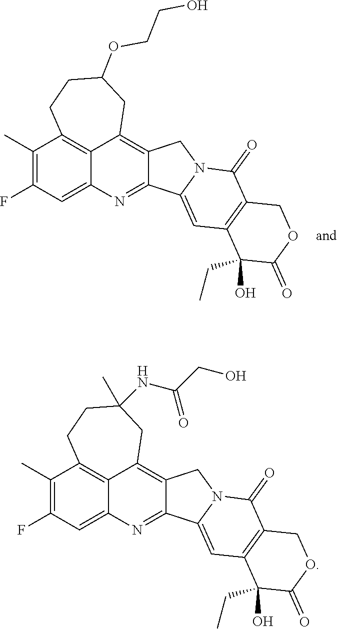 [SMILES: CC[C@@]1(O)C(=O)OCc2c1cc1n(c2=O)Cc2c-1nc1cc(F)c(C)c3c1c2CC(OCCO)CC3 ; CC[C@@]1(O)C(=O)OCc2c1cc1n(c2=O)Cc2c-1nc1cc(F)c(C)c3c1c2CC(C)(NC(=O)CO)CC3]

In some embodiments, a compound of Formula (IV), or a pharmaceutically acceptable salt thereof, cannot be selected from:

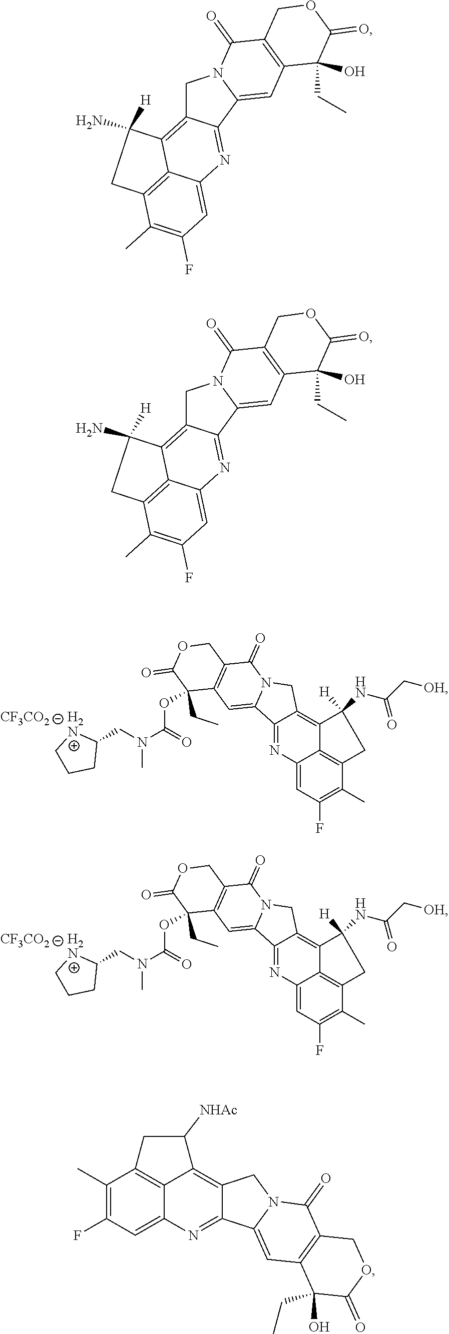

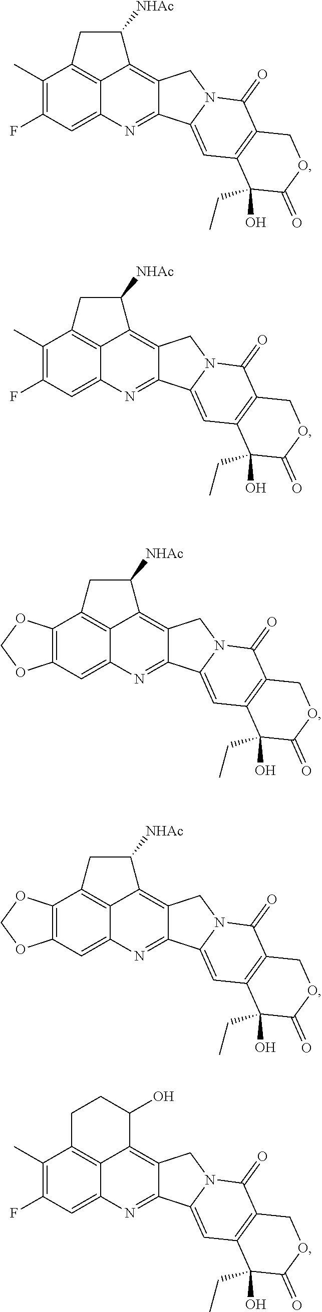 [SMILES: CC[C@@]1(O)C(=O)OCc2c1cc1n(c2=O)Cc2c-1nc1cc(F)c(C)c3c1c2[C@@H](NC(C)=O)C3 ; CC[C@@]1(O)C(=O)OCc2c1cc1n(c2=O)Cc2c-1nc1cc(F)c(C)c3c1c2[C@H](NC(C)=O)C3 ; CC[C@@]1(O)C(=O)OCc2c1cc1n(c2=O)Cc2c-1nc1cc3c(c4c1c2[C@H](NC(C)=O)C4)OCO3 ; CC[C@@]1(O)C(=O)OCc2c1cc1n(c2=O)Cc2c-1nc1cc3c(c4c1c2[C@@H](NC(C)=O)C4)OCO3 ; CC[C@@]1(O)C(=O)OCc2c1cc1n(c2=O)Cc2c-1nc1cc(F)c(C)c3c1c2C(O)CC3]

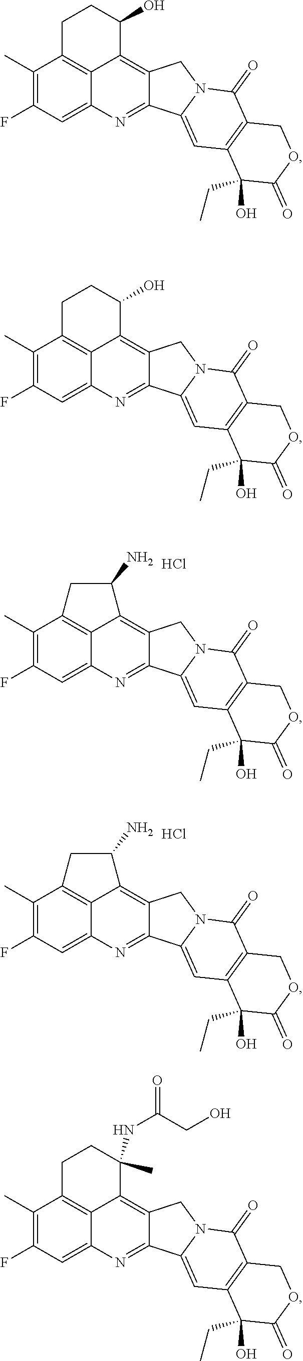

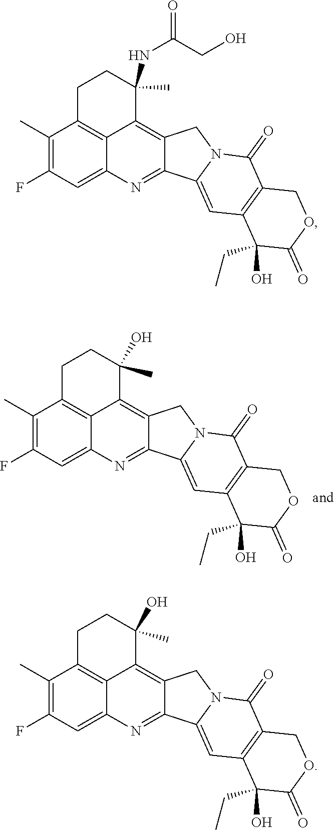 [SMILES: CC[C@@]1(O)C(=O)OCc2c1cc1n(c2=O)Cc2c-1nc1cc(F)c(C)c3c1c2[C@](C)(NC(=O)CO)CC3 ; CC[C@@]1(O)C(=O)OCc2c1cc1n(c2=O)Cc2c-1nc1cc(F)c(C)c3c1c2[C@@](C)(O)CC3 ; CC[C@@]1(O)C(=O)OCc2c1cc1n(c2=O)Cc2c-1nc1cc(F)c(C)c3c1c2[C@](C)(O)CC3]

In various embodiments, a compound of Formula (IV) can be represented by a structure selected from the following, or a pharmaceutically acceptable salt thereof:

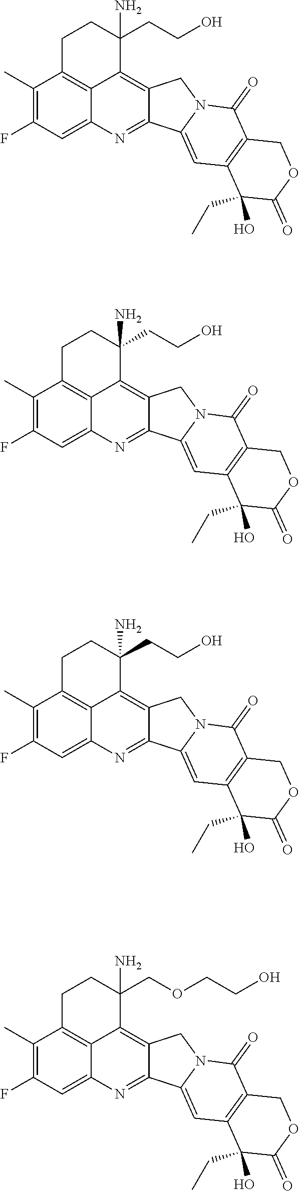 [SMILES: CC[C@@]1(O)C(=O)OCc2c1cc1n(c2=O)Cc2c-1nc1cc(F)c(C)c3c1c2C(N)(CCO)CC3 ; CC[C@@]1(O)C(=O)OCc2c1cc1n(c2=O)Cc2c-1nc1cc(F)c(C)c3c1c2[C@@](N)(CCO)CC3 ; CC[C@@]1(O)C(=O)OCc2c1cc1n(c2=O)Cc2c-1nc1cc(F)c(C)c3c1c2[C@](N)(CCO)CC3 ; CC[C@@]1(O)C(=O)OCc2c1cc1n(c2=O)Cc2c-1nc1cc(F)c(C)c3c1c2C(N)(COCCO)CC3]

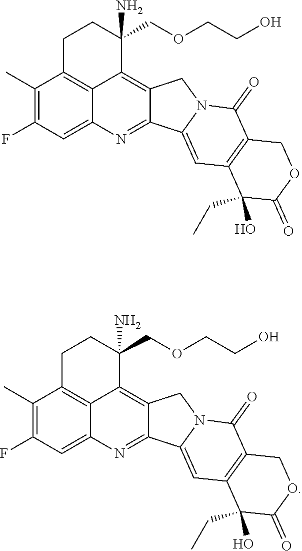 [SMILES: CC[C@@]1(O)C(=O)OCc2c1cc1n(c2=O)Cc2c-1nc1cc(F)c(C)c3c1c2[C@@](N)(COCCO)CC3 ; CC[C@@]1(O)C(=O)OCc2c1cc1n(c2=O)Cc2c-1nc1cc(F)c(C)c3c1c2[C@](N)(COCCO)CC3]

In various embodiments, a compound of Formula (IV) can be represented by a structure selected from the following, or a pharmaceutically acceptable salt thereof:

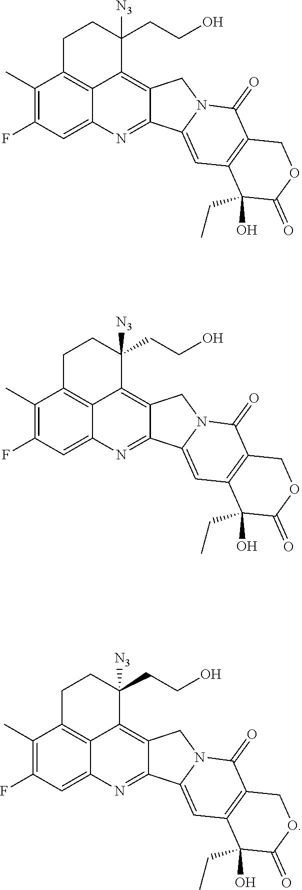 [SMILES: CC[C@@]1(O)C(=O)OCc2c1cc1n(c2=O)Cc2c-1nc1cc(F)c(C)c3c1c2C(CCO)(N=[N+]=[N-])CC3 ; CC[C@@]1(O)C(=O)OCc2c1cc1n(c2=O)Cc2c-1nc1cc(F)c(C)c3c1c2[C@](CCO)(N=[N+]=[N-])CC3 ; CC[C@@]1(O)C(=O)OCc2c1cc1n(c2=O)Cc2c-1nc1cc(F)c(C)c3c1c2[C@@](CCO)(N=[N+]=[N-])CC3]

In various embodiments, a compound of Formula (IV) can be represented by a structure selected from the following compounds in TABLE A, or a pharmaceutically acceptable salt thereof

TABLE A

| Compound | |
|---|---|
| No. | Structure |
| 1-7A | 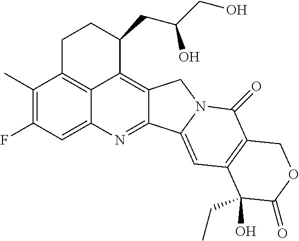 [SMILES: CC[C@@]1(O)C(=O)OCc2c1cc1n(c2=O)Cc2c-1nc1cc(F)c(C)c3c1c2[C@H](C[C@H](O)CO)CC3] |
| 1-8A | 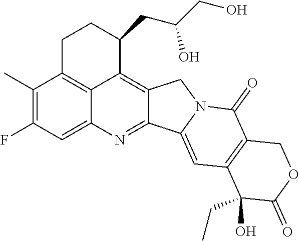 [SMILES: CC[C@@]1(O)C(=O)OCc2c1cc1n(c2=O)Cc2c-1nc1cc(F)c(C)c3c1c2[C@H](C[C@@H](O)CO)CC3] |
| 1-9A | 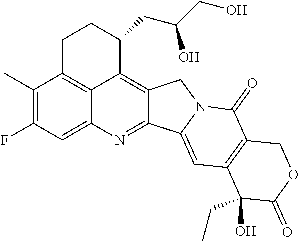 [SMILES: CC[C@@]1(O)C(=O)OCc2c1cc1n(c2=O)Cc2c-1nc1cc(F)c(C)c3c1c2[C@@H](C[C@H](O)CO)CC3] |
| 1-10A | 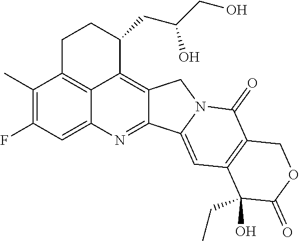 [SMILES: CC[C@@]1(O)C(=O)OCc2c1cc1n(c2=O)Cc2c-1nc1cc(F)c(C)c3c1c2[C@@H](C[C@@H](O)CO)CC3] |
| 1-5 (Isomer 1) | 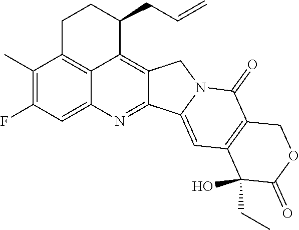 [SMILES: C=CC[C@@H]1CCc2c(C)c(F)cc3nc4c(c1c23)Cn1c-4cc2c(c1=O)COC(=O)[C@]2(O)CC] |
| 1-5 (Isomer 2) | 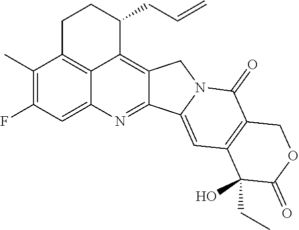 [SMILES: C=CC[C@H]1CCc2c(C)c(F)cc3nc4c(c1c23)Cn1c-4cc2c(c1=O)COC(=O)[C@]2(O)CC] |
| 2-15 | 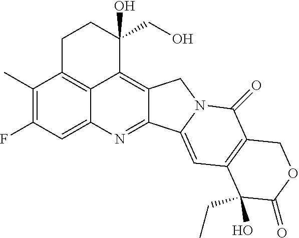 [SMILES: CC[C@@]1(O)C(=O)OCc2c1cc1n(c2=O)Cc2c-1nc1cc(F)c(C)c3c1c2[C@@](O)(CO)CC3] |
| 2-16 | 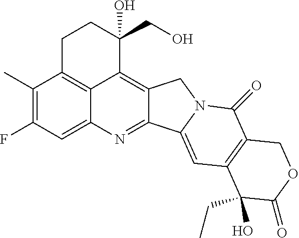 [SMILES: CC[C@@]1(O)C(=O)OCc2c1cc1n(c2=O)Cc2c-1nc1cc(F)c(C)c3c1c2[C@](O)(CO)CC3] |
| 3-24 | 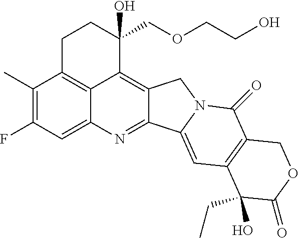 [SMILES: CC[C@@]1(O)C(=O)OCc2c1cc1n(c2=O)Cc2c-1nc1cc(F)c(C)c3c1c2[C@@](O)(COCCO)CC3] |
| 3-25 | 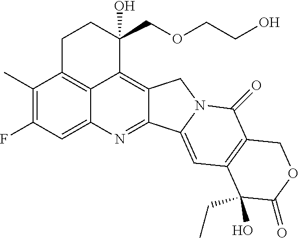 [SMILES: CC[C@@]1(O)C(=O)OCc2c1cc1n(c2=O)Cc2c-1nc1cc(F)c(C)c3c1c2[C@](O)(COCCO)CC3] |
| 4-29 | 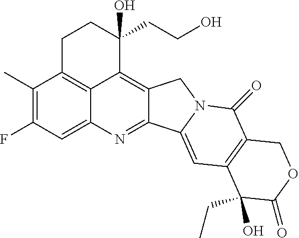 [SMILES: CC[C@@]1(O)C(=O)OCc2c1cc1n(c2=O)Cc2c-1nc1cc(F)c(C)c3c1c2[C@@](O)(CCO)CC3] |
| 4-30 | 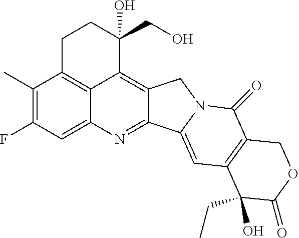 [SMILES: CC[C@@]1(O)C(=O)OCc2c1cc1n(c2=O)Cc2c-1nc1cc(F)c(C)c3c1c2[C@](O)(CO)CC3] |
| 5-38 | 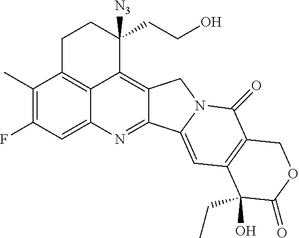 [SMILES: CC[C@@]1(O)C(=O)OCc2c1cc1n(c2=O)Cc2c-1nc1cc(F)c(C)c3c1c2[C@](CCO)(N=[N+]=[N-])CC3] |
| 5-39 | 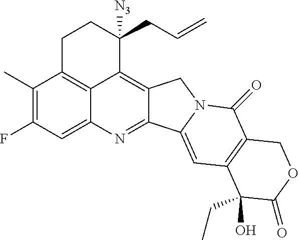 [SMILES: C=CC[C@]1(N=[N+]=[N-])CCc2c(C)c(F)cc3nc4c(c1c23)Cn1c-4cc2c(c1=O)COC(=O)[C@]2(O)CC] |
| 6-45 | 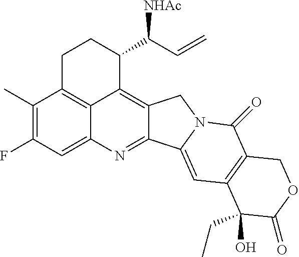 [SMILES: C=C[C@H](NC(C)=O)[C@H]1CCc2c(C)c(F)cc3nc4c(c1c23)Cn1c-4cc2c(c1=O)COC(=O)[C@]2(O)CC] |
| 6-46 | 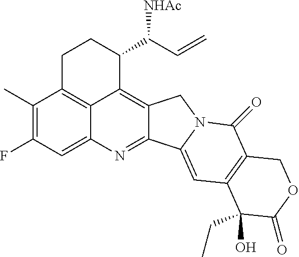 [SMILES: C=C[C@@H](NC(C)=O)[C@H]1CCc2c(C)c(F)cc3nc4c(c1c23)Cn1c-4cc2c(c1=O)COC(=O)[C@]2(O)CC] |
| 6-47 | 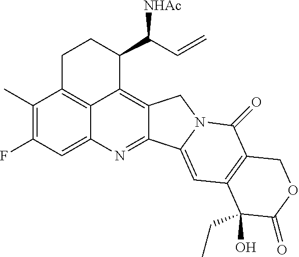 [SMILES: C=C[C@H](NC(C)=O)[C@@H]1CCc2c(C)c(F)cc3nc4c(c1c23)Cn1c-4cc2c(c1=O)COC(=O)[C@]2(O)CC] |
| 6-48 | 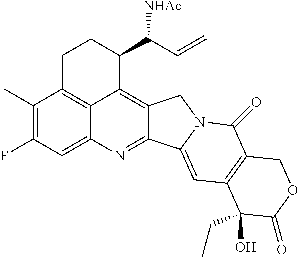 [SMILES: C=C[C@@H](NC(C)=O)[C@@H]1CCc2c(C)c(F)cc3nc4c(c1c23)Cn1c-4cc2c(c1=O)COC(=O)[C@]2(O)CC] |
| 7-50 | 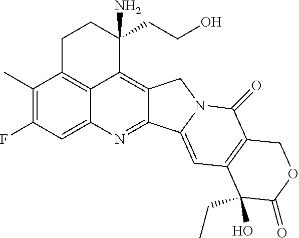 [SMILES: CC[C@@]1(O)C(=O)OCc2c1cc1n(c2=O)Cc2c-1nc1cc(F)c(C)c3c1c2[C@@](N)(CCO)CC3] |
| 7-51 | 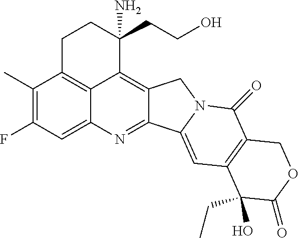 [SMILES: CC[C@@]1(O)C(=O)OCc2c1cc1n(c2=O)Cc2c-1nc1cc(F)c(C)c3c1c2[C@](N)(CCO)CC3] |
| 8-57 | 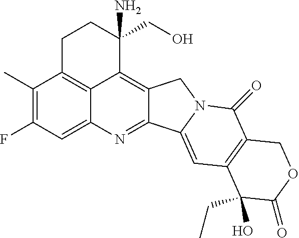 [SMILES: CC[C@@]1(O)C(=O)OCc2c1cc1n(c2=O)Cc2c-1nc1cc(F)c(C)c3c1c2[C@@](N)(CO)CC3] |
| 8-58 | 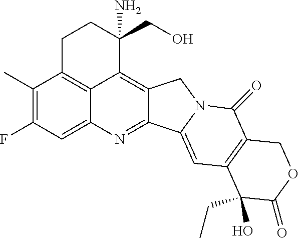 [SMILES: CC[C@@]1(O)C(=O)OCc2c1cc1n(c2=O)Cc2c-1nc1cc(F)c(C)c3c1c2[C@](N)(CO)CC3] |
| 9-64 | 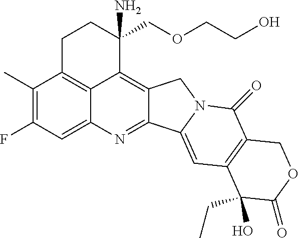 [SMILES: CC[C@@]1(O)C(=O)OCc2c1cc1n(c2=O)Cc2c-1nc1cc(F)c(C)c3c1c2[C@@](N)(COCCO)CC3] |
| 9-65 | 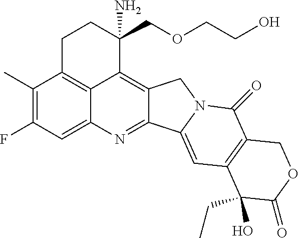 [SMILES: CC[C@@]1(O)C(=O)OCc2c1cc1n(c2=O)Cc2c-1nc1cc(F)c(C)c3c1c2[C@](N)(COCCO)CC3] |
| 10-68 | 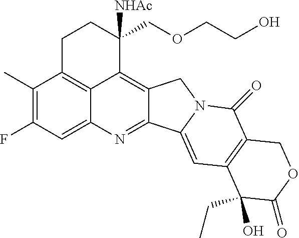 [SMILES: CC[C@@]1(O)C(=O)OCc2c1cc1n(c2=O)Cc2c-1nc1cc(F)c(C)c3c1c2[C@](COCCO)(NC(C)=O)CC3] |
| 10-69 | 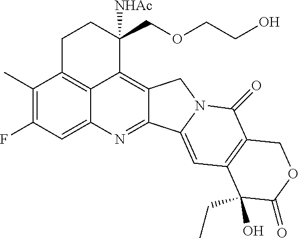 [SMILES: CC[C@@]1(O)C(=O)OCc2c1cc1n(c2=O)Cc2c-1nc1cc(F)c(C)c3c1c2[C@@](COCCO)(NC(C)=O)CC3] |
| 11-76 | 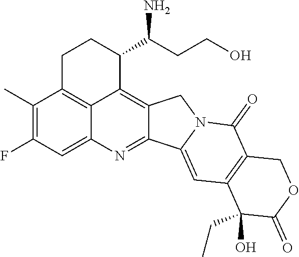 [SMILES: CC[C@@]1(O)C(=O)OCc2c1cc1n(c2=O)Cc2c-1nc1cc(F)c(C)c3c1c2[C@@H]([C@@H](N)CCO)CC3] |
| 11-78 | 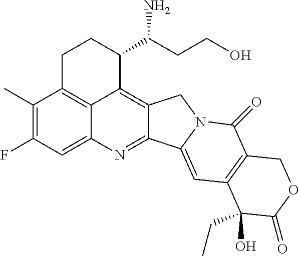 [SMILES: CC[C@@]1(O)C(=O)OCc2c1cc1n(c2=O)Cc2c-1nc1cc(F)c(C)c3c1c2[C@@H]([C@H](N)CCO)CC3] |
| 11-80 | 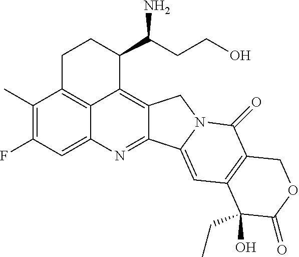 [SMILES: CC[C@@]1(O)C(=O)OCc2c1cc1n(c2=O)Cc2c-1nc1cc(F)c(C)c3c1c2[C@H]([C@@H](N)CCO)CC3] |
| 11-82 | 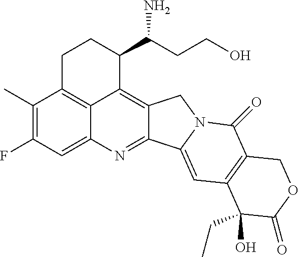 [SMILES: CC[C@@]1(O)C(=O)OCc2c1cc1n(c2=O)Cc2c-1nc1cc(F)c(C)c3c1c2[C@H]([C@H](N)CCO)CC3] |
| 12-86 | 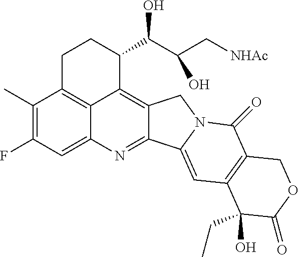 [SMILES: CC[C@@]1(O)C(=O)OCc2c1cc1n(c2=O)Cc2c-1nc1cc(F)c(C)c3c1c2[C@@H]([C@@H](O)[C@H](O)CNC(C)=O)CC3] |
| 12-87 | 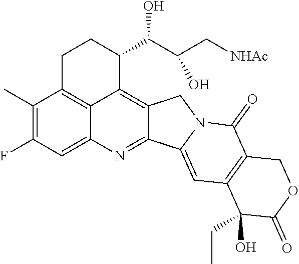 [SMILES: CC[C@@]1(O)C(=O)OCc2c1cc1n(c2=O)Cc2c-1nc1cc(F)c(C)c3c1c2[C@@H]([C@H](O)[C@@H](O)CNC(C)=O)CC3] |
| 13-88 | 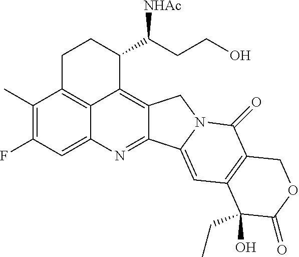 [SMILES: CC[C@@]1(O)C(=O)OCc2c1cc1n(c2=O)Cc2c-1nc1cc(F)c(C)c3c1c2[C@@H]([C@H](CCO)NC(C)=O)CC3] |
| 13-89 | 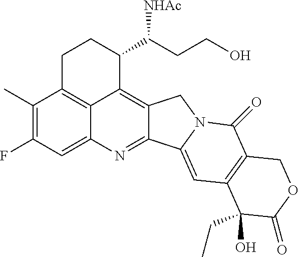 [SMILES: CC[C@@]1(O)C(=O)OCc2c1cc1n(c2=O)Cc2c-1nc1cc(F)c(C)c3c1c2[C@@H]([C@@H](CCO)NC(C)=O)CC3] |
| 13-90 | 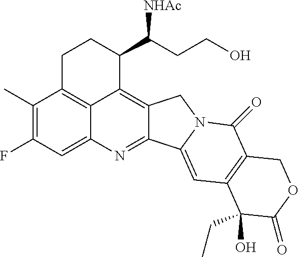 [SMILES: CC[C@@]1(O)C(=O)OCc2c1cc1n(c2=O)Cc2c-1nc1cc(F)c(C)c3c1c2[C@H]([C@H](CCO)NC(C)=O)CC3] |
| 13-91 | 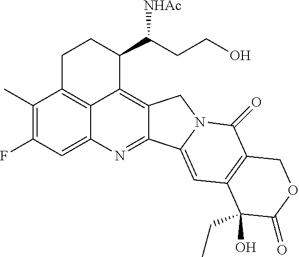 [SMILES: CC[C@@]1(O)C(=O)OCc2c1cc1n(c2=O)Cc2c-1nc1cc(F)c(C)c3c1c2[C@H]([C@@H](CCO)NC(C)=O)CC3] |
| 14-92 | 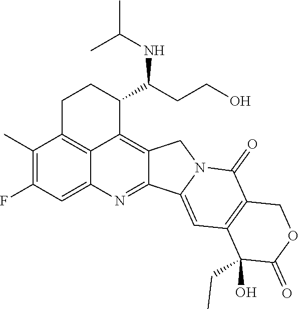 [SMILES: CC[C@@]1(O)C(=O)OCc2c1cc1n(c2=O)Cc2c-1nc1cc(F)c(C)c3c1c2[C@@H]([C@H](CCO)NC(C)C)CC3] |
| 14-93 | 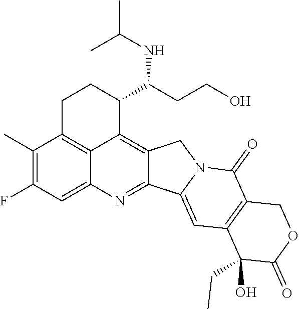 [SMILES: CC[C@@]1(O)C(=O)OCc2c1cc1n(c2=O)Cc2c-1nc1cc(F)c(C)c3c1c2[C@@H]([C@@H](CCO)NC(C)C)CC3] |
| 14-94 | 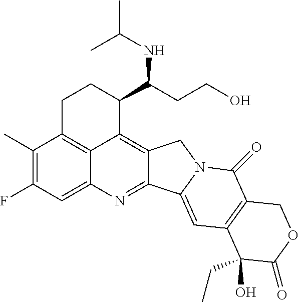 [SMILES: CC[C@@]1(O)C(=O)OCc2c1cc1n(c2=O)Cc2c-1nc1cc(F)c(C)c3c1c2[C@H]([C@H](CCO)NC(C)C)CC3] |
| 14-95 | 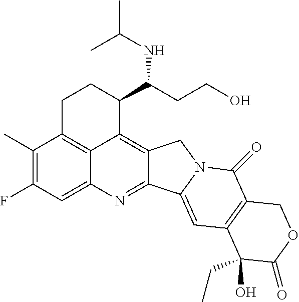 [SMILES: CC[C@@]1(O)C(=O)OCc2c1cc1n(c2=O)Cc2c-1nc1cc(F)c(C)c3c1c2[C@H]([C@@H](CCO)NC(C)C)CC3] |
| 15-96 | 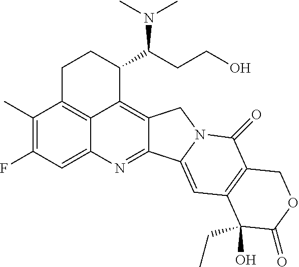 [SMILES: CC[C@@]1(O)C(=O)OCc2c1cc1n(c2=O)Cc2c-1nc1cc(F)c(C)c3c1c2[C@@H]([C@H](CCO)N(C)C)CC3] |
| 15-97 | 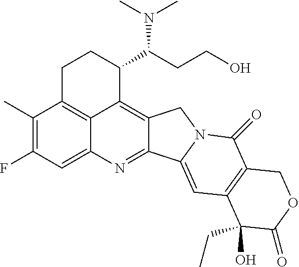 [SMILES: CC[C@@]1(O)C(=O)OCc2c1cc1n(c2=O)Cc2c-1nc1cc(F)c(C)c3c1c2[C@@H]([C@@H](CCO)N(C)C)CC3] |
| 15-98 | 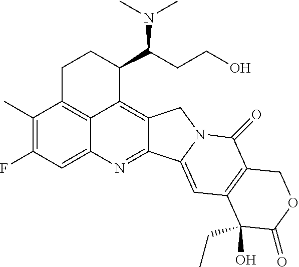 [SMILES: CC[C@@]1(O)C(=O)OCc2c1cc1n(c2=O)Cc2c-1nc1cc(F)c(C)c3c1c2[C@H]([C@H](CCO)N(C)C)CC3] |
| 15-99 | 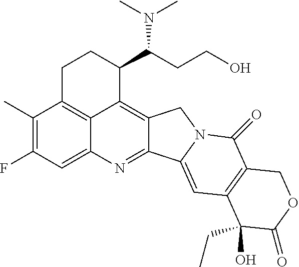 [SMILES: CC[C@@]1(O)C(=O)OCc2c1cc1n(c2=O)Cc2c-1nc1cc(F)c(C)c3c1c2[C@H]([C@@H](CCO)N(C)C)CC3] |
| 16-102 | 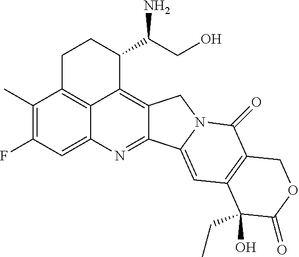 [SMILES: CC[C@@]1(O)C(=O)OCc2c1cc1n(c2=O)Cc2c-1nc1cc(F)c(C)c3c1c2[C@@H]([C@@H](N)CO)CC3] |
| 16-105 | 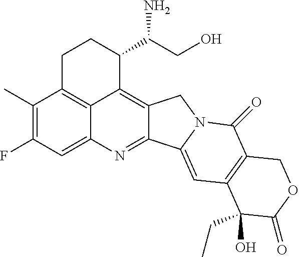 [SMILES: CC[C@@]1(O)C(=O)OCc2c1cc1n(c2=O)Cc2c-1nc1cc(F)c(C)c3c1c2[C@@H]([C@H](N)CO)CC3] |
| 16-108 | 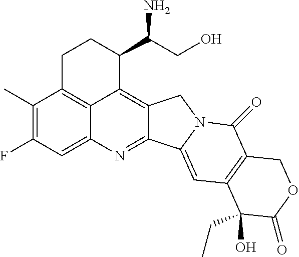 [SMILES: CC[C@@]1(O)C(=O)OCc2c1cc1n(c2=O)Cc2c-1nc1cc(F)c(C)c3c1c2[C@H]([C@@H](N)CO)CC3] |
| 16-111 | 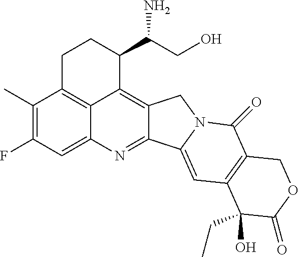 [SMILES: CC[C@@]1(O)C(=O)OCc2c1cc1n(c2=O)Cc2c-1nc1cc(F)c(C)c3c1c2[C@H]([C@H](N)CO)CC3] |
| 17-112 | 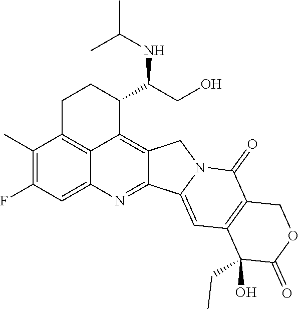 [SMILES: CC[C@@]1(O)C(=O)OCc2c1cc1n(c2=O)Cc2c-1nc1cc(F)c(C)c3c1c2[C@@H]([C@H](CO)NC(C)C)CC3] |
| 17-113 | 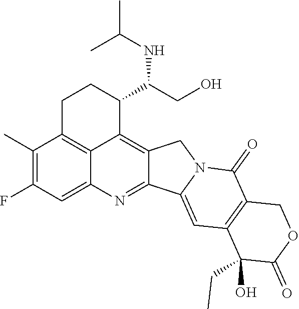 [SMILES: CC[C@@]1(O)C(=O)OCc2c1cc1n(c2=O)Cc2c-1nc1cc(F)c(C)c3c1c2[C@@H]([C@@H](CO)NC(C)C)CC3] |
| 17-114 | 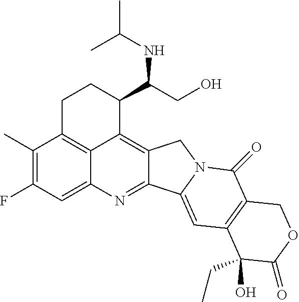 [SMILES: CC[C@@]1(O)C(=O)OCc2c1cc1n(c2=O)Cc2c-1nc1cc(F)c(C)c3c1c2[C@H]([C@H](CO)NC(C)C)CC3] |
| 17-115 | 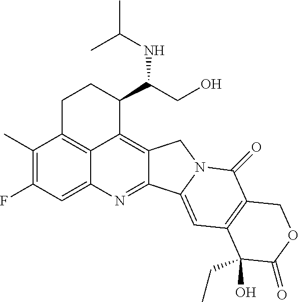 [SMILES: CC[C@@]1(O)C(=O)OCc2c1cc1n(c2=O)Cc2c-1nc1cc(F)c(C)c3c1c2[C@H]([C@@H](CO)NC(C)C)CC3] |

### Conjugates

Various embodiments disclosed herein relate to a conjugate of Formula (III), having the structure:

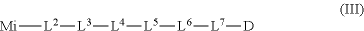

or a pharmaceutically acceptable salt thereof.

In various embodiments, Mi in Formula (III) is

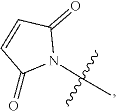 [SMILES: *N1C(=O)C=CC1=O]

D is a drug moiety and -L²-L³-L⁴-L³-L⁶-L′- is a linker that connects Mi to D.

In various embodiments, L² in Formula (III) is absent,

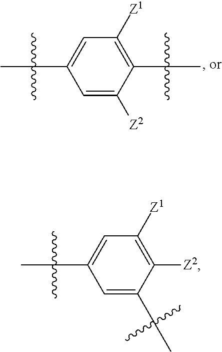

where Z¹ and Z² are each individually hydrogen, halogen, —NO₂, —O—(C₁-C₆ alkyl), or C₁-C₆ alkyl. In an embodiment, L² in Formula (III) is absent. In an embodiment, L² in Formula (III) is

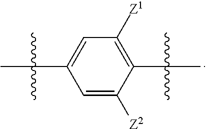

In an embodiment, L² in Formula (III) is

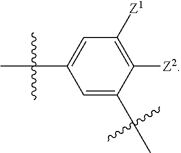

In various embodiments, Z¹ and Z² in Formula (III) are each individually hydrogen, halogen, —NO₂, —O—(C₁-C₆ alkyl), or C₁-C₆ alkyl. In an embodiment, at least one of Z¹ and Z² is hydrogen. In an embodiment, at least one of Z¹ and Z² is halogen. In an embodiment, at least one of Z¹ and Z² is —NO₂. In an embodiment, at least one of Z¹ and Z² is —O—(C₁-C₆ alkyl). For example, in an embodiment, at least one of Z¹ and Z² is methoxy. In an embodiment, at least one of Z¹ and Z² is C₁-C₆ alkyl. For example, in an embodiment, at least one of Z¹ and Z² is methyl.

In various embodiments, L³ in Formula (III) is —(CH₂)n¹-C(═O)— or —(CH₂CH₂O)n¹-(CH₂)n¹C(═O)—, where n¹ are independently integers of 0 to 12. In an embodiment, L³ is —(CH₂)n¹-C(═O)—. For example, in an embodiment, L³ is —C(═O)—. In an embodiment. L³ is —(CH₂CH₂O)n¹-(CH₂)n¹C(═O)—. For example, in an embodiment. L³ is —CH₂C(═O)—. In embodiment, n¹ is an integer of 1 to 12, such as 1 to 6 or 1 to 3.

In various embodiments, L⁴ in Formula (III) is a tetrapeptide residue. For example, in an embodiment, L⁴ is a tetrapeptide residue selected from GGFG (gly-gly-phe-gly), EGGF (glu-gly-gly-phe), SGGF (ser-gly-gly-phe), and KGGF (lys-gly-gly-phe).

In various embodiments, L⁵ in Formula (III) is absent or —[NH(CH₂)n²]n³-, where n² is an integer of 0 to 6 and n³ is an integer of 0 to 2. In an embodiment, L⁵ is absent. In an embodiment, L⁵ is —[NH(CH₂)n²]n³-. For example, in an embodiment, L⁵ is —NH—. In another embodiment. L⁵ is —NHCH₂—.

In various embodiments, L⁶ in Formula (III) is absent or

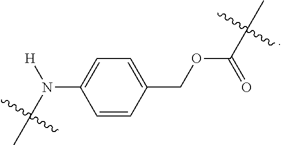 [SMILES: *Nc1ccc(COC(*)=O)cc1]

In an embodiment, L⁶ is absent. In another embodiment, L⁶ is

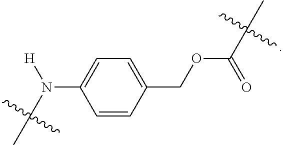 [SMILES: *Nc1ccc(COC(*)=O)cc1]

In various embodiments, L⁷ in Formula (III) is absent,

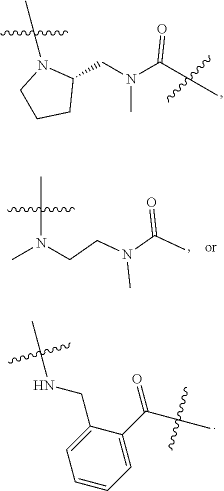

In an embodiment, L⁷ is absent. In an embodiment, L⁷ is

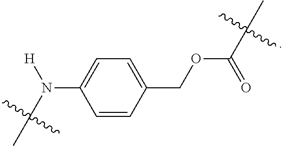 [SMILES: *Nc1ccc(COC(*)=O)cc1]

In an embodiment, L⁷ is

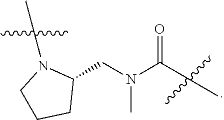 [SMILES: *C(=O)N(C)C[C@@H]1CCCN1*]

In an embodiment, L⁷ is

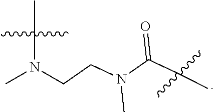 [SMILES: *C(=O)N(C)CCN(*)C]

In an embodiment, L⁷ is

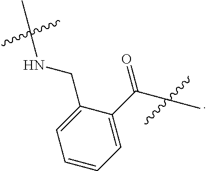 [SMILES: *NCc1ccccc1C(*)=O]

In various embodiments, D in the conjugate of Formula (III) is a drug moiety as described herein (e.g., under the heading “Drug Moieties” below). In an embodiment, D is a cytotoxic anti-cancer drug moiety.

In various embodiments, a conjugate of Formula (III) is represented by a structure selected from the following:

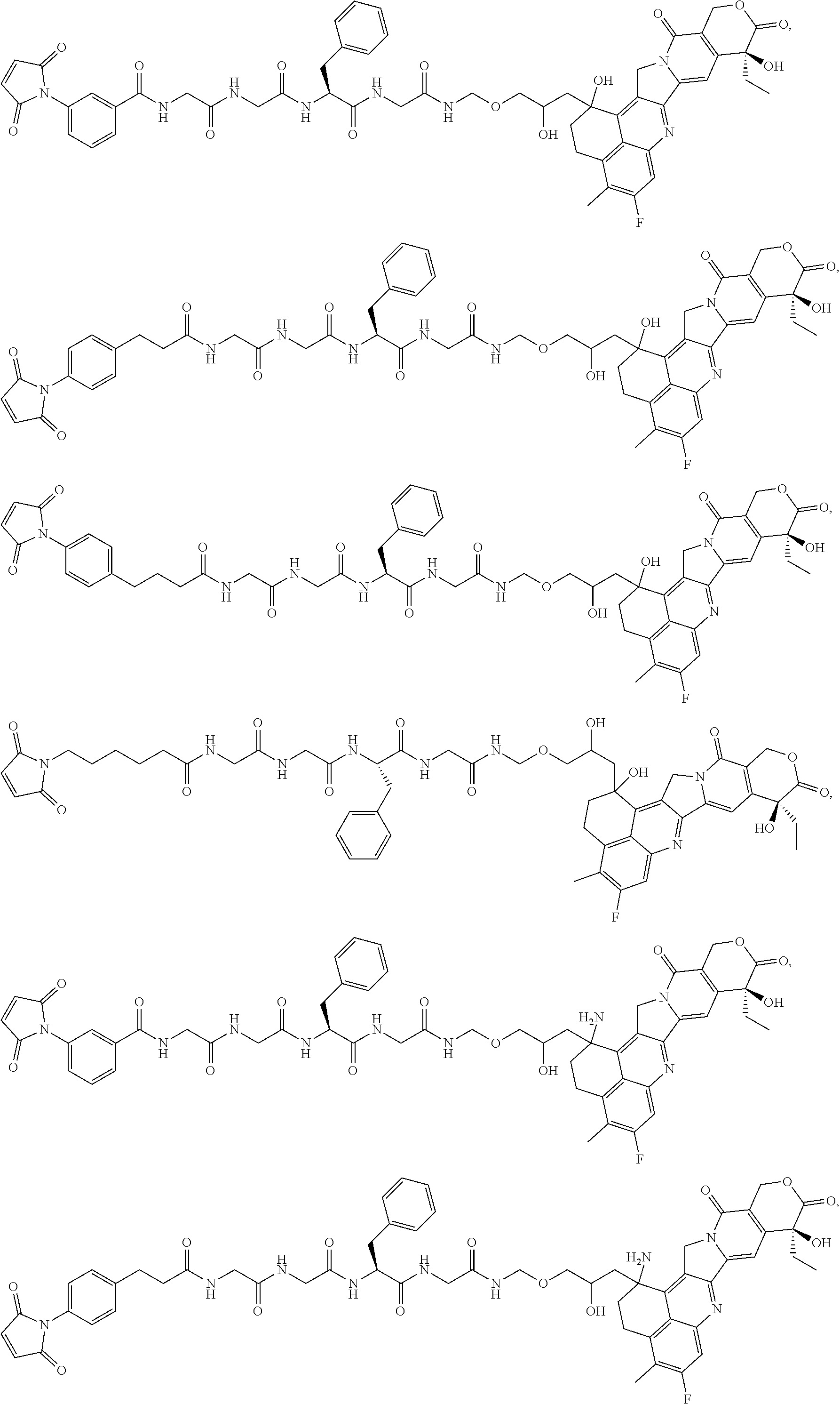

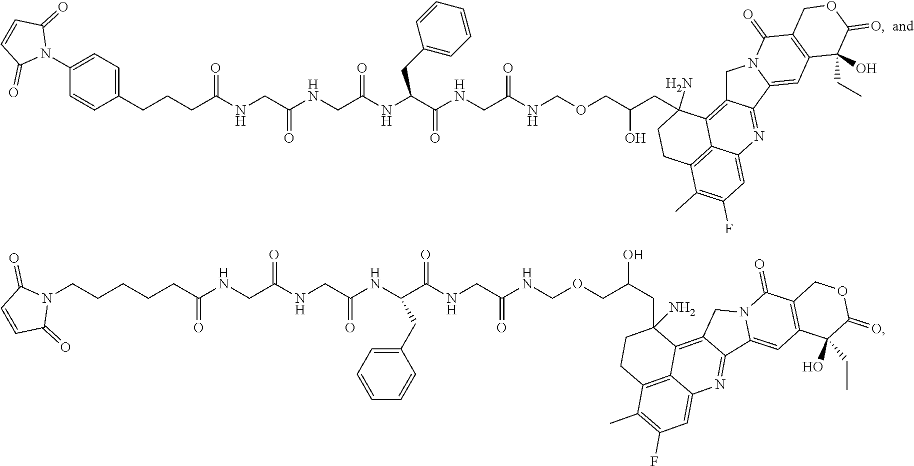

or a pharmaceutically acceptable salt of any of the foregoing.

In various embodiments, a conjugate of Formula (III) is represented by a structure selected from the following:

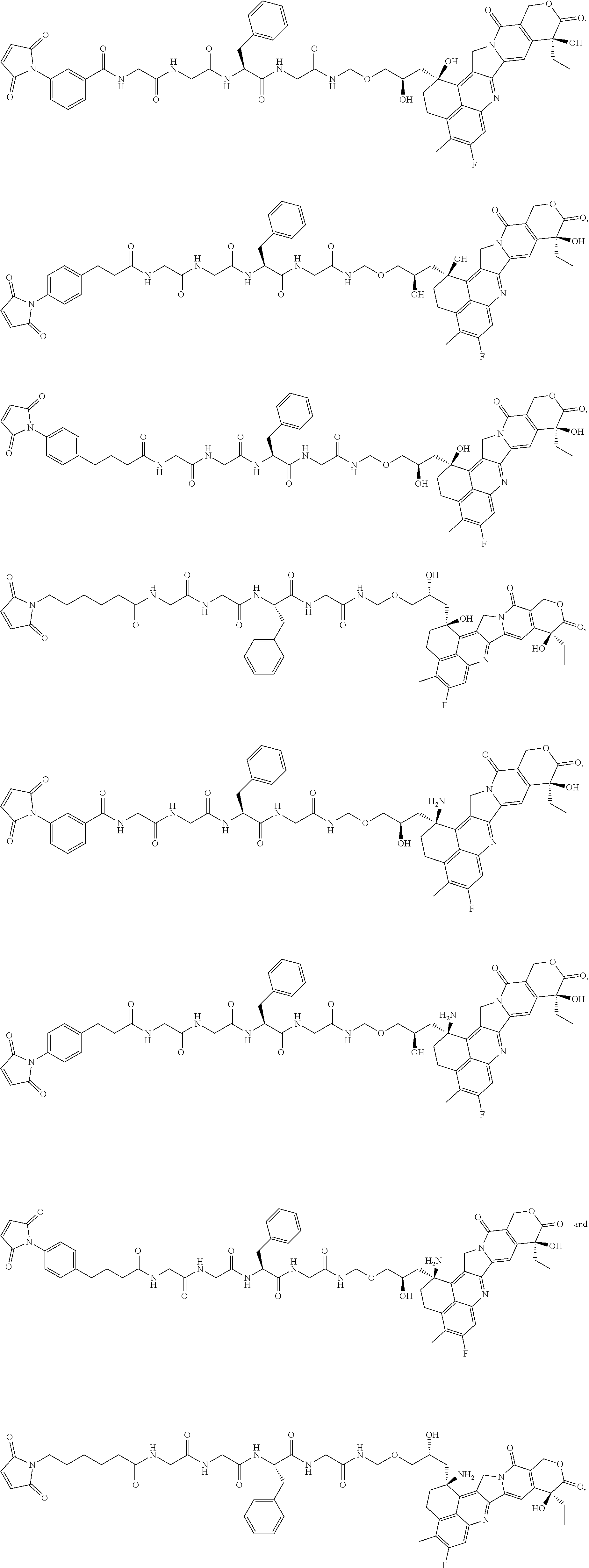

or a pharmaceutically acceptable salt of any of the foregoing.

In some embodiments, a conjugate of Formula (III), or a pharmaceutically acceptable salt thereof, cannot be selected from:

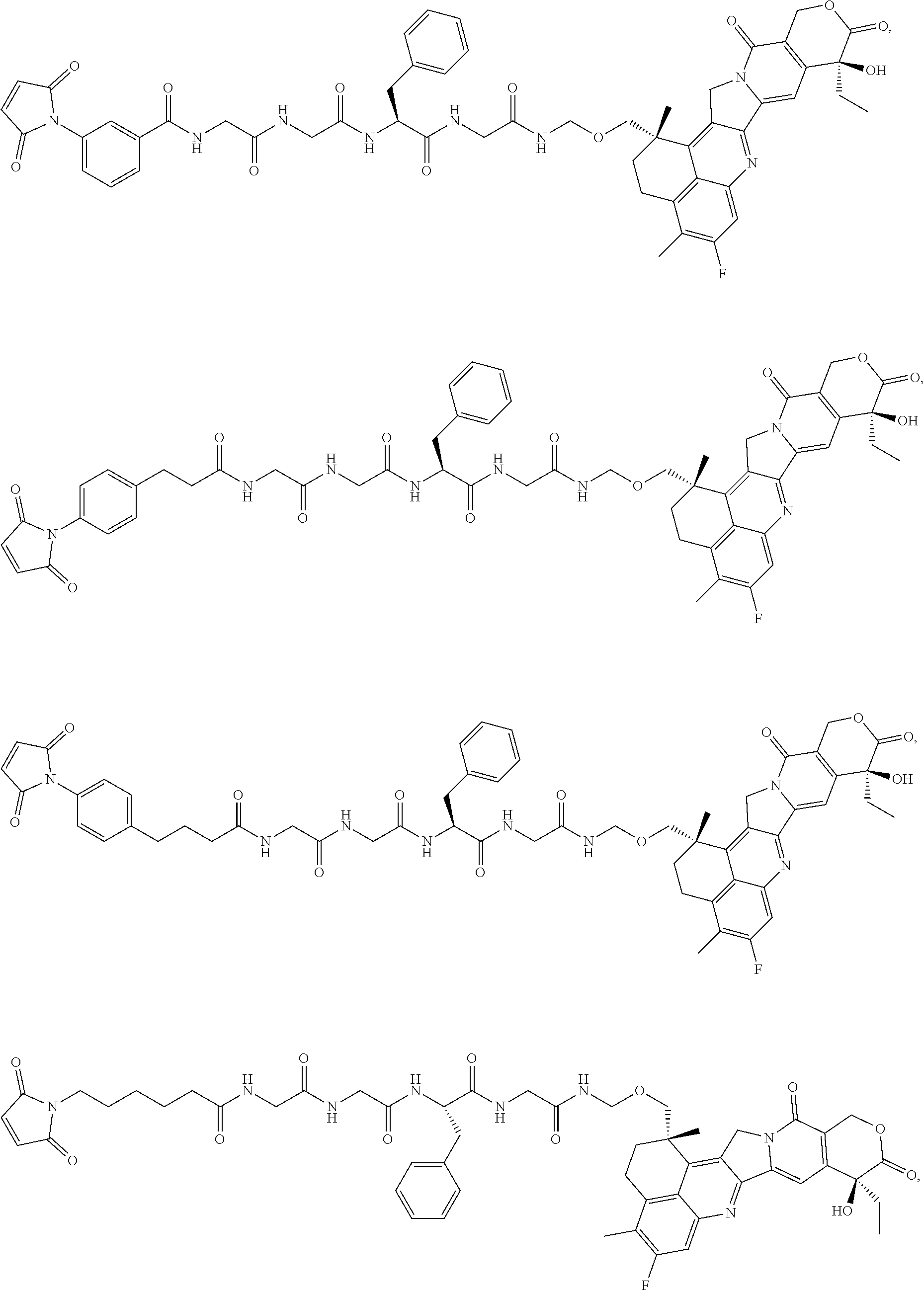

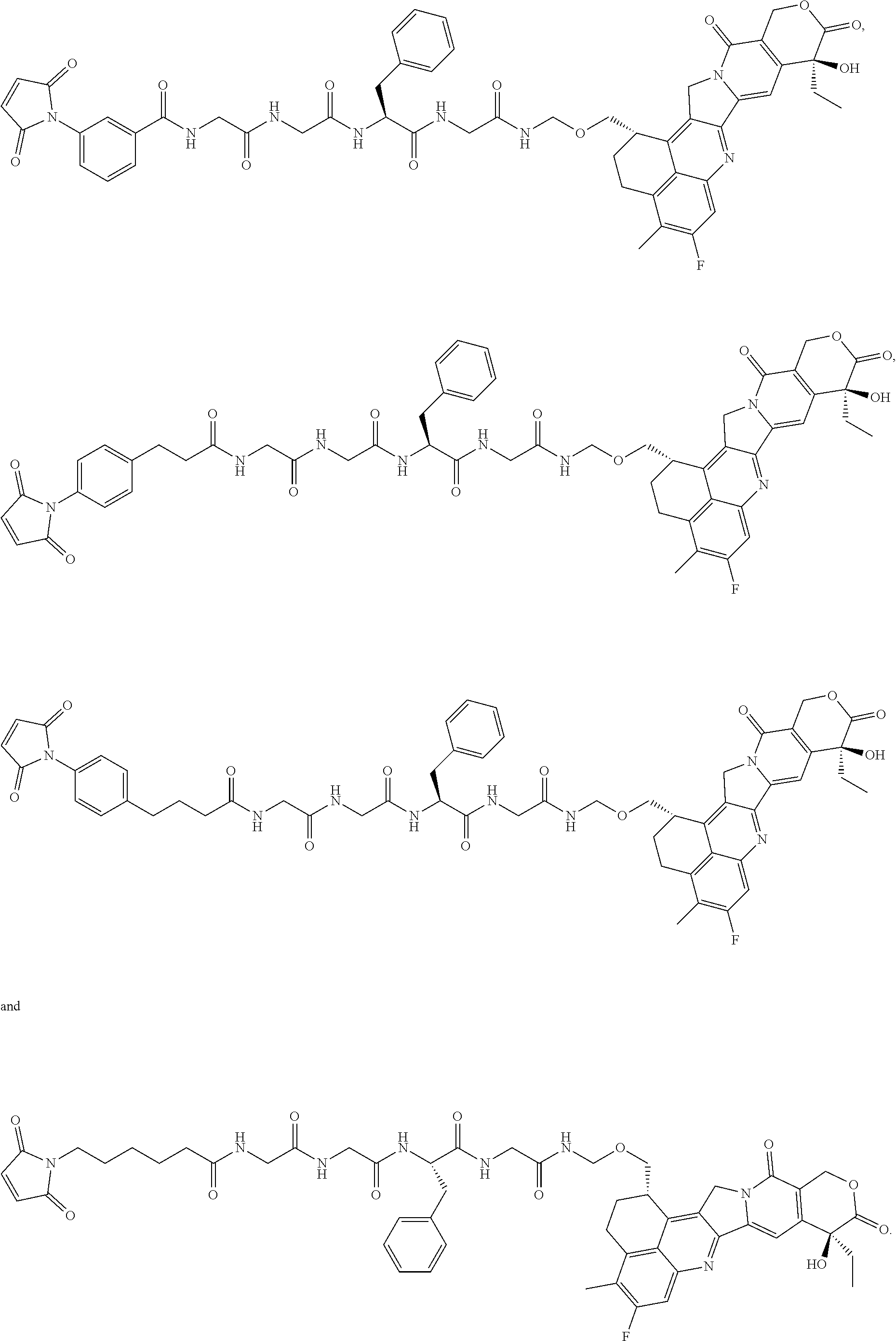

In some embodiments, a conjugate of Formula (III), or a pharmaceutically acceptable salt thereof, cannot be selected from:

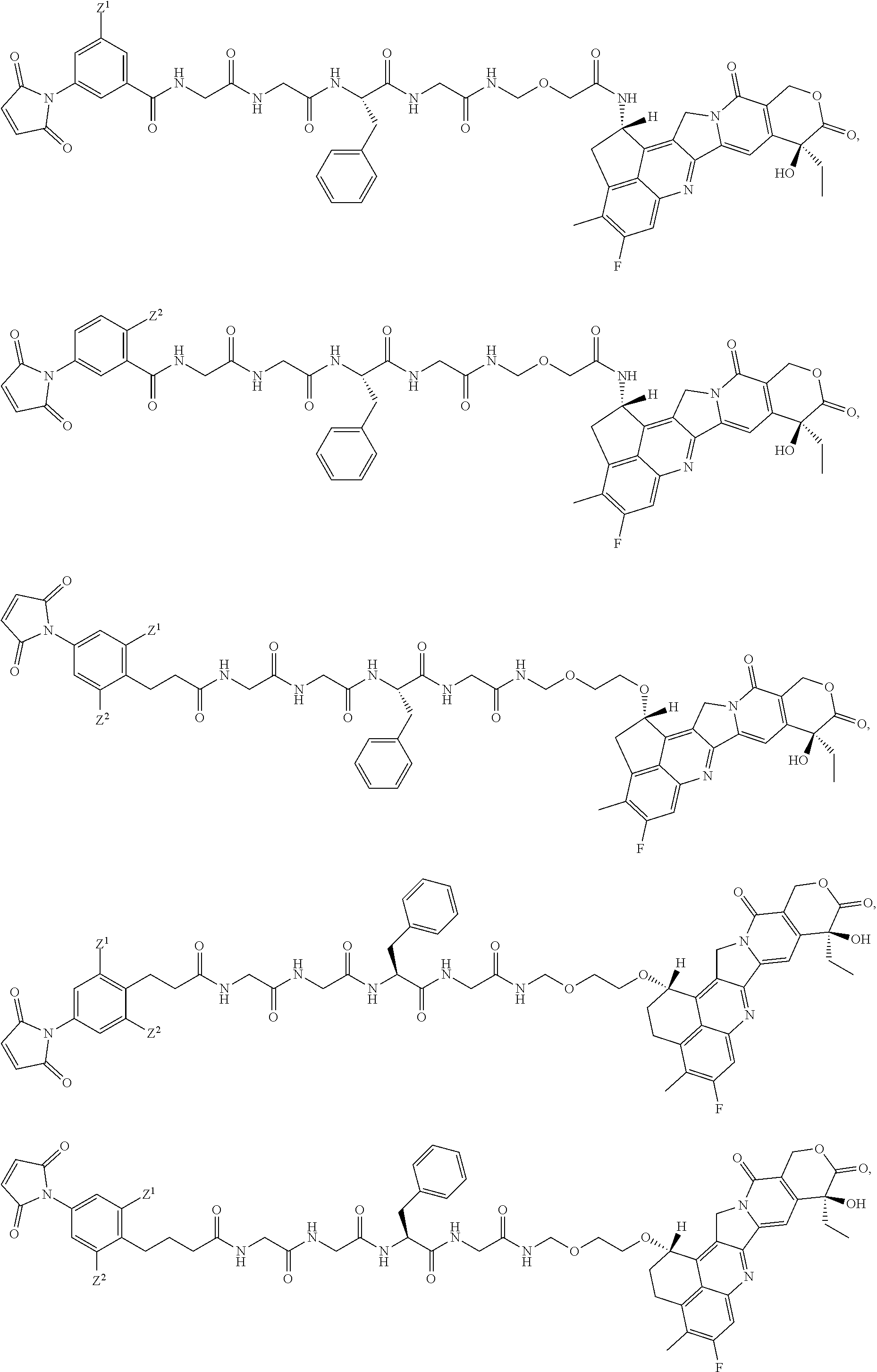

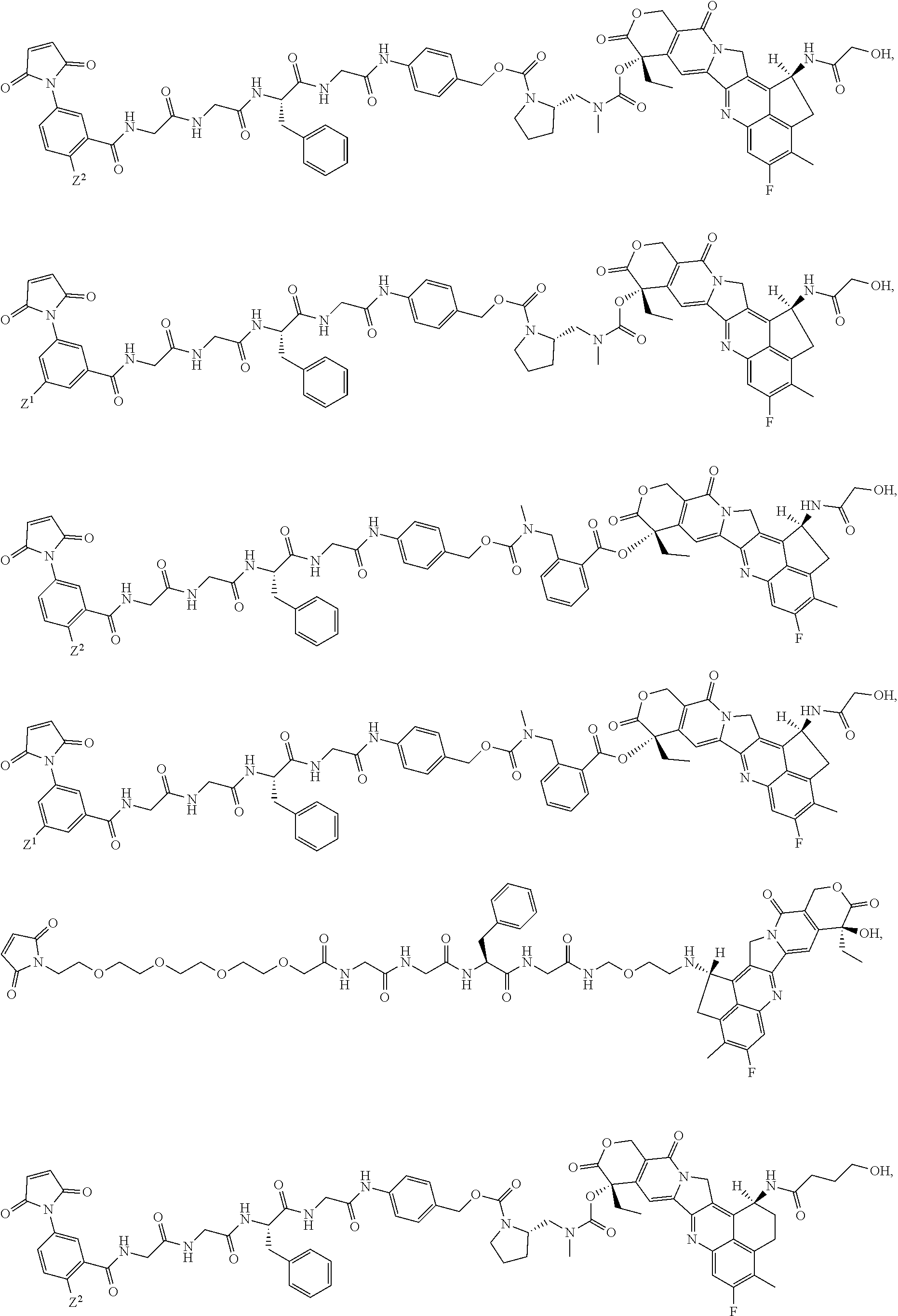

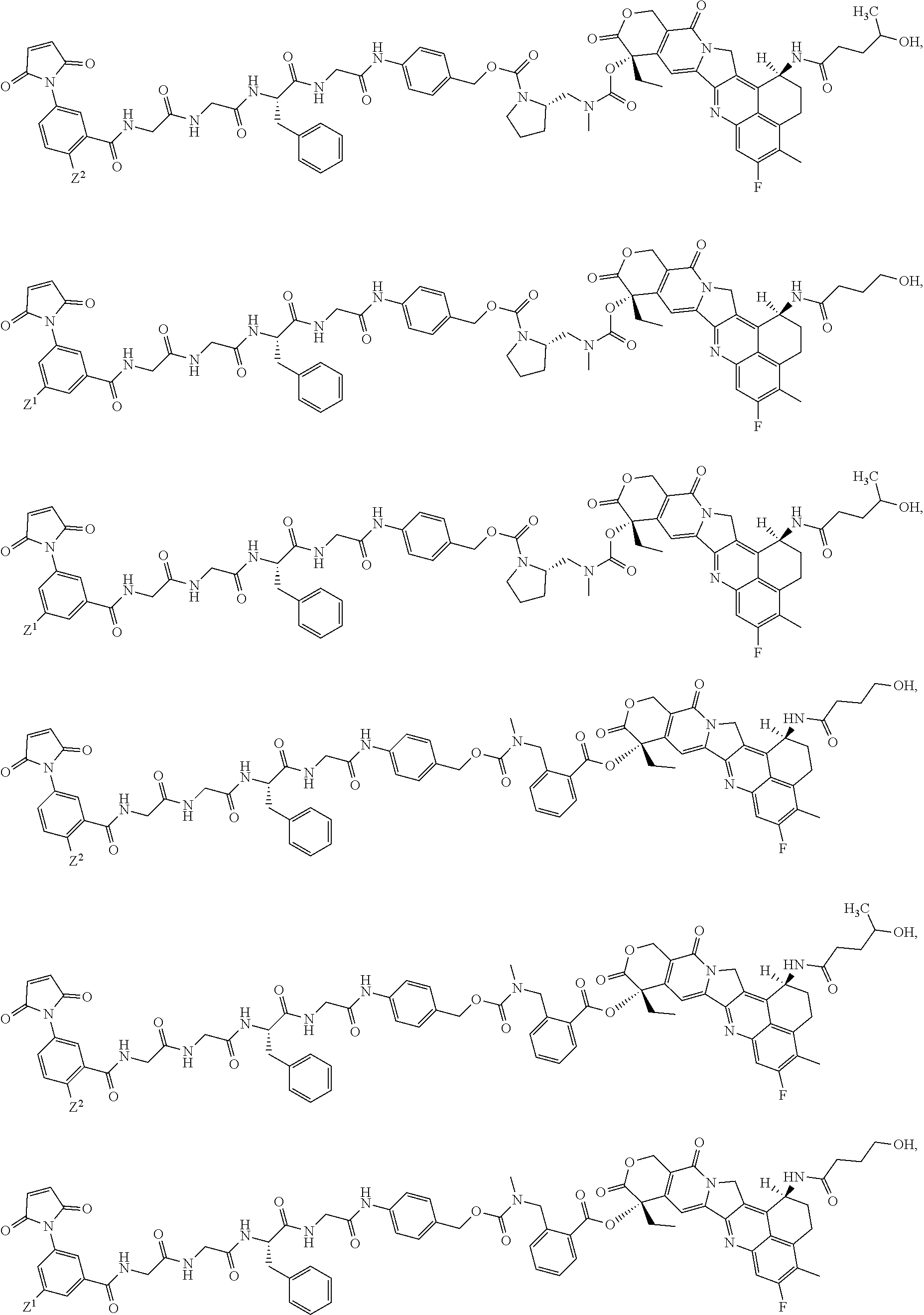

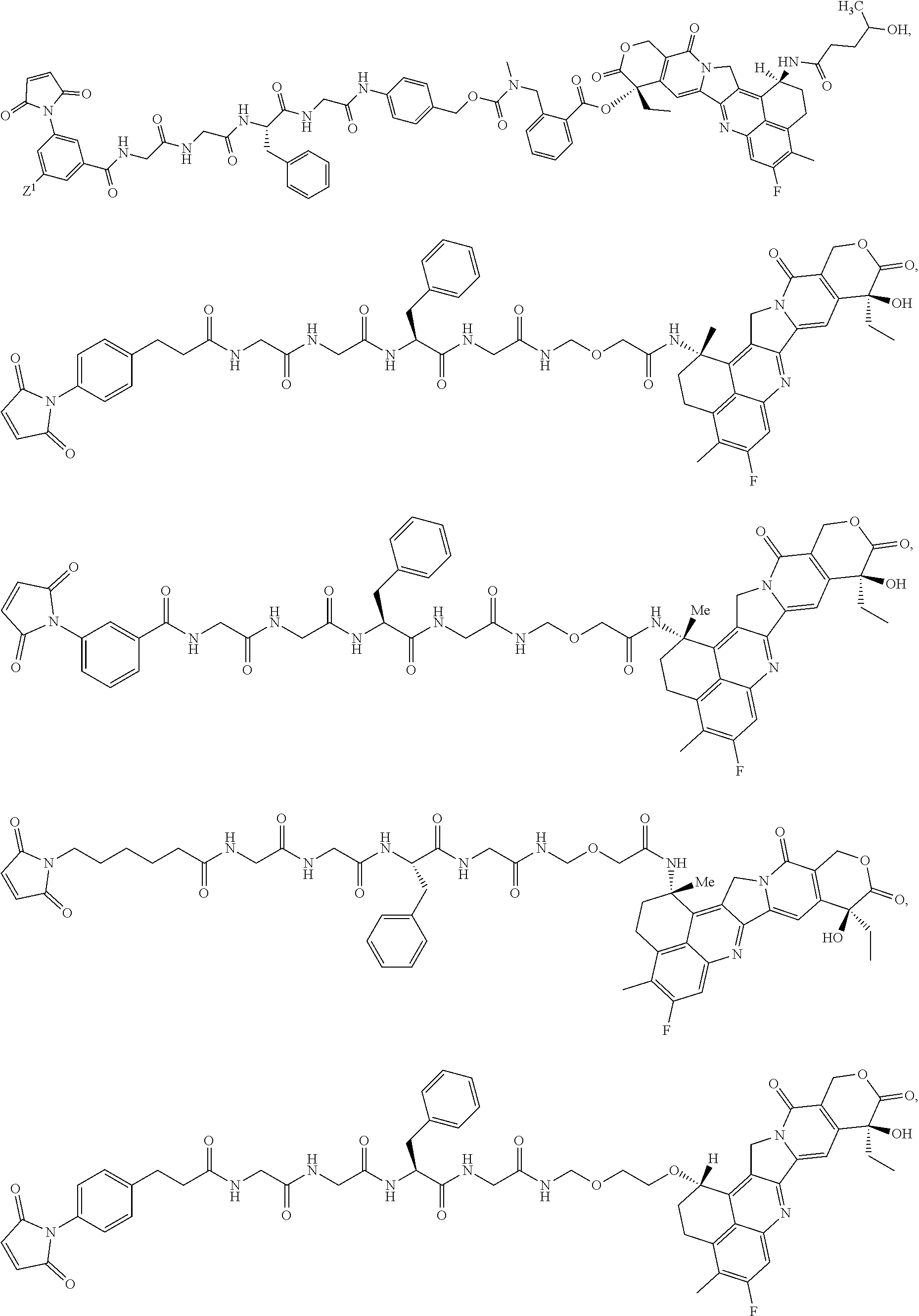

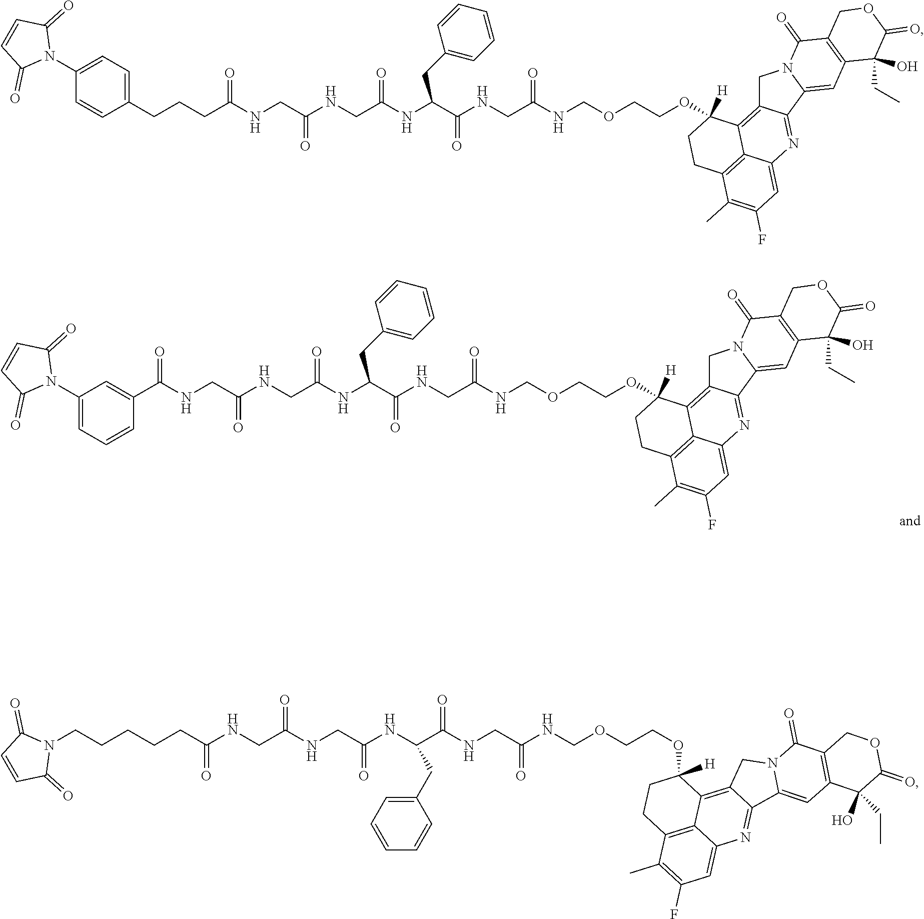

wherein Z¹ and Z² are each individually selected from hydrogen, fluoro, chloro, —NO₂, and —OCH₃.

In some embodiments, a conjugate of Formula (III), cannot be selected from:

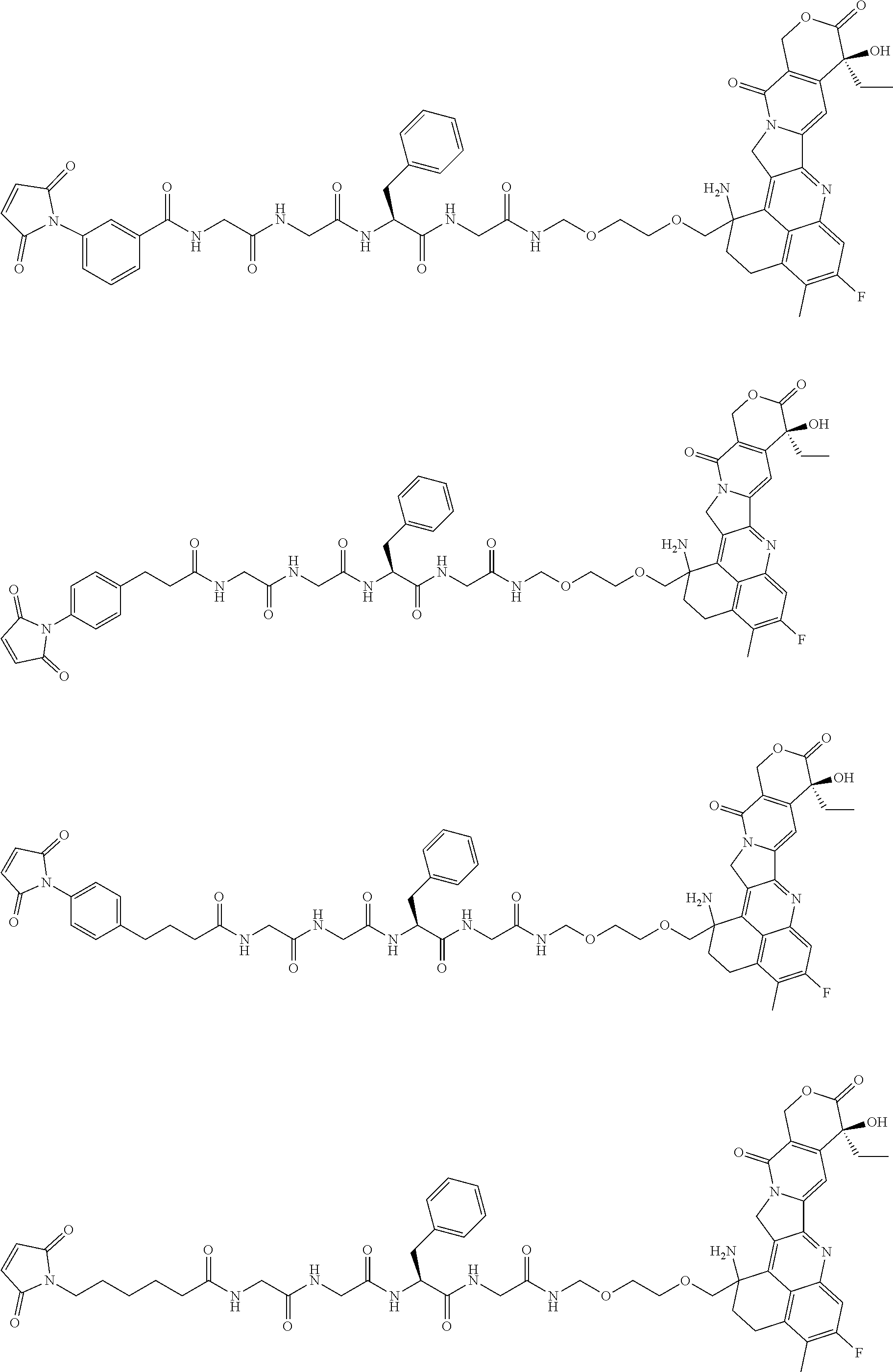

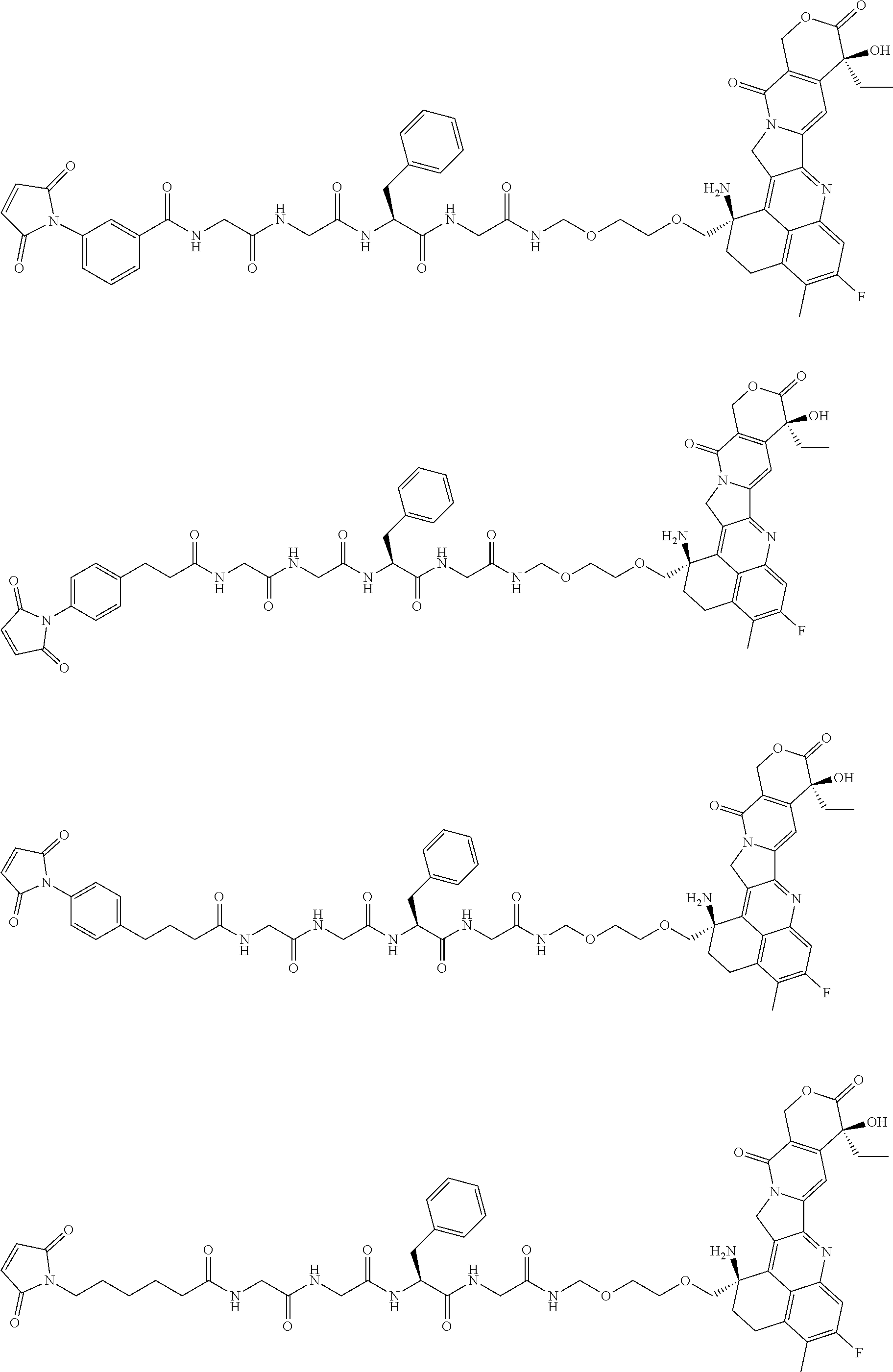

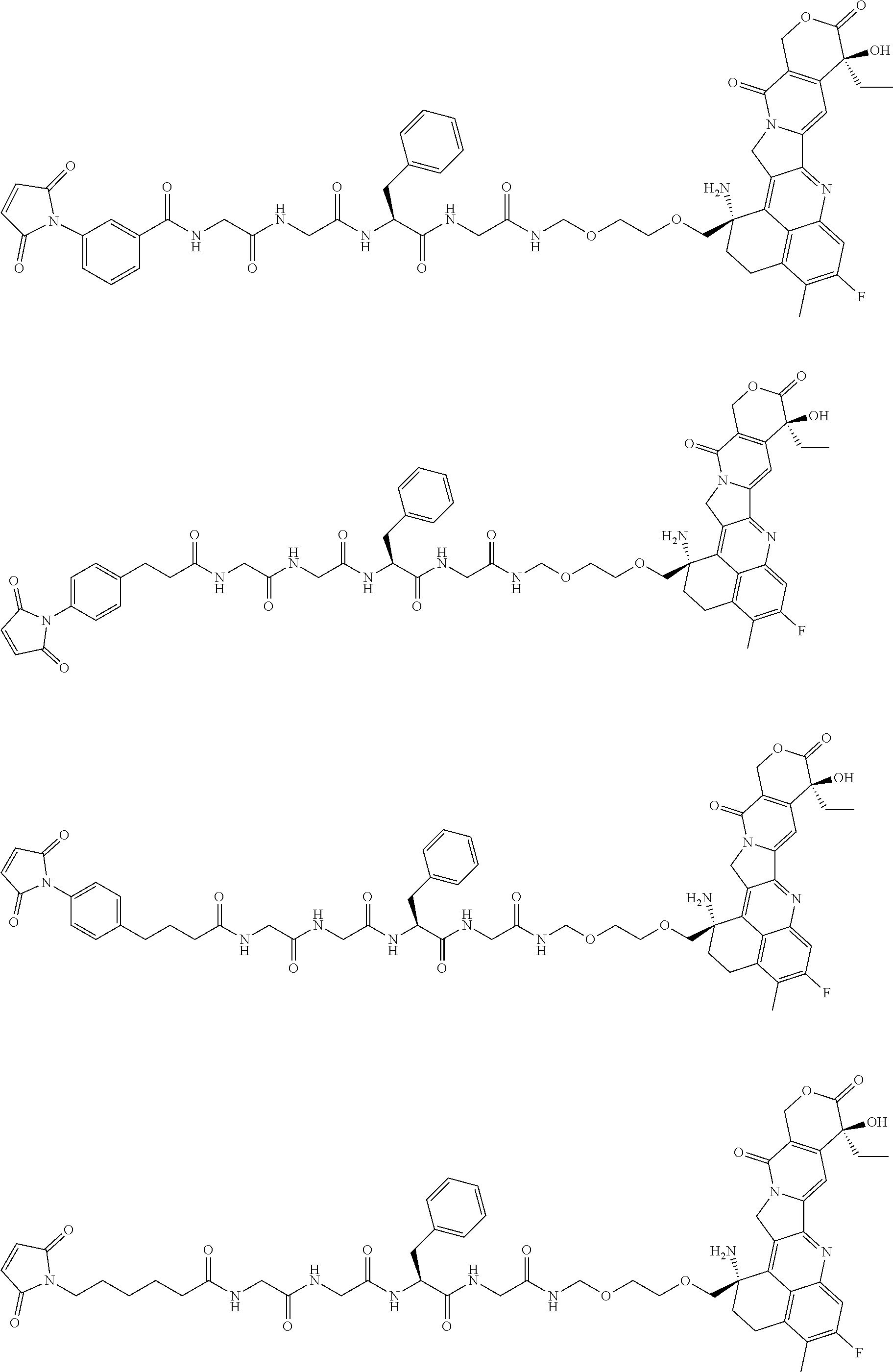

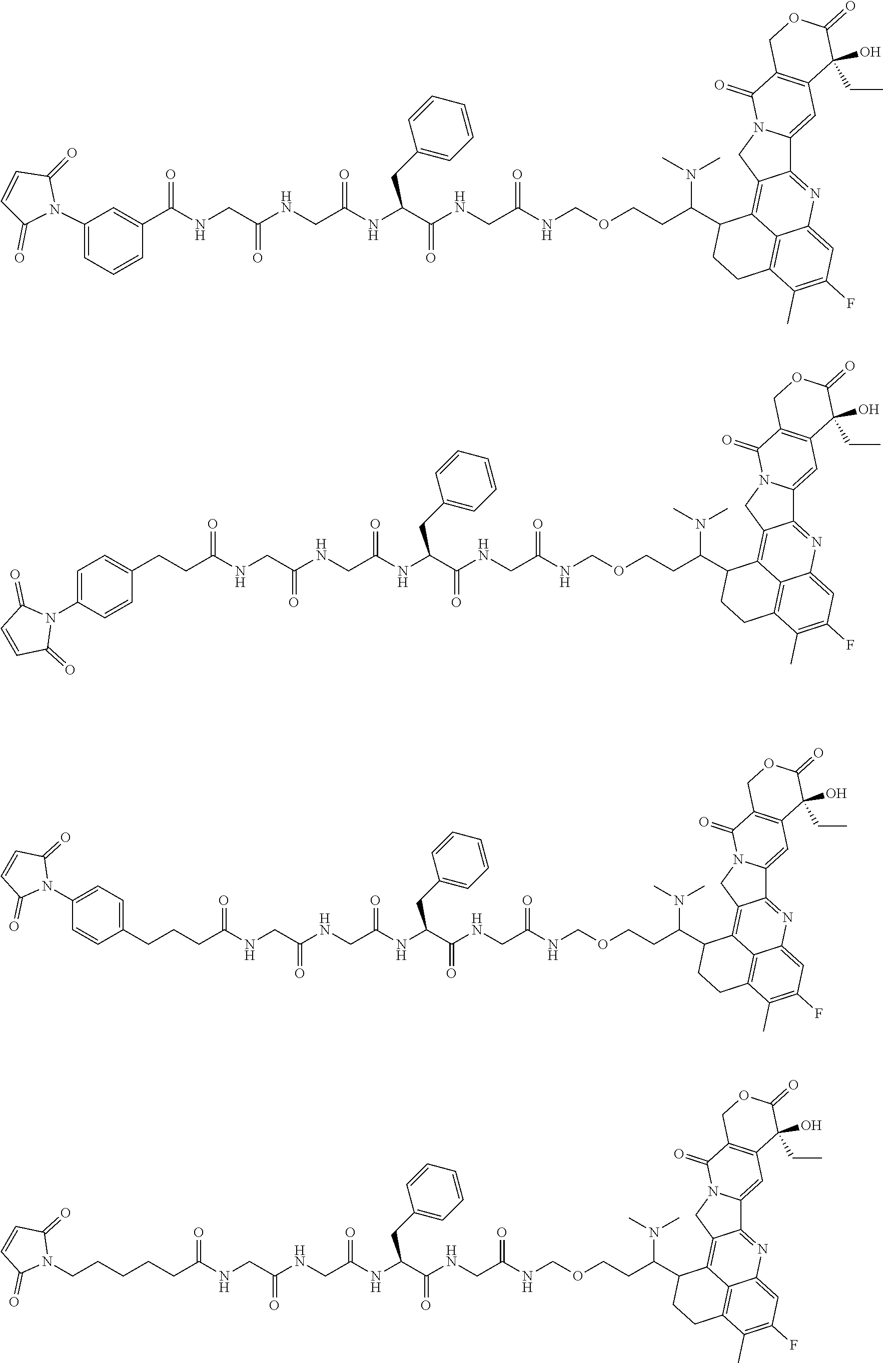

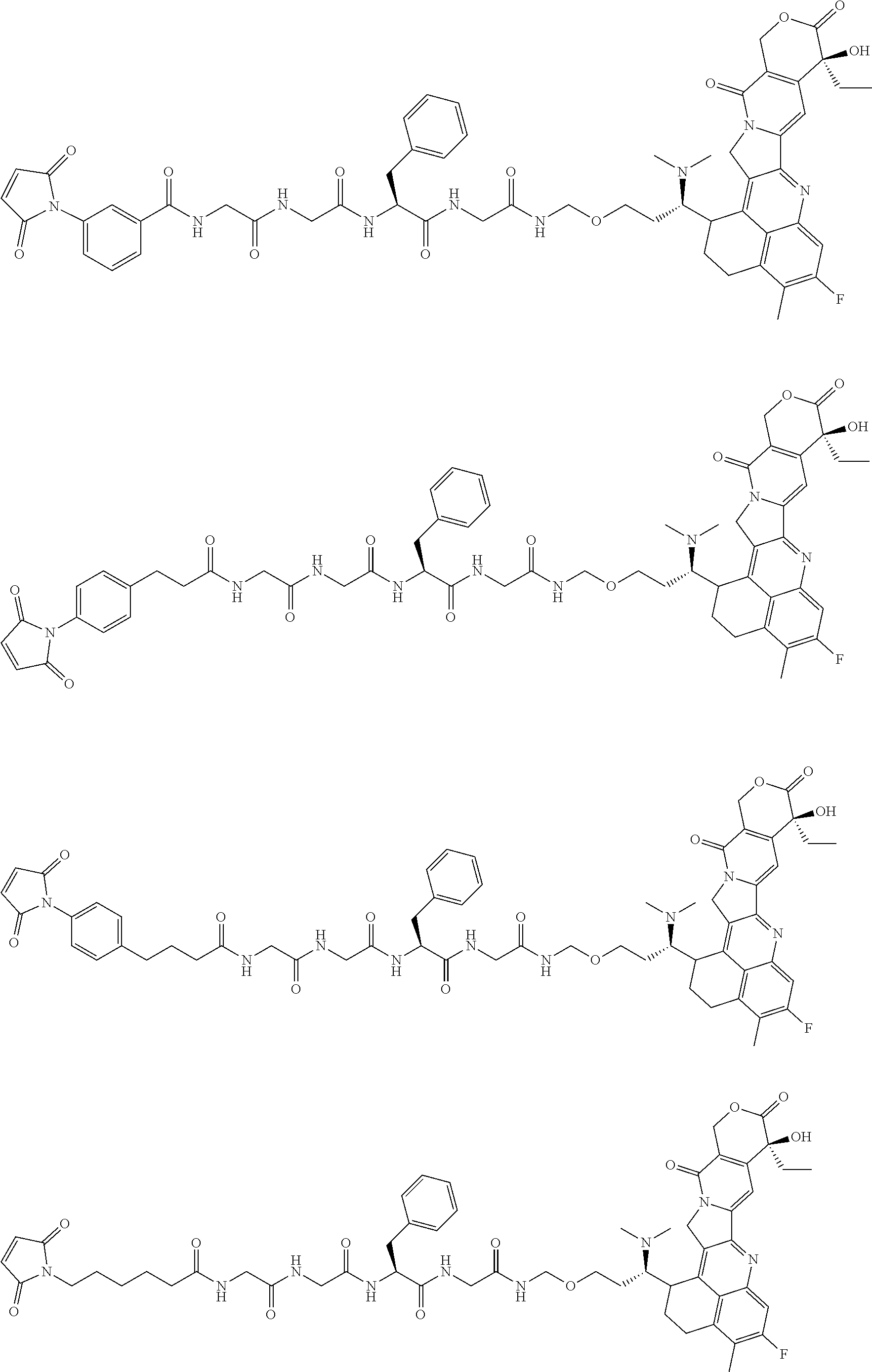

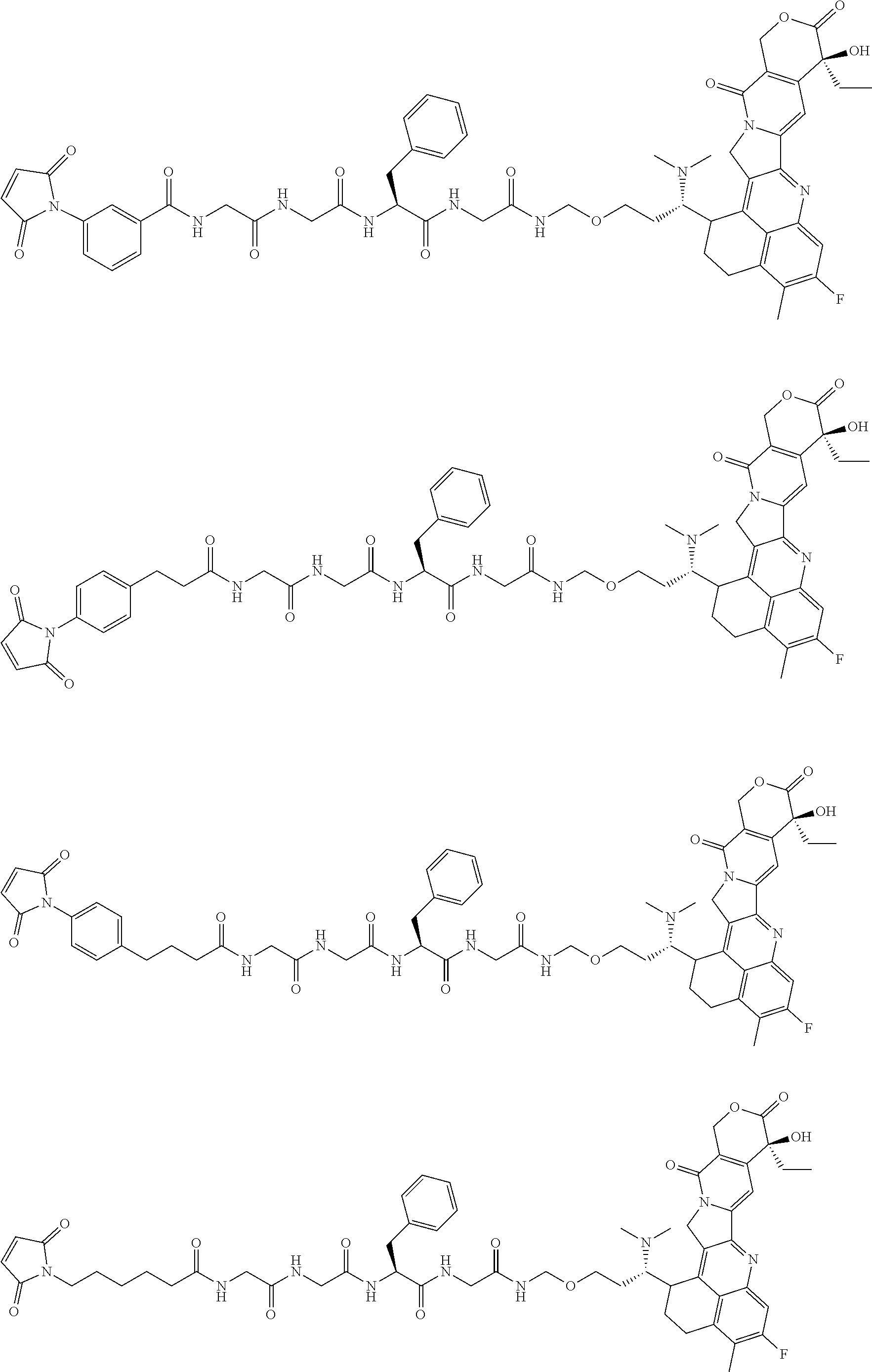

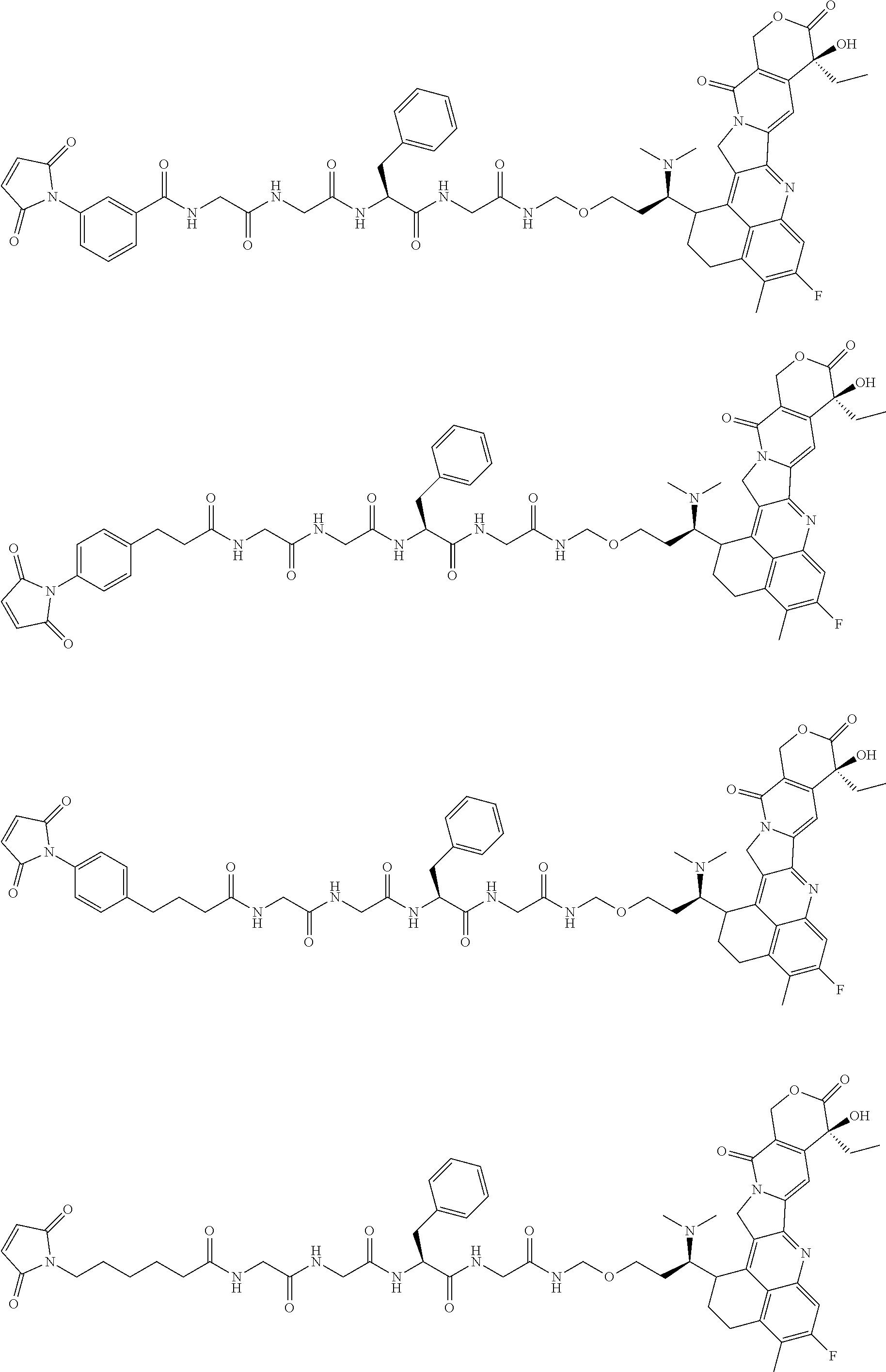

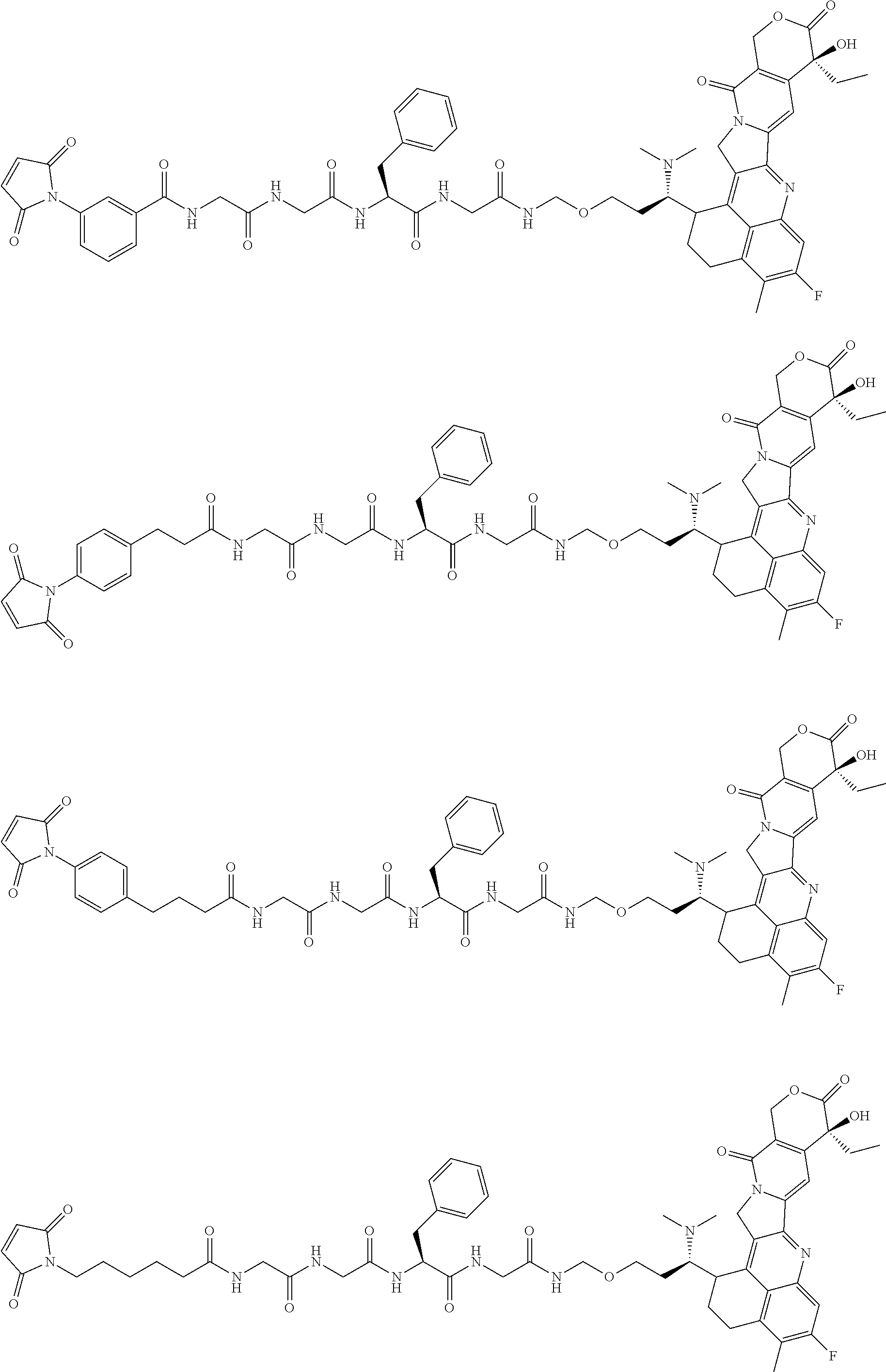

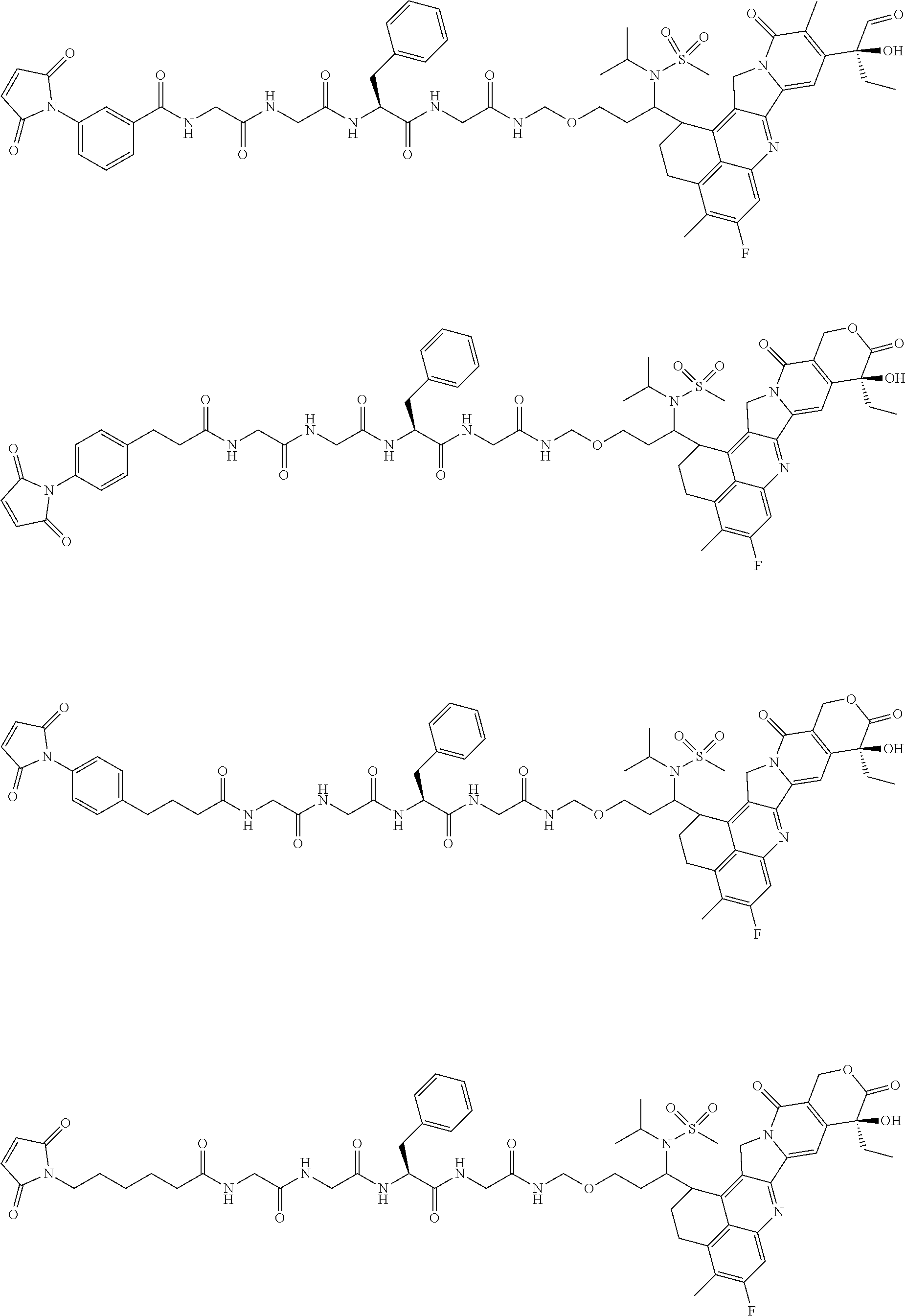

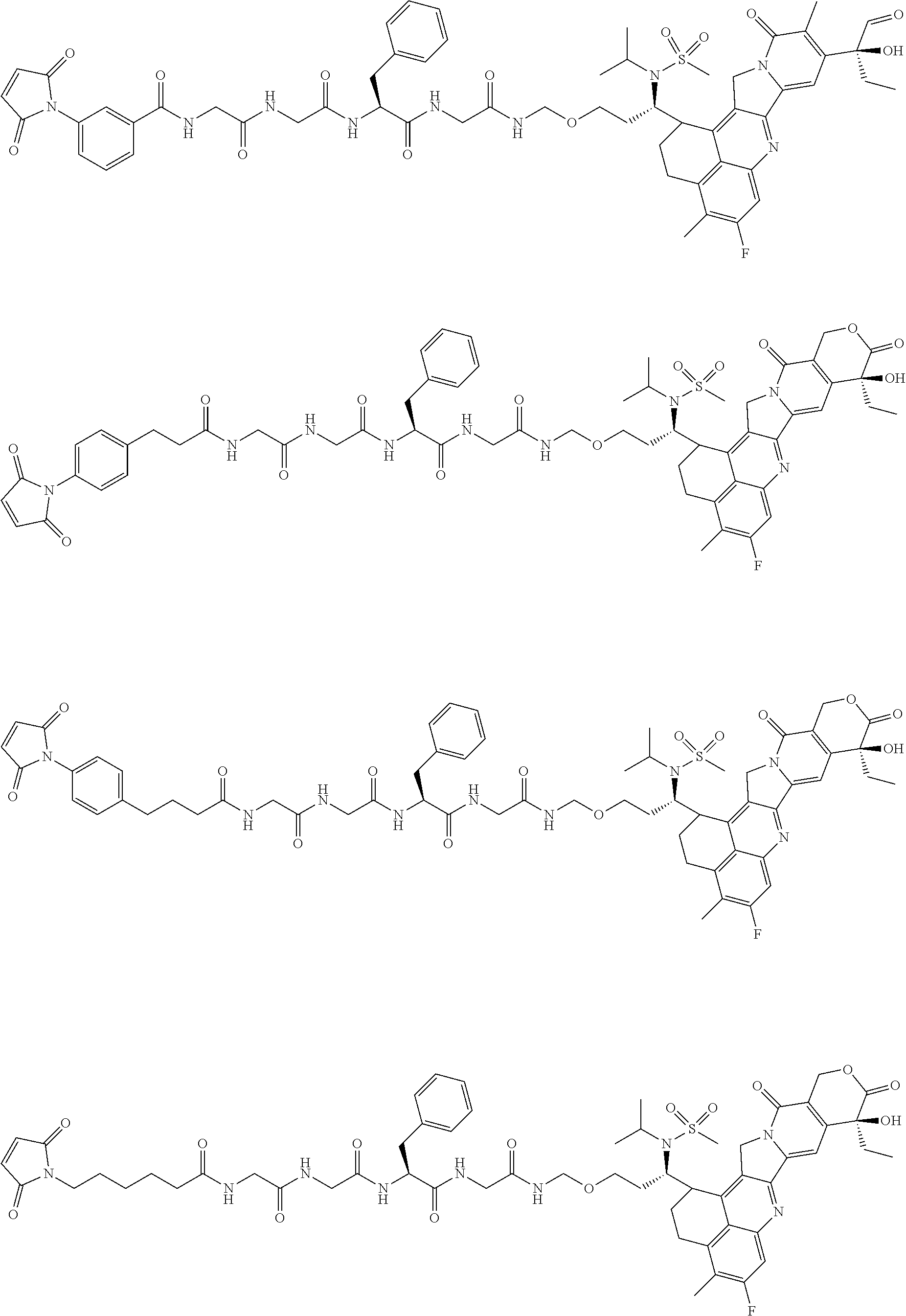

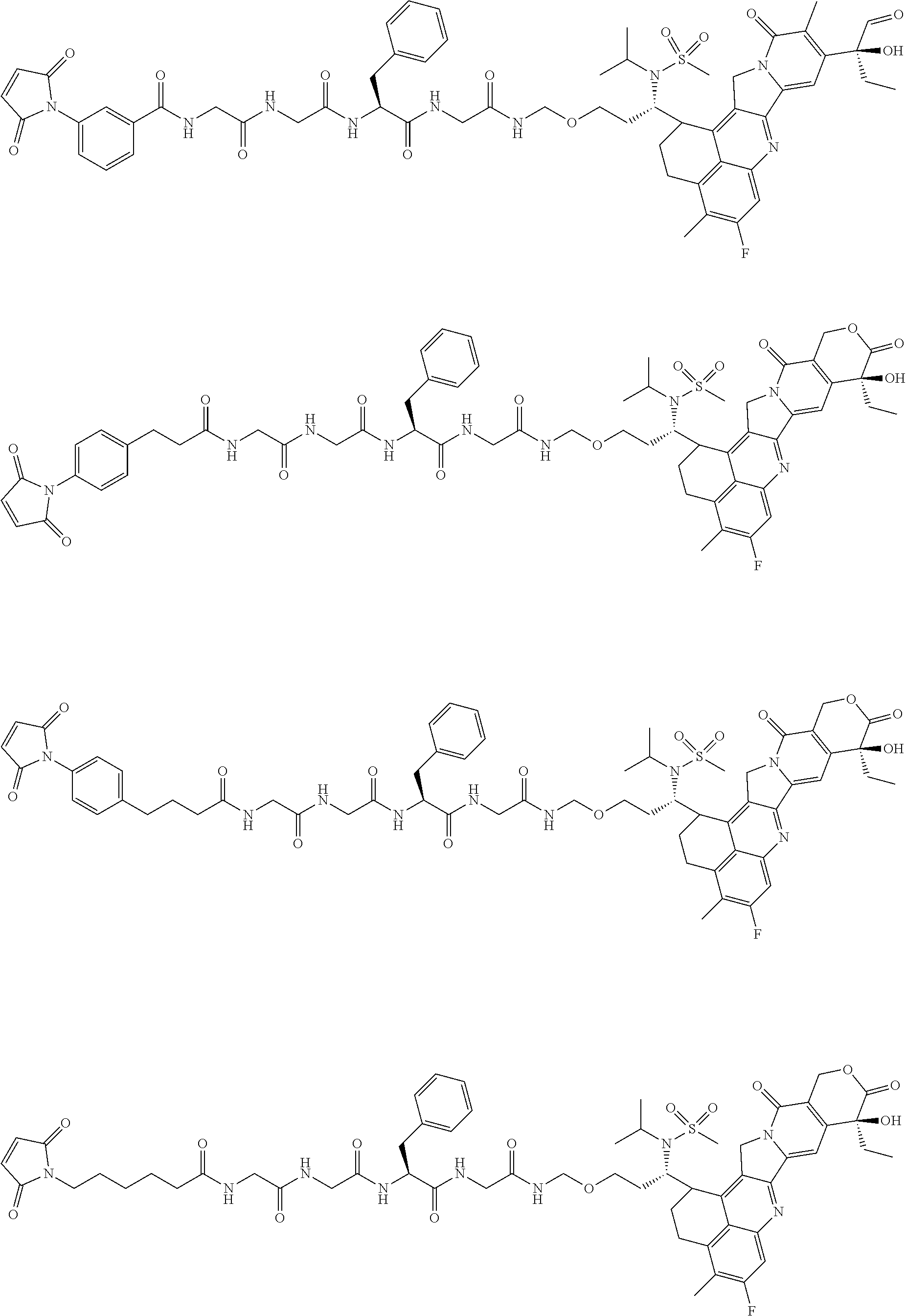

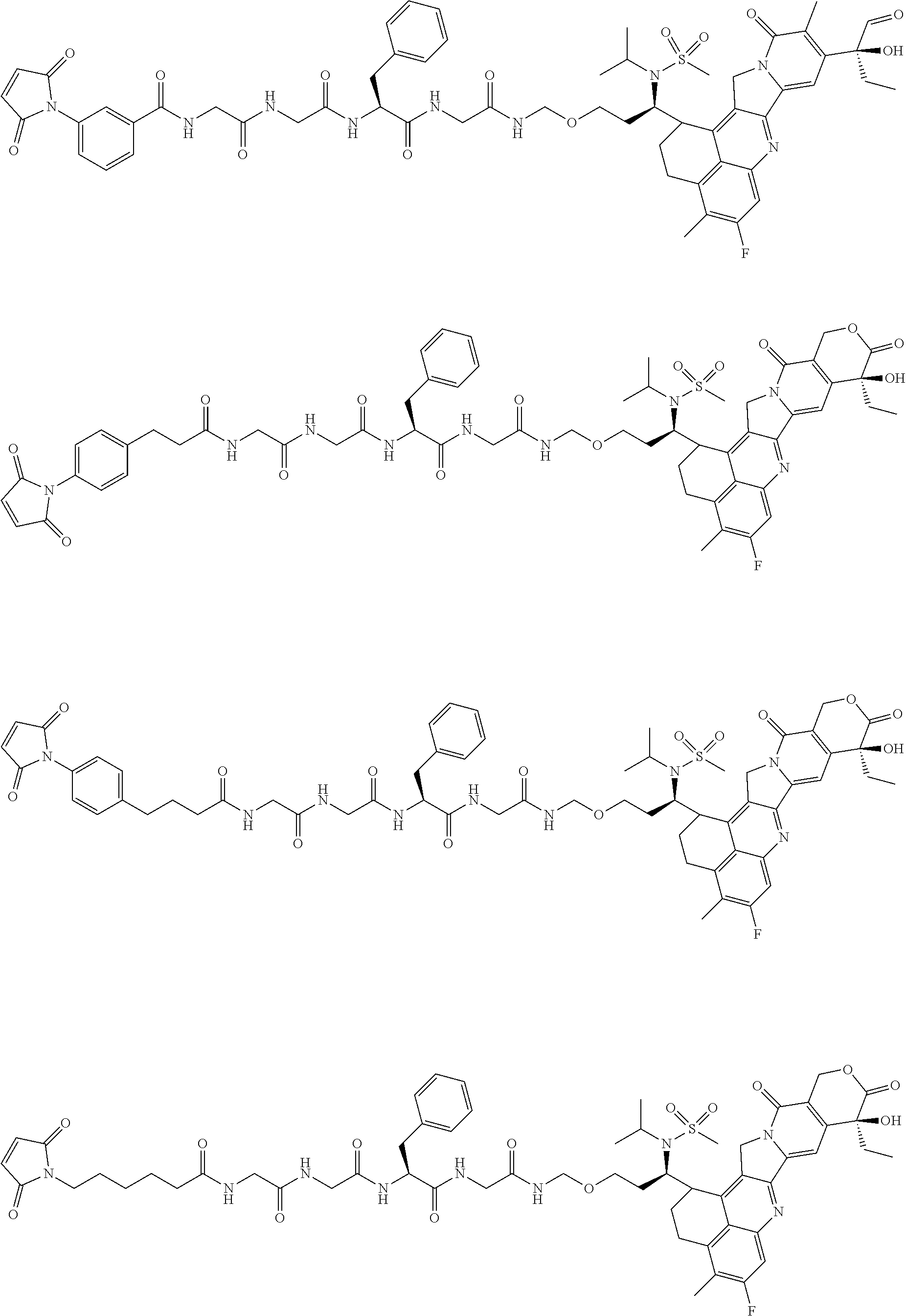

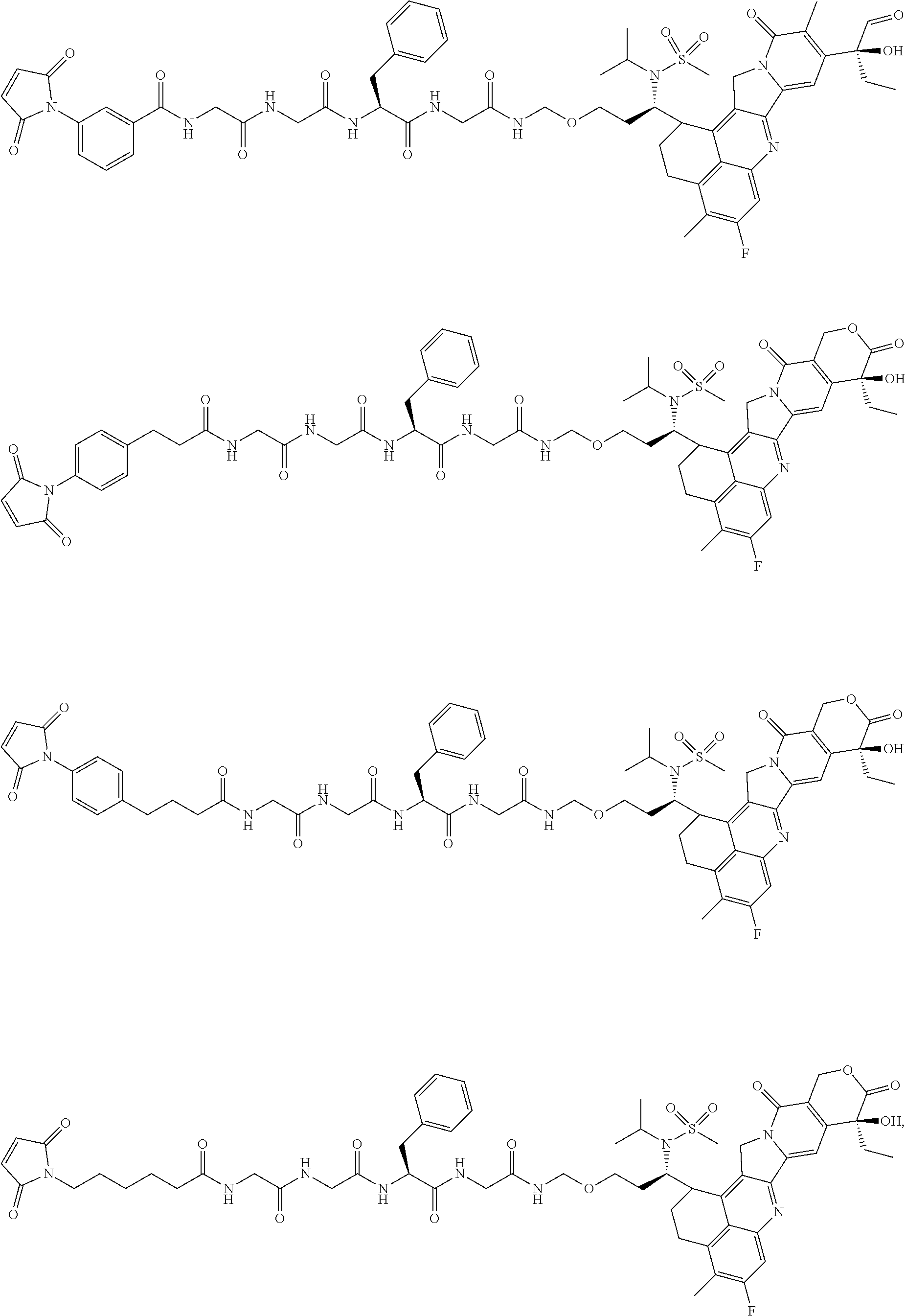

or a pharmaceutically acceptable salt of any of the foregoing.

### Drug Moieties

In various embodiments, D in the immunoconjugate of Formula (I) or in the conjugate of Formula (III) is a drug moiety. The drug moiety may be any compound of the Formula (IV) as described herein (e.g., as described above under the heading “Compounds”), with appropriate modification so that the linker-L²-L³-L⁴-L⁵-L⁶-L⁷-connects to D. For example, in various embodiments the drug moiety D is a compound of Formula (II), or a pharmaceutically acceptable salt thereof, having the structure:

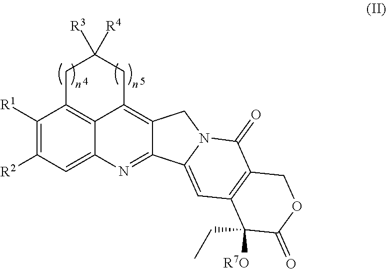

Those skilled in the art will appreciate that the compound of Formula (II) connects to the linker-L²-L³-L⁴-L⁵-L⁶-L⁷- via R³ or R⁴ (when defined to include X² and thus R⁹) or via R⁷ as described below.

In various embodiments, R¹ and R² in Formula (II) are each individually selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, or a substituted or an unsubstituted —O—(CR⁵R⁶)ₘ—O-such that R¹ and R² taken together form a ring. In an embodiment, at least one of R¹ and R² is hydrogen. In an embodiment, at least one of R¹ and R² is halogen. For example, in an embodiment, at least one of R¹ and R² is fluoro. In an embodiment, at least one of R¹ and R² is —CN. In an embodiment, at least one of R¹ and R² is —OR³, wherein R⁵ is hydrogen, halogen, a substituted or an unsubstituted C₁-C₆ alkyl, a substituted or an unsubstituted C₁-C₆ haloalkyl, or —[(CY₂)ₚO(CY₂)_{q}]ₜCY₃. For example, in an embodiment, at least one of R¹ and R² is methoxy.

In an embodiment, at least one of R¹ and R² in Formula (II) is —NR⁵R⁶, wherein R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl.

In an embodiment, at least one of R¹ and R² is a substituted or an unsubstituted C₁-C₆ alkyl. In an embodiment, at least one of R¹ and R² is C₁-C₃ alkyl. For example, in an embodiment, at least one of R¹ and R² is methyl. In an embodiment, at least one of R¹ and R² is C₁-C₃ alkyl and the other is a halogen. For example, in an embodiment, at least one of R¹ and R² is methyl and the other is fluoro.

In an embodiment, at least one of R¹ and R² is a substituted or an unsubstituted C₁-C₆ haloalkyl. For example, in an embodiment, at least one of R¹ and R² is difluoromethyl. In an embodiment, at least one of R¹ and R² is a substituted or an unsubstituted —O—(C₁-C₆ alkyl). For example, in an embodiment, at least one of R¹ and R² is methoxy. In an embodiment, at least one of R¹ and R² is —[(CY₂)ₚO(CY₂)_{q}]ₜCY₃. In an embodiment. R¹ and R² are a substituted or an unsubstituted —O—(CR⁵R⁶)ₘ—O— such that R¹ and R² taken together form a ring in which the ends of the —O—(CR⁵R⁶)ₘ—O— are covalently bonded to the phenyl ring at the R¹ and R² positions of Formula (II) to form a heterocyclic ring.

In an embodiment, one of R¹ and R² in Formula (II) is hydrogen and the other of R¹ and R² is halogen. In an embodiment, one of R¹ and R² is hydrogen and the other of R¹ and R² is a substituted or an unsubstituted C₁-C₆ alkyl. In an embodiment, one of R¹ and R² is hydrogen and the other of R¹ and R² is a substituted or an unsubstituted C₁-C₆ haloalkyl. In an embodiment, one of R¹ and R² is hydrogen and the other of R¹ and R² is a substituted or an unsubstituted —O—(C₁-C₆ alkyl). In an embodiment, both R¹ and R² are hydrogen. In an embodiment, neither R¹ nor R² is hydrogen.

In an embodiment, one of R¹ and R² in Formula (II) is halogen and the other of R¹ and R² is a substituted or an unsubstituted C₁-C₆ alkyl. In an embodiment, one of R¹ and R² is halogen and the other of R¹ and R² is a substituted or an unsubstituted C₁-C₆ haloalkyl. In an embodiment, one of R¹ and R² is halogen and the other of R¹ and R² is a substituted or an unsubstituted —O—(C₁-C₆ alkyl). In an embodiment, both R¹ and R² are independently halogen. In an embodiment, neither R¹ nor R² is halogen.

In an embodiment, one of R¹ and R² in Formula (II) is a substituted or an unsubstituted C₁-C₆ alkyl and the other of R¹ and R² is a substituted or an unsubstituted C₁-C₆ haloalkyl. In an embodiment, one of R¹ and R² is a substituted or an unsubstituted C₁-C₆ alkyl and the other of R¹ and R² is a substituted or an unsubstituted —O—(C₁-C₆ alkyl). In an embodiment, both R¹ and R² are independently a substituted or an unsubstituted C₁-C₆ alkyl. In an embodiment, neither R¹ nor R² is a substituted or an unsubstituted C₁-C₆ alkyl.

In an embodiment, one of R¹ and R² in Formula (II) is a substituted or an unsubstituted C₁-C₆ haloalkyl and the other of R¹ and R² is a substituted or an unsubstituted —O—(C₁-C₆ alkyl). In an embodiment, both R¹ and R² are independently a substituted or an unsubstituted C₁-C₆ haloalkyl. In an embodiment, neither R¹ nor R² is a substituted or an unsubstituted C₁-C₆ haloalkyl.

In an embodiment, one of R¹ and R² in Formula (II) is a substituted or an unsubstituted —O—(C₁-C₆ alkyl). In an embodiment, both R¹ and R² are independently a substituted or an unsubstituted —O—(C₁-C₆ alkyl). In an embodiment, neither R¹ nor R² is a substituted or an unsubstituted —O—(C₁-C₆ alkyl). In an embodiment, R¹ and R² are a substituted or an unsubstituted —O—(CR⁵R⁶)ₘ—O— such that R¹ and R² taken together form a ring. In various embodiments, R¹ and R² are each individually selected from the group consisting of hydrogen, fluoro, methoxy, methyl, difluoromethyl, and O—(CH₂)—O— such that R¹ and R² taken together form a ring.

In various embodiments, R³ in Formula (II) is hydrogen, —OH, —N₃, —NH₂, —NH(C═O)CH₃, a substituted or an unsubstituted C₁-C₆ alkyl, a substituted or an unsubstituted C₂-C₆ alkenyl or [(CY₂)ₚO(CY₂)_{q}]ₜOH, with the proviso that R³ and R⁴ are not both hydrogen. In an embodiment, R³ is —OH. In an embodiment, R³ is —N₃. In an embodiment, R³ is —NH₂. In an embodiment, R³ is —NH(C═O)—CH₂—R^{3D}, wherein R^{3D} can be selected from H, —CH₃, —OH and —CH₂Y¹, wherein Y¹ is halogen. For example, R³ can be —NH(C═O)—CH₃, —NH(C═O)—CH₂CH₃, —NH(C═O)—CH₂OH, —NH(C═O)—CH₂CH₂—Y¹. In some embodiments, Y¹ can be F or Cl. In an embodiment, R³ is an unsubstituted C₁-C₆ alkyl. In an embodiment. R³ is a substituted C₁-C₆ alkyl. Examples of C₁-C₆ alkyls include methyl, ethyl, n-propyl, iso-propyl, n-butyl, iso-butyl, sec-butyl, tert-butyl, pentyl (straight-chained or branched) and hexyl (straight-chained or branched). In an embodiment, R³ is a substituted C₂-C₆ alkenyl. In an embodiment, R³ is an unsubstituted C₂-C₆ alkenyl. When R³ is a substituted C₁-C₆ alkyl or a substituted C₂-C₆ alkenyl, the C₁-C₆ alkyl and/or C₂-C₆ alkenyl can be substituted with one or more R^{3A} groups (such as 1, 2 or 3 R^{3A} groups) individually selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl). In some embodiments, R³ is a substituted C₁-C₆ alkyl substituted by —OH and —NR^{3B}R^{3C}. For example, in an embodiment. R³ is methyl, —CH₂OH, —CH₂CH₂OH, —CH₂CH(OH)CH₂OH, —CH(NH₂))(CH₂OH), —CH(NH₂)(CH₂CH₂OH), —CH(NH(CH₃))(CH₂OH), —CH(NH(CH₃)(CH₂CH₂OH), —CH(N(CH₃)₂)(CH₂OH), —CH(N(CH₃)₂)(CH₂CH₂OH), —CH(NH(isopropyl))(CH₂OH), —CH(NH(isopropyl))(CH₂CH₂OH), —CH₂CH═CH₂, —CH(NH—(C(═O)CH₃)(CH₂OH), —CH(NH—(C(═O)CH₃))(CH₂CH₂OH) and —CH(NH—(C(═O)CH₃)(CH₂CH═CH₂). In some embodiments, R³ is a substituted C₁-C₆ alkyl substituted by —OH and —NR^{3B}R^{3C}. In some embodiments, R³ is a substituted C₁-C₆ alkyl substituted —C(═O) (an unsubstituted C₁-C₆ alkyl). Examples of suitable C₁-C₆ alkyls are described herein. In some embodiments. R³ is a substituted C₁-C₆ alkyl substituted by one or more OH groups (such as 1, 2, 3 or 4 OH groups). In an embodiment. R³ is —[(CY₂)ₚO(CY₂)_{q}]ₜOH. Exemplary-[(CY₂)ₚO(CY₂)_{q}]ₜOH groups for R³ include-CH₂OCH₂CH₂OH.

In various embodiments, R⁴ in Formula (II) is hydrogen, —OH, —N₃, —NH₂, —NH(C═O)CH₃, a substituted or an unsubstituted C₁-C₆ alkyl, a substituted or an unsubstituted C₂-C₆ alkenyl or [(CY₂)ₚO(CY₂)_{q}]ₜOH, with the proviso that R³ and R⁴ are not both hydrogen. In an embodiment, R⁴ is hydrogen. In an embodiment, R⁴ is —OH. In an embodiment, R⁴ is —N₃. In an embodiment, R⁴ is —NH₂. In an embodiment, R⁴ is —NH(C═O)—CH₂—R^{3D}, wherein R^{3D} can be selected from H, —CH₃, —OH and —CH₂Y¹, wherein Y¹ is halogen. For example, R⁴ can be —NH(C═O)—CH₃, —NH(C═O)—CH₂CH₃, —NH(C═O)—CH₂OH, —NH(C═O)—CH₂CH₂—Y¹. In some embodiments, Y¹ can be F or Cl. In an embodiment, R⁴ is an unsubstituted C₁-C₆ alkyl. In an embodiment, R⁴ is a substituted C₁-C₆ alkyl. Examples of C₁-C₆ alkyls include methyl, ethyl, n-propyl, iso-propyl, n-butyl, iso-butyl, sec-butyl, tert-butyl, pentyl (straight-chained or branched) and hexyl (straight-chained or branched). In an embodiment, R⁴ is a substituted C₂-C₆ alkenyl. In an embodiment, R⁴ is an unsubstituted C₂-C₆ alkenyl. When R⁴ is a substituted C₁-C₆ alkyl or a substituted C₂-C₆ alkenyl, the C₁-C₆ alkyl and/or C₂-C₆ alkenyl can be substituted with one or more R^{3A} groups (such as 1, 2 or 3 R^{3A} groups) individually selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl). In some embodiments, R⁴ is a substituted C₁-C₆ alkyl substituted by —OH and —NR^{3B}R^{3C}. For example, in an embodiment. R⁴ is methyl, —CH₂OH, —CH₂CH₂OH, —CH₂CH(OH)CH₂OH, —CH(NH₂))(CH₂OH), —CH(NH₂)(CH₂CH₂OH), —CH(NH(CH₃))(CH₂OH), —CH(NH(CH₃))(CH₂CH₂OH), —CH(N(CH₃)₂)(CH₂OH), —CH(N(CH₃)₂)(CH₂CH₂OH), —CH(NH(isopropyl))(CH₂OH), —CH(NH(isopropyl))(CH₂CH₂OH), —CH₂CH═CH₂, —CH(NH—(C(═O)CH₃))(CH₂OH), —CH(NH—(C(═O)CH₃))(CH₂CH₂OH) and —CH(NH—(C(═O)CH₃))(CH₂CH═CH₂). In some embodiments. R⁴ is a substituted C₁-C₆ alkyl substituted by —OH and —NR^{3B}R^{3C}. In some embodiments, R⁴ is a substituted C₁-C₆ alkyl substituted —C(═O) (an unsubstituted C₁-C₆ alkyl). Examples of suitable C₁-C₆ alkyls are described herein. In some embodiments. R⁴ is a substituted C₁-C₆ alkyl substituted by one or more OH groups (such as 1, 2, 3 or 4 OH groups). In an embodiment. R⁴ is —[(CY₂)ₚO(CY₂)_{q}]ₜOH. Exemplary —[(CY₂)ₚO(CY₂)_{q}]ₜOH groups for R⁴ include-CH₂OCH₂CH₂OH.

In some embodiments, one of R³ and R⁴ in Formula (II) is hydrogen, and the other of R³ and R⁴ is a substituted or an unsubstituted C₁-C₆ alkyl. In some embodiments, one of R³ and R⁴ in Formula (II) is hydrogen, and the other of R³ and R⁴ is a substituted C₁-C₆ alkyl. In some embodiments, one of R³ and R⁴ in Formula (II) is hydrogen, and the other of the other of R³ and R⁴ is a substituted C₁-C₆ alkyl substituted by —OH and —NR^{3B}R^{3C}, wherein R^{3B} and R^{3C} are each individually hydrogen, a substituted or an unsubstituted C₁-C₆ alkyl or C(═O) (an unsubstituted C₁-C₆ alkyl). In some embodiments, one of R³ and R⁴ in Formula (II) is hydrogen, and the other of R³ and R⁴ is a substituted or an unsubstituted C₂₋₆ alkenyl. In other embodiments, one of R³ and R⁴ in Formula (II) is —N₃, and the other of R³ and R⁴ is a substituted or an unsubstituted C₂₋₆ alkenyl. In still other embodiments, one of R³ and R⁴ in Formula (II) is —OH, and the other of R³ and R⁴ is a substituted or an unsubstituted C₂₋₆ alkenyl. In yet still other embodiments, one of R³ and R⁴ in Formula (II) is —NH₂, and the other of R³ and R⁴ is a substituted or an unsubstituted C₂₋₆ alkenyl. For example, one of R³ and R⁴ in Formula (II) is —OH—N₃, —NH₂ or —NH(C═O)—CH₂—R^{3D}, and the other of R³ and R⁴ is —CH₂CH—CH₂. In some embodiments, one of R³ and R⁴ in Formula (II) is hydrogen, and the other of the other of R³ and R⁴ is —CH(NH₂)(CH₂OH), —CH(NH₂)(CH₂CH₂OH), —CH(NH(CH₃)(CH₂OH), —CH(NH(CH₃)(CH₂CH₂OH), —CH(N(CH₃)₂)(CH₂OH), —CH(N(CH₃)₂)(CH₂CH₂OH), —CH(NH(isopropyl))(CH₂OH), —CH(NH(isopropyl))(CH₂CH₂OH), —CH(NH—(C(═O)CH₃))(CH₂OH), —CH(NH—(C(═O)CH₃))(CH₂CH₂OH) or —CH(NH—(C(═O)CH₃))(CH₂CH═CH₂). In some embodiments, one of R³ and R⁴ in Formula (II) is —OH, and the other of R³ and R⁴ is a substituted or an unsubstituted C₁-C₆ alkyl. In other embodiments, one of R³ and R⁴ in Formula (II) is —NH₂, and the other of R³ and R⁴ is a substituted or an unsubstituted C₁-C₆ alkyl. In still other embodiments, one of R³ and R⁴ in Formula (II) is —NH(C═O)—CH₂—R^{3D}, and the other of R³ and R⁴ is a substituted or an unsubstituted C₁-C₆ alkyl. In some embodiments, one of R³ and R⁴ in Formula (II) is —OH, and the other of R³ and R⁴ is a hydroxy-substituted C₁-C₆ alkyl, such as —CH₂OH, —CH₂CH₂OH and —CH₂CH(OH)CH₂OH. In other embodiments, one of R³ and R⁴ in Formula (II) is —NH₂, and the other of R³ and R⁴ is a hydroxy-substituted C₁-C₆ alkyl, such as CH₂OH, CH₂CH₂OH and —CH₂CH(OH)CH₂OH. As provided herein, in some embodiments, one or more hydroxy groups can be present on a hydroxy-substituted C₁-C₆ alkyl, such as 1, 2, 3 or 4 OH groups. In some embodiments, when R³ is —OH, then R⁴ cannot be an unsubstituted C₁₋₆ alkyl, such as methyl; and when R⁴ is —OH, then R³ cannot be an unsubstituted C₁₋₆ alkyl, such as methyl. In some embodiments, when R³ is —NH₂, then R⁴ cannot be an unsubstituted C₁₋₆ alkyl, such as methyl, and R³ is —NH₂, then R⁴ cannot be an unsubstituted C₁₋₆ alkyl, such as methyl. In some embodiments, one of R³ and R⁴ is CH₃, with the proviso that R³ and R⁴ are not both-CH₃. In some embodiments, R³ and R⁴ are not each an unsubstituted C₁₋₆ alkyl, for example, CH₃. In some embodiment, R³ and/or R⁴ cannot be selected from —CH₂OH, —CH₂CH₂OH and —CH₂CH₂CH₂OH. In some embodiments of this paragraph. R¹ can be a substituted or an unsubstituted C₁-C₆ alkyl (for example, methyl, ethyl, n-propyl, iso-propyl, n-butyl, iso-butyl, sec-butyl, tert-butyl, pentyl (straight-chained or branched) and hexyl (straight-chained or branched)); and R² can be halogen. In some embodiments of this paragraph. R¹ can be an unsubstituted C₁-C₆ alkyl; and R² can be halogen (such as F or Cl). In some embodiments of this paragraph, R⁷ can be H.

In some embodiments, one of R³ and R⁴ is a substituted or an unsubstituted —(C₁-C₆ alkyl)-X², wherein when the —(C₁-C₆ alkyl)-X² is substituted, the —(C₁-C₆ alkyl)-X² is substituted with one or more R^{3A} groups (such as 1, 2 or 3 R^{3A} groups) selected from —OH and —NR^{3B}R^{3C}, wherein R^{3B} and R^{3C} are each individually hydrogen, a substituted or an unsubstituted C₁-C₆ alkyl or —C(—O) (an unsubstituted C₁-C₆ alkyl). In other embodiments, one of R³ and R⁴ is a substituted or an unsubstituted —(C₂-C₆ alkenyl)-X², wherein when the —(C₂-C₆ alkenyl)-X² is substituted, the —(C₂-C₆ alkenyl)-X² is substituted with one or more R^{3A} groups (such as 1, 2 or 3 R^{3A} groups) selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl). In some embodiments, —NR^{3B}R^{3C} can be NH₂. In other embodiments, —NR^{3B}R^{3C} can be —NH (an unsubstituted C₁-C₆ alkyl). In still other embodiments, —NR^{3B}R^{3C} can be N (an unsubstituted C₁-C₆ alkyl) 2. In some embodiments, —NR^{3B}R^{3C} can be NH (a substituted C₁-C₆ alkyl). In other embodiments, —NR^{3B}R^{3C} can be N (a substituted C₁-C₆ alkyl) 2. In some embodiments, —NR^{3B}R^{3C} can be —NH(—C(═O) (an unsubstituted C₁-C₆ alkyl)). In other embodiments, —NR^{3B}R^{3C} can be N (an unsubstituted C₁-C₆ alkyl) (—C(═O) (an unsubstituted C₁-C₆ alkyl)). In some embodiments, one of R³ and R⁴ can be a substituted or an unsubstituted —(C₁-C₆ alkyl)-X², substituted by one or more (for example, 1, 2 or 3) hydroxys. In some embodiments, one of R³ and R⁴ can be a substituted or an unsubstituted —(C₁-C₆ alkyl)-X², substituted by —NR^{3B}R^{3C}. In some embodiments, one of R³ and R⁴ can be a substituted or an unsubstituted —(C₁-C₆ alkyl)-X², substituted by —NR^{3B}R^{3C}, and one or more (for example, 1, 2 or 3) hydroxys. In some embodiments, one of R³ and R⁴ can be a substituted or an unsubstituted —(C₂-C₆ alkenyl)-X², substituted by one or more (for example, 1, 2 or 3) hydroxys. In some embodiments, one of R³ and R⁴ can be a substituted or an unsubstituted —(C₂-C₆ alkenyl)-X², substituted by —NR^{3B}R^{3C}. In some embodiments, one of R³ and R⁴ can be a substituted or an unsubstituted —(C₂-C₆ alkenyl)-X², substituted by —NR^{3B}R^{3C} and one or more (for example, 1, 2 or 3) hydroxys. For example, one of R³ and R⁴ can be —(CH₂)—CH(OH)—(CH₂)—X². In various embodiments. X² is —OR⁹, —SR⁹, or —NHR⁹, wherein R⁹ is H, —COR⁸, —CO₂R⁸, —(CO)—NHR⁸, L⁴, L′, L⁶, or L⁷, with the proviso that exactly one of R⁷ and R⁹ is L⁴, L⁵, L⁶, or L⁷. In such embodiments, the compound of Formula (II) connects to the linker-L²-L³-L⁴-L⁵-L⁶-L⁷- via R³ or R⁴ when R³ or R⁴, respectively, includes X² and R⁹ is L⁴, L⁵, L⁶, or L⁷.

In some embodiments, a compound of Formula (II), or a pharmaceutically acceptable salt thereof, can have the structure of Formula (II-a), or a pharmaceutically acceptable salt thereof:

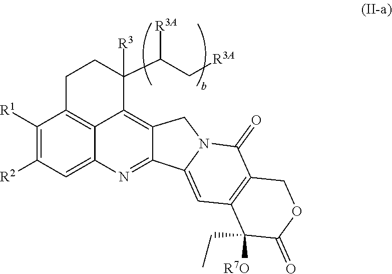

wherein:
R¹, R², R^{3A} and R⁷ are as provided herein for Formula (II);
R³ is —OH, —CH₃, —NH₂ or —NH(C═O)CH₂R^{3D}; and
b is individually 1, 2, or 3.

In some embodiments, each R^{3A} can be OH. In some embodiments of this paragraph, b can be 1. In other embodiments of this paragraph, b can be 2. In still other embodiments of this paragraph, b can be 3.

In each such embodiment in which R³ or R⁴ includes X², the option for R⁹ to be L⁴, L⁵, L⁶, or L⁷ is provided, thus providing the option to thereby connect the compound of Formula (II) to the linker-L²-L³-L⁴-L⁵-L⁶-L⁷- via R³ or R⁴.

In various embodiments, R⁷ in Formula (II) is H, —COR⁸, —CO₂R⁸, —(CO)—NHR⁸. L⁴, L⁵, L⁶, or L⁷, where each R⁸ is individually a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³. In an embodiment, R⁷ is H. In an embodiment, R⁷ is —COR⁸. In an embodiment, R⁷ is —CO₂R⁸. In an embodiment, R⁷ is —(CO)—NHR⁸. Those skilled in the art will appreciate that when R⁷ is H, —COR⁸, —CO₂R⁸, or —(CO)—NHR⁸, connection of the compound of Formula (II) to the linker-L²-L³-L⁴-L⁵-L⁶-L⁷- is via R³ or R⁴ (and thus R⁹) as described elsewhere herein.

In various embodiments, each R⁸ in Formula (II) is individually a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³, where X³ is —H, —OH, —SH, or —NH₂. In an embodiment, each R⁸ is individually a substituted or an unsubstituted C₁-C₆ alkyl-X³. In an embodiment, each R⁸ is individually a substituted or an unsubstituted C₁-C₆ haloalkyl-X³. In an embodiment, each R⁸ is individually —[(CY₂)ₚO(CY₂)_{q}]ₜCY₂—X³.

In various embodiments, X² in Formula (II) is —OR⁹, —SR⁹, or —NHR⁹, where R⁹ is H, —COR⁸, —CO₂R⁸, —(CO)—NHR⁸, L⁴, L⁵, L⁶, or L⁷. In an embodiment, X² is —OR⁹. In an embodiment, X² is —SR⁹. In an embodiment. X² is —NHR⁹.

In various embodiments, R⁹ in Formula (II) is H, —COR⁸, —CO₂R⁸, —(CO)—NHR⁸, L⁴, L⁵, L⁶, or L⁷, where R⁸ is a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³. In an embodiment, R⁹ is H. In an embodiment, R⁹ is —COR⁸. In an embodiment, R⁹ is —CO₂R⁸. In an embodiment. R⁹ is —(CO)—NHR⁸. Those skilled in the art will appreciate that when R⁹ is H, —COR⁸, —CO₂R⁸, or —(CO)—NHR⁸, connection of the compound of Formula (II) to the linker-L²-L³-L⁴-L⁵-L⁶-L⁷- is via R⁷ as described elsewhere herein.

In various embodiments, R⁹ in Formula (II) is L⁴, L⁵, L⁶, or L⁷. In an embodiment, R⁹ is L⁴. In an embodiment, R⁹ is L⁵. In an embodiment, R⁹ is L⁶. In an embodiment. R⁹ is L⁷. Those skilled in the art will appreciate that when R⁹ is L⁴, L⁵, L⁶, or L⁷, connection of the compound of Formula (II) to the linker-L²-L³-L⁴-L⁵-L⁶-L⁷- is via R³ or R⁴ as described elsewhere herein. In an embodiment, exactly one of R⁷ and R⁹ is L⁴, L⁵, L⁶, or L⁷, in which case a covalent bond links the drug D to the linker-L²-L³-L⁴-L⁵-L⁶-L⁷ and thereby to Mi.

In various embodiments, each X³ in Formula (II) is individually —H, —OH, —SH, or —NH₂. In an embodiment, X³ is —H. In an embodiment, X³ is —OH. In an embodiment, X³ is —SH. In an embodiment. X³ is —NH₂.

In various embodiments, m in Formula (II) is 1 or 2. In an embodiment, mis 1. In another embodiment, m is 2.

In various embodiments, n⁴ and n⁵ in Formula (II) are each individually 0, 1 or 2, with the proviso that n⁴ and n⁵ are not both 0). In an embodiment, n⁴ and n⁵ are both 1. In an embodiment, n⁴ is 0) and n⁵ is 1. In an embodiment, n⁴ is (and n⁵ is 2. In an embodiment, n⁴ is 1 and n⁵ is 0. In an embodiment, n⁴ is 2 and n⁵ is 0.

In various embodiments, each Y in Formula (II) is individually H or halogen. In an embodiment, each Y is hydrogen. In an embodiment, —CY₂ is —CH₂. In an embodiment, —CY₃ is —CH₃. In an embodiment, —CY₃ is —CHF₂. In an embodiment, —CY₃ is —CH₂F. In an embodiment, —CY₃ is —CF₃.

In various embodiments, each p in Formula (II) is individually 1, 2, 3, 4, 5, or 6. In an embodiment, p is 1. In an embodiment, p is 2.

In various embodiments, each q in Formula (II) is individually 0, 1, 2, 3, 4, 5, or 6. In an embodiment, q is 1. In an embodiment, q is 2.

In various embodiments, each t in Formula (II) is individually 1, 2, 3, 4, 5, or 6. In an embodiment, t is 1. In an embodiment, p is t.

In some embodiments, a compound of Formula (II) can be where: R¹ and R² are each individually selected from hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, or a substituted or an unsubstituted —O—(CR⁵R⁶)ₘ—O-such that R¹ and R² taken together form a ring; R³ and R⁴ are each individually selected from hydrogen, —OH, —N₃, —NH₂, —NH(C═O)—CH₂—R^{3D}, a substituted or an unsubstituted C₂-C₆ alkenyl and [(CY₂)ₚO(CY₂)_{q}]ₜOH, with the proviso that R³ and R⁴ are not both hydrogen, wherein when the C₁-C₆ alkyl or C₂-C₆ alkenyl is substituted, the C₁-C₆ alkyl or C₂-C₆ alkenyl is substituted with one or more R^{3A} groups selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl); or one of R³ and R⁴ is a substituted or an unsubstituted —(C₁-C₆ alkyl)-X² or a substituted or an unsubstituted —(C₂-C₆ alkenyl)-X², wherein when-(C₁-C₆ alkyl)-X² or —(C₁-C₆ alkenyl)-X² is substituted, the —(C₁-C₆ alkyl)-X² or the (C₁-C₆ alkenyl)-X² is substituted with one or more R^{3A} groups selected from —OH and —NR^{3B}R^{3C}, wherein R^{3B} and R^{3C} are each individually hydrogen, a substituted or an unsubstituted C₁-C₆ alkyl or —C(—O) (an unsubstituted C₁-C₆ alkyl); R^{3D} is selected from H, —CH₃, —OH and —CH₂Y¹, wherein Y¹ is halogen; X² is —OR⁹, —SR⁹, or —NHR⁹; R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl; n⁴ and n⁵ are each individually 0, 1 or 2, with the proviso that n⁴ and n⁵ are not both 0; each Y is individually H or halogen; each m is individually 1 or 2; each p is individually 1, 2, 3, 4, 5, or 6; each q is individually 0, 1, 2, 3, 4, 5, or 6; each t is individually 1, 2, 3, 4, 5, or 6; R⁷ is H, —COR⁸, —CO₂R⁸, —(CO)—NHR⁸, L⁴, L⁵, L⁶, or L⁷; R⁸ is a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³; R⁹ is H, —COR⁸, —CO₂R⁸, —(CO)—NHR⁸, L⁴, L⁵, L⁶, or L⁷, with the proviso that exactly one of R′ and R⁹ is L⁴, L⁵, L⁶, or L⁷; and each X³ is individually —H, —OH, —SH, or —NH₂.

### Immunoconjugates

Various embodiments disclosed herein relate to an immunoconjugate of Formula (I), having the structure:

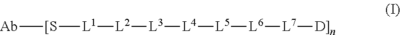

or a pharmaceutically acceptable salt thereof.

In various embodiments, L¹ in Formula (III) is L¹ is

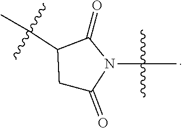 [SMILES: *C1CC(=O)N(*)C1=O]

In various embodiments, L² in Formula (III) is absent,

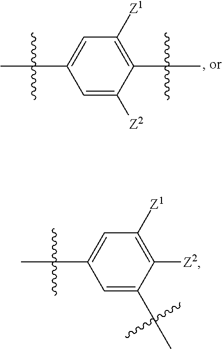

where Z¹ and Z² are each individually hydrogen, halogen, —NO₂, —O—(C₁-C₆ alkyl), or C₁-C₆ alkyl. In an embodiment, L² in Formula (III) is absent. In an embodiment, L² in Formula (III) is

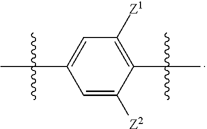

In an embodiment, L² in Formula (III) is

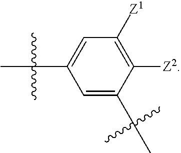

In various embodiments, Z¹ and Z² in Formula (III) are each individually hydrogen, halogen, —NO₂, —O—(C₁-C₆ alkyl), or C₁-C₆ alkyl. In an embodiment, at least one of Z¹ and Z² is hydrogen. In an embodiment, at least one of Z¹ and Z² is halogen. In an embodiment, at least one of Z¹ and Z² is —NO₂. In an embodiment, at least one of Z¹ and Z² is —O—(C₁-C₆ alkyl). For example, in an embodiment, at least one of Z¹ and Z² is methoxy. In an embodiment, at least one of Z¹ and Z² is C₁-C₆ alkyl. For example, in an embodiment, at least one of Z¹ and Z² is methyl.

In various embodiments, L³ in Formula (III) is —(CH₂)n¹-C(═O)— or —(CH₂CH₂O)n¹-(CH₂)n¹C(═O)—, where n¹ are independently integers of 0 to 12. In an embodiment, L³ is —(CH₂)n¹-C(═O)—. For example, in an embodiment, L³ is —C(═O)—. In an embodiment, L³ is —(CH₂CH₂O) n¹-(CH₂)n¹C(—O)—. For example, in an embodiment, L³ is —CH₂C(═O)—. In embodiment, n¹ is an integer of 1 to 12, such as 1 to 6 or 1 to 3.

In various embodiments, L⁴ in Formula (III) is a tetrapeptide residue. For example, in an embodiment, L⁴ is a tetrapeptide residue selected from GGFG (gly-gly-phe-gly), EGGF (glu-gly-gly-phe). SGGF (ser-gly-gly-phe), and KGGF (lys-gly-gly-phe).

In various embodiments, L⁵ in Formula (III) is absent or —[NH(CH₂)n²]n³-, where n² is an integer of 0 to 6 and n³ is an integer of 0 to 2. In an embodiment, L⁵ is absent. In an embodiment, L⁵ is —[NH(CH₂)n²]n³-. For example, in an embodiment, L⁵ is —NH—. In another embodiment. L⁵ is —NHCH₂—.

In various embodiments, L⁶ in Formula (III) is absent or

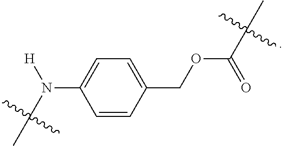 [SMILES: *Nc1ccc(COC(*)=O)cc1]

In an embodiment, L⁶ is absent. In another embodiment, L⁶ is

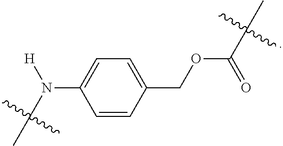 [SMILES: *Nc1ccc(COC(*)=O)cc1]

In various embodiments, L⁷ in Formula (III) is absent.

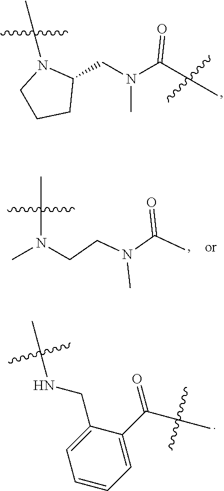

In an embodiment. L⁷ is absent. In an embodiment, L⁷ is

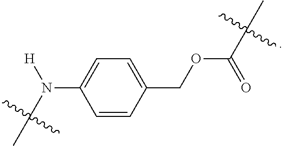 [SMILES: *Nc1ccc(COC(*)=O)cc1]

In an embodiment, L⁷ is

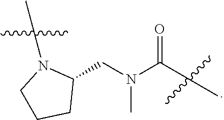 [SMILES: *C(=O)N(C)C[C@@H]1CCCN1*]

In an embodiment, L⁷ is

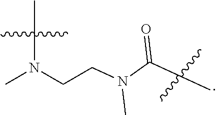 [SMILES: *C(=O)N(C)CCN(*)C]

In an embodiment, L⁷ is

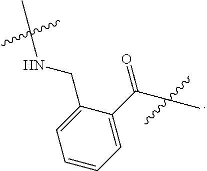 [SMILES: *NCc1ccccc1C(*)=O]

In various embodiments, D in the immunoconjugate of Formula (I) is a drug moiety as described herein (e.g., under the heading “Drug Moieties” above). The “S” (indicated with bold lettering) in Formula (I) (Ab-[S-L¹-L²-L³-L⁴-L³-L⁶-L⁷-D]ₙ) can be the sulfur present in a cysteine (for example, a cysteine that can be present in the antibody itself, a fragment of the antibody, the antigen-binding fragment itself, a portion of the antigen-binding fragment and/or a linker bound to the antibody or antigen-binding fragment). In an embodiment, D is a cytotoxic anti-cancer drug moiety. In an embodiment, the drug moiety is exatecan.

In various embodiments, Ab in Formula (III) is an antibody or an antigen-binding fragment. In an embodiment, Ab specifically binds to human receptor tyrosine kinase like orphan receptor 1 (ROR1), Her2, TROP2, Her3, B7-H3, GPR20 or CEACAM5. In an embodiment, Ab binds to a cancer cell surface. In an embodiment, Ab is an anti-HER2 antibody.

In various embodiments, a immunoconjugate compound of Formula (I) can be selected from:

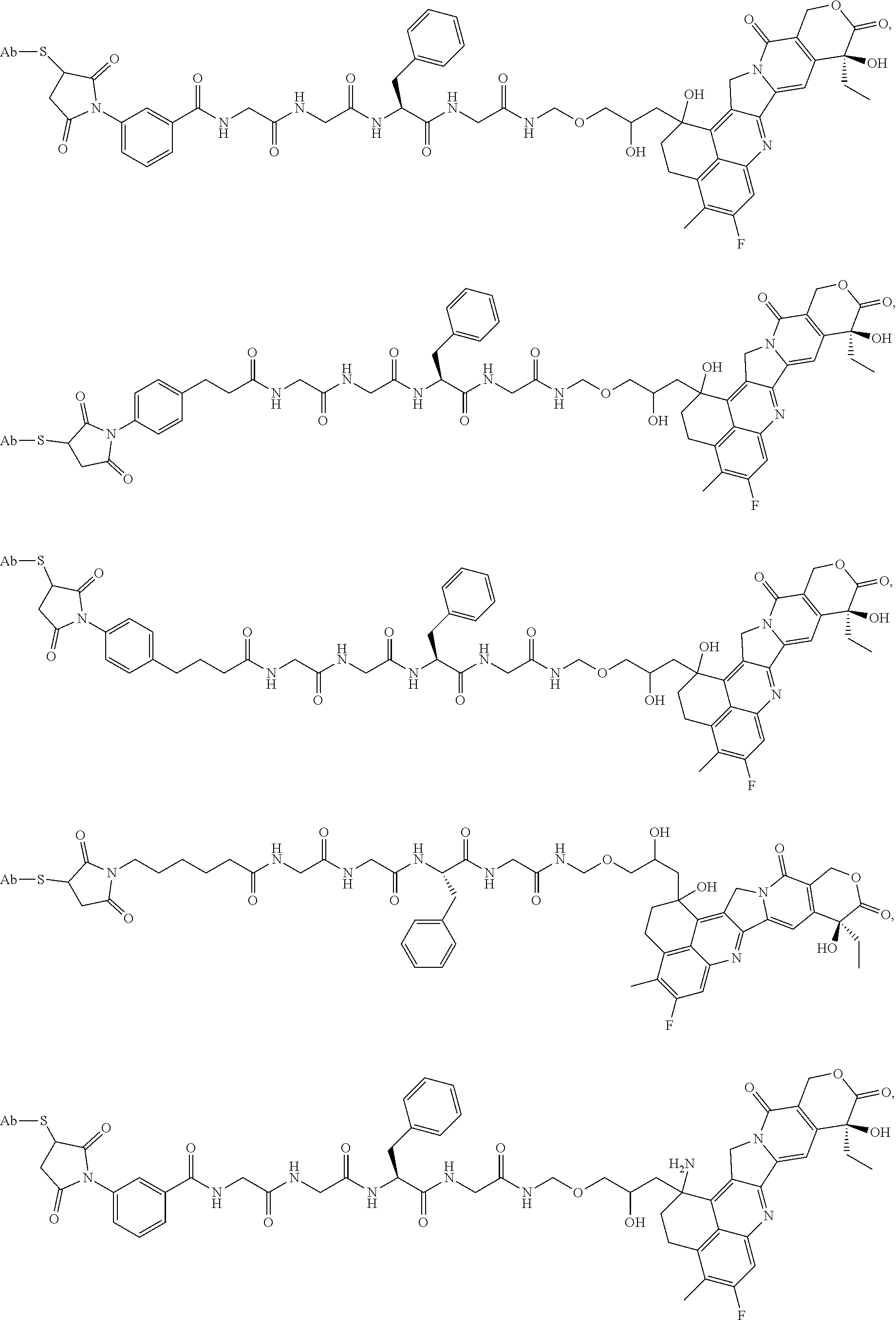

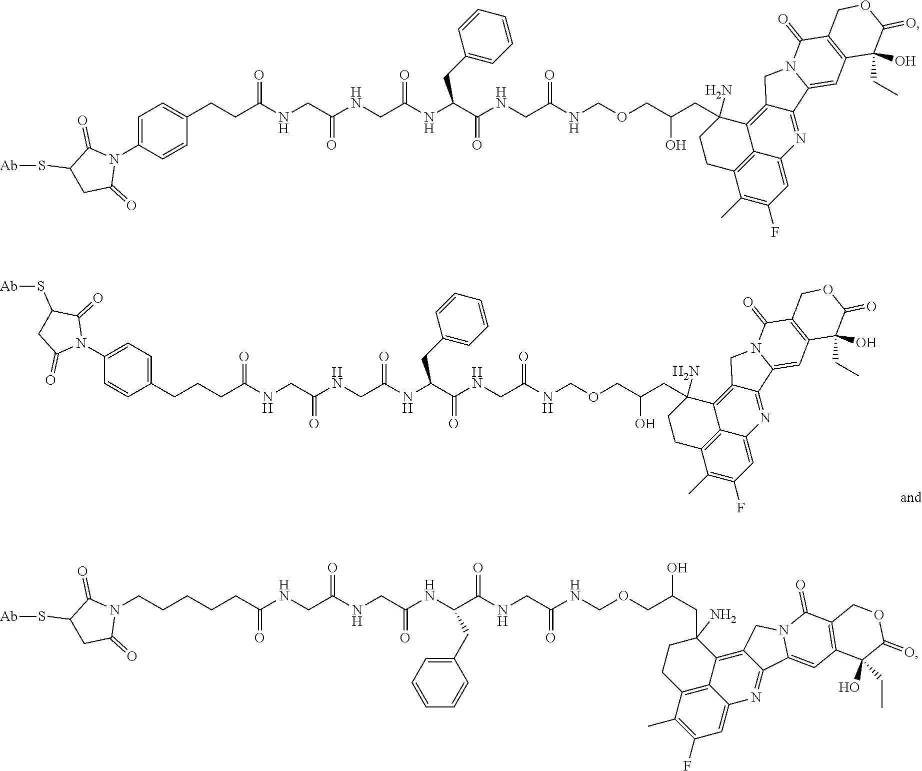

or a pharmaceutically acceptable salt of any of the foregoing.

In various embodiments, a immunoconjugate compound of Formula (I) can be selected from:

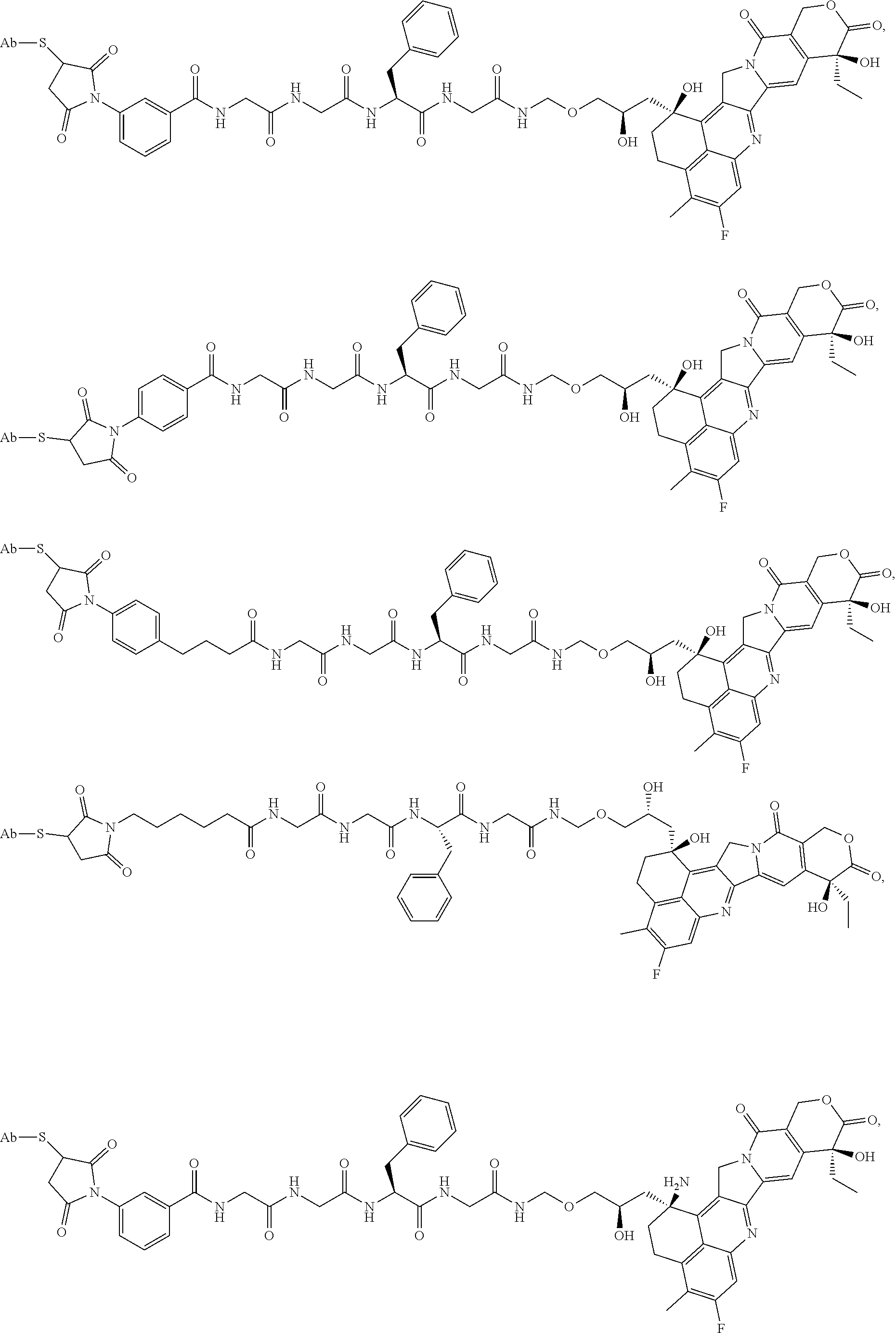

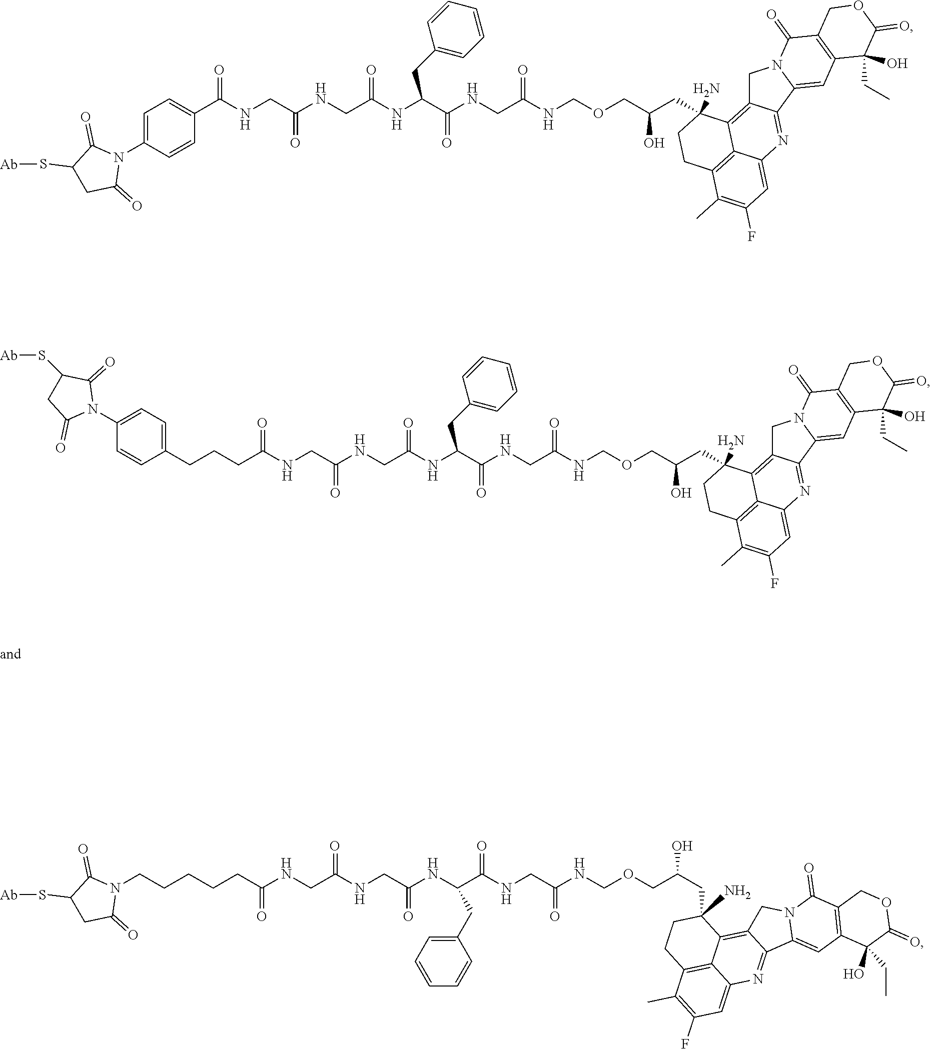

or a pharmaceutically acceptable salt of any of the foregoing.

In various embodiments, an immunoconjugate compound of Formula (I) cannot be selected from:

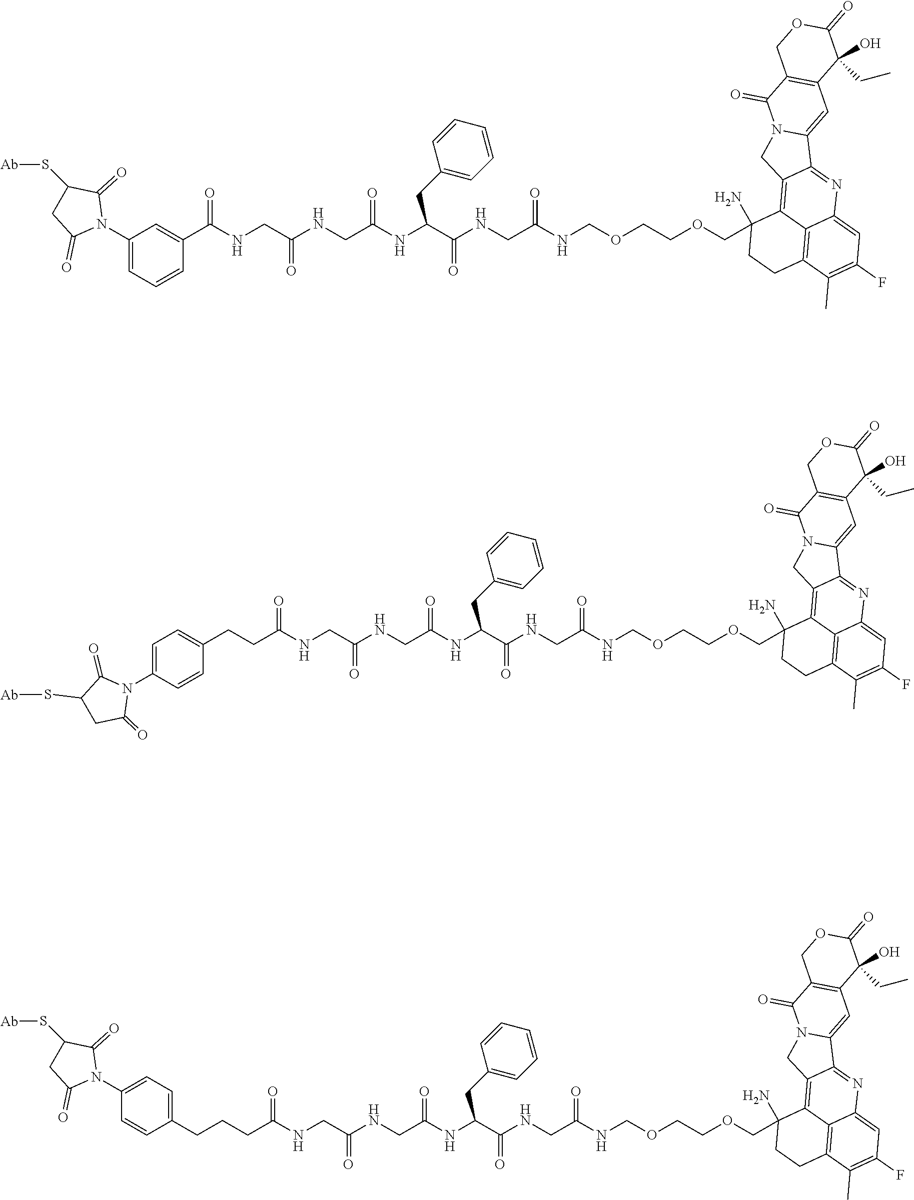

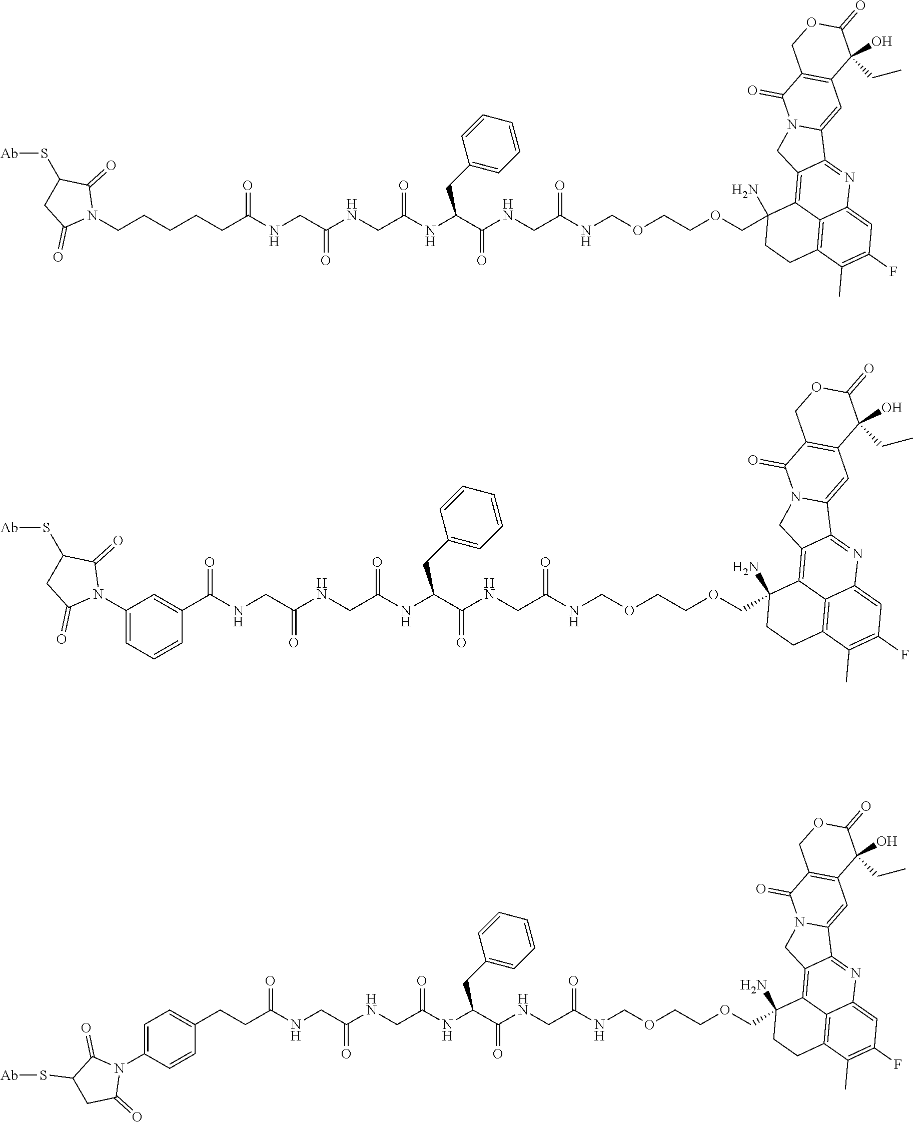

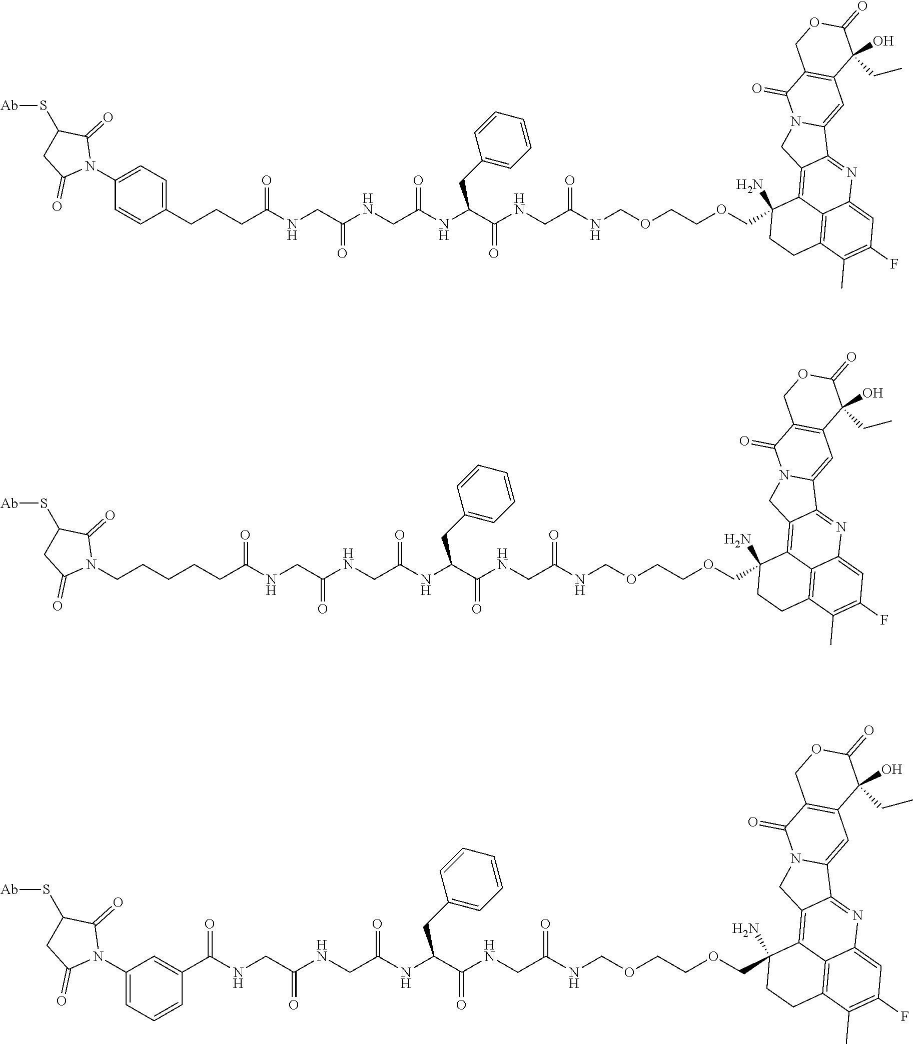

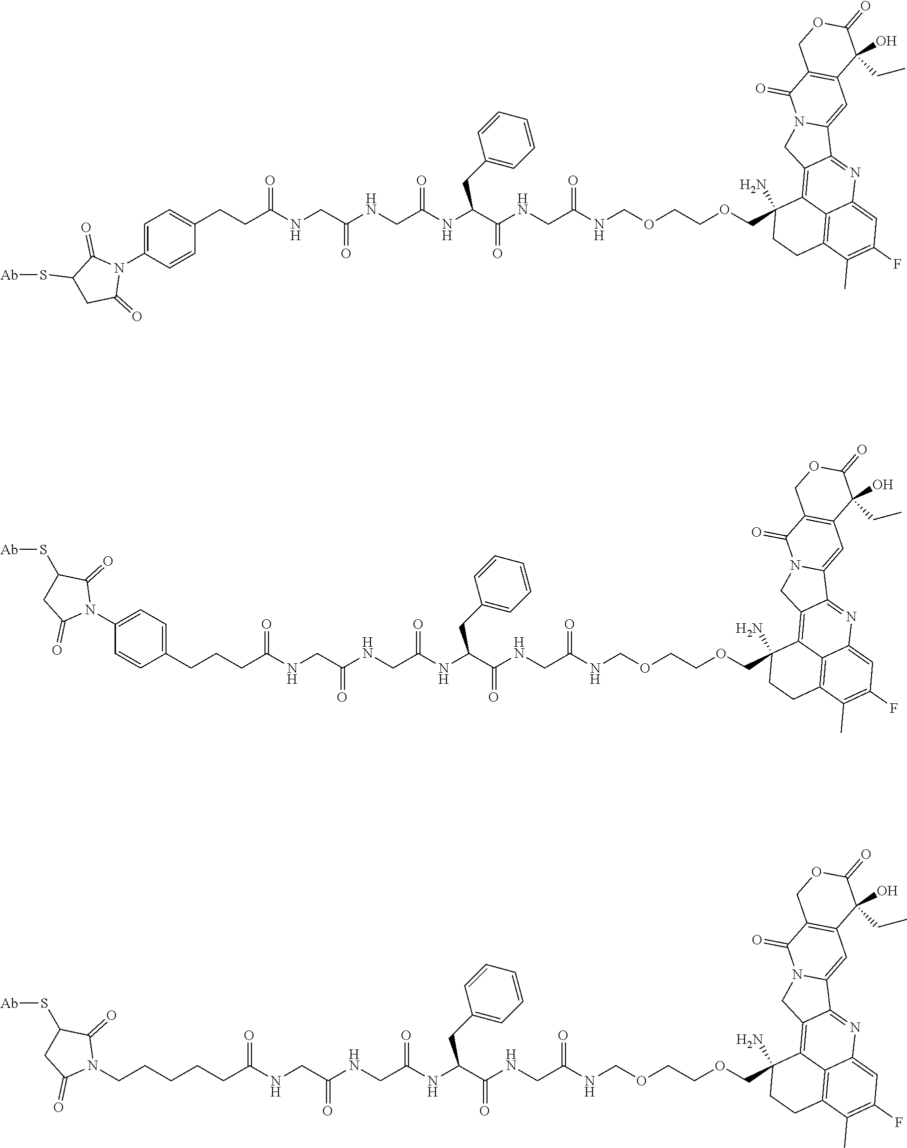

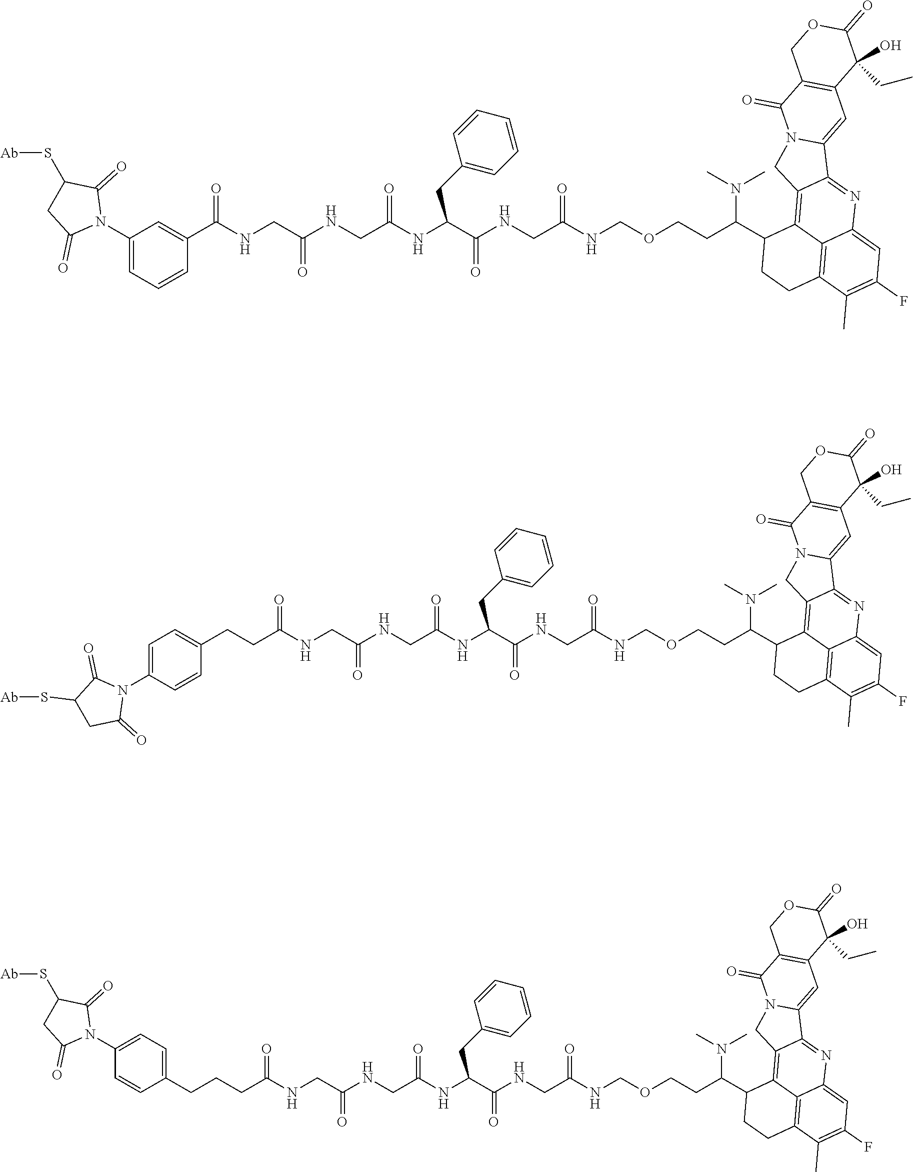

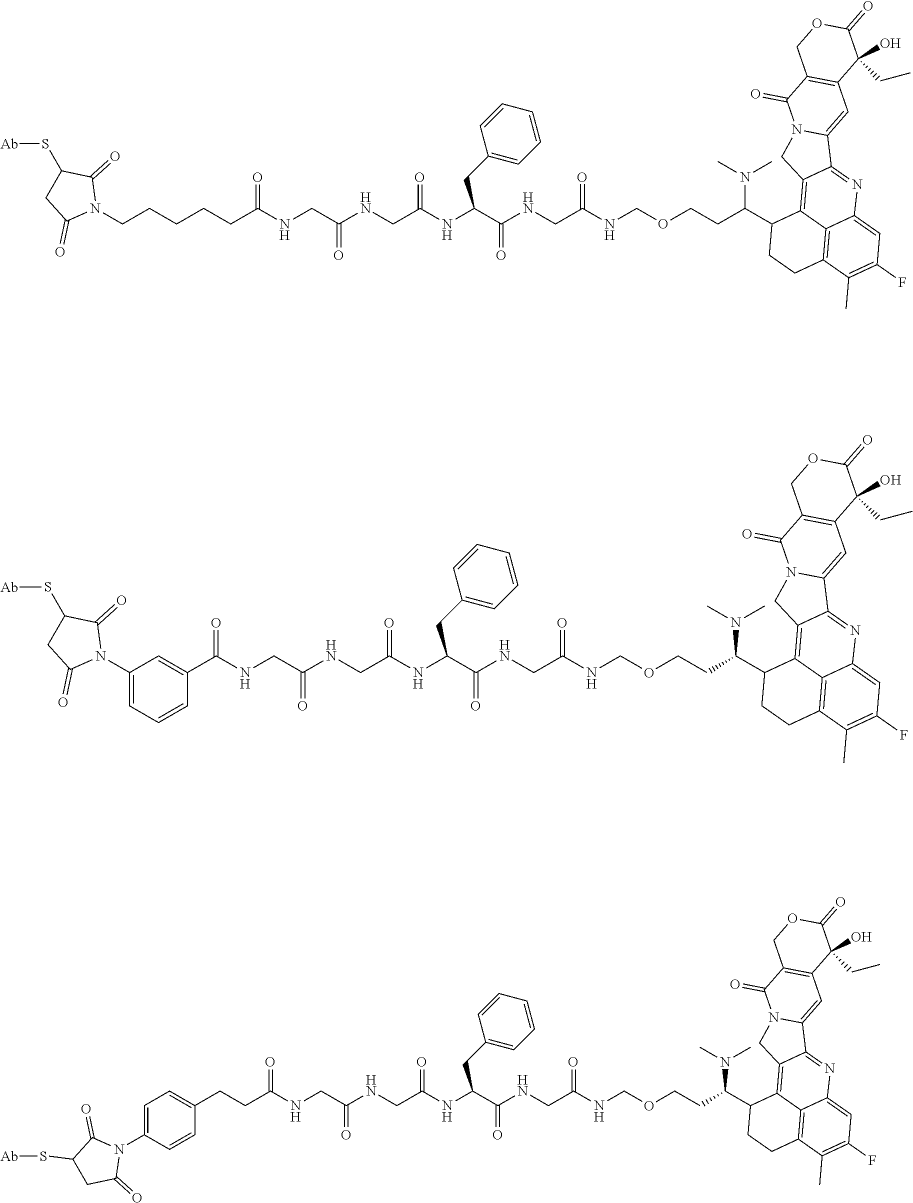

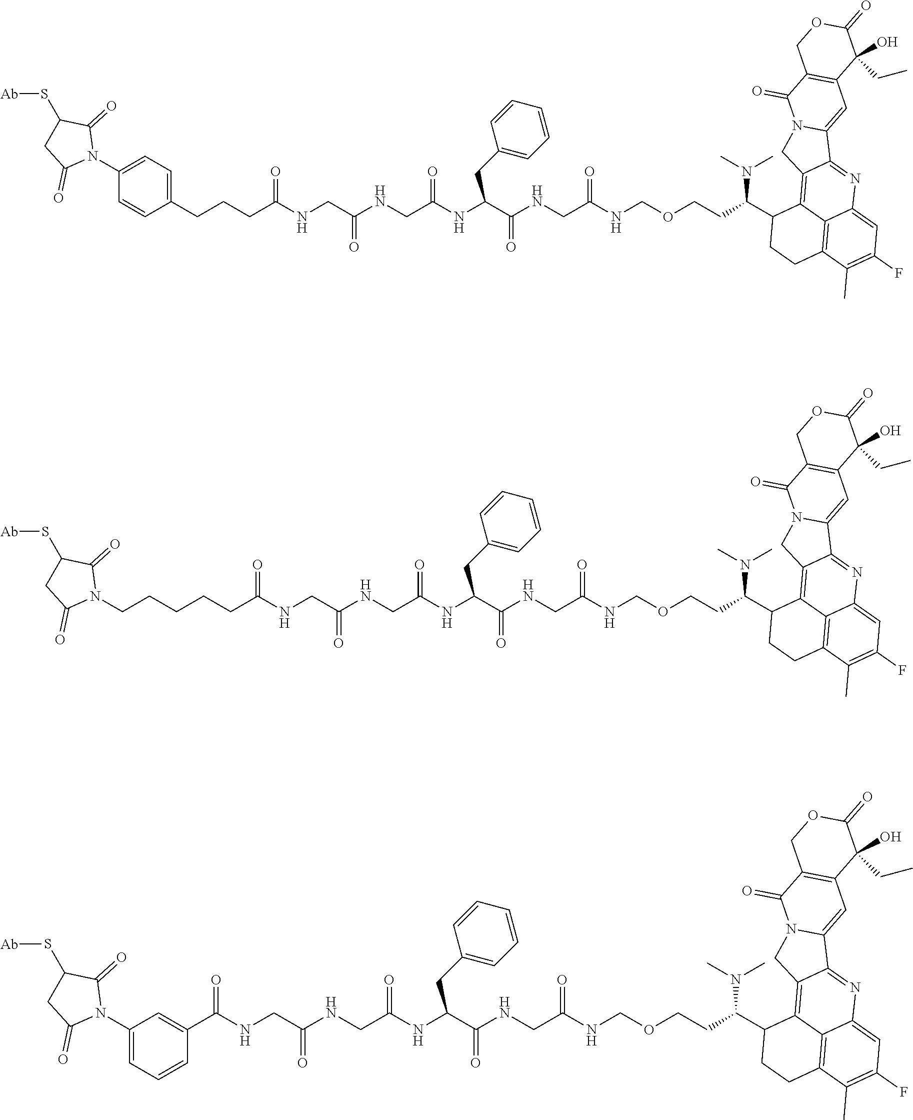

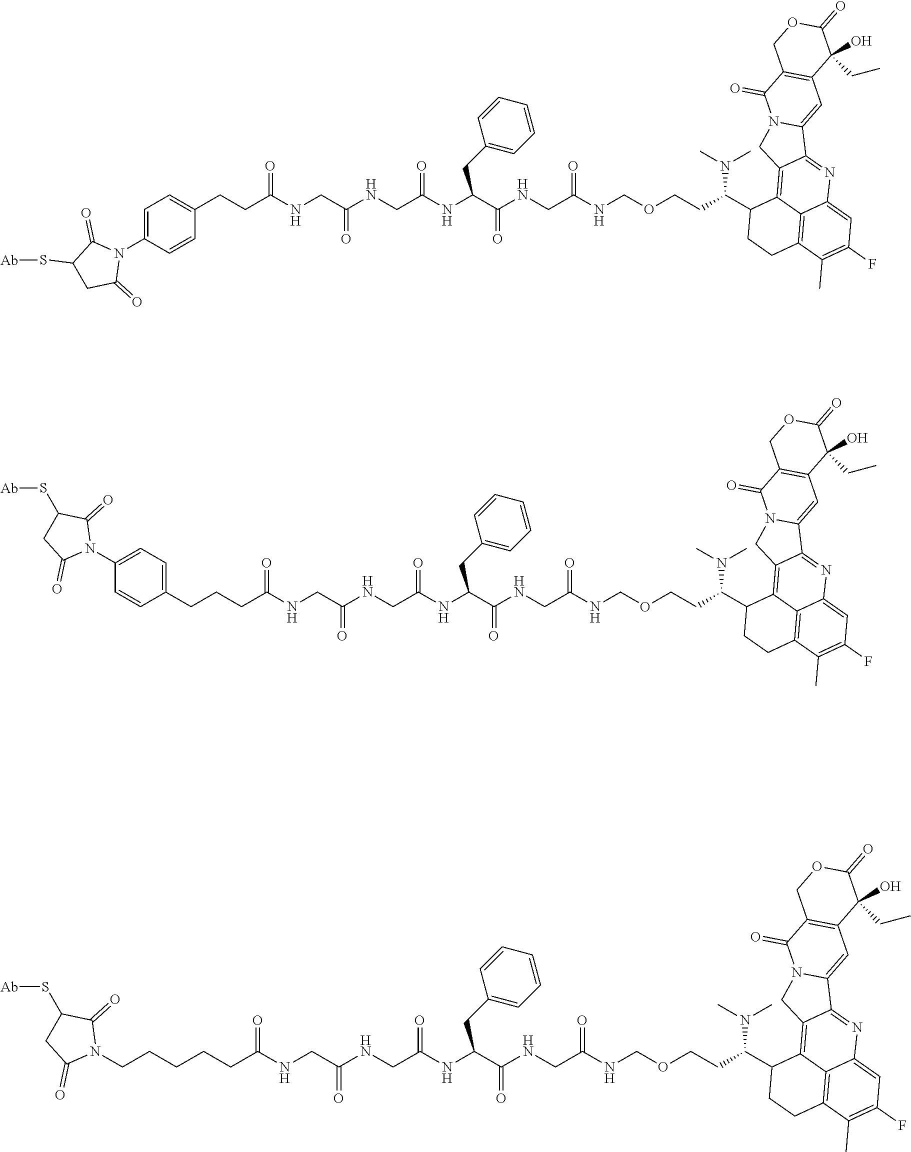

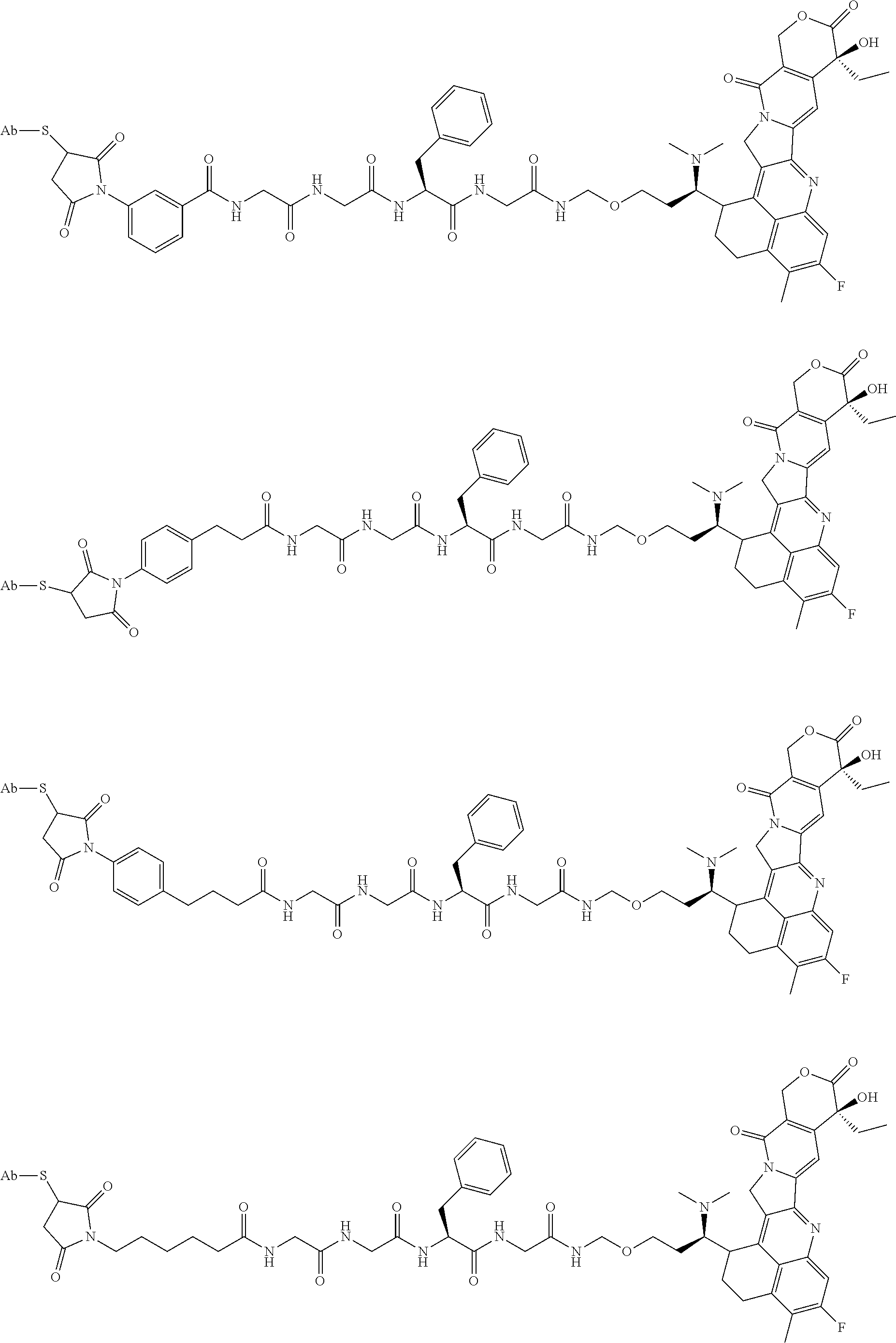

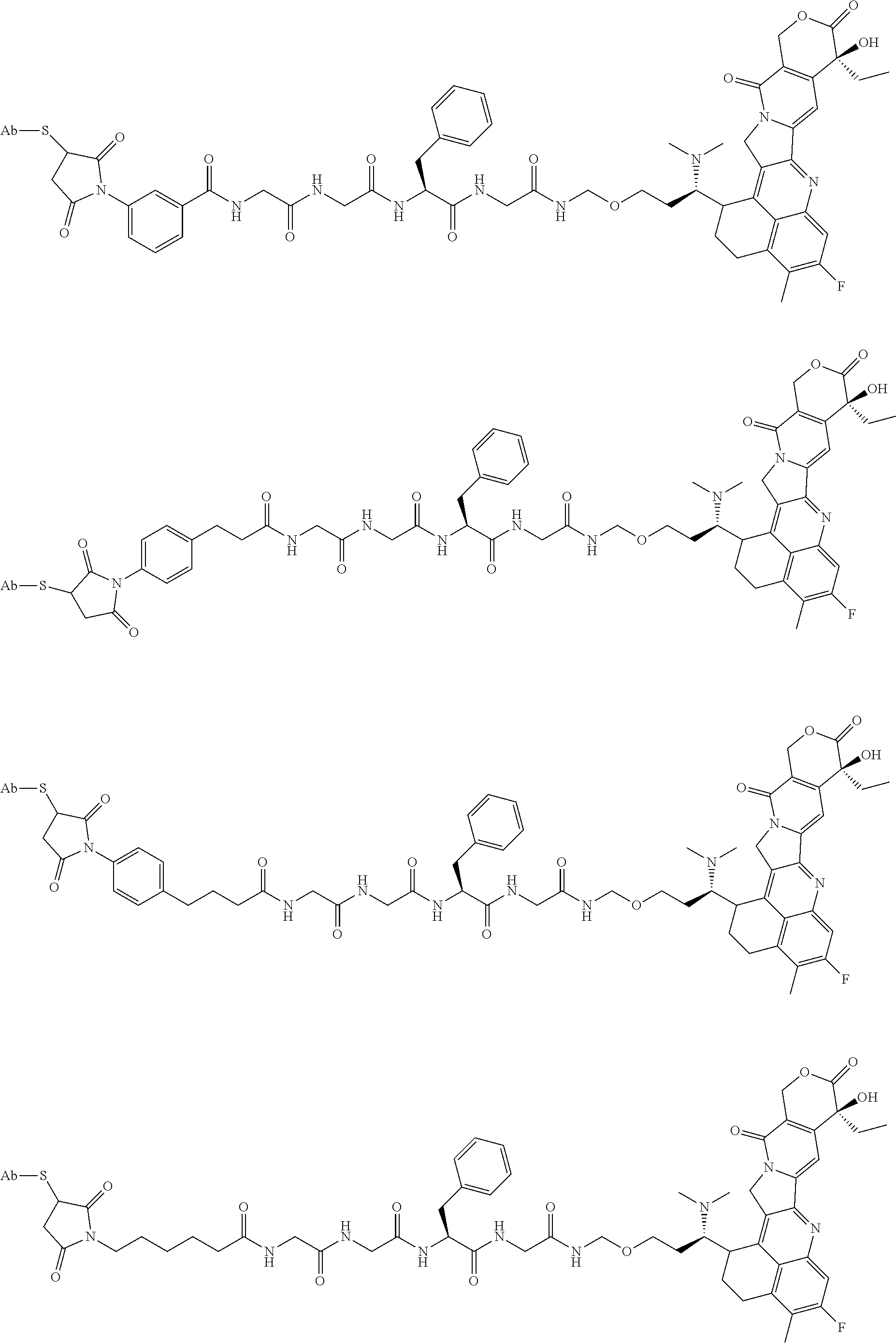

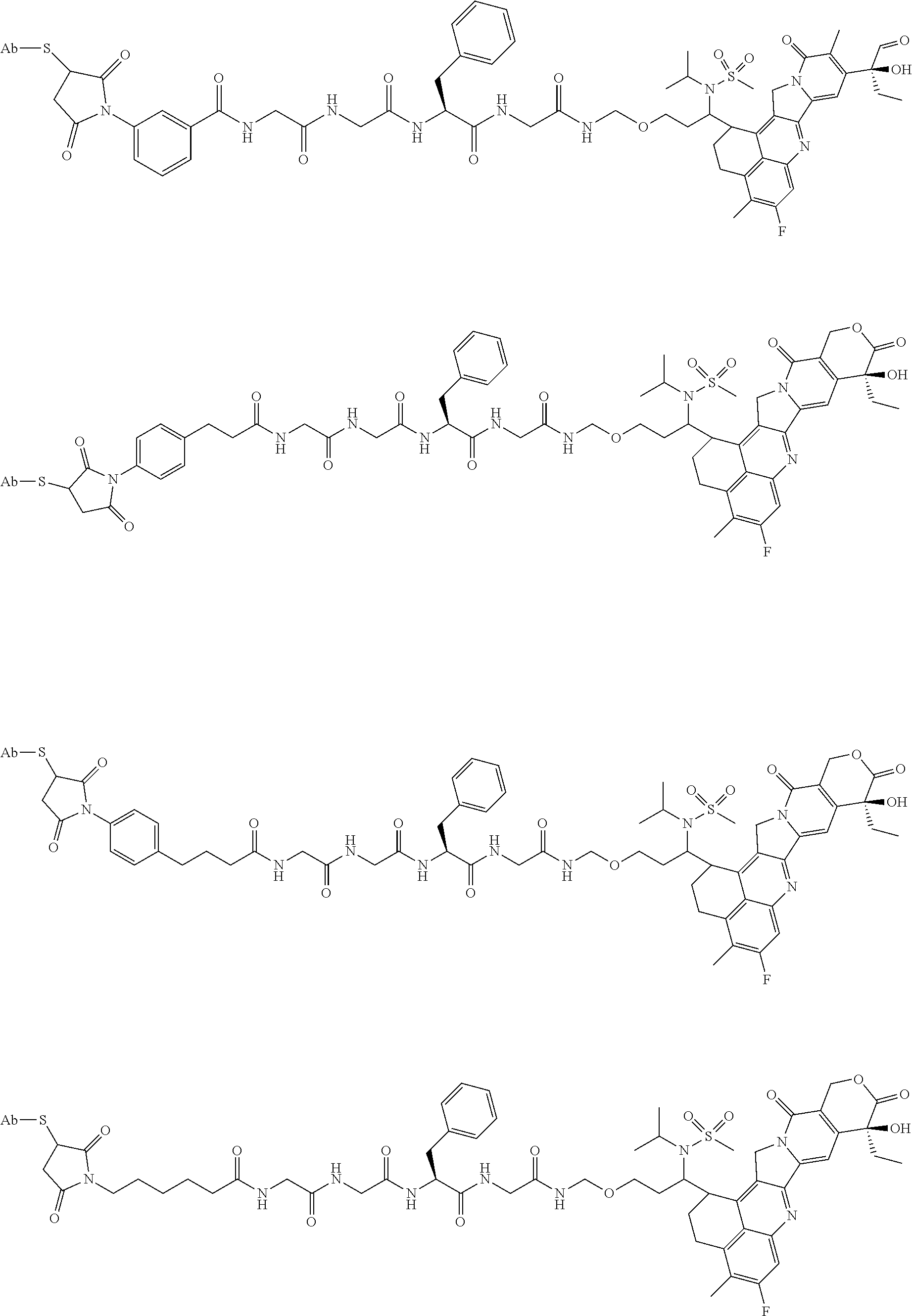

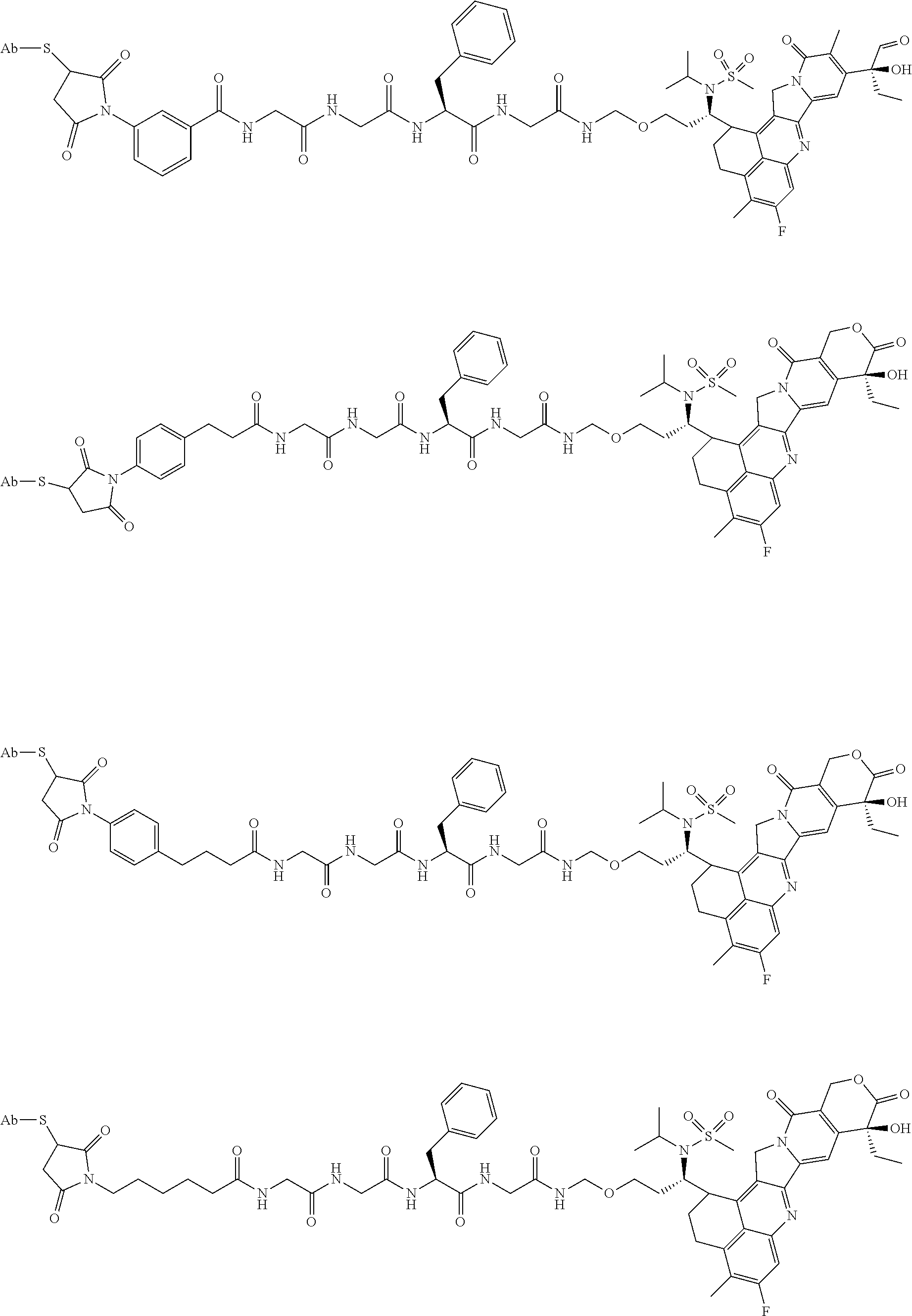

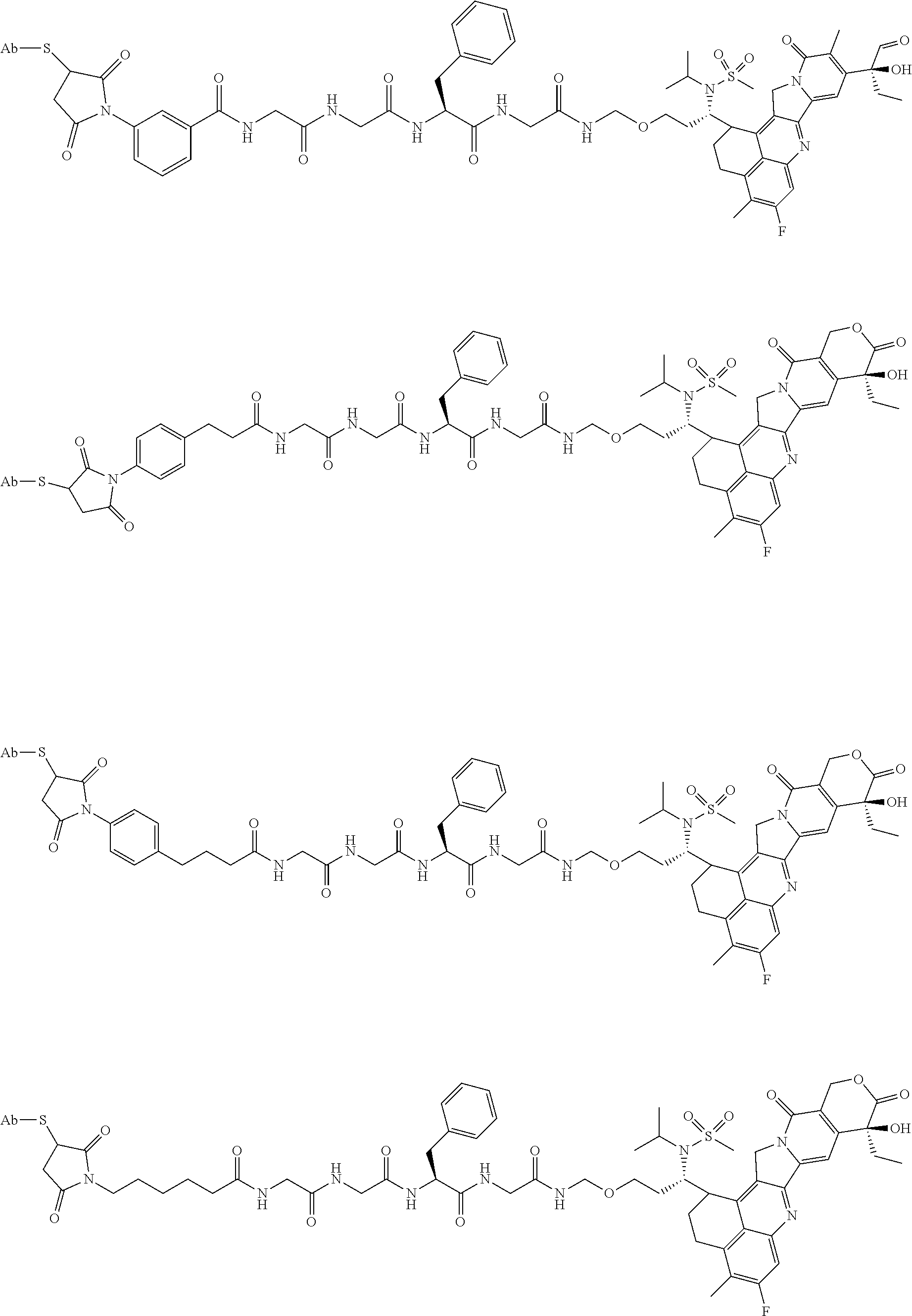

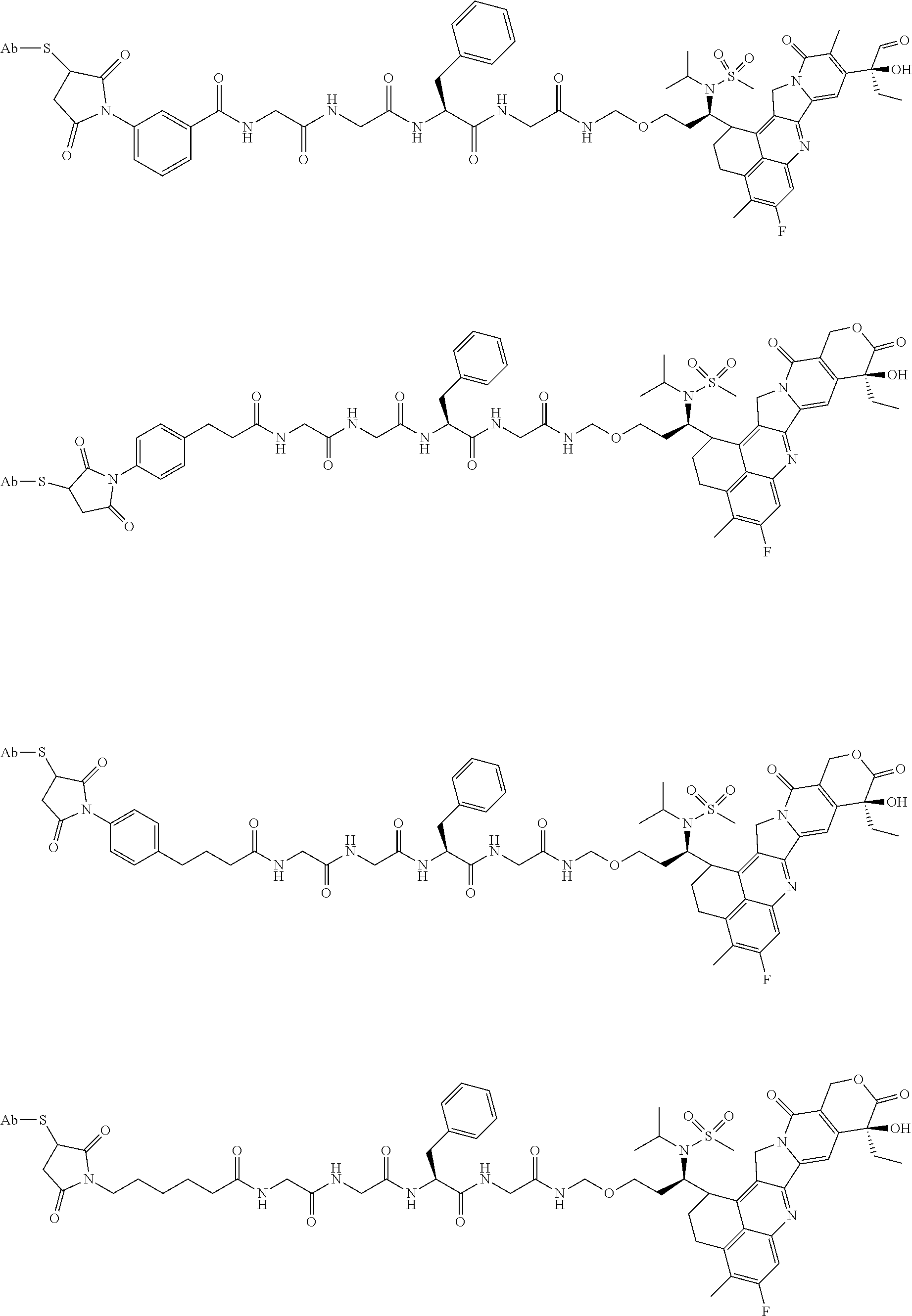

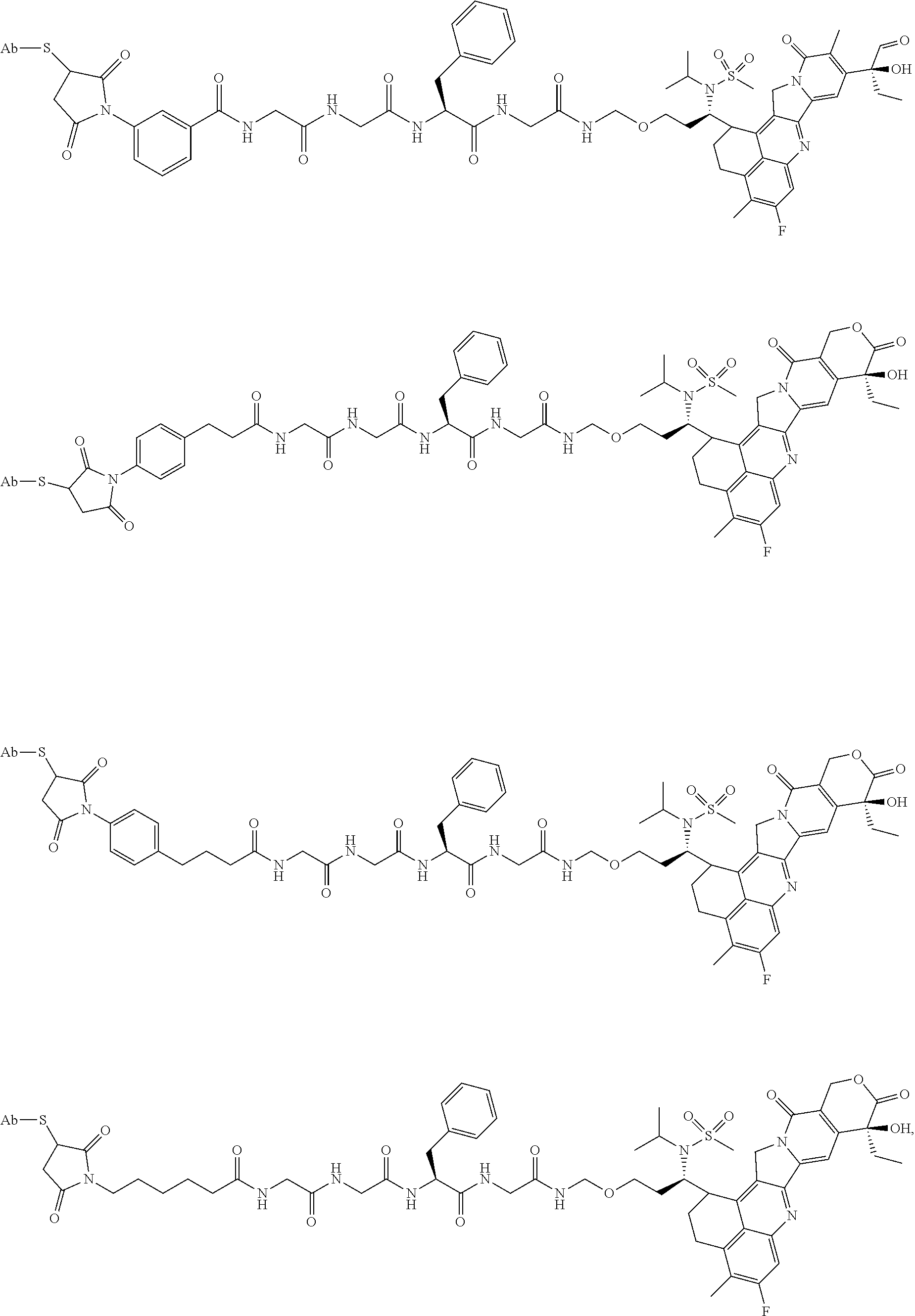

or a pharmaceutically acceptable salt of any of the foregoing.

In various embodiments, an immunoconjugate compound of Formula (I) cannot be selected from:

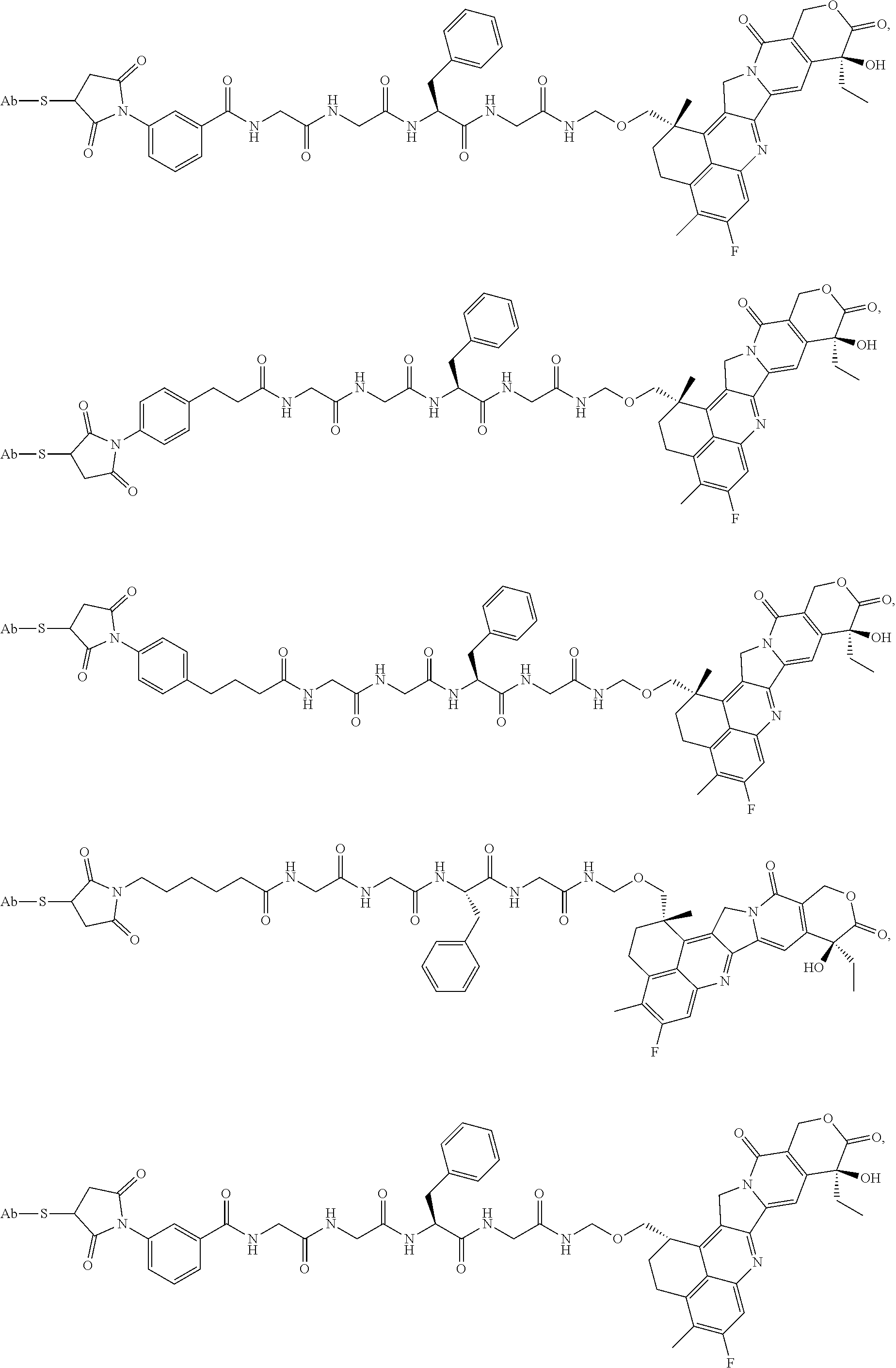

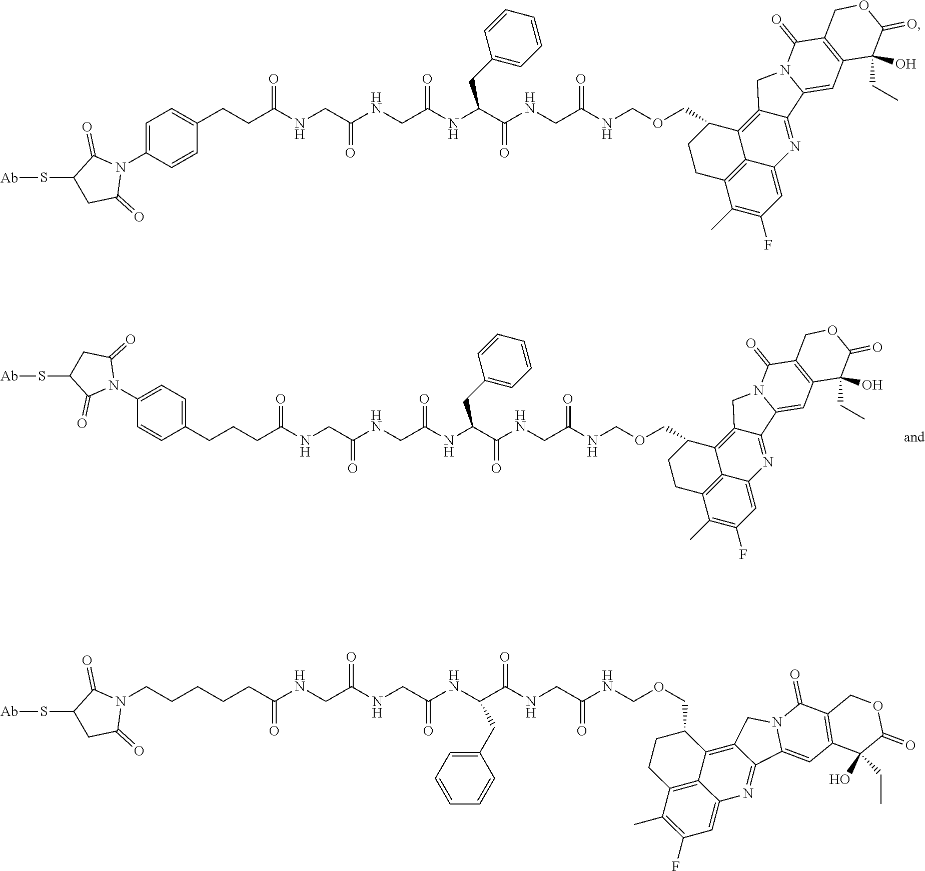

or a pharmaceutically acceptable salt thereof.

In various embodiments, an immunoconjugate compound of Formula (I), or a pharmaceutically acceptable salt thereof, cannot be selected from:

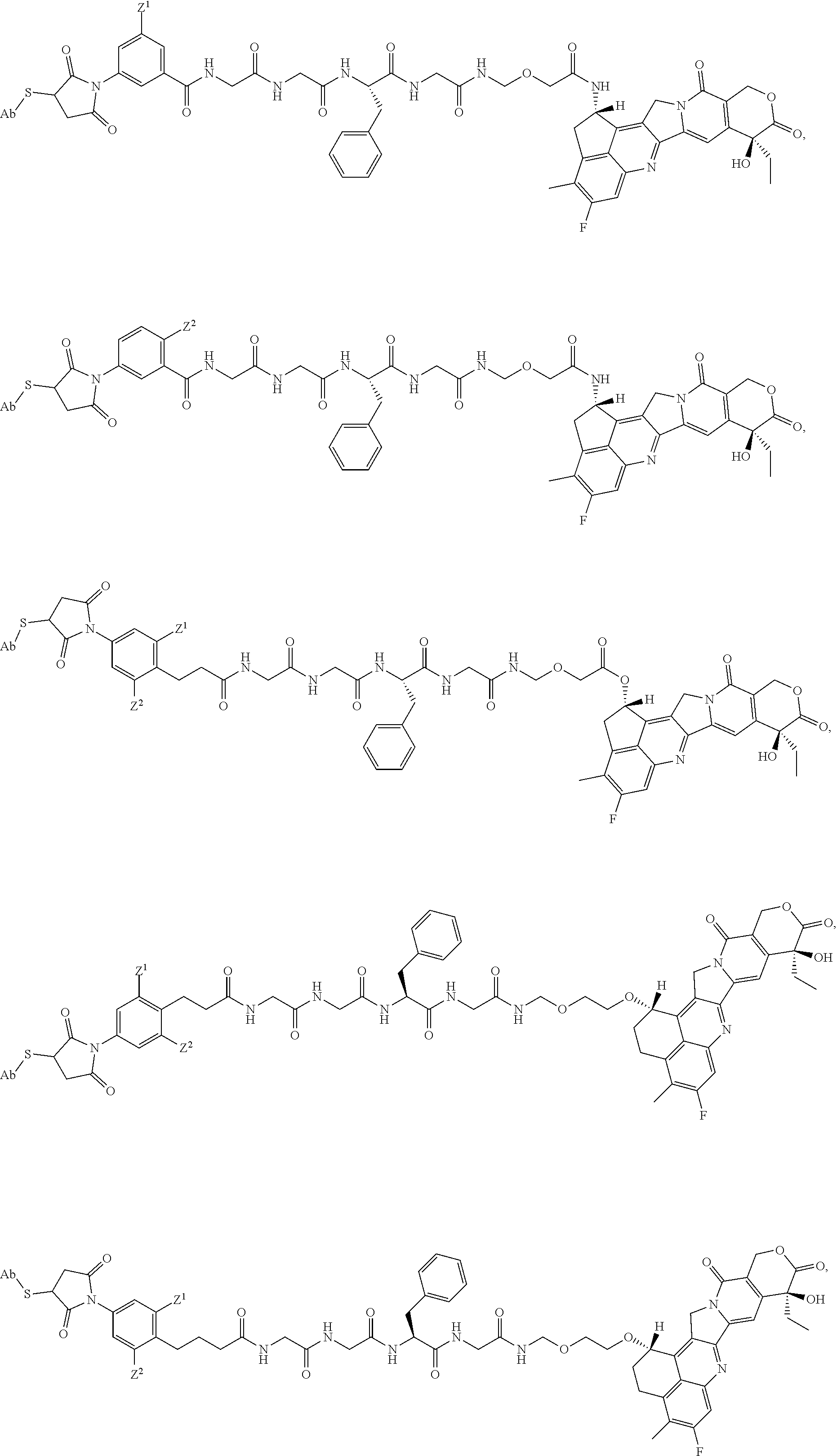

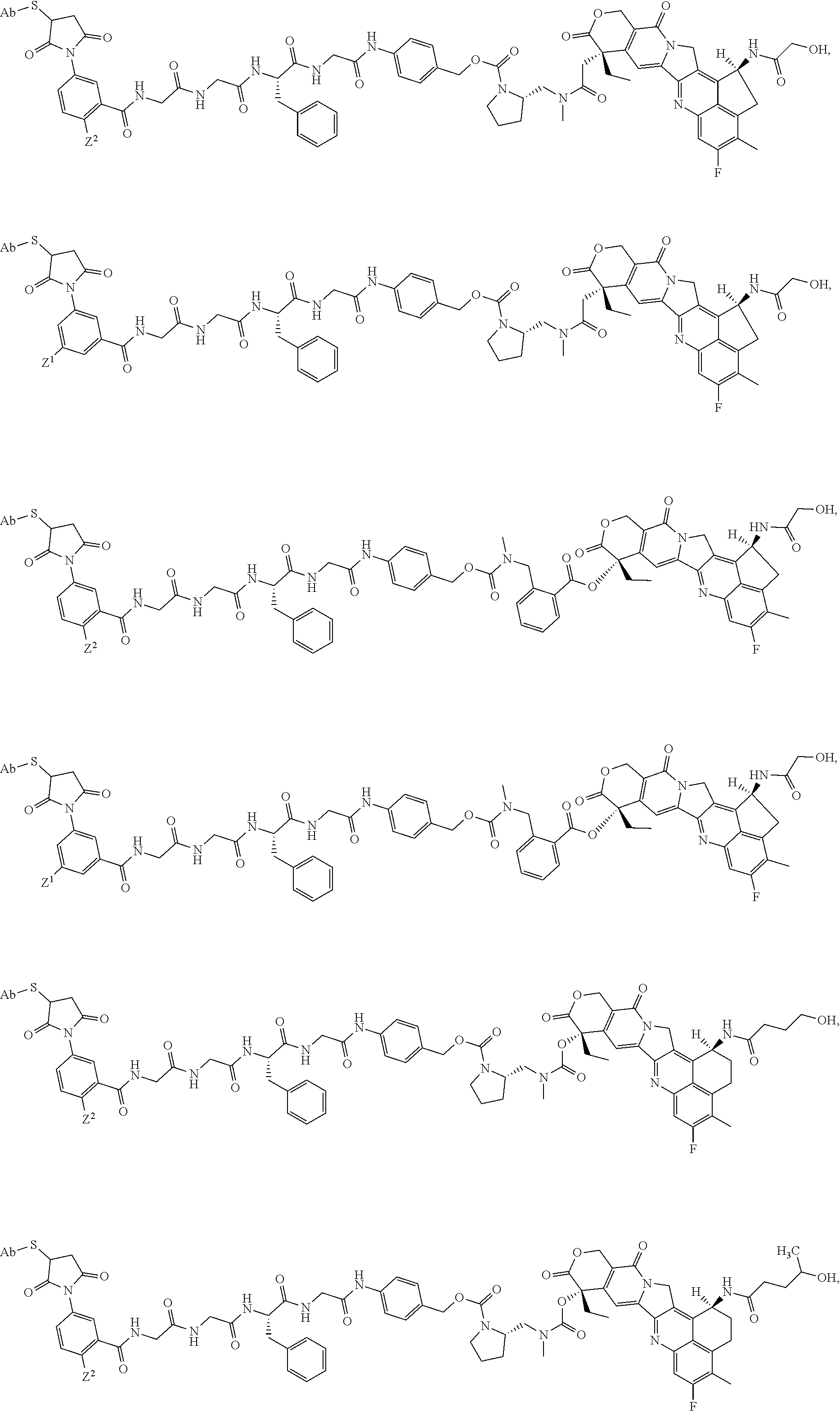

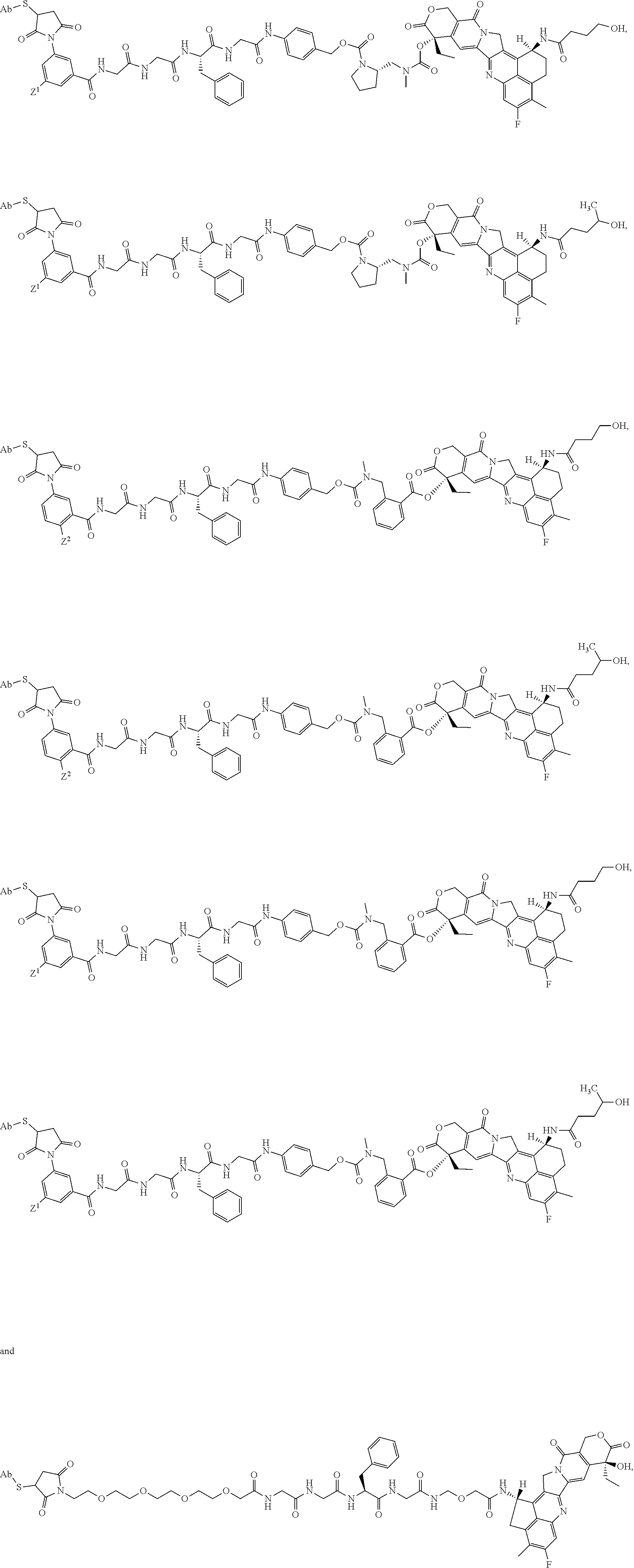

wherein Z¹ and Z² are each individually selected from hydrogen, fluoro, chloro, —NO₂, and —OCH₃.

### Pharmaceutical Compositions

Some embodiments described herein relate to a pharmaceutical composition, which can include an effective amount of one or more compounds described herein (for example, an immunoconjugate compound of Formula (I), a drug compound of the Formula (IV), or a pharmaceutically acceptable salt thereof) and a pharmaceutically acceptable carrier, diluent, excipient or combination thereof.

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. In certain embodiments, the composition is lyophilized, and then reconstituted, for example, in buffered saline, at the time of administration. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a targeting ligand to a specific cell or tissue type. The liposomes will be targeted to and taken up selectively by the targeted cell or tissue.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container and labeled for treatment of an indicated condition.

### Uses and Methods of Therapy

Some embodiments described herein relate to a method for treating, inhibiting, or ameliorating a cancer or a tumor, which can include administering an effective amount of a compound described herein (for example, an immunoconjugate compound of Formula (I), a drug compound of the Formula (IV), or a pharmaceutically acceptable salt thereof) or a pharmaceutical composition that includes a compound described herein (for example, an immunoconjugate compound of Formula (I), a drug compound of the Formula (IV), or a pharmaceutically acceptable salt thereof) to a subject having the cancer or tumor. Other embodiments described herein relate to the use of an effective amount of a compound described herein (for example, an immunoconjugate compound of Formula (I), a drug compound of the Formula (IV), or a pharmaceutically acceptable salt thereof) or a pharmaceutical composition that includes a compound described herein (for example, an immunoconjugate compound of Formula (I), a drug compound of the Formula (IV), or a pharmaceutically acceptable salt thereof) in the manufacture of a medicament for treating, inhibiting, or ameliorating a cancer or a tumor described herein. Still other embodiments described herein relate to an effective amount of a compound described herein (for example, an immunoconjugate compound of Formula (I), a drug compound of the Formula (IV), or a pharmaceutically acceptable salt thereof) or a pharmaceutical composition that includes a compound described herein (for example, an immunoconjugate compound of Formula (I), a drug compound of the Formula (IV), or a pharmaceutically acceptable salt thereof) for treating, inhibiting, or ameliorating a cancer or a tumor described herein.

Examples of cancers and tumors contemplated to respond to one or more therapies described herein include but are not limited to: lung cancer, urothelial cancer, colorectal cancer, prostate cancer, ovarian cancer, pancreatic cancer, breast cancer, bladder cancer, gastric cancer, gastrointestinal stromal tumor, uterine cervix cancer, esophageal cancer, squamous cell carcinoma, peritoneal cancer, liver cancer, hepatocellular cancer, colon cancer, rectal cancer, colorectal cancer, endometrial cancer, uterine cancer, salivary gland cancer, kidney cancer, vulval cancer, thyroid cancer, penis cancer, leukemia, malignant lymphoma, plasmacytoma, myeloma, or sarcoma.

As used herein, a “subject” refers to an animal that is the object of treatment or therapy, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical agent, which elicits the biological or medicinal response indicated. For example, a therapeutically effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound is the amount that results in: (a) the reduction, alleviation or disappearance of one or more symptoms caused by the cancer, (b) the reduction of tumor size. (c) the elimination of the tumor, and/or (d) long-term disease stabilization (growth arrest) of the tumor. In the treatment of lung cancer (such as non-small cell lung cancer), a therapeutically effective amount is that amount that alleviates or eliminates cough, shortness of breath and/or pain.

The amount of the immunoconjugate compound of Formula (I), drug compound of the Formula (IV), or pharmaceutically acceptable salt thereof, required for use in therapy will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

In general, however, a suitable dose will often be in the range of from about 0.5 mg/kg to about 10 mg/kg. For example, a suitable dose may be in the range from about 1.0 mg/kg to about 7.5 mg/kg of body weight every three weeks, or weekly, such as about 1.5 mg/kg to about 5.0 mg/kg of body weight of the recipient every three weeks or weekly, about 2.0 mg/kg to 4.0 mg/kg of body weight of the recipient every three weeks or weekly, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, which is the dosage levels necessary to achieve the desired result, can be accomplished by one skilled in the art using routine methods, for example, human clinical trials, in vivo studies and in vitro studies. For example, useful dosages of an immunoconjugate compound of Formula (I), a drug compound of the Formula (IV), or a pharmaceutically acceptable salt thereof, can be determined by comparing their in vitro activity and in vivo activity in animal models. Such comparison can be done by comparison against an established drug, such as cisplatin and/or gemcitabine.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Synthesis

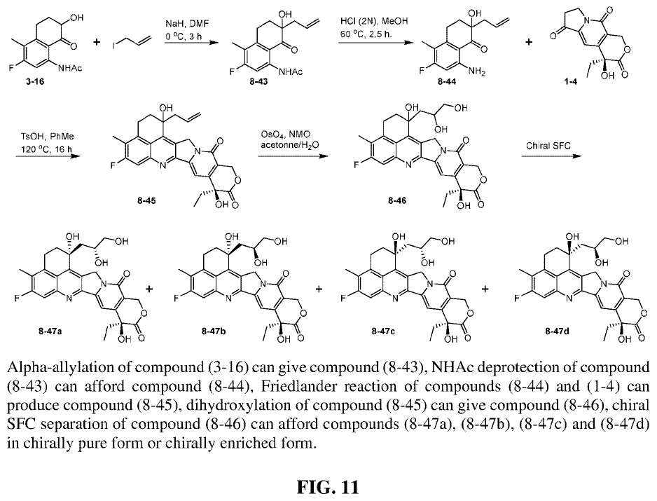

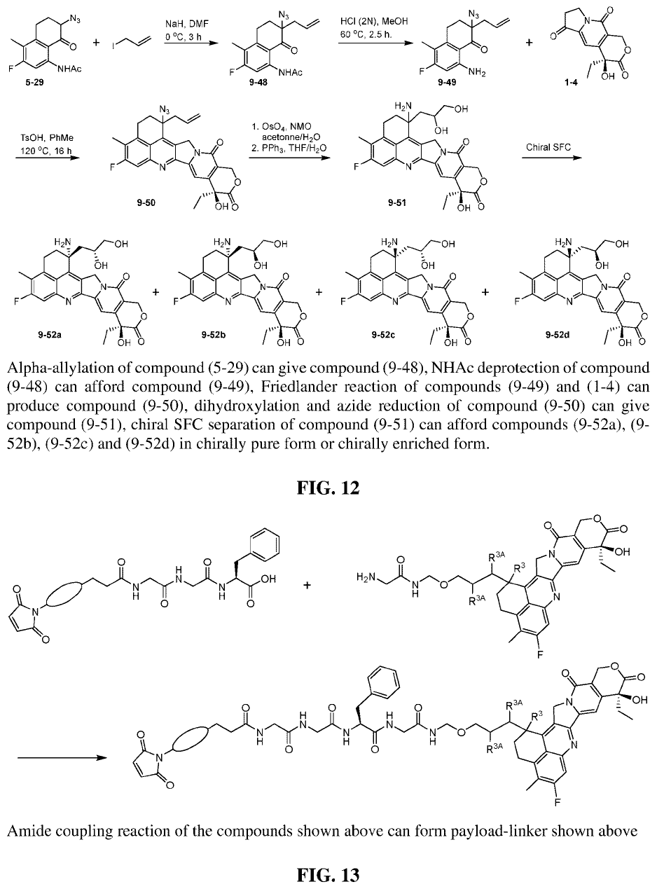

Drug compounds of the Formula (IV), or pharmaceutically acceptable salts thereof, can be made in various ways by those skilled using known techniques as guided by the detailed teachings provided herein. For example, in an embodiment, drug compounds of the Formula (IV) are prepared in accordance with the general schemes illustrated in FIGS. 2 and 4-12.

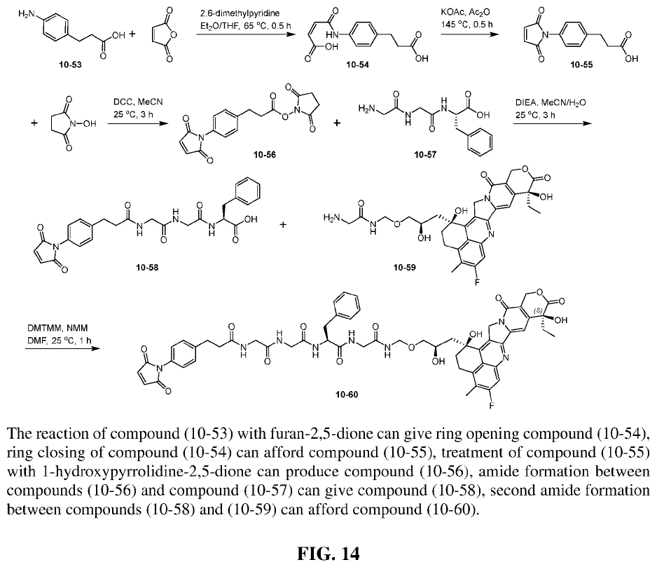

Conjugates of the Formula (III) can be made in various ways by those skilled using known techniques as guided by the detailed teachings provided herein. For example, in an embodiment, conjugates of the Formula (II) are prepared in accordance with the general schemes illustrated in FIGS. 13 and 14. Although illustrated with specific linkers and payloads, those skilled in the art will appreciate that other linkers and/or payloads may be used in similar manners.

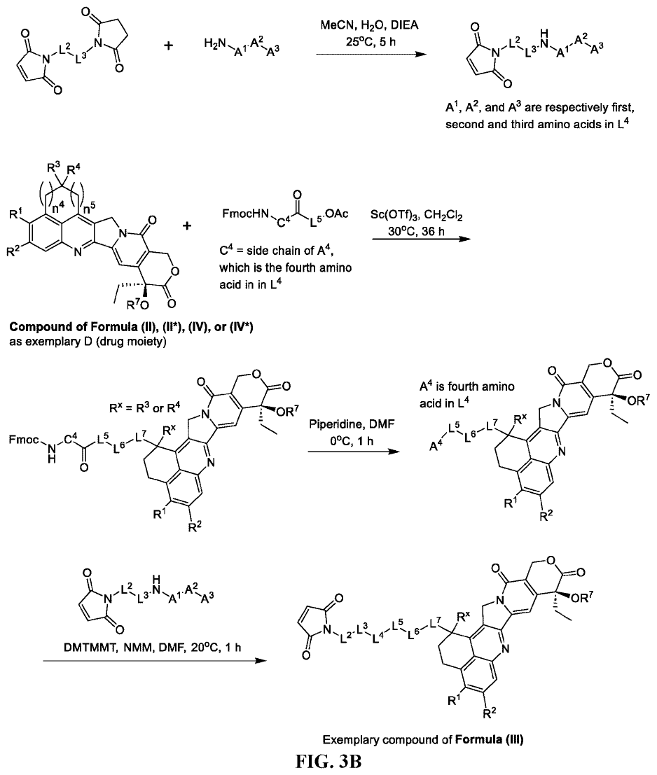

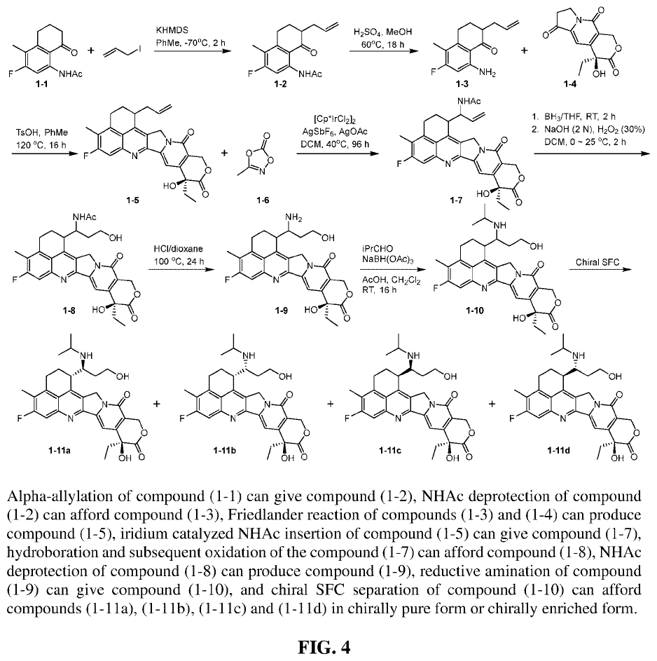

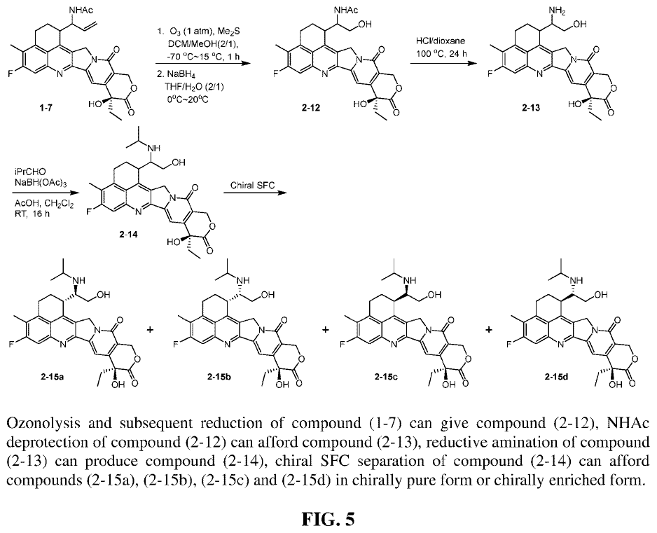

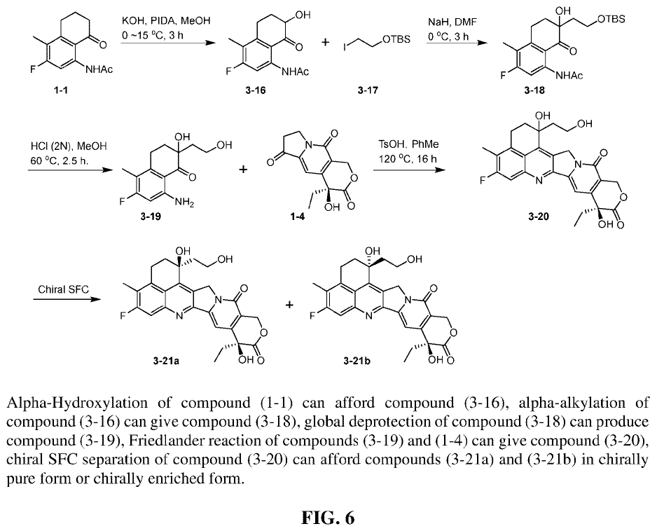

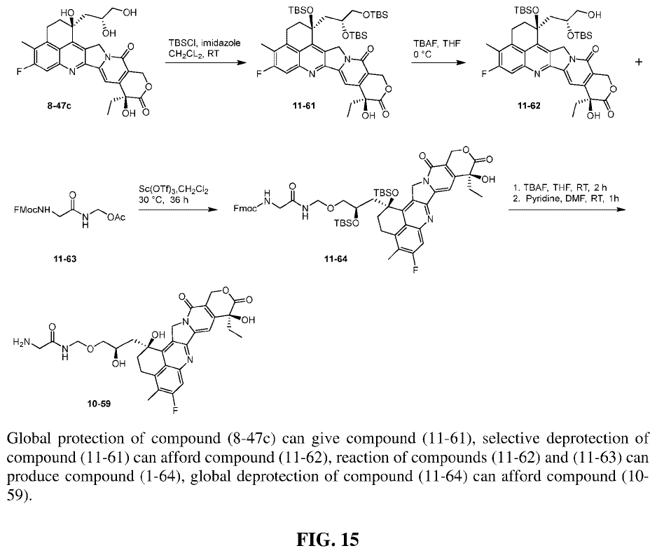

Immunoconjugates of the Formula (I) can be made in various ways by those skilled using known techniques as guided by the detailed teachings provided herein. For example, in an embodiment, immunoconjugates of the Formula (I) are prepared in accordance with the general scheme illustrated in FIG. 3. An example of an intermediate that can be used to prepare conjugates of Formula (III) and immunoconjugates of the Formula (I) is provided in FIG. 15. In an embodiment, a process of producing an immunoconjugate as described herein comprises reacting an effective amount of a thiol-functionalized antibody or antigen-binding fragment with a conjugate as described herein under reaction conditions effective to form the immunoconjugate. In an embodiment, the process further comprises reducing an antibody or an antigen-binding fragment under reducing conditions effective to form the thiol-functionalized antibody or antigen-binding fragment.

### EXAMPLES

Additional embodiments are disclosed in further detail in the following examples, which are not in any way intended to limit the scope of the claims.

The following abbreviations may be used herein and have the indicated definitions: Ac is acetyl (—C(═O)CH₃). AcOH is acetic acid. Ac₂O is acetic anhydride, DCM is dichloromethane, AgOAc is silver acetate, DMAP is 4-dimethylaminopyridine, DMF is N,N-dimethylformamide, DMFDMA is N,N-dimethylformamide dimethyl acetal, DMSO is dimethyl sulfoxide. ESI is electrospray ionization. EtOAc is ethyl acetate, h is hour, HCHO is formaldehyde, HPLC is high performance liquid chromatography. KHMDS is potassium bis(trimethylsilyl)amide, LCMS is liquid chromatography/mass spectrometry. MeOH is methanol, MsOH is p-toluenesulfonic acid, NaBH(OAc)₃ is sodium triacetoxy borohydride. NMO is N-methylmorpholine N-oxide, NMO is N-methylmorpholine N-oxide. NMR is nuclear magnetic resonance. OTBS is tert-butyldimethylsilyl ethers. PhMe is toluene. PIDA is (diacetoxyiodo)benzene, P(OEt)₃ is triethyl phosphite, PPh₃ is triphenylphosphine. PyH is pyridinium, PPTS is pyridinium p-toluenesulfonate, SFC is supercritical fluid chromatography, t-BuOOH is tert-butyl hydroperoxide. TEA is triethylamine. TLC is thin-layer chromatography. THF is tetrahydrofuran, and TsOH is p-toluenesulfonic acid.

### Example 1

### Synthesis of: (1S,9S)-1-((S)-2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-7A), (1S,9S)-1-((R)-2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-8A), (1R,9S)-1-((S)-2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-9A) and (1R,9S)-1-((R)-2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-10A)

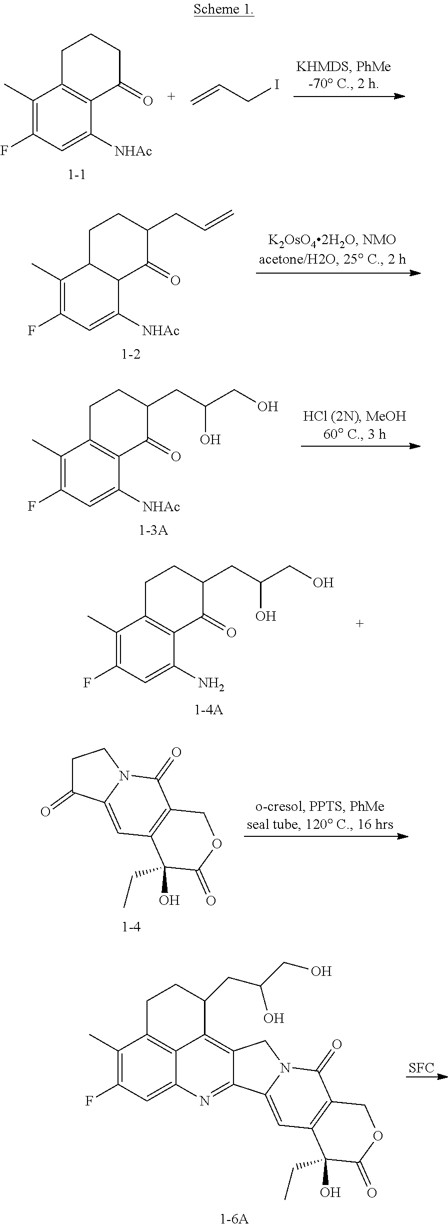

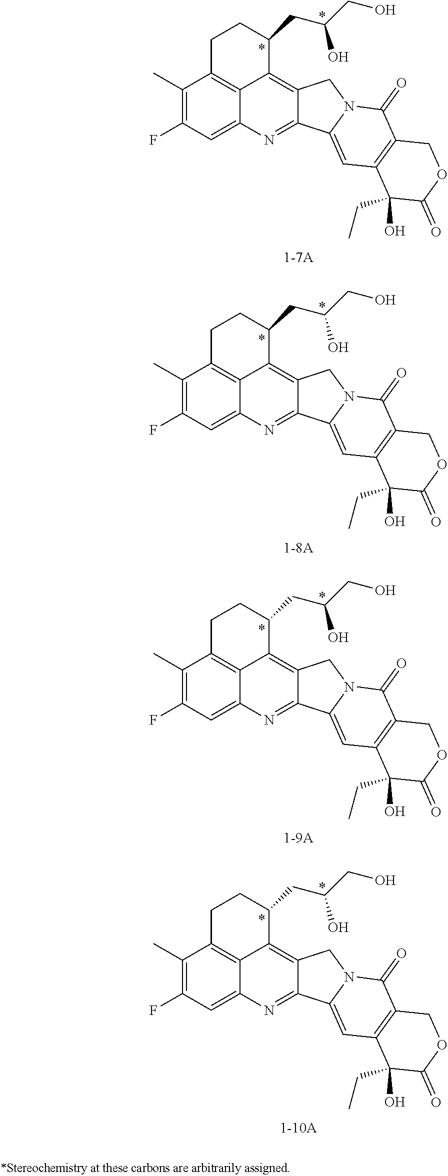 [SMILES: CC[C@@]1(O)C(=O)OCc2c1cc1n(c2=O)Cc2c-1nc1cc(F)c(C)c3c1c2[C@H](C[C@H](O)CO)CC3 ; CC[C@@]1(O)C(=O)OCc2c1cc1n(c2=O)Cc2c-1nc1cc(F)c(C)c3c1c2[C@H](C[C@@H](O)CO)CC3 ; CC[C@@]1(O)C(=O)OCc2c1cc1n(c2=O)Cc2c-1nc1cc(F)c(C)c3c1c2[C@@H](C[C@H](O)CO)CC3 ; CC[C@@]1(O)C(=O)OCc2c1cc1n(c2=O)Cc2c-1nc1cc(F)c(C)c3c1c2[C@@H](C[C@@H](O)CO)CC3]

### N-(7-Allyl-3-fluoro-4-methyl-8-oxo-5,6,7,8-tetrahydronaphthalen-1-yl)acetamide (1-2)

To a stirred mixture of N-(3-fluoro-4-methyl-8-oxo-5,6,7,8-tetrahydronaphthalen-1-yl)acetamide (1-1) (7.50 g, 31.9 mmol, 1.0 equiv) in toluene (150 mL) was added potassium bis(trimethylsilyl)amide) (1 M, 63.8 mL, 2.0 equiv) at −70° C. After stirring at −70° C., for 1 h, a solution of 3-iodoprop-1-ene (5.36 g, 31.9 mmol, 2.91 mL, 1.0 equiv) in toluene (75.0 mL) was added, and the mixture stirred at −70° C., for 1 h. Another reaction of the same scale was run in parallel. The two reactions were combined for work-up. It was quenched by dropwise addition of H₂O (200 mL) at −70° C., slowly warmed to 25° C., then the mixture extracted with ethyl acetate (3× 200 mL). The combined organic layers were washed with brine (500 mL), dried over Na₂SO₄, filtered, concentrated and the residue purified by silica gel column chromatography (petroleum ether/ethyl acetate) to give N-(7-allyl-3-fluoro-4-methyl-8-oxo-5,6,7,8-tetrahydronaphthalen-1-yl)acetamide (1-2) (9.70 g, 55% yield). ¹H NMR (400 MHz, DMSO-d₆) δ ppm 12.09 (br s, 1H) 8.29 (d, J=13.13 Hz, 1H) 5.69-5.97 (m, 1H) 4.98-5.21 (m, 2H) 2.93-3.07 (m, 1H) 2.77-2.90 (m, 1H) 2.64-2.74 (m, 1H) 2.54-2.63 (m, 1H) 2.05-2.26 (m, 8H) 1.65-1.78 (m, 1H), ¹⁹F NMR (376 MHz, DMSO-D₆) δ ppm −104.42. LCMS (ESI+) m/z: [MH]⁺ calcd for C₁₆H₁₉FNO₂⁺: 276.1, found: 276.1.

### N-(7-(2,3-Dihydroxypropyl)-3-fluoro-4-methyl-8-oxo-5,6,7,8-tetrahydronaphthalen-1-yl)acetamide (1-3A)

To a stirred mixture of N-(7-allyl-3-fluoro-4-methyl-8-oxo-5,6,7,8-tetrahydronaphthalen-1-yl)acetamide (1-2) (1.0 g, 3.63 mmol, 1.0 equiv) in acetone (20 mL) and water (1 mL) was added potassium osmate(VI) dihydrate (535 mg, 1.45 mmol, 0.4 equiv) and 4-MethylmorpholineN-oxidemonohydrate (850 mg, 7.26 mmol, 766 μL, 2.0 equiv). After stirring at 25° C., for 2 h, the reaction mixture was quenched by addition of water (200 mL) and saturated sodium thiosulfate solution (20 mL) at 25° C., and extracted with ethyl acetate (3×100 mL). The combined organic layers were dried over anhydrous sodium sulfate, filtered, concentrated, and the residue purified by silica gel column chromatography (petroleum ether/ethyl acetate) to give N-(7-(2,3-dihydroxypropyl)-3-fluoro-4-methyl-8-oxo-5,6,7,8-tetrahydronaphthalen-1-yl)acetamide (1-3A) (700 mg, 62% yield). ¹H NMR (400 MHz, DMSO-D₆) δ ppm 11.97-12.22 (m, 1H), 8.28 (d, J=13.18 Hz, 1H), 4.45-4.60 (m, 2H), 3.53-3.66 (m, 1H), 3.23-3.30 (m, 1H), 2.93-3.05 (m, 1H), 2.81-2.91 (m, 1H), 2.71-2.80 (m, 1H), 2.08-2.25 (m, 8H), 1.67-1.91 (m, 2H), 1.40-1.50 (m, 1H), ¹⁹F NMR (376 MHz, DMSO-D₆) δ ppm −104.74. LCMS (ESI+) m/z: [M+Na]⁺ calcd for C₁₆H₂₀FNO₄Na⁻: 332.1, found: 332.1.

### 8-Amino-2-(2,3-Dihydroxypropyl)-6-fluoro-5-methyl-3,4-dihydronaphthalen-1(2H)-one (1-4A)

To a stirred mixture of N-(7-(2,3-dihydroxypropyl)-3-fluoro-4-methyl-8-oxo-5,6,7,8-tetrahydronaphthalen-1-yl)acetamide (1-3A) (700 mg, 2.25 mmol, 1.0 equiv) in methanol (28 mL) was added hydrochloric acid (2 M, 28 mL). After stirring at 60° C., for 3 h, it was quenched by addition of saturated sodium bicarbonate solution (56 mL) at 25° C. to adjust pH to 7 and extracted with ethyl acetate (3×30 mL). The combined organic layers were dried over anhydrous sodium sulfate, filtered, concentrated, and the residue purified by silica gel column chromatography (petroleum ether/ethyl acetate) to give 8-amino-2-(2,3-Dihydroxy propyl)-6-fluoro-5-methyl-3,4-dihydronaphthalen-1(2H)-one (1-4A) (270 mg, 65% yield). ¹H NMR (400 MHz, DMSO-D₆) δ ppm 7.22-7.54 (m, 2H), 6.28-6.46 (m, 1H), 4.37-4.60 (m, 2H), 3.55-3.73 (m, 1H), 3.17-3.29 (m, 2H), 2.79-2.91 (m, 1H), 2.63-2.76 (m, 2H), 2.08-2.16 (m, 1H), 1.93-2.04 (m, 4H), 1.68-1.89 (m, 1H), 1.33-1.59 (m, 1H), ¹⁹F NMR (376 MHz, DMSO-D₆) δ ppm −104.74. LCMS (ESI+) m/z: [MH]⁺ calcd for C₁₄H₁₉FNO₃⁺: 268.1, found: 268.1.

### (9S)-1-(2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-6A)

After the mixture of 8-amino-2-(2,3-dihydroxypropyl)-6-fluoro-5-methyl-3,4-dihydronaphthalen-1(2H)-one (1-4A) (400 mg, 1.20 mmol, 1.0 equiv), (S)-4-ethyl-4-hydroxy-7,8-dihydro-1H-pyrano[3,4-f]indolizine-3,6,10(4H)-trione (1-4) (346 mg, 1.32 mmol, 1.1 equiv), o-cresol (945 mg, 8.74 mmol, 90 μL, 7.3 equiv) and pyridinium 4-toluenesulfonate (45.13 mg, 179 μmol, 0.15 equiv) in toluene (20 mL) was degassed and purged with argon for 3 times, it was stirred at 120° C., for 16 h under argon atmosphere. Then, it was quenched by addition of water (15 mL) at 25° C., and extracted with ethyl acetate (3× 20 mL). The combined organic layers were dried over anhydrous sodium sulfate filtered, concentrated, and the residue purified by silica gel column chromatography (ethyl acetate/methanol) to give (9S)-1-(2,3-dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-6A) (300 mg, 28% yield). ¹H NMR (400 MHz, DMSO-D₆) δ ppm 7.74 (br d, J=11.37 Hz, 1H), 7.30 (s, 1H), 6.51 (s, 1H), 5.28-5.52 (m, 3H), 4.23-5.01 (m, 2H), 3.53-3.74 (m, 2H), 2.90-3.25 (m, 4H), 2.28-2.42 (m, 4H), 1.57-2.06 (m, 5H), 1.29-1.49 (m, 1H), 0.75-0.96 (m, 3H). ¹⁹F NMR (376 MHz, DMSO-D₆) δ ppm −111.99, −111.98, −111.84. LCMS (ESI+) m/z: [MH]⁺ calcd for C₂₇H₂₈FN₂O₆⁺: 495.2, found: 495.2. SFC (retention time=0.720 min. 0.827 min. 1.749 min. 2.134 min).

### (1S,9S)-1-(S)-2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-7A). (1S,9S)-1-((R)-2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-8A), (1R,9S)-1-((S)-2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino [1,2-b]quinoline-10,13-dione (1-9A), (1R,9S)-1-((R)-2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-10A)

(9S)-1-(2,3-dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-6A) (300 mg, 606 μmol) was first separated by chiral SFC to give a mixture of (1S,9S)-1-((S)-2,3-dihydroxypropyl)-9-ethyl-5-fluoro-9-hydroxy-4-methyl-1,2,3,9,12,15-hexahydro-10H,13H-benzo[de]pyrano[3′,4′:6,7] indolizino[1,2-b]quinoline-10,13-dione (1-7A) and (1S,9S)-1-((R)-2,3-Dihydroxypropyl)-9-ethyl-5-fluoro-9-hydroxy-4-methyl-1,2,3,9,12,15-hexahydro-10H,13H-ben 70 [de]pyrano[3′,4′:6,7]indolizino[1,2-b]quinoline-10,13-dione (1-8A), (1R,9S)-1-((S)-2,3-Dihydroxy propyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-9A) (45.4 mg, 15% yield). (1R,9S)-1-((R)-2,3-Dihydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1-10A) (45.6 mg, 15% yield). Note: The stereochemistry of four products is arbitrarily assigned. SFC separation method: Instrument: Waters SFC80Q preparative SFC; Column: DAICEL CHIRALCEL OD (250 mm*30 mm, 10 um): Mobile phase: A for CO₂ and B for methanol; Gradient: B %=38% isocratic elution mode: Flow rate: 65 g/min; Wavelength: 220 nm: Column temperature: 40° C.; System back pressure: 100 bar.

The mixture of 1-7A and 1-8A (70.2 mg) was separated by chiral SFC again to give 1-7A (23.29 mg, 7.6% yield) and 1-8A as a mixture with 1-7A. SFC separation method: Instrument: Waters SFC80 preparative SFC; Column: REGIS(s,s) WHELK-01 (250 mm*30 mm, 10 um): Mobile phase: A for CO₂ and B for IPA; Gradient: B %=55% isocratic elution mode: Flow rate: 60 g/min; Wavelength: 220 nm: Column temperature: 40° C.; System back pressure: 100 bar.

Then the above mixture of 1-8A and 1-7A was further separated by SFC to give 1-8A (9.5 mg, 3.1% yield). SFC separation method: Instrument: Waters SFC150AP preparative SFC: Column: REGIS (s,s) WHELK-01 (250 mm*30 mm, 10 um); Mobile phase: A for CO₂ and B for MeOH: Gradient: B %=50% isocratic elution mode: Flow rate: 70 g/min; Wavelength: 220 nm: Column temperature: 35 degrees centigrade: System back pressure: 120 bar.

Spectra for 1-7A: ¹H NMR (400 MHz, DMSO-D₆) δ ppm 7.74 (br d, J=10.88 Hz, 1H), 7.30 (s, 1H), 6.51 (s, 1H), 5.18-5.53 (m, 4H), 4.65-4.75 (m, 1H), 4.43 (br t, J=5.13 Hz, 1H), 3.58 (br s, 2H), 3.16-3.28 (m, 2H), 3.07-3.15 (m, 2H), 2.27-2.41 (m, 4H), 1.95-2.11 (m, 1H), 1.80-1.94 (m, 2H), 1.69-1.78 (m, 1H), 1.53-1.66 (m, 1H), 0.87 (br t, J=7.25 Hz, 3H). ¹⁹F NMR (376 MHz, DMSO-D₆) δ ppm −111.996. LCMS (ESI+) m/z: [MH]⁺ calcd for C₂₇H₂₈O₆N₂F⁺: 495.2, found: 495.3. SFC (retention time=1.654 min).

Spectra for 1-8A: ¹H NMR (400 MHz, DMSO-D₆) δ ppm 7.74 (br d, J=11.13 Hz, 1H), 7.30 (s, 1H), 6.51 (s, 1H), 5.43 (s, 2H), 5.28 (s, 2H), 4.88 (d, J=5.75 Hz, 1H), 4.54 (t, J=5.63 Hz, 1H), 3.67-3.76 (m, 1H), 3.59 (br d, J=11.76 Hz, 1H), 3.36-3.41 (m, 1H), 3.22 (dt, J=11.07, 5.72 Hz, 1H), 3.10-3.18 (m, 1H), 2.92-3.08 (m, 1H), 2.29-2.42 (m, 4H), 1.70-1.98 (m, 4H), 1.33-1.49 (m, 1H), 0.83-0.92 (m, 3H). ¹⁹F NMR (376 MHz, DMSO-D₆) δ ppm −111.849. LCMS (ESI+) m/z: [MH]⁺ calcd for C₂₇H₂₈O₆N₂F⁺: 495.2, found: 495.3. SFC (retention time=2.032 min).

Spectra for 1-9A: ¹H NMR (400 MHz, DMSO-D₆) δ ppm 7.74 (d, J=11.13 Hz, 1H), 7.30 (s, 1H), 6.52 (s, 1H), 5.24-5.52 (m, 4H), 4.73 (br s, 1H), 4.45 (br s, 1H), 3.64-3.95 (m, 1H), 3.57 (br s, 1H), 3.18-3.24 (m, 1H), 3.04-3.14 (m, 2H), 2.38 (s, 3H), 2.31 (br d, J=12.96 Hz, 1H), 1.95-2.10 (m, 1H), 1.81-1.92 (m, 2H), 1.69-1.80 (m, 1H), 1.54-1.66 (m, 1H), 1.11-1.27 (m, 1H), 0.87 (br t, J=7.21 Hz, 3H). ¹⁹F NMR (376 MHz, DMSO-D₆) δ ppm −111.988. LCMS (ESI+) m/z: [MH]⁺ calcd for C₂₇H₂₈O₆N₂F⁺: 495.2, found: 495.3. SFC (retention time=1.538 min).

Spectra for 1-10A: ¹H NMR (400 MHz, DMSO-D₆) δ ppm 7.73 (d, J=11.13 Hz, 1H), 7.29 (s, 1H), 6.51 (s, 1H), 5.43 (s, 2H), 5.28 (s, 2H), 4.87 (br d, J=5.50 Hz, 1H), 4.54 (br s, 1H), 3.67-3.77 (m, 1H), 3.54-3.64 (m, 1H), 3.36-3.42 (m, 1H), 3.19-3.27 (m, 1H), 3.09-3.18 (m, 1H), 2.93-3.07 (m, 1H), 2.27-2.43 (m, 4H), 1.70-1.98 (m, 4H), 1.34-1.47 (m, 1H), 0.87 (t, J=7.34 Hz, 3H). ¹⁹F NMR (376 MHz, DMSO-D₆) δ ppm-111.849. LCMS (ESI+) m/z: [MH]⁺ calcd for C₂₇H₂₈O₆N₂F⁺: 495.2, found: 495.3. SFC (retention time=1.679 min).

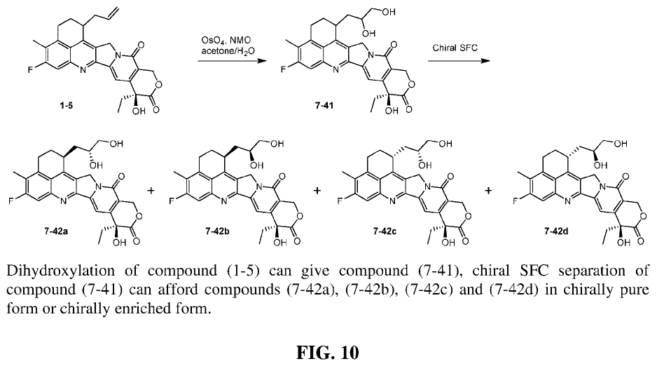

Alternative and/or additional synthesis routes for the compounds described in Example 1 can be found in FIG. 10.

### Example 2

### Synthesis of: (1R,9S)-9-ethyl-5-fluoro-1,9-dihydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (2-15) and (1S,9S)-9-ethyl-5-fluoro-1,9-dihydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (2-16)

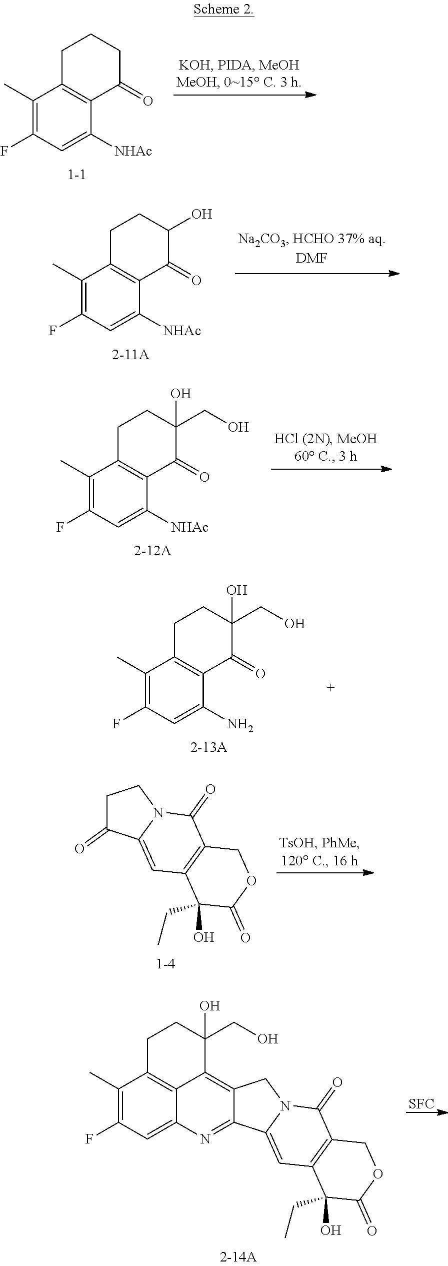

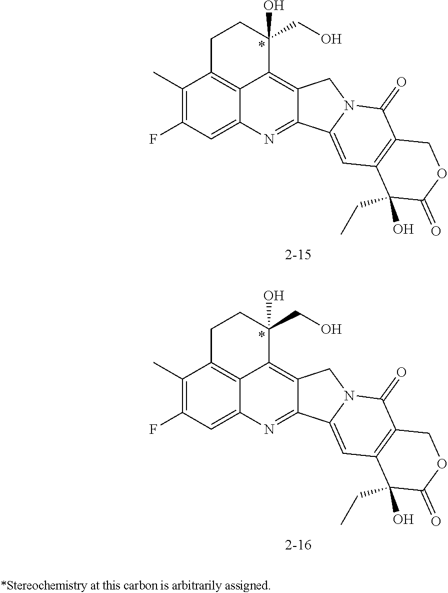 [SMILES: CC[C@@]1(O)C(=O)OCc2c1cc1n(c2=O)Cc2c-1nc1cc(F)c(C)c3c1c2[C@@](O)(CO)CC3 ; CC[C@@]1(O)C(=O)OCc2c1cc1n(c2=O)Cc2c-1nc1cc(F)c(C)c3c1c2[C@](O)(CO)CC3]

### 8-Amino-6-fluoro-2-hydroxy-2-(hydroxymethyl)-5-methyl-3,4-dihydronaphthalen-1(2H)-one (2-13A)

To a stirred mixture of N-(3-fluoro-7-hydroxy-7-(hydroxymethyl)-4-methyl-8-oxo-5,6,7,8-tetrahydronaphthalen-1-yl)acetamide (2-12A) (150 mg, 177 μmol, 1.0 equiv) in methanol (6.00 mL) was added hydrochloric acid (2 M, 6.00 mL). After stirring at 60° C. for 3 h, it was cooled to room temperature, adjusted to pH˜4 by addition of saturated aqueous sodium bicarbonate and extracted with ethyl acetate (4×20 mL). The combined organic layers were dried over anhydrous sodium sulfate, filtered, concentrated, and the residue purified by silica gel column chromatography (petroleum ether/ethyl acetate) to give 8-amino-6-fluoro-2-hydroxy-2-(hydroxymethyl)-5-methyl-3,4-dihydronaphthalen-1(2H)-one (2-13A) (95 mg, 74% yield). ¹H NMR (400 MHz, DMSO-D₆) δ ppm 7.41 (br s, 2H) 6.37 (d, J=12.51 Hz, 1H) 4.93 (s, 1H) 4.66 (t, J=5.94 Hz, 1H) 3.60 (dd, J=11.01, 6.00 Hz, 1H) 3.35 (dd, J=11.01, 5.88 Hz, 1H) 2.70-2.86 (m, 2H) 2.16 (dt, J=13.35, 5.58 Hz, 1H) 1.97 (d, J=1.13 Hz, 3H) 1.79-2.02 (m, 1H). LCMS (ESI+) m/z: [MH]⁺ calcd for C₁₂H₁₅FNO₃⁺: 240.1, found: 240.3.

### (9S)-9-Ethyl-5-fluoro-1,9-dihydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (2-14A)

To a stirred mixture of 8-amino-6-fluoro-2-hydroxy-2-(hydroxymethyl)-5-methyl-3,4-dihydronaphthalen-1(2H)-one (2-13A) (80 mg, 334 μmol, 1.0 equiv) and(S)-4-ethyl-4-hydroxy-7,8-dihydro-1H-pyrano[3,4-f]indolizine-3,6,10(4H)-trione (1-4) (176 mg, 668 μmol, 2.0 equiv) in toluene (4 mL) was added toluene-4-sulfonic acid (23.0 mg, 133 μmol, 0.4 equiv). After stirring at 120° C., for 16 h, it was cooled to room temperature, quenched by addition of H₂O (10 mL) at 20° C., and extracted with dichloromethane (3×20 mL). The combined organic layers were washed with brine (50 mL), dried over Na₂SO₄, filtered, concentrated, and the residue was purified by silica gel column chromatography (ethyl acetate/methanol) to give (9S)-9-ethyl-5-fluoro-1,9-dihydroxy-1-(hydroxymethyl)-4-methyl-1,2,3,9,12,15-hexahydro-10H,13H-benzo[de]pyrano[3′,4′:6,7]indol izino[1,2-b]quinoline-10,13-dione (2-14A) (80 mg, 46% yield). ¹H NMR (400 MHz, DMSO-D₆) δ ppm 7.76 (d, J=10.88 Hz, 1H) 7.31 (s, 1H) 6.50 (d, J=2.13 Hz, 1H) 5.73 (d, J=2.50 Hz, 1H) 5.44 (br d, J=7.63 Hz, 4H) 4.88-5.02 (m, 1H) 3.48-3.65 (m, 2H) 3.14-3.24 (m, 1H) 2.94-3.10 (m, 1H) 2.44-2.49 (m, 1H) 2.36 (s, 3H) 1.94-2.03 (m, 1H) 1.82-1.91 (m, 2H) 0.84-0.90 (m, 3H). ¹⁹F NMR (376 MHz, DMSO-D₆) δ ppm-112.28. LCMS (ESI+) m/z: [MH]⁺ calcd for C₂₅H₂₄FN₂O₆⁺: 467.1, found: 467.2. SFC (RT=2.284 min, 2.571 min).

### (1R,9S)-9-Ethyl-5-fluoro-1,9-dihydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (2-15) and (15.95)-9-ethyl-5-fluoro-1,9-dihydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (2-16)

(9S)-9-ethyl-5-fluoro-1,9-dihydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (2-14A) (80 mg, 171 μmol) was separated by SFC (Instrument: Waters SFC150AP preparative SFC: Column: IH (250 mm*30 mm, 10 um); Mobile phase: A for CO₂ and B for Gradient: B %=35% isocratic elution mode: Flow rate: 70 g/min; methanol; Wavelength: 220 nm: Column temperature: 35 degrees centigrade: System back pressure:

120 bar.) to afford (1R,9S)-9-ethyl-5-fluoro-1,9-dihydroxy-1-(hydroxymethyl)-4-methyl-1,2,3,9,12,15-hexahydro-10H,13H-benzo[de]pyrano[3′,4′:6,7]indol izino[1,2-b]quinoline-10,13-dione (2-15) (compound 2-15 may be the opposite enantiomer of that depicted) (17.2 mg) and (1S,9S)-9-ethyl-5-fluoro-1,9-dihydroxy-1-(hydroxymethyl)-4-methyl-1,2,3,9,12,15-hexahydro-10H,13H-benzo[de]pyrano [3′,4′:6,7]indolizino[1,2-b]quinoline-10,13-dione (2-16) (compound 2-16 may be the opposite enantiomer of that depicted) (23.5 mg). Note: The stereochemistry of the two products is arbitrarily assigned.

Spectra for 2-15: ¹H NMR (400 MHz, DMSO-D₆) δ ppm 7.76 (d, J=10.88 Hz, 1H) 7.31 (s, 1H) 6.50 (s, 1H) 5.74 (s, 1H) 5.44 (br d, J=9.01 Hz, 4H) 4.96 (t, J=6.19 Hz, 1H) 3.45-3.67 (m, 2H) 3.14-3.24 (m, 1H) 2.95-3.10 (m, 1H) 2.39-2.46 (m, 1H) 2.36 (s, 3H) 1.98 (td, J=13.13, 5.50 Hz, 1H) 1.79-1.92 (m, 2H) 0.87 (t, J=7.32 Hz, 3H), 19F NMR (376 MHz, DMSO-D₆) δ ppm −112.25. LCMS (ESI+) m/z: [MH]⁺ calcd for C₂₅H₂₄FN₂O₆⁺: 467.1, found: 467.3. SFC (RT=2.292 min).

Spectra for 2-16: ¹H NMR (400 MHz, DMSO-D₆) δ ppm 7.77 (d, J=10.92 Hz, 1H) 7.31 (s, 1H) 6.51 (s, 1H) 5.74 (s, 1H) 5.44 (d, J=7.91 Hz, 4H) 4.91-5.03 (m, 1H) 3.50-3.66 (m, 2H) 3.13-3.26 (m, 1H) 2.96-3.11 (m, 1H) 2.39-2.46 (m, 1H) 2.36 (s, 3H) 1.98 (td. J=13.02, 5.83 Hz, 1H) 1.80-1.92 (m, 2H) 0.88 (t, J=7.28 Hz, 3H). ¹⁹F NMR (376 MHz, DMSO-D₆) δ ppm −112.27. LCMS (ESI+) m/z: [MH]⁺ calcd for C₂₆H₂₆FN₂O₅⁺: 467.1, found: 467.3. SFC (RT=2.580 min).

### Example 3

### Synthesis of: (1R,9S)-9-Ethyl-5-fluoro-1,9-dihydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione (3-24) and (1S,9S)-9-ethyl-5-fluoro-1,9-dihydroxy-1-((2-hydroxyethoxy)methyl)-4-methyl-1,2,3,9,12,15-hexahydro-10H,13H-benzo[de]pyrano[3′,4′:6,7] indolizino[1,2-b]quinoline-10,13-dione (3-25)

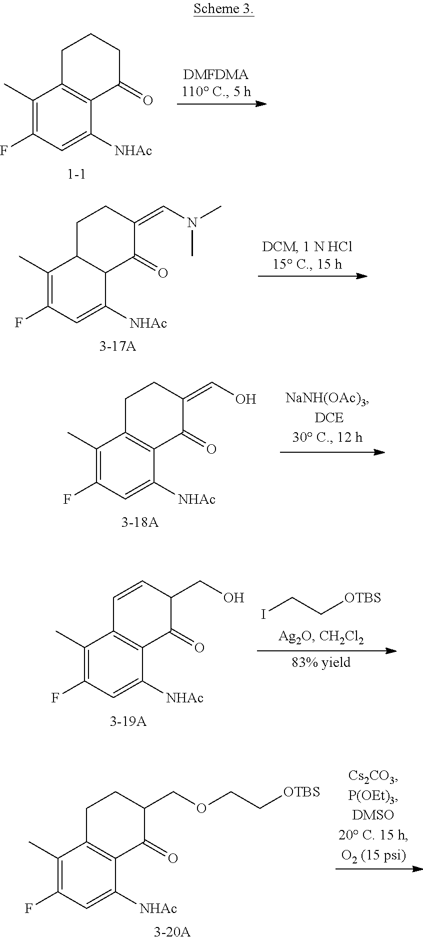

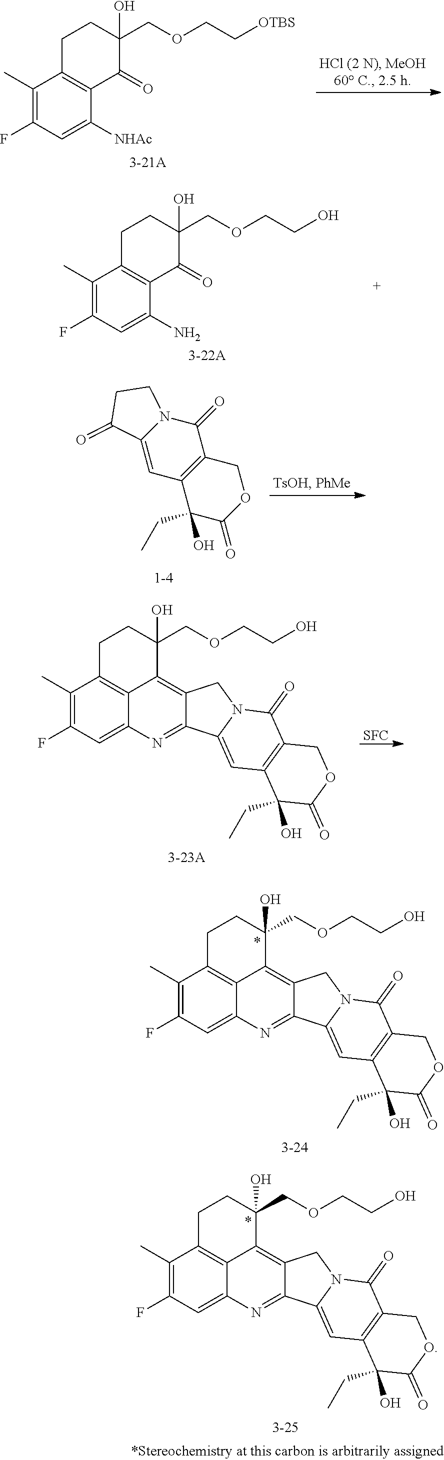

### N-(7-((Dimethylamino)methylene)-3-fluoro-4-methyl-8-oxo-5,6,7,8-tetrahydronaphthalen-1-yl)acetamide (3-17A)

After a stirred mixture of N-(3-fluoro-4-methyl-8-oxo-5,6,7,8-tetrahydronaphthalen-1-yl)acetamide (1-1) (40.0 g, 170 mmol, 1.0 equiv) in 1,1-dimethoxy-N,N-dimethylmethanamine (400 mL) was heated at 110° C., for 5 h, it was cooled to 15° C., and concentrated to give N-(7-((dimethylamino)methylene)-3-fluoro-4-methyl-8-oxo-5,6,7,8-tetrahydronaphthalen-1-yl)acetamide (3-17A) (46.6 g, 94% yield), which was used directly in the next step without further purification. ¹H NMR (400 MHz, DMSO-D₆) δ ppm 13.07 (s, 1H), 8.20 (d, J=13.26 Hz, 1H), 7.73 (s, 1H), 3.15 (s, 6H), 2.68-2.81 (m, 4H), 2.12 (d, J=1.75 Hz, 3H), 2.07 (s, 3H). ¹⁹F NMR (376 MHz, DMSO-D₆) δ ppm −108.358. LCMS (ESI+) m/z: [MH]⁺ calcd for C₁₆H₂₀FN₂O₂⁺: 291.1, found: 291.2.

### N-(3-Fluoro-7-(hydroxymethylene)-4-methyl-8-oxo-5,6,7,8-tetrahydronaphthalen-1-yl)acetamide (3-18A)

To a stirred mixture of N-(7-((dimethylamino)methylene)-3-fluoro-4-methyl-8-oxo-5,6,7,8-tetrahydronaphthalen-1-yl)acetamide (3-17A) (46.0 g, 158 mmol, 1.0 equiv) in dichloromethane (920 mL) was added 1 N hydrochloric acid solution (920 mL) at 15° C. After stirring at 15° C., for 15 h, it was extracted with dichloromethane (3×600 mL), combined organic layers washed with brine, dried over Na₂SO₄, filtered, and concentrated to give N-(3-fluoro-7-(hydroxymethylene)-4-methyl-8-oxo-5,6,7,8-tetrahydronaphthalen-1-yl)acetamide (3-18A) (41.0 g, 98% yield), which was used in the next step directly without further purification. ¹H NMR (400 MHz, DMSO-D₆) δ ppm 12.55 (br s, 1H), 11.45 (br s, 1H), 8.26 (d, J=13.26 Hz, 1H), 7.87 (s, 1H), 2.80 (br t, J=6.69 Hz, 2H), 2.55 (br t, J=6.50 Hz, 2H), 2.10-2.16 (m, 6H). ¹⁹F NMR (376 MHz, DMSO-D₆) δ ppm −105.54. LCMS (ESI+) m/z: [MH]⁺ calcd for C₁₄H₁₅FNO₃⁺: 264.1, found: 264.5.

### N-(3-Fluoro-7-(hydroxymethyl)-4-methyl-8-oxo-5,6,7,8-tetrahydronaphthalen-1-yl)acetamide (3-19A)

To a stirred mixture of N-(3-fluoro-7-(hydroxymethylene)-4-methyl-8-oxo-5,6,7,8-tetrahydronaphthalen-1-yl)acetamide (3-18A) (41.0 g, 155 mmol, 1.0 equiv) in dichloromethane (420 mL) and acetic acid (4.2 mL), was added sodium triacetoxyhydroborate (39.6 g, 93.4 mmol, 1.2 equiv) portion wise at 20° C. After stirring at 30° C., for 15 h under nitrogen atmosphere, it was quenched by addition of water (250) mL) and extracted with dichloromethane (3×350 mL). The combined organic layers were washed with brine, dried over Na₂SO₄, filtered, concentrated, and the residue purified by silica gel column chromatography (petroleum ether/ethyl acetate) to give N-(3-fluoro-7-(hydroxymethyl)-4-methyl-8-oxo-5,6,7,8-tetrahydronaphthalen-1-yl)acetamide (3-19A) (34.0 g, 82% yield). ¹H NMR (400 MHz, DMSO-D₆) δ ppm 12.13 (s, 1H), 8.29 (d, J=13.26 Hz, 1H), 4.68 (t, J=5.44 Hz, 1H), 3.62-3.82 (m, 2H), 3.03 (dt, J=17.51, 4.63 Hz, 1H), 2.76-2.88 (m, 1H), 2.63-2.73 (m, 1H), 2.17-2.24 (m, 1H), 2.15 (s, 3H), 2.13 (d, J=1.50 Hz, 3H), 1.82-1.93 (m, 1H), ¹⁹F NMR (376 MHz, DMSO-D₆) δ ppm-104.25. LCMS (ESI+) m/z: [MH]⁺ calcd for C₁₄H₁₇FNO₃⁺: 266.1, found: 266.2.

### N-(7-((2-((Tert-butyldimethylsilyl)oxy)ethoxy)methyl)-3-fluoro-4-methyl-8-oxo-5,6,7,8-tetrahydronaphthalen-1-yl)acetamide (3-20A)

To a stirred mixture of N-[7-fluoro-3-(hydroxymethyl)-8-methyl-4-oxo-tetralin-5-yl]acetamide (3-19A) (2.5 g, 9.42 mmol, 1 equiv) in dichloromethane (25 mL) was added Ag₂O (21.8 g, 94.2 mmol, 10 equiv) and tert-butyl-(2-iodoethoxy)-dimethyl-silane (53.95 g, 188.48 mmol, 20 equiv). After stirring at 60° C., for 36 h under nitrogen atmosphere and cooled down to room temperature, it was filtered to remove Ag₂O. It was concentrated and the residue purified by silica gel column chromatography (petroleum ether/ethyl acetate) to give N-(7-((2-((tert-butyldimethylsilyl)oxy)ethoxy)methyl)-3-fluoro-4-methyl-8-oxo-5,6,7,8-tetrahydronaphthalen-1-yl)acetamide (3-20A) (3.35 g, 83% yield). ¹H NMR (400 MHz, DMSO-D₆) δ ppm 12.10 (s, 1H), 8.29 (d, J=13.13 Hz, 1H), 3.66-3.79 (m, 4H), 3.43-3.51 (m, 2H), 3.03 (dt, J=17.45, 4.22 Hz, 1H), 2.76-2.94 (m, 2H), 2.17-2.26 (m, 1H), 2.11-2.16 (m, 6H), 1.88 (qd, J=12.09, 4.38 Hz, 1H), 0.84 (s, 9H), 0.01 (s, 6H). ¹⁹F NMR (376 MHz, DMSO-D₆) δ ppm −104.08. LCMS (ESI+) m/z: [MH]⁺ calcd for C₂₂H₃₅FNO₄Si⁺: 424.2, found: 424.3.

### N-(7-((2-((tert-butyldimethylsilyl)oxy)ethoxy)methyl)-3-fluoro-7-hydroxy-4-methyl-8-oxo-5,6,7,8-tetrahydronaphthalen-1-yl)acetamide (3-21A)

To a stirred mixture of N-(7-((2-((tert-butyldimethylsilyl)oxy)ethoxy)methyl)-3-fluoro-4-methyl-8-oxo-5,6,7,8-tetrahydronaphthalen-1-yl)acetamide (3-20A) (1.1 g, 2.60 mmol, 1 equiv) and cesium carbonate (169 mg, 519 μmol, 0.2 equiv) in DMSO (22 mL), was added triethyl phosphite (862 mg, 5.19 mmol, 890 μL, 2.0 equiv). After the reaction mixture was degassed under vacuum and purged with O₂ three times, it was stirred under O₂ (15 psi) at 25° C., for 3 h. Then it was quenched by addition of water (30 mL) and extracted with ethyl acetate (3×30 mL). The combined organic layers were washed with brine, dried over sodium sulfate, filtered, concentrated and the residue purified by silica gel column chromatography (petroleum ether/ethyl acetate) to give N-(7-((2-((tert-butyldimethylsilyl)oxy)ethoxy)methyl)-3-fluoro-7-hydroxy-4-methyl-8-oxo-5,6,7,8-tetrahydronaphthalen-1-yl)acetamide (3-21A) (250 mg, 21% yield). ¹H NMR (400 MHz, CDCl₃) δ ppm 11.85 (br s, 1H), 8.46 (d, J=12.8 Hz, 1H), 4.09 (s, 1H), 3.64-3.77 (m, 4H), 3.51-3.62 (m, 2H), 2.95-3.05 (m, 1H), 2.80-2.92 (m, 1H), 2.38-2.51 (m, 1H), 2.24 (s, 3H), 2.07-2.18 (m, 4H), 0.87 (s, 9H), 0.03 (d, J=8.9 Hz, 6H). ¹⁹F NMR (376 MHz, CDCl₃) δ ppm −100.715. LCMS (ESI+) m/z: [MH]⁺ calcd for C₂₂H₃₅FNO₅Si⁺: 440.2, found: 440.2.

### 8-Amino-6-fluoro-2-hydroxy-2-((2-hydroxyethoxy)methyl)-5-methyl-3,4-dihy dronaphthalene-1(2H)-one (3-22A)

After a mixture of N-(7-((2-((tert-butyldimethylsilyl)oxy)ethoxy)methyl)-3-fluoro-7-hydroxy-4-methyl-8-oxo-5,6,7,8-tetrahydronaphthalen-1-yl)acetamide (3-21A) (450 mg, 1.02 mmol, 1 equiv) in HCl (2 M, 18 mL) and methanol (18 mL) was stirred at 60° C. for 2.5 h, it was cooled to room temperature, concentrated to remove methanol and quenched by addition of saturated sodium bicarbonate solution at 25° C., to adjust pH 7 and extracted with ethyl acetate (3×30 mL). The combined organic layers were dried over Na₂SO₄, filtered, concentrated to give 8-amino-6-fluoro-2-hydroxy-2-(2-hydroxyethoxymethyl)-5-methyl-tetralin-1-one (3-22A) (200 mg, 68% yield), which was used directly for the next step without any purification. LCMS (ESI+) m/z: [MH]⁺ calcd for C₁₄H₁₉FNO₄: 384.1, found: 384.1.

### (9S)-9-Ethyl-5-fluoro-1,9-dihydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione

### N-(3-Fluoro-7-hydroxy-4,7-dimethyl-8-oxo-5,6,7,8-tetrahydronaphthalen-1-yl)acetamide (3-23A)

To a stirred mixture of 8-amino-6-fluoro-2-hydroxy-2-(2-hydroxyethoxymethyl)-5-methyl-tetralin-1-one (3-22A) (200 mg, 705 μmol, 1.0 equiv) and (4S)-4-ethyl-4-hydroxy-7,8-dihydro-1H-pyrano[3,4-f]indolizine-3,6,10-trione (1-4) (371 mg, 1.41 mmol, 2.0 equiv) in toluene (10 mL) was added 4-methylbenzenesulfonic acid (48.6 mg, 282 μmol, 0.4 equiv). After the mixture was degassed and purged with argon 3 times, it was stirred at 120° C., for 3 h under argon, cooled down to room temperature, diluted with water (20 mL) and extracted with ethyl acetate (3×30 mL). The combined organic layers were dried over Na₂SO₄, filtered, concentrated, and the residue purified by silica gel column chromatography (ethyl acetate/methanol) to give (9S)-9-ethyl-5-fluoro-1,9-dihydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione (3-23A) (100 mg, 27% yield). LCMS (ESI+) m/z: [MH]⁺ calcd for C₂₇H₂₈FN₂O₇⁺: 511.2, found: 511.2. SFC (retention time=1.845 min, 2.002 min).

### (1R,9S)-9-Ethyl-5-fluoro-1,9-dihydroxy-1-((2-hydroxyethoxy)methyl)-4-meth yl-1,2,3,9,12,15-hexahydro-10H,13H benzo[de]pyrano[3′,4′:6,7]indolizino[1,2-b]quinoline-10,13-dione (3-24) and (1S,9S)-9-ethyl-5-fluoro-1,9-dihydroxy-1-((2-hydroxyethoxy)methyl)-4-methyl-1,2,3,9,12,15-hexahydro-10H,13H-benzo[de]pyrano [3′,4′:6,7]indolizino[1,2-b]quinoline-10,13-dione (3-25)

(9S)-9-ethyl-5-fluoro-1,9-dihydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione (3-23A) (100 mg) was separated by chiral HPLC (column: DAICEL CHIRALCEL OD (250 mm*30 mm, 10 um): mobile phase: [CO₂-methanol]: B %: 35%, isocratic elution mode) Gradient: B %=50% isocratic elution mode: Flow rate: 70 g/min; Wavelength: 220 nm: Column temperature: 35 degrees centigrade: System back pressure: 120 bar) to give (1R,9S)-9-ethyl-5-fluoro-1,9-dihydroxy-1-((2-hydroxy ethoxy)methyl)-4-methyl-1,2,3,9,12,15-hexahydro-10H,13H-benzo[de]pyrano[3′,4′:6,7]indolizino[1,2-b]quinoline-10,13-dione (3-24) (28.0 mg) (compound 3-24 may be the opposite enantiomer of that depicted) and ((1S,9S)-9-ethyl-5-fluoro-1,9-dihydroxy-1-((2-hydroxyethoxy)methyl)-4-methyl-1,2,3,9,12,15-hexahydro-10H, 13H-benzo[de]pyrano[3′,4′:6,7]indolizino[1,2-b]quinolone-10,13-dione (3-25) (20.8 mg) (compound 3-25 may be the opposite enantiomer of that depicted). Note: The stereochemistry of the two products is arbitrarily assigned.

Spectra for 3-24: ¹H NMR (400 MHz, CD₃OD): δ ppm 7.57 (s, 1H), 7.51 (d, J=10.8 Hz, 1H), 5.49-5.62 (m, 3H), 5.37 (d, J=16.3 Hz, 1H), 3.54-3.83 (m, 6H), 3.20-3.29 (m, 1H), 3.04-3.18 (m, 1H), 2.69 (br dd, J=12.8, 3.3 Hz, 1H), 2.39 (s, 3H), 2.10 (td, J=13.2, 5.6 Hz, 1H), 1.97 (qd, J=7.2, 2.3 Hz, 2H), 1.02 (t, J=7.3 Hz, 3H). ¹⁹F NMR (376 MHz, CD₃OD) δ ppm −113.051. LCMS (ESI+) m/z: [MH]⁺ calcd for C₂₇H₂₈FN₂O₇⁺: 511.2, found: 511.3. SFC (retention time=2.005 min).

Spectra for 3-25: ¹H NMR (400 MHz, CD₃OD): 7.59 (s, 1H), 7.53 (br d, J=10.6 Hz, 1H), 5.45-5.61 (m, 3H), 5.35 (d, J=16.3 Hz, 1H), 3.47-3.75 (m, 6H), 3.26 (br d, J=4.5 Hz, 1H), 3.00-3.17 (m, 1H), 2.69 (br dd, J=12.9, 3.3 Hz, 1H), 2.40 (s, 3H), 2.18 (td. J=13.2, 5.6 Hz, 1H), 1.88-2.02 (m, 2H), 0.99 (t, J=7.4 Hz, 3H). ¹⁹F NMR (376 MHz, CD₃OD) δ ppm −112.862. LCMS (ESI+) m/z: [MH]⁺ calcd for C₂₇H₂₈FN₂O₇⁺: 511.2, found: 511.3. SFC (retention time=1.847 min).

Alternative and/or additional synthesis routes for the compounds described in Example 3 can be found in FIG. 7.

### Example 4

### Synthesis of: (1S,9S)-9-Ethyl-5-fluoro-1,9-dihydroxy-1-(2-hydroxyethyl)-4-methyl-1,2,3,9,12,15-hexahydro-10H,13H-benzo[de]pyrano[3′,4′:6,7]indolizino[1,2-6]quinoline-10,13-dione (4-29) and (1R,9S)-9-ethyl-5-fluoro-1,9-dihydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (4-30)

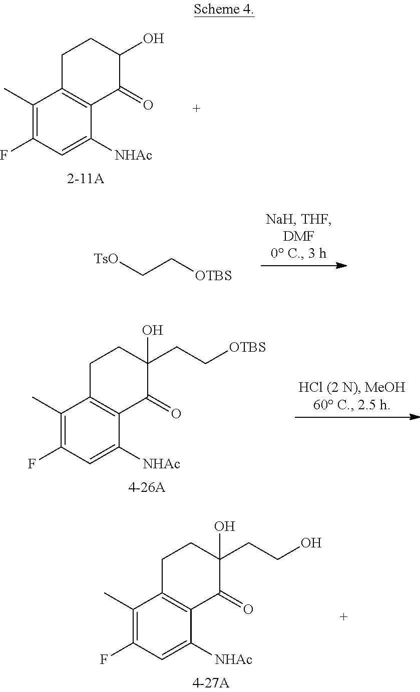 [SMILES: CC(=O)Nc1cc(F)c(C)c2c1C(=O)C(O)CC2 ; Cc1ccc(S(=O)(=O)OCCO[Si](C)(C)C(C)(C)C)cc1 ; CC(=O)Nc1cc(F)c(C)c2c1C(=O)C(O)(CCO[Si](C)(C)C(C)(C)C)CC2 ; CC(=O)Nc1cc(F)c(C)c2c1C(=O)C(O)(CCO)CC2]

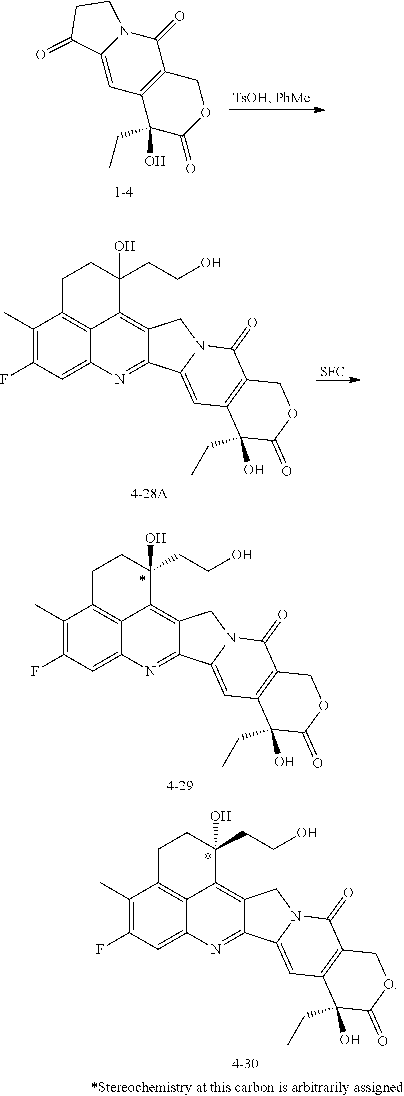

### N-(7-(2-((tert-Butyldimethylsilyl)oxy)ethyl)-3-fluoro-7-hydroxy-4-methyl-8-oxo-5,6,7,8-tetrahydronaphthalen-1-yl)acetamide (4-26A)

To a mixture of N-(3-fluoro-7-hydroxy-4-methyl-8-oxo-5,6,7,8-tetrahydronaphthalen-1-yl)acetamide (2-11A) (4.00 g, 15.9 mmol, 1.0 equiv) in N,N-dimethylformamide (40 mL) and tetrahydrofuran (40 mL), was added sodium hydride (2.55 g, 63.6 mmol, 60 wt %, 4.0 equiv) at 0° C., stirred at 0° C., for 0.5 h, and 2-[tert-butyl(dimethyl) silyl]oxyethyl 4-methylbenzenesulfonate (10.5 g, 31.8 mmol, 2.0 equiv) added at 0° C., and stirred at 0° C., for 2.5 h. Then the reaction mixture was quenched by addition of saturated ammonium chloride (70 mL), critic acid (0.1 mol/L, 30 mL), and extracted with ethyl acetate (4×100 mL). The combined organic layers were dried over anhydrous sodium sulfate, filtered, concentrated, and the residue purified by silica gel column chromatography (petroleum ether/ethyl acetate) to give N-[3-[2-[tert-butyl(dimethyl)silyl]oxyethyl]-7-fluoro-3-hydroxyl-8-methyl-4-oxo-tetralin-5-yl]acetamide (4-26A) (1.50 g, 13% yield). ¹H NMR (DMSO-D₆, 400 MHz): δ ppm 11.88 (s, 1H), 8.29 (d, J=13.0 Hz, 1H), 5.35 (s, 1H), 3.44-3.48 (m, 2 H), 2.53-2.57 (m, 2H), 2.29 (t, J=4.9 Hz, 1H), 2.20 (br d, J=5.4 Hz, 1H), 2.15 (s, 3H), 2.11 (d, J=1.3 Hz, 3H), 1.74-1.85 (m, 2H), 0.80 (s, 9H), −0.02 (s, 6H) LCMS (ESI+) m/z: [MH]⁺ calcd for C₂₁H₃₃FNO₄Si⁺: 410.2, found: 410.3.

### 8-Amino-6-fluoro-2-hydroxy-2-(2-hydroxyethyl)-5-methyl-3,4-dihydronaphthalen-1(2H)-one (4-27A)

To a stirred mixture of N-[3-[2-[tert-butyl(dimethyl)silyl]oxyethyl]-7-fluoro-3-hydroxy-8-methyl-4-oxo-tetralin-5-yl]acetamide (4-26A) (1.50 g, 3.66 mmol, 1.0 equiv) in methanol (60 mL) was added HCl (2 M, 60 mL) at 60° C. After stirring at 60° C., for 2.5 h, it was cooled down to room temperature, adjusted to pH˜4 by addition of saturated sodium hydrogen carbonate, and extracted with ethyl acetate (4×100 mL). The combined organic layers were dried over anhydrous sodium sulfate, filtered, concentrated, and the residue purified by silica gel column chromatography (petroleum ether/ethyl acetate) to give 8-amino-6-fluoro-2-hydroxy-2-(2-hydroxyethyl)-5-methyl-3,4-dihydronaphthalen-1(2H)-one (4-27A) (310 mg, 33% yield). ¹H NMR (400 MHz, DMSO-D₆) δ ppm 7.43 (br s, 2H) 6.38 (d, J=12.51 Hz, 1H) 4.96 (s, 1H) 4.30-4.39 (m, 1H) 3.54-3.63 (m, 1H) 3.37-3.46 (m, 1H) 2.70-2.88 (m, 2H) 2.04-2.14 (m, 1H) 1.97 (s, 3H) 1.82-1.93 (m, 1H) 1.60-1.77 (m, 2H). LCMS (ESI+) m/z: [MH−H₂O]⁺ calcd for C₁₃H₁₅FNO₂⁺: 254.1, found: 236.2.

### (9S)-9-Ethyl-5-fluoro-1,9-dihydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (4-28A)

After the stirred mixture of 8-amino-6-fluoro-2-hydroxy-2-(2-hydroxyethyl)-5-methyl-3,4-dihydronaphthalen-1(2H)-one (4-27A) (310 mg, 1.22 mmol, 1.0 equiv). (4S)-4-ethyl-4-hydroxy-7,8-dihydro-1H-pyrano[3,4-f]indolizine-3,6,10-trione (1-4) (644 mg, 2.45 mmol, 2.0 equiv) and 4-methylbenzenesulfonic acid (84.3 mg, 489 μmol, 0.4 equiv) in toluene (15.5 mL) was degassed and purged with argon for 3 times, it was stirred at 120° C., for 16 h. It was cooled down to room temperature, diluted with water (30 mL) and extracted with ethyl acetate (3× 60 mL). The combined organic layers were dried over Na₂SO₄, filtered, concentrated, and the residue purified by prep-HPLC (column: Phenomenex luna C18 (250*70 mm, 15 um): mobile phase: [H₂O (0.2% formic acid)-acetonitrile]; gradient: 25%-55% B over 20.0 min) to give (9S)-9-ethyl-5-fluoro-1,9-dihydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (4-28A) (40 mg, 6% yield). LCMS (ESI+) m/z: [MH]⁺ calcd for C₂₆H₂₆FN₂O₆⁺: 481.1, found: 481.4.

### (1S,9S)-9-Ethyl-5-fluoro-1,9-dihydroxy-1-(2-hydroxyethyl)-4-methyl-1,2,3,9,12,15-hexahydro-10H,13H-benzo [de]pyrano[3′,4′:6,7]indolizino[1,2-b]quinoline-10,13-dione (4-29) and (1R,9S)-9-ethyl-5-fluoro-1,9-dihydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (4-30)

(9S)-9-ethyl-5-fluoro-1,9-dihydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (4-28A) (40.0 mg, 83.2 μmol) was separated by SFC (Instrument: undefined; Column: REGIS (s,s) WHELK-01 (250 mm*30 mm, 5 um): (Mobile phase: A for CO₂ and B for methanol; Gradient: B %=50.00% isocratic elution mode: Flow rate: 70.00 g/min; Monitor wavelength: 220&254 nm: Column temperature: 40° C.: System back pressure: 100 bar) to afford (1S,9S)-9-ethyl-5-fluoro-1,9-dihydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (4-29) (compound 4-29 may be the opposite enantiomer of that depicted (10.2 mg) and (1R,9S)-9-ethyl-5-fluoro-1,9-dihydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (4-30) (compound 4-30 may be the opposite enantiomer of that depicted) (5.00 mg). Note: The stereochemistry of the two products is arbitrarily assigned.

Spectra for 4-29: ¹H NMR (400 MHz, DMSO-D₆) δ ppm 7.76 (d, J=10.8 Hz, 1H), 7.31 (s, 1H), 6.50 (s, 1H), 5.81 (s, 1H), 5.28-5.52 (m, 4H), 4.47 (t, J=4.9 Hz, 1H), 3.70-3.81 (m, 1H), 3.57-3.67 (m, 1H), 3.05-3.26 (m, 2H), 2.34-2.46 (m, 1H), 2.37 (s, 3H), 1.95-2.03 (m, 1H), 1.77-1.90 (m, 4H), 0.88 (br t, J=7.3 Hz, 3H). ¹⁹F NMR (376 MHz, DMSO-D₆) δ ppm −112.26. LCMS (ESI+) m/z: [MH]⁺ calcd for C₂₆H₂₆FN₂O₆⁺: 481.1, found: 481.2. SFC (retention time=1.879 min).

Spectra for 4-30: ¹H NMR (400 MHz, DMSO-D₆) δ ppm 7.76 (d, J=10.8 Hz, 1H), 7.31 (s, 1H), 6.50 (s, 1H), 5.81 (s, 1H), 5.28-5.52 (m, 4H), 4.47 (t, J=4.9 Hz, 1H), 3.70-3.81 (m, 1H), 3.57-3.67 (m, 1H), 3.05-3.26 (m, 2H), 2.34-2.46 (m, 4H), 1.77-2.08 (m, 5H), 0.88 (br t, J=7.3 Hz, 3H). ¹⁹F NMR (376 MHz, DMSO-D₆) δ ppm −112.27. LCMS (ESI+) m/z: [MH]⁺ calcd for C₂₆H₂₆FN₂O₆⁺: 481.1, found: 481.3. SFC (retention time=2.091 min).

Alternative and/or additional synthesis routes for the compounds described in Example 4 can be found in FIG. 6.

### Example 5

### Synthesis of: (1S,9S)-1-Azido-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (5-38) and (1R,9S)-1-azido-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (5-39)

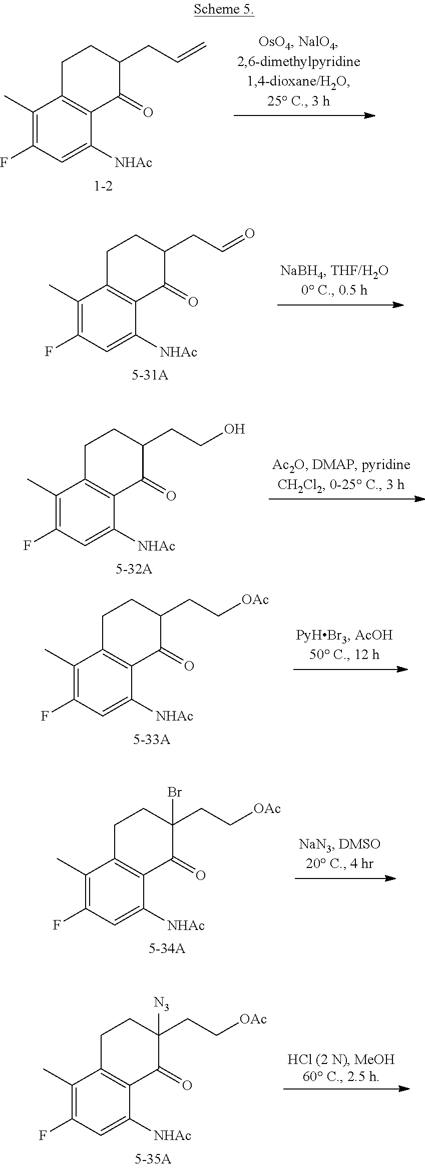

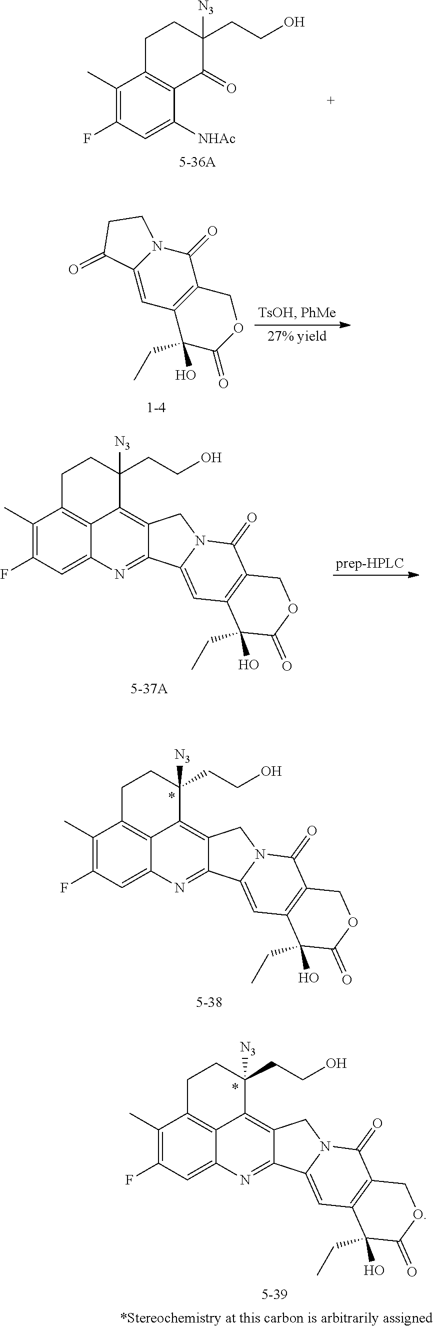 [SMILES: CC(=O)Nc1cc(F)c(C)c2c1C(=O)C(CCO)(N=[N+]=[N-])CC2 ; CC[C@@]1(O)C(=O)OCc2c1cc1n(c2=O)CCC1=O ; CC[C@@]1(O)C(=O)OCc2c1cc1n(c2=O)Cc2c-1nc1cc(F)c(C)c3c1c2C(CCO)(N=[N+]=[N-])CC3 ; "prep-HPLC" ; CC[C@@]1(O)C(=O)OCc2c1cc1n(c2=O)Cc2c-1nc1cc(F)c(C)c3c1c2[C@](CCO)(N=[N+]=[N-])CC3 ; CC[C@@]1(O)C(=O)OCc2c1cc1n(c2=O)Cc2c-1nc1cc(F)c(C)c3c1c2[C@@](CCO)(N=[N+]=[N-])CC3]

### N-(3-Fluoro-4-methyl-8-oxo-7-(2-oxoethyl)-5,6,7,8-tetrahydronaphthalen-1-yl)acetamide (5-31A)

To a stirred mixture of N-(7-allyl-3-fluoro-4-methyl-8-oxo-5,6,7,8-tetrahydronaphthalen-1-yl)acetamide (1-2) (5.0 g, 18.1 mmol, 1.0 equiv) in water (33.7 mL) and 1,4-dioxane (101 mL), was added 2,6-dimethylpyridine (3.89 g, 36.3 mmol, 4.23 mL, 2.0 equiv), osmium tetroxide (92.3 mg, 363 μmol, 18.8 L, 0.02 equiv) and sodium periodate (15.5 g, 72.6 mmol, 4.03 mL, 4.0 equiv). Two more vials were set up as described above and all three reaction mixtures were combined. After stirring at 25° C., for 3 h, it was quenched by addition of water (50 mL) and extracted with dichloromethane (4×200 mL). The combined organic layers were dried over anhydrous sodium sulfate, filtered, concentrated, and the residue purified by silica gel column chromatography (petroleum ether/ethyl acetate) to give N-(3-fluoro-4-methyl-8-oxo-7-(2-oxoethyl)-5,6,7,8-tetrahydronaphthalen-1-yl)acetamide (5-31A) (8.0 g, 69% yield). ¹H NMR (400 MHz, DMSO-D₆) δ ppm 11.95 (s, 1H) 9.74 (d, J=1.00 Hz, 1H) 8.28 (d, J=13.26 Hz, 1H) 3.15-3.26 (m, 1H) 2.97-3.06 (m, 1H) 2.82-2.93 (m, 2H) 2.58 (dd, J=17.39, 5.00 Hz, 1H) 2.11-2.15 (m, 7H) 1.80-1.92 (m, 1H). LCMS (ESI+) m/z: [MH]⁺ calcd for C₁₅H₁₇FNO₃⁺: 278.1, found: 278.3.

### N-(3-Fluoro-7-(2-hydroxyethyl)-4-methyl-8-oxo-5,6,7,8-tetrahydronaphthalen-1-yl)acetamide (5-32A)

To a stirred mixture of N-(3-fluoro-4-methyl-8-oxo-7-(2-oxoethyl)-5,6,7,8-tetrahydronaphthalen-1-yl)acetamide (5-31A) (7.6 g, 23.8 mmol, 1.0 equiv) in tetrahydrofuran (152 mL) and water (76 mL) was added sodium borohydride (180 mg, 4.76 mmol, 0.2 equiv) at 0° C. After stirring at 0° C., for 0.5 h, it was extracted with ethyl acetate (4×200 mL). The combined organic layers were dried over anhydrous sodium sulfate, filtered, concentrated, and the residue purified by silica gel column chromatography (petroleum ether/ethyl acetate) to give N-(3-fluoro-7-(2-hydroxyethyl)-4-methyl-8-oxo-5,6,7,8-tetrahydronaphthalen-1-yl) acetamide (5-32A) (4.83 g, 72% yield). ¹H NMR (400 MHz, DMSO-D₆) δ ppm 12.08 (s, 1H) 8.26 (d, J=13.26 Hz, 1H) 4.49 (t, J=5.19 Hz, 1H) 3.47-3.59 (m, 2H) 2.96 (dt, J=17.60, 4.64 Hz, 1H) 2.75-2.88 (m, 1H) 2.61-2.72 (m, 1H) 2.15 (s, 3H) 2.10 (s, 3H) 1.98-2.06 (m, 1H) 1.68-1.80 (m, 1H) 1.44-1.56 (m, 1H), ¹⁹F NMR (376 MHz, DMSO-D₆) δ ppm −104.67. LCMS (ESI+) m/z: [M+Na]⁺ calcd for C₁₅H₁₈FNO₃Na⁺: 302.1, found: 302.1.

### 2-(8-Acetamido-6-fluoro-5-methyl-1-oxo-1,2,3,4-tetrahydronaphthalen-2-yl)ethyl acetate (5-33A)

To a stirred mixture of N-(3-fluoro-7-(2-hydroxyethyl)-4-methyl-8-oxo-5,6,7,8-tetrahydronaphthalen-1-yl)acetamide (5-32A) (4.0 g, 14.3 mmol, 1.0 equiv) in dichloromethane (80 mL) was added pyridine (27.2 g, 343 mmol, 27.7 mL, 24 equiv), N,N-dimethylpyridin-4-amine (174 mg, 1.43 mmol, 0.1 equiv) and Ac₂O (23.3 g, 229 mmol, 21.5 mL, 16 equiv) at 0° C. After stirring at 25° C., for 3 h, the reaction mixture was quenched by addition of ice water (60 mL) at 0° C., adjusted to pH 7 by addition of HCl aqueous (1 M) at 0° C., and extracted with dichloromethane (4×100 mL). The combined organic layers were dried over anhydrous sodium sulfate, filtered, concentrated, and the residue purified by silica gel column chromatography (petroleum ether/ethyl acetate) to give 2-(8-acetamido-6-fluoro-5-methyl-1-oxo-1,2,3,4-tetrahydronaphthalen-2-yl)ethyl acetate (5-33A) (3.2 g, 69% yield). ¹H NMR (400 MHz, DMSO-D₆) δ ppm 12.01 (s, 1H) 8.26 (d, J=13.26 Hz, 1H) 4.06-4.21 (m, 2H) 2.93-3.05 (m, 1H) 2.78-2.91 (m, 1H) 2.63-2.74 (m, 1H) 2.13-2.22 (m, 5H) 2.11 (s, 3H) 1.99 (s, 3H) 1.64-1.86 (m, 2H). ¹⁹F NMR (376 MHz, DMSO-D₆) δ ppm −104.47. LCMS (ESI+) m/z: [MH]⁺ calcd for C₁₇H₂₁FNO₄⁺: 322.1, found: 322.2.

### 2-(8-Acetamido-2-bromo-6-fluoro-5-methyl-1-oxo-1,2,3,4-tetrahydronaphthalen-2-yl)ethyl acetate (5-34A)

To a stirred mixture of 2-(8-acetamido-6-fluoro-5-methyl-1-oxo-1,2,3,4-tetrahydronaphthalen-2-yl)ethyl acetate (5-33A) (3.2 g, 10.0 mmol, 1.0 equiv) in acetic acid (64 mL) was added pyridinium tribromide (3.52 g, 11.0 mmol, 1.1 equiv). After stirring at 50° C., for 12 h, it was quenched by addition of ice water (60 mL), adjusted pH to 7 by addition of saturated aqueous sodium bicarbonate solution at 0° C., and extracted with dichloromethane (3×100 mL). The combined organic layers were dried over anhydrous sodium sulfate, filtered, concentrated, and the residue purified by silica gel column chromatography (petroleum ether/ethyl acetate) to give 2-(8-acetamido-2-bromo-6-fluoro-5-methyl-1-oxo-1,2,3,4-tetrahydronaphthalen-2-yl)ethyl acetate (5-34A) (2.4 g, 58% yield). ¹H NMR (400 MHz, DMSO-D₆) δ ppm 11.81 (s, 1H) 8.33-8.40 (m, 1H) 4.16-4.37 (m, 2H) 3.03-3.13 (m, 1H) 2.84-2.96 (m, 1H) 2.52-2.69 (m, 3H) 2.29-2.40 (m, 1H) 2.19 (s, 3H) 2.14-2.18 (m, 3H) 1.99 (s, 3H). ¹⁹F NMR (376 MHz, DMSO-D₆) δ ppm −104.47. LCMS (ESI+) m/z: [MH]⁺ calcd for C₁₇H₂₀BrFNO₄⁺: 400.0, 402.0 found: 400.1, 402.1.

### 2-(8-Acetamido-2-azido-6-fluoro-5-methyl-1-oxo-1,2,3,4-tetrahydronaphthalen-2-yl)ethyl acetate (5-35A)

To a stirred mixture of 2-(8-acetamido-2-bromo-6-fluoro-5-methyl-1-oxo-1,2,3,4-tetrahydronaphthalen-2-yl)ethyl acetate (5-34A) (500 mg, 1.25 mmol, 1.0 equiv) in dimethyl sulfoxide (10 mL) was added sodium azide (162 mg, 2.50 mmol, 2 equiv). After stirring at 20° C., for 4 h under argon atmosphere, it was quenched with ice water (40 mL), adjusted to pH 8 by addition of saturated aqueous sodium bicarbonate solution at 0° C., and extracted with ethyl acetate (3×50 mL). The combined organic layers were washed with brine (30 mL), dried over anhydrous sodium sulfate, filtered, concentrated, and the residue purified by silica gel column chromatography (petroleum ether/ethyl acetate) to give 2-(8-acetamido-2-azido-6-fluoro-5-methyl-1-oxo-1,2,3,4-tetrahydronaphthalen-2-yl) ethyl acetate (5-35A) (184 mg, 38% yield). ¹H NMR (400 MHz, DMSO-D₆) δ ppm 11.54 (s, 1H) 8.29 (d, J=13.01 Hz, 1H) 4.14-4.20 (m, 2H) 2.94-3.00 (m, 2H) 2.21 (br d, J=5.13 Hz, 2H) 2.19 (s, 3H) 2.14-2.18 (m, 2H) 2.12 (d, J=1.38 Hz, 3H) 1.93 (s, 3H). ¹⁹F NMR (376 MHz, DMSO-D₆) δ ppm −102.81. LCMS (ESI+) m/z: [MH]⁺ calcd for C₁₇H₂₀FN₄O₄⁺: 363.1, found: 363.1.

### 8-Amino-2-azido-6-fluoro-2-(2-hydroxyethyl)-5-methyl-3,4-dihydronaphthalen-1(2H)-one (5-36A)

To a stirred mixture of 2-(8-acetamido-2-azido-6-fluoro-5-methyl-1-oxo-1,2,3,4-tetrahydronaphthalen-2-yl)ethyl acetate (5-35A) (180 mg, 496 μmol, 1 equiv) in methanol (7.2 mL) was added hydrochloric acid (2 M, 7.20 mL, 28.9 equiv). After stirring at 60° C. for 2.5 h and cooled to room temperature, it was quenched by addition of saturated sodium bicarbonate solution (˜12 mL) to adjust to pH 7 and extracted with ethyl acetate (3×30.0 mL). The combined organic layers were dried over Na₂SO₄, filtered, and concentrated to give 8-amino-2-azido-6-fluoro-2-(2-hydroxyethyl)-5-methyl-3,4-dihydronaphthalen-1(2H)-one (5-36A) (130 mg, 94% yield), which was used directly in the next step without further purification. ¹H NMR (400 MHz, DMSO-D₆) δ ppm 7.36-7.63 (m, 2H) 6.42 (d, J=12.51 Hz, 1H) 4.64 (t, J=5.19 Hz, 1H) 3.48-3.65 (m, 2H) 2.79 (br t, J=5.94 Hz, 2H) 2.10-2.21 (m, 2H) 1.97 (s, 3H). ¹⁹F NMR (376 MHz, DMSO-D₆) δ ppm −106.57. LCMS (ESI+) m/z: [M+Na]⁺ calcd for C₁₃H₁₅FN₄O₂Na⁺: 301.1, found: 301.1.

### (9S)-1-Azido-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (5-37A)

After the stirred mixture of 8-amino-2-azido-6-fluoro-2-(2-hydroxyethyl)-5-methyl-3,4-dihydronaphthalen-1(2H)-one (5-36A) (130 mg, 467 μmol, 1.0 equiv). (S)-4-ethyl-4-hydroxy-7,8-dihydro-1H-pyrano[3,4-f]indolizine-3,6,10(4H)-trione (1-4) (245 mg, 934 μmol, 2.0 equiv) and 4-methylbenzenesulfonic acid (32.1 mg, 186 μmol, 0.4 equiv) in toluene (6.5 mL) was degassed and purged with argon for 3 times, it was stirred at 120° C. for 16 h under argon atmosphere. It was cooled to room temperature, quenched by addition of water (20 mL), and extracted with ethyl acetate (5×30 mL). The combined organic layers were dried over anhydrous sodium sulfate, filtered and concentrated to give (9S)-1-azido-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (5-37A) (150 mg, 27% yield). LCMS (ESI+) m/z: [MH]⁺ calcd for C₂₆H₂₅FN₅O₅⁺: 506.1, found: 506.3.

### (1S,9S)-1-Azido-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (5-38) and (1R,9S)-1-azido-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (5-39)

(9S)-1-azido-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (5-37A) (150 mg, crude) was separated by HPLC (column: Phenomenex Luna C18 100*30 mm*5 um; mobile phase: [H₂O (0.2% formic)-acetonitrile]: gradient: 30%-50% B over 12.0 min) to afford (1S,9S)-1-azido-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (5-38) (compound 5-38 may be the opposite enantiomer of that depicted) (11.4 mg) and (1R,9S)-1-azido-9-ethyl-5-fluoro-9-hydroxy-1-(2-hydroxyethyl)-4-methyl-1,2,3,9,12,15-hexahydro-10H,13H-benzo[de]pyrano [3′,4′:6,7]indolizino[1,2-b]quinolone-10,13-dione (5-39) (compound 5-39 may be the opposite enantiomer of that depicted) (19.8 mg). Note: The stereochemistry for two products are randomly assigned.

Spectra for 5-38: ¹H NMR (400 MHz, DMSO-D₆) δ ppm 7.83 (d, J=10.63 Hz, 1H) 7.32 (s, 1H) 6.53 (s, 1H) 5.37-5.47 (m, 4H) 4.66 (t, J=5.00 Hz, 1H) 3.53-3.71 (m, 2H) 3.33-3.39 (m, 1H) 3.14-3.28 (m, 1H) 2.71-2.80 (m, 1H) 2.40 (s, 3H) 2.24-2.30 (m, 1H) 1.98 (br t, J=6.25 Hz, 2H) 1.79-1.92 (m, 2H) 0.83-0.91 (m, 3H). ¹⁹F NMR (376 MHz, DMSO-D₆) δ ppm −111.66. LCMS (ESI+) m/z: [MH]⁺ calcd for C₂₆H₂₅FN₅O₅⁺: 506.1, found: 506.3. SFC (retention time=1.500 min).

Spectra for 5-39: ¹H NMR (400 MHz, DMSO-D₆) δ ppm 7.83 (d, J=10.76 Hz, 1H) 7.32 (s, 1H) 6.48-6.58 (m, 1H) 5.43 (d, J=7.88 Hz, 4H) 4.66 (t, J=5.07 Hz, 1H) 3.53-3.72 (m, 2H) 3.35 (br s, 1H) 3.14-3.27 (m, 1H) 2.72-2.80 (m, 1H) 2.40 (s, 3H) 2.27 (td, J=12.66, 5.07 Hz, 1H) 1.99 (br t, J=6.13 Hz, 2H) 1.79-1.93 (m, 2H) 0.84-0.92 (m, 3 H). ¹⁹F NMR (376 MHz, DMSO-D₆) δ ppm −111.66. LCMS (ESI+) m/z: [MH]⁺ calcd for C₂₆H₂₅FN₅O₅⁺: 506.1, found: 506.3. SFC (retention time=1.903 min).

### Example 6

### Synthesis of: N—((S)-1-((1S,9S)-9-Ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)allyl)acetamide (6-45), N—((R)-1-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)allyl)acetamide (6-46), N—((S)-1-((1R,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)allyl)acetamide (6-47) and N—((R)-1-((1R,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)allyl)acetamide (6-48)

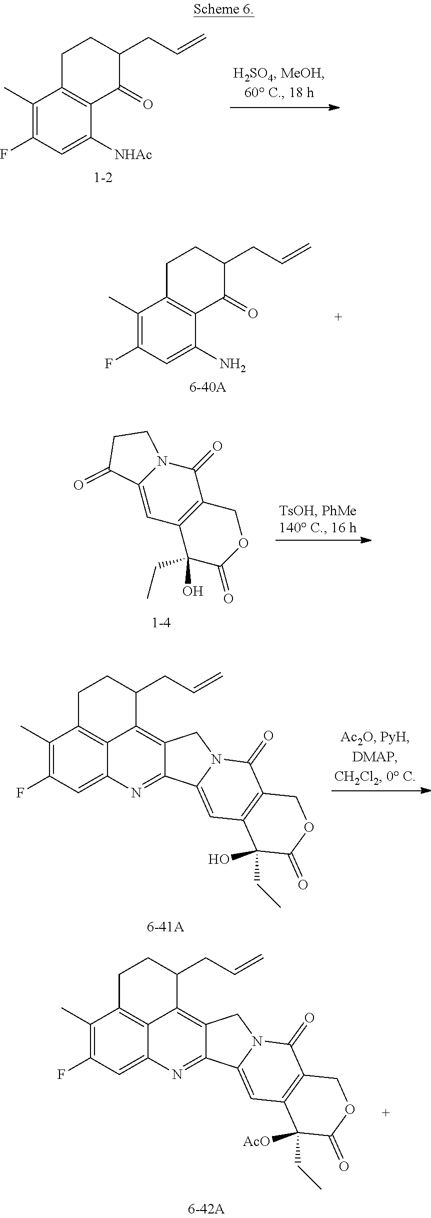 [SMILES: C=CCC1CCc2c(C)c(F)cc(NC(C)=O)c2C1=O ; C=CCC1CCc2c(C)c(F)cc(N)c2C1=O ; CC[C@@]1(O)C(=O)OCc2c1cc1n(c2=O)CCC1=O ; C=CCC1CCc2c(C)c(F)cc3nc4c(c1c23)Cn1c-4cc2c(c1=O)COC(=O)[C@]2(O)CC ; C=CCC1CCc2c(C)c(F)cc3nc4c(c1c23)Cn1c-4cc2c(c1=O)COC(=O)[C@@]2(CC)OC(C)=O]

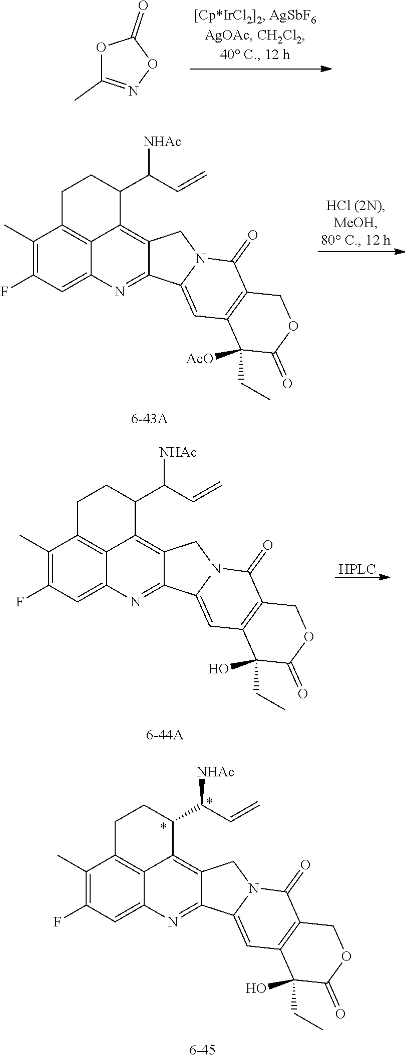

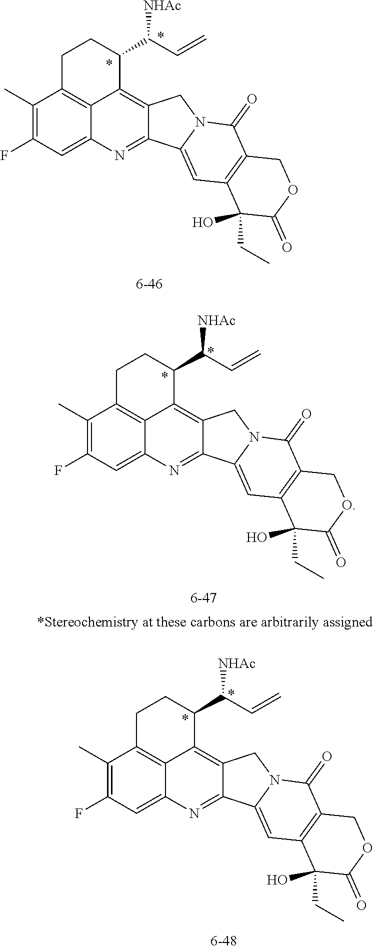 [SMILES: C=C[C@@H](NC(C)=O)[C@H]1CCc2c(C)c(F)cc3nc4c(c1c23)Cn1c-4cc2c(c1=O)COC(=O)[C@]2(O)CC ; C=C[C@H](NC(C)=O)[C@@H]1CCc2c(C)c(F)cc3nc4c(c1c23)Cn1c-4cc2c(c1=O)COC(=O)[C@]2(O)CC ; C=C[C@@H](NC(C)=O)[C@@H]1CCc2c(C)c(F)cc3nc4c(c1c23)Cn1c-4cc2c(c1=O)COC(=O)[C@]2(O)CC]

### 2-Allyl-8-amino-6-fluoro-5-methyl-3,4-dihydronaphthalen-1(2H)-one (6-40A)

To a stirred mixture of N-(7-allyl-3-fluoro-4-methyl-8-oxo-5,6,7,8-tetrahydronaphthalen-1-yl)acetamide (1-2) (1.0 g, 3.63 mmol, 1.0 equiv) in methanol (30 mL) was added sulfuric acid (2.0 mL). An additional five vials were set up as described above, and all six reaction mixtures were combined for work up. After stirring at 60° C., for 18 h under argon atmosphere and cooled to room temperature, the reaction mixture was quenched with water (150 mL), adjusted to pH 7 by addition of saturated bicarbonate solution at 0° C., and extracted with ethyl acetate (3×300 mL). The combined organic layers were washed with brine, dried over Na₂SO₄, filtered, concentrated, and the residue purified by silica gel column chromatography (petroleum ether/ethyl acetate) to give 2-allyl-8-amino-6-fluoro-5-methyl-3,4-dihydronaphthalen-1(2H)-one (6-40A) (3.2 g, 62% yield). ¹H NMR (400 MHz, DMSO-D₆) δ ppm 7.42 (br s, 2H), 6.35 (d, J=12.7 Hz, 1H), 5.59-5.99 (m, 1H), 4.93-5.23 (m, 2H), 2.87 (dt, J=17.3, 4.5 Hz, 1H), 2.52-2.74 (m, 2H), 2.48 (br s, 1H), 1.93-2.21 (m, 5H), 1.54-1.70 (m, 1H). LCMS (ESI+) m/z: [MH]⁺ calcd for C₁₄H₁₇FNO⁺: 234.1, found: 234.4.

### (9S)-1-Allyl-9-ethyl-5-fluoro-9-hydroxy-4-methyl-1,2,3,9,12,15-hexahydro-10H,13H benzo[de]pyrano[3′,4′:6,7]indolizino[1,2-b]quinoline-10,13-dione (6-41A)

To a suspension of 2-allyl-8-amino-6-fluoro-5-methyl-3,4-dihydronaphthalen-1(2H)-one (6-40A) (200 mg, 857 μmol, 1.0 equiv) and(S)-4-ethyl-4-hydroxy-7,8-dihydro-1H-pyrano[3,4-f]indolizine-3,6,10(4H)-trione (1-4) (451 mg, 1.71 mmol, 2.0 equiv) in toluene (10 mL) was added 4-methylbenzenesulfonic acid (59.1 mg, 342 μmol, 0.4 equiv) at 140° C. Additional two vials were set up as described above, and all three reaction mixtures were combined for work up. After stirring at 140° C., for 16 h with Dean-Stark, it was cooled to room temperature, concentrated and the residue purified by silica gel column chromatography (petroleum ether/ethyl acetate) to afford (9S)-1-allyl-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (6-41A) (300 mg, 21% yield). ¹H NMR (400 MHz, DMSO-D₆) δ ppm 7.74 (br d, J=10.9 Hz, 1H), 7.30 (s, 1H), 6.51 (s, 1H), 5.88-6.12 (m, 1H), 5.27-5.51 (m, 4H), 5.02-5.25 (m, 2H), 3.45 (br d, J=3.3 Hz, 1H), 3.08 (br s, 2H), 2.34-2.47 (m, 4H), 2.18-2.33 (m, 2H), 1.83-1.95 (m, 3H), 0.87 (br t, J=7.0 Hz, 3H). LCMS (ESI+) m/z: [MH]⁺ calcd for C₂₇H₂₆FN₂O₄⁺: 461.1, found: 461.4. SFC (retention time=1.020 min, 2.034 min).

### (9S)-1-Allyl-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (6-42A)

To a stirred mixture of (9S)-1-allyl-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (6-41A) (15.1 g, 32.8 mmol, 1 equiv) in dichloromethane (300 mL), were added N,N-dimethylpyridin-4-amine (400 mg, 3.28 mmol, 0.1 equiv), pyridine (62.2 g, 787 mmol, 63.5 mL, 24 equiv), and Ac₂O (53.5 g, 525 mmol, 49.3 mL, 16 equiv) at 0° C. After stirring at 0° C., for 2 h, it was quenched by addition of H₂O (500 mL) and extracted with dichloromethane (3×300 mL). The combined organic layers were washed with brine, dried over Na₂SO₄, filtered, concentrated, and the residue purified by silica gel column chromatography (petroleum ether/ethyl acetate) to give (9S)-1-allyl-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (6-42A) (9.3 g, 56% yield). ¹H NMR (400 MHz, DMSO-D₆) δ ppm 7.72 (dd, J=11.07, 3.44 Hz, 1H) 7.01 (s, 1H) 5.89-6.05 (m, 1H) 5.49 (s, 2H) 5.22-5.40 (m, 2H) 5.07-5.20 (m, 2H) 3.44 (br d, J=4.88 Hz, 1H) 2.93-3.17 (m, 2H) 2.39-2.46 (m, 1H) 2.36 (s, 3H) 2.21-2.31 (m, 5H) 2.07-2.19 (m, 2H) 1.91-1.99 (m, 1H) 0.91 (t, J=7.38 Hz, 3H). ¹⁹F NMR (376 MHz, DMSO-D₆) δ ppm −111.53. LCMS (ESI+) m/z: [MH]⁺ calcd for C₂₉H₂₈FN₂O₅⁺: 503.1, found: 503.4.

### (9S)-1-(1-Acetamidoallyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (6-43A)

After the stirred mixture of (9S)-1-allyl-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (6-42A) (9.30 g, 18.5 mmol, 1.0 equiv), 3-methyl-1,4,2-dioxazol-5-one (18.7 g, 185 mmol, 10 equiv), dichloroiridium-1,2,3,4,5-pentamethylcyclopentane (4.48 g, 5.55 mmol, 0.3 equiv), AgSbF₆ (5.09 g, 14.8 mmol, 0.8 equiv) and AgOAc (5.56 g, 33.3 mmol, 1.71 mL, 1.8 equiv) in dichloromethane (100 mL) was degassed and purged with nitrogen for 3 times, it was stirred at 40° C., for 12 h under nitrogen atmosphere. It was cooled to room temperature, diluted with dichloromethane (200 mL), filtered, concentrated, and the residue purified by silica gel column chromatography (petroleum ether/ethyl acetate) to give (9S)-1-(1-acetamidoallyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (6-43A) (8.3 g, 68% yield). ¹H NMR (400 MHz, DMSO-D₆) δ ppm 8.16-8.29 (m, 1H) 7.66-7.86 (m, 1H) 6.91-7.09 (m, 1H) 5.62-5.95 (m, 1H) 5.43-5.55 (m, 2H) 4.74-5.40 (m, 3H) 4.47-4.62 (m, 1H) 4.12 (br s, 1H) 3.50-3.69 (m, 1H) 2.90-3.17 (m, 2H) 2.29-2.42 (m, 4H) 2.22 (d, J=4.77 Hz, 3H) 2.02-2.17 (m, 3H) 1.70-1.78 (m, 3H) 0.83-0.99 (m, 3H). ¹⁹F NMR (376 MHz, DMSO-D₆) δ ppm −111.12. LCMS (ESI+) m/z: [MH]⁺ calcd for C₃₁H₃₁FN₃O₆⁺: 560.3, found: 560.2.

### N-(1-((9S)-9-Ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)allyl)acetamide (6-44A)

To a stirred mixture of (9S)-1-(1-acetamidoallyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (6-43A) (700 mg, 1.25 mmol, 1.0 equiv) in methanol (14 mL) was added HCl (2 M, 28 mL). After stirring at 80° C., for 12 h, it was cooled to 20° C., extracted with ethyl acetate (3×50 mL), combined organic layers washed with brine, dried over Na₂SO₄, filtered, concentrated, and the residue purified by prep-HPLC (Instrument: Gilson 281 Semi-preparative HPLC system, Column: Phenomenex Luna C18 100*30 mm*5 um, Mobile phase: A: H₂O (0.2% formic acid); B: acetonitrile, Gradient: B from 20.00% to 50.00% in 8.00 min. Flow rate: 60.00 ml/min. Monitor wavelength: 220&254 nm) to give N-(1-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)allyl)acetamide (6-44A) (120 mg, 18% yield). LCMS (ESI+) m/z: [MH]⁺ calcd for C₂₉H₂₉FN₃O₅⁺: 518.2, found: 518.3.

### N—((S)-1-((1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)allyl)acetamide (6-45), N—((R)-1-((1S,9S)-9-ethyl-5-fluoro-9-hydroxy-4-methyl-10,13-dioxo-2,3,9,10,13,15-hexahydro-1H,12H-benzo [de]pyrano[3′,4′:6,7]indolizino[1,2-b]quinolin-1-yl)allyl)acetamide (6-46), N—((S)-1-((1R,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)allyl)acetamide (6-47) and N—((R)-1-((1R,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)allyl)acetamide (6-48)

N-(1-((9S)-9-ethyl-5-fluoro-9-hydroxy-4-methyl-10,13-dioxo-2,3,9,10,13,15-hexahydro1H,12H-benzo[de]pyrano[3′,4′:6,7]indolizino[1,2-b]quinolin-1-yl)allyl)acetamide (6-44A) (120 mg, 0.23 mmol) was separated by HPLC (Instrument: Gilson 281 Semi-preparative HPLC system, Column: Phenomenex Luna C18 100*30 mm*5 um, Mobile phase: A: H₂O (0.2% formic acid); B: acetonitrile, Gradient: B from 25.00% to 45.00% in 12.00 min. Flow rate: 60.00 ml/min. Monitor wavelength: 220&254 nm) to give N—((S)-1-((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)allyl)acetamide (6-45) (compound 6-45 may be the opposite enantiomer of that depicted) (9.2 mg), N—((R)-1-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)allyl)acetamide (6-46) (compound 6-46 may be the opposite enantiomer of that depicted) (10.3 mg), N—((S)-1-((1R,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)allyl)acetamide (6-47) (compound 6-47 may be the opposite enantiomer of that depicted) (3.6 mg) and N—((R)-1-((1R,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)allyl) acetamide (6-48) (compound 6-48 may be the opposite enantiomer of that depicted) (3.6 mg). Note: The stereochemistry of the four products is arbitrarily assigned.

Spectra for 6-45: ¹H NMR (400 MHz, DMSO-D₆) δ ppm 8.22 (d, J=9.26 Hz, 1H) 7.75 (d, J=11.01 Hz, 1H) 7.30 (s, 1H) 6.52 (br s, 1H) 5.70 (ddd, J=16.85, 10.54, 6.00 Hz, 1H) 5.42 (s, 2H) 5.33 (d, J=18.64 Hz, 1H) 5.11 (d, J=18.64 Hz, 1H) 4.78-4.94 (m, 2H) 4.48-4.62 (m, 1H) 3.31-3.40 (m, 1H) 3.11-3.25 (m, 1H) 2.97-3.10 (m, 1H) 2.36 (s, 3H) 2.32 (br s, 1H) 1.78-1.98 (m, 6H) 0.89 (t, J=7.32 Hz, 3H). ¹⁹F NMR (376 MHz, DMSO-D₆) δ ppm −111.76. LCMS (ESI+) m/z: [MH]⁺ calcd for C₂₉H₂₉FN₃O₅⁺: 518.2, found: 518.3. SFC (retention time=1.368 min).

Spectra for 6-46: ¹H NMR (400 MHz, DMSO-D₆) δ ppm 8.22 (br d. J=9.13 Hz, 1H) 7.76 (br d, J=10.88 Hz, 1H) 7.30 (s, 1H) 6.53 (s, 1H) 5.71 (ddd, J=16.76, 10.26, 6.25 Hz, 1H) 5.43 (s, 2H) 5.33 (br d, J=18.64 Hz, 1H) 5.12 (br d, J=18.64 Hz, 1H) 4.74-4.93 (m, 2H) 4.49-4.60 (m, 1H) 3.31-3.40 (m, 1H) 3.09-3.20 (m, 1H) 2.97-3.08 (m, 1H) 2.36 (br s, 3H) 2.32 (br s, 1H) 1.77-1.99 (m, 6H) 0.87 (br t, J=7.13 Hz, 3H), ¹⁹F NMR (376 MHz, DMSO-D₆) δ ppm −111.76. LCMS (ESI+) m/z: [MH]⁺ calcd for C₂₉H₂₉FN₃O₅⁺: 518.2, found: 518.3. SFC (retention time=2.727 min).

Spectra for 6-47: ¹H NMR (400 MHz, DMSO-D₆) δ ppm 8.28 (d, J=9.26 Hz, 1H) 7.74 (d, J=11.01 Hz, 1H) 7.31 (s, 1H) 6.53 (s, 1H) 5.92 (ddd, J=17.42, 9.91, 7.94 Hz, 1H) 5.31-5.54 (m, 4H) 5.28 (br d, J=17.14 Hz, 1H) 5.16 (d, J=10.38 Hz, 1H) 4.60 (q, J=8.88 Hz, 1H) 3.36-3.42 (m, 1H) 3.02-3.18 (m, 2H) 2.39 (s, 3H) 2.29-2.36 (m, 1H) 1.83-2.00 (m, 3H) 1.52 (s, 3H) 0.89 (t, J=7.32 Hz, 3H). ¹⁹F NMR (376 MHz, DMSO-D₆) δ ppm −112.29. LCMS (ESI+) m/z: [MH]⁺ calcd for C₂₉H₂₉FN₃O₅⁺: 518.2, found: 518.3. SFC (retention time=1.859 min).

Spectra for 6-48: ¹H NMR (400 MHz, DMSO-D₆) δ ppm 8.25 (d, J=9.26 Hz, 1H) 7.73 (d, J=11.01 Hz, 1H) 7.30 (s, 1H) 6.52 (s, 1H) 5.85-5.99 (m, 1H) 5.30-5.50 (m, 4H) 5.27 (d, J=17.13 Hz, 1H) 5.15 (d, J=10.51 Hz, 1H) 4.60 (q, J=8.80 Hz, 1H) 3.34-3.36 (m, 1H) 3.00-3.18 (m, 2H) 2.38 (s, 3H) 2.27-2.35 (m, 1H) 1.81-2.01 (m, 3H) 1.49 (s, 3H) 0.86 (t, J=7.32 Hz, 3H). ¹⁹F NMR (376 MHz, DMSO-D₆) δ ppm −112.28. LCMS (ESI+) m/z: [MH]⁺ calcd for C₂₉H₂₉FN₃O₅⁺: 518.2, found: 518.2. SFC (retention time=2.897 min).

### Example 7

### Synthesis of: (1S,9S)-1-amino-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (7-50) and (1R,9S)-1-amino-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (7-51)

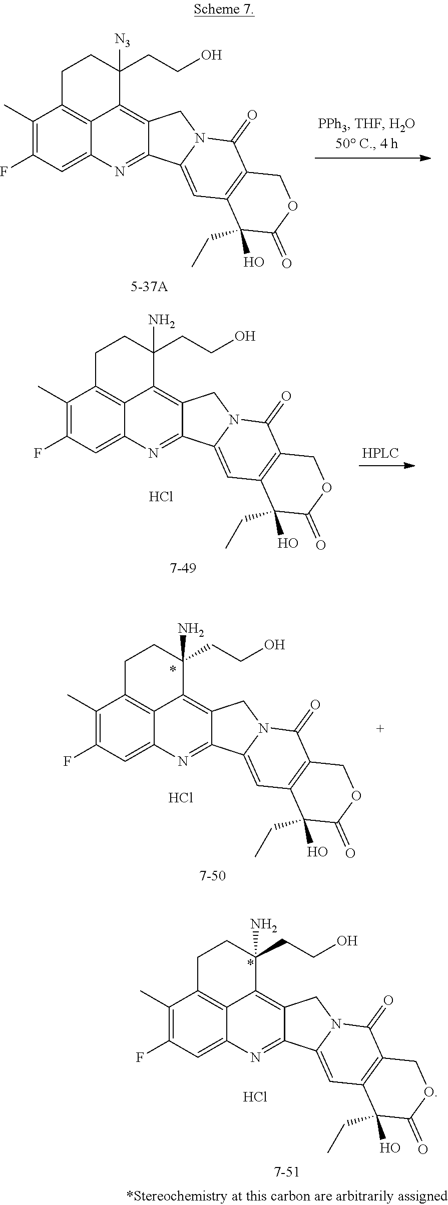

### (9S)-1-Amino-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione hydrochloride (7-49)

To a stirred mixture of (9S)-1-azido-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (5-37A) (450 mg, 489 μmol, 1.0 equiv) in tetrahydrofuran (9 mL) and water (2.25 mL) was added PPh₃ (256 mg, 979 μmol, 2.0 equiv). After stirring at 50° C., for 4 h and cooled to room temperature, it was quenched by addition of water (20 mL), adjusted to pH 2 by addition of 1 N hydrochloric acid solution at 0° C. After separation, the aqueous layer was washed with ethyl acetate (2× 20 mL), and the aqueous layer concentrated to give (9S)-1-amino-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinolone-10,13-dione hydrochloride (7-49) (230 mg, 86% yield). LCMS (ESI+) m/z: [MH]⁺ calcd for C₂₆H₂₇FN₃O₅⁺: 480.1, found: 480.3.

### (1S,9S)-1-Amino-9-ethyl-5-fluoro-9-hydroxy-1-(2-hydroxyethyl)-4-methyl-1,2,3,9,12,15-hexahydro-10H,13H-benzo [de]pyrano[3′ 4′:6,7]indolizino[1,2-b]quinoline-10,13-dione hydrochloride (7-50) and (1R,9S)-1-amino-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione hydrochloride (7-51)

(9S)-1-amino-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dion e hydrochloride (7-49) (200 mg, 417 μmol) was separated by HPLC (column: Phenomenex Luna C18 100*30 mm*5 um; mobile phase: [H₂O (0.2% formic acid)-acetonitrile]; gradient: 1%-30% B over 8.0 min) to give (1S,9S)-1-amino-9-ethyl-5-fluoro-9-hydroxy-1-(2-hydroxyethyl)-4-methyl-1,2,3,9,12,15-hexahydro-10H,13H-ben zo[de]pyrano[3′,4′:6,7]indolizino[1,2-b]quinoline-10,13-dione hydrochloride (7-50) and (1R,9S)-1-amino-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione hydrochloride (7-51). Each product was suspended in water (0.04% hydrochloric acid, 2 mL) and lyophilized to give (7-50) (compound 7-50 may be the opposite enantiomer of that depicted) (5.0 mg) and (7-51) (compound 7-51 may be the opposite enantiomer of that depicted) (10.3 mg).

Spectra for 7-50: ¹H NMR (400 MHz, DMSO-D₆) δ ppm 7.74-7.82 (m, 1H) 7.29-7.35 (m, 1H) 6.49-6.54 (m, 1H) 5.69 (br d, J=19.76 Hz, 1H) 5.40-5.53 (m, 3H) 3.59 (br t, J=6.19 Hz, 2H) 3.08-3.20 (m, 2H) 2.30-2.41 (m, 4H) 2.00-2.12 (m, 1H) 1.79-1.98 (m, 4H) 0.88 (t, J=7.32 Hz, 3H). ¹⁹F NMR (376 MHz, DMSO-D₆) δ ppm-112.45. LCMS (ESI+) m/z: [MH]⁺ calcd for C₂₆H₂₇FN₃O₅⁺: 480.1, found: 480.3. SFC (retention time=3.114 min).

Spectra for 7-51: ¹H NMR (400 MHz, DMSO-D₆) δ ppm 7.84 (d, J=10.63 Hz, 1H) 7.31-7.36 (m, 1H) 6.53 (s, 1H) 5.68 (d, J=19.2 Hz, 1H) 5.53 (d, J=18.8 Hz, 1H) 5.45 (s, 2H) 3.60-3.67 (m, 1H) 3.51-3.60 (m, 1H) 3.15-3.20 (m, 2H) 2.50-2.54 (m, 1H) 2.39 (s, 3H) 1.99-2.24 (m, 3H) 2.04-2.09 (m, 1H) 1.91-2.03 (m, 2H) 1.80-1.91 (m, 2H) 0.88 (t, J=7.32 Hz, 3H). ¹⁹F NMR (376 MHz, DMSO-D₆) δ ppm −111.75. LCMS (ESI+) m/z: [MH]⁺ calcd for C₂₆H₂₇FN₃O₅⁺: 480.1, found: 480.3 SFC (retention time=1.822 min).

Alternative and/or additional synthesis routes for the compounds described in Example 7 can be found in FIG. 8.

### Example 8

### Synthesis of: (1R,9S)-1-Amino-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione hydrochloride (8-57) and (1S,9S)-1-amino-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione hydrochloride (8-58)

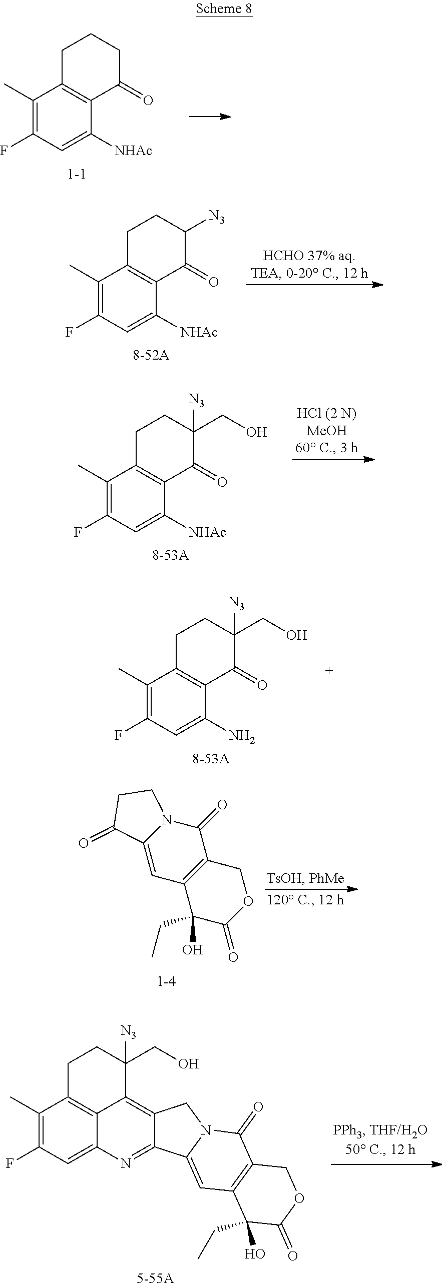 [SMILES: CC(=O)Nc1cc(F)c(C)c2c1C(=O)CCC2 ; CC(=O)Nc1cc(F)c(C)c2c1C(=O)C(N=[N+]=[N-])CC2 ; CC(=O)Nc1cc(F)c(C)c2c1C(=O)C(CO)(N=[N+]=[N-])CC2 ; Cc1c(F)cc(N)c2c1CCC(CO)(N=[N+]=[N-])C2=O ; CC[C@@]1(O)C(=O)OCc2c1cc1n(c2=O)CCC1=O ; CC[C@@]1(O)C(=O)OCc2c1cc1n(c2=O)Cc2c-1nc1cc(F)c(C)c3c1c2C(CO)(N=[N+]=[N-])CC3]

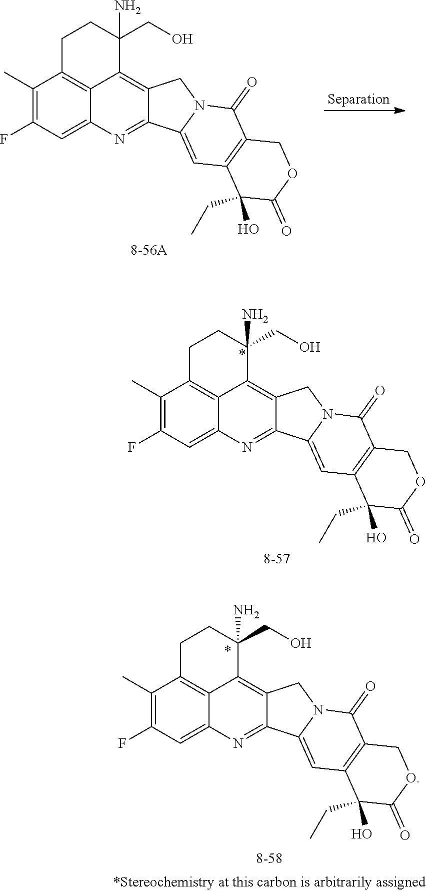 [SMILES: CC[C@@]1(O)C(=O)OCc2c1cc1n(c2=O)Cc2c-1nc1cc(F)c(C)c3c1c2C(N)(CO)CC3 ; CC[C@@]1(O)C(=O)OCc2c1cc1n(c2=O)Cc2c-1nc1cc(F)c(C)c3c1c2[C@@](N)(CO)CC3 ; CC[C@@]1(O)C(=O)OCc2c1cc1n(c2=O)Cc2c-1nc1cc(F)c(C)c3c1c2[C@](N)(CO)CC3]

### N-(7-Azido-3-fluoro-7-(hydroxymethyl)-4-methyl-8-oxo-5,6,7,8-tetrahydronaphthalen-1-yl)acetamide (8-53A)

To a stirred mixture of N-(7-azido-3-fluoro-7-(hydroxymethyl)-4-methyl-8-oxo-5,6,7,8-tetrahydronaphthalen-1-yl)acetamide (8-52A) (900 mg, 3.26 mmol, 1.0 equiv) in formaldehyde (25.6 g, 316 mmol, 37 wt %, 97 equiv) was added triethylamine (329 mg, 3.26 mmol, 1.0 equiv) at 0° C. After stirring at 20° C., for 12 h, it was quenched by addition of water (20 mL) and extracted with dichloromethane (3×30 mL). The combined organic layers were dried over Na₂SO₄, filtered, concentrated, and the residue purified by silica gel column chromatography (petroleum ether/ethyl acetate) to give N-(7-azido-3-fluoro-7-(hydroxymethyl)-4-methyl-8-oxo-5,6,7,8-tetrahydronaphthalen-1-yl)acetamide (8-53A) (700 mg, 21% yield). ¹H NMR (400 MHz, DMSO-D₆) δ ppm 11.63 (s, 1H), 8.30 (d, J=13.01 Hz, 1H), 5.47 (t, J=5.88 Hz, 1H), 3.81 (d, J=5.88 Hz, 2H), 2.93 (br d, J=6.13 Hz, 2H), 2.12-2.20 (m, 4H), 2.11 (d, J=1.38 Hz, 3H), 1.98-2.08 (m, 1H), ¹⁹F NMR (376 MHz, DMSO-D₆) δ ppm −102.781. LCMS (ESI+) m/z: [MH]⁺ calcd for C₁₄H₁₆FN₄O₃⁺: 307.1, found: 307.5.

### 8-Amino-2-azido-6-fluoro-2-(hydroxymethyl)-5-methyl-3,4-dihydronaphthalen-1(2H)-one (8-54A)

To a stirred mixture of N-(7-azido-3-fluoro-7-(hydroxymethyl)-4-methyl-8-oxo-5,6,7,8-tetrahydronaphthalen-1-yl)acetamide (8-53A) (700 mg, 2.29 mmol, 1.0 equiv) in methanol (28 mL) was added hydrochloric acid (2 M, 28 mL). After stirring at 60° C., for 3 h, it was cooled to room temperature, adjusted to pH 8 with saturated sodium bicarbonate solution, and extracted with ethyl acetate (3× 100 mL). The combined organic layers were washed with brine, dried over Na₂SO₄, filtered, concentrated, and the residue purified by silica gel column chromatography (petroleum ether/ethyl acetate) to give 8-amino-2-azido-6-fluoro-2-(hydroxymethyl)-5-methyl-3,4-dihydronaphthalen-1(2H)-one (8-54A) (500 mg, 41% yield). ¹H NMR (400 MHz, CDCl₃) δ ppm 6.31-6.85 (m, 2H), 6.23 (d, J=11.51 Hz, 1H), 3.96 (d, J=1.63 Hz, 2H), 2.76-2.99 (m, 3H), 2.08-2.18 (m, 1H), 2.07 (s, 3H), 1.85-2.01 (m, 1H), ¹⁹F NMR (376 MHz, CDCl₃) δ ppm −103.729. LCMS (ESI+) m/z: [MH]⁺ calcd for C₁₂H₁₄FN₄O₂⁺: 265.1, found: 265.5.

### (9S)-1-Azido-9-ethyl-5-fluoro-9-hydroxy-1-(hydroxymethyl)-4-methyl-1,2,3,9,12,15-hexa hydro-10H,13H-benzo[de]pyrano[3′,4′:6,7]indolizino[1,2-b]quinoline-10,13-dione (8-55A)

To a stirred mixture of 8-amino-2-azido-6-fluoro-2-(hydroxymethyl)-5-methyl-3,4-dihydronaphthalen-1(2H)-one (8-54A) (500 mg, 1.89 mmol, 1.0 equiv) and(S)-4-ethyl-4-hydroxy-7,8-dihydro-1H-pyrano[3,4-f]indolizine-3.6.10 (4H)-trione (1-4) (996 mg, 3.78 mmol, 2.0 equiv) in toluene (25 mL) was added 4-methylbenzenesulfonic acid (130 mg, 756 μmol, 0.4 equiv). After stirring at 120° C., for 12 h, it was cooled to room temperature, diluted with water (30 mL) and extracted with ethyl acetate (3×100 mL). The combined organic layers were dried over Na₂SO₄, filtered, concentrated, and the residue purified by silica gel chromatography (ethyl acetate/methanol) to give (9S)-1-azido-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (8-55A) (280 mg, 12% yield). ¹H NMR (400 MHz, DMSO-D₆) δ ppm 7.81 (d, J=10.8 Hz, 1H), 7.31 (d, J=2.1 Hz, 1H), 6.52 (s, 1H), 5.71 (t, J=5.8 Hz, 1H), 5.41-5.50 (m, 3H), 4.13 (t, J=6.7 Hz, 1H), 3.66-3.85 (m, 2H), 3.03-3.18 (m, 1H), 2.80-2.94 (m, 2H), 2.39 (s, 3H), 2.12-2.26 (m, 1H), 1.80-1.89 (m, 2H), 0.83-0.91 (m, 3H). ¹⁹F NMR (376 MHz, DMSO-D₆) δ ppm −111.5. LCMS (ESI+) m/z: [MH]⁺ calcd for C₂₅H₂₃FN₅O₅⁺: 492.1, found: 492.4.

### (9S)-1-Amino-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione hydrochloride (8-56A)

To a stirred mixture of (9S)-1-azido-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (8-55A) (230 mg, 467 μmol, 1.0 equiv) in tetrahydrofuran (4.6 mL) and water (1.1 mL) was added PPh₃ (245 mg, 935 μmol, 2.0 equiv). After stirring at 50° C., for 12 h, it was cooled to room temperature, diluted with water (20 mL) and extracted with ethyl acetate (3×30 mL). The combined organic layers were dried over Na₂SO₄, filtered, concentrated, and the residue purified by prep-HPLC (column: Phenomenex Luna C18 75*30 mm*3 um; mobile phase: [H₂O (0.04% HCl)-acetonitrile]: gradient: 10%-35% B over 8.0 min) to give (9S)-1-amino-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione hydrochloride (8-56A) (100 mg, 45% yield). ¹H NMR (400 MHz, CD₃OD) δ ppm 7.75 (dd, J=10.32, 7.07 Hz, 1H), 7.65 (d, J=4.13 Hz, 1H), 5.56-5.69 (m, 2H), 5.36-5.50 (m, 2H), 3.95-4.10 (m, 2H), 3.34-3.44 (m, 1H), 3.17-3.29 (m, 1H), 2.75 (dt, J=13.07, 4.53 Hz, 1H), 2.46 (br s, 3H) 2.29-2.42 (m, 1H), 1.91-2.03 (m, 2H), 1.01 (td. J=7.32, 2.25 Hz, 3H). ¹⁹F NMR (376 MHz, CD₃OD) δ ppm −111.89. LCMS (ESI+) m/z: [MH]⁺ calcd for C₂₅H₂₅FN₃O₅⁺: 466.1, found: 466.1.

### (1R,9S)-1-Amino-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione hydrochloride (8-57) and (1S,9S)-1-amino-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione hydrochloride (8-58)

(9S)-1-amino-9-ethyl-5-fluoro-9-hydroxy-1-(hydroxy methyl)-4-methyl-1,2,3,9,12,15-hexahydro-10H,13H-benzo[de]pyrano[3′,4′:6,7]indolizino[1,2-b]quinoline-10,13-dione hydrochloride (8-56A) (47 mg) was dissolved in methanol and separated by prep-HPLC to afford (1R,9S)-1-amino-9-ethyl-5-fluoro-9-hydroxy-1-(hydroxymethyl)-4-methyl-1,2,3,9,12,15-hexahydro-10H, 13H-benzo[de]pyrano[3′,4′:6,7]indolizino[1,2-b]quinoline-10,13-dione hydrochloride (8-57) (compound 8-57 may be the opposite enantiomer of that depicted) (11 mg) and (1S,9S)-1-amino-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione hydrochloride (8-58) (compound 8-58 may be the opposite enantiomer of that depicted) (15 mg). Note: the stereochemistry at this carbon is arbitrarily assigned.

Spectra for 8-57: ¹H NMR (400 MHz, DMSO-D₆) δ ppm 9.08 (br s, 3H), 7.88 (d, J=10.51 Hz, 1H), 7.36 (s, 1H), 6.56 (br s, 1H), 5.85 (br s, 1H), 5.60 (d, J=6.88 Hz, 2H), 5.45 (s, 2H), 3.80-3.92 (m, 2H), 3.14-3.25 (m, 2H), 2.56-2.65 (m, 1H), 2.40 (s, 3H), 2.17-2.27 (m, 1H), 1.88 (dt, J=14.10, 6.89 Hz, 2H), 0.88 (t, J=7.32 Hz, 3H). ¹⁹F NMR (376 MHz, DMSO-D₆) δ ppm −111.32. LCMS (ESI+) m/z: [MH]⁺ calcd for C₂₅H₂₅FN₃O₅⁺: 466.1, found: 466.3. SFC (RT=2.594 min).

Spectra for 8-58: ¹H NMR (400 MHz, DMSO-D₆) δ ppm 9.06 (br s, 3H), 7.88 (d, J=10.51 Hz, 1H), 7.36 (s, 1H), 6.48 (br s, 1H), 5.87 (br s, 1H), 5.59 (s, 2H), 5.46 (s, 2H), 3.80-3.90 (m, 2H), 3.15-3.27 (m, 2H), 2.61 (dt, J=13.07, 4.66 Hz, 1H), 2.40 (s, 3H), 2.16-2.26 (m, 1H), 1.80-1.94 (m, 2H), 0.87 (t, J=7.32 Hz, 3H), 1° F. NMR (376 MHz, DMSO-D₆) δ ppm −111.28. LCMS (ESI+) m/z: [MH]⁺ calcd for C₂₅H₂₅FN₃O₅⁺: 466.1, found: 466.3. SFC (RT=2.374 min).

Alternative and/or additional synthesis routes for the compounds described in Example 8 can be found in FIG. 9.

### Example 9

### Synthesis of: (1R,9S)-1-Amino-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione hydrochloride (9-64) and (1S,9S)-1-amino-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione hydrochloride (9-65)

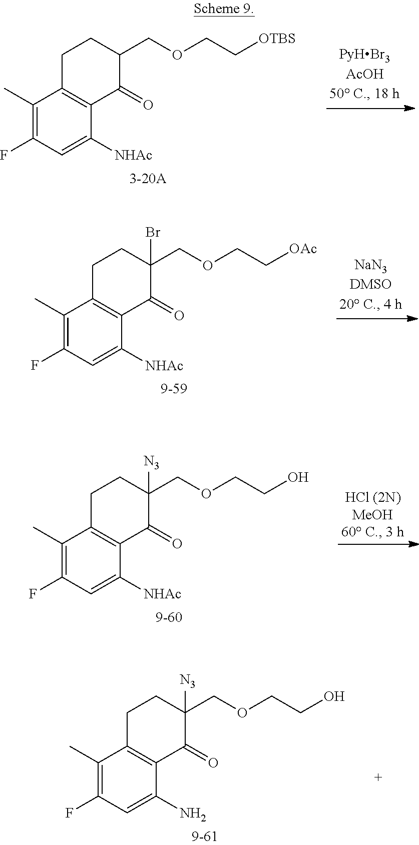 [SMILES: CC(=O)Nc1cc(F)c(C)c2c1C(=O)C(COCCO[Si](C)(C)C(C)(C)C)CC2 ; CC(=O)Nc1cc(F)c(C)c2c1C(=O)C(Br)(COCCOC(C)=O)CC2 ; CC(=O)Nc1cc(F)c(C)c2c1C(=O)C(COCCO)(N=[N+]=[N-])CC2 ; Cc1c(F)cc(N)c2c1CCC(COCCO)(N=[N+]=[N-])C2=O]

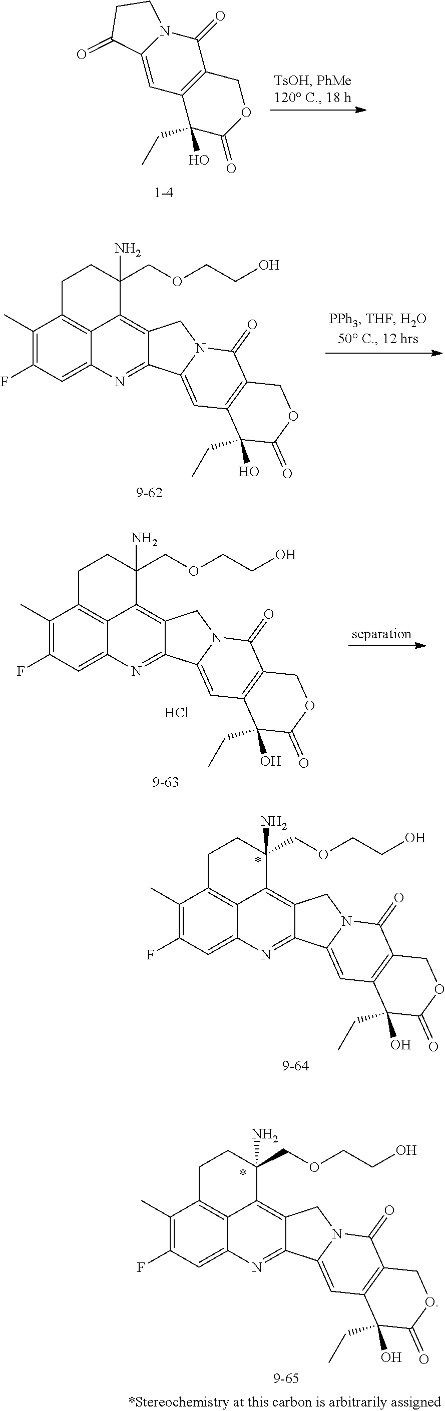 [SMILES: CC[C@@]1(O)C(=O)OCc2c1cc1n(c2=O)CCC1=O ; CC[C@@]1(O)C(=O)OCc2c1cc1n(c2=O)Cc2c-1nc1cc(F)c(C)c3c1c2C(N)(COCCO)CC3 ; CC[C@@]1(O)C(=O)OCc2c1cc1n(c2=O)Cc2c-1nc1cc(F)c(C)c3c1c2C(N)(COCCO)CC3 ; Cl ; CC[C@@]1(O)C(=O)OCc2c1cc1n(c2=O)Cc2c-1nc1cc(F)c(C)c3c1c2[C@@](N)(COCCO)CC3 ; CC[C@@]1(O)C(=O)OCc2c1cc1n(c2=O)Cc2c-1nc1cc(F)c(C)c3c1c2[C@](N)(COCCO)CC3]

### 2-((8-Acetamido-2-bromo-6-fluoro-5-methyl-1-oxo-1,2,3,4-tetrahydronaphthalen-2-yl)methoxy)ethyl acetate (9-59)

To a stirred mixture of N-(7-((2-((tert-butyldimethylsilyl)oxy)ethoxy)methyl)-3-fluoro-4-methyl-8-oxo-5,6,7,8-tetrahydronaphthalen-1-yl)acetamide (3-20A) (6.50 g, 12.7 mmol, 1 equiv) in acetic acid (130 mL) was added pyridinium tribromide (4.48 g, 14.01 mmol, 1.1 equiv). After stirring at 50° C., for 18 h under nitrogen atmosphere, the reaction mixture was cooled to room temperature, filtered, concentrated, and the residue purified by silica gel column chromatography (petroleum ether/ethyl acetate) to give 2-((8-acetamido-2-bromo-6-fluoro-5-methyl-1-oxo-1,2,3,4-tetrahydronaphthalen-2-yl) methoxy)ethyl acetate (9-59) (4.20 g, 76% yield). ¹H NMR (400 MHz, DMSO-D₆) δ ppm 11.85 (s, 1H). 8.37 (d, J=13.13 Hz, 1H), 4.21 (d, J=10.13 Hz, 1H), 4.11-4.16 (m, 2H), 3.94 (d, J=10.13 Hz, 1H), 3.71-3.78 (m, 2H), 3.12 (dt, J=17.92, 3.55 Hz, 1H), 2.83-2.95 (m, 1H), 2.42 (dd, J=8.25, 3.63 Hz, 2H), 2.19 (s, 3H), 2.16 (d, J=1.38 Hz, 3H), 1.98 (s, 3H). ¹⁹F NMR (376 MHz, DMSO-D₆) δ ppm −101.87. LCMS (ESI+) m/z: [MH]⁺ calcd for C₁₈H₂₂FNO₅Br⁺: 430.1, 432.1 found: 430.2, 432.2.

### 2-((8-Acetamido-2-azido-6-fluoro-5-methyl-1-oxo-1,2,3,4-tetrahydronaphthalen-2-yl)methoxy)ethyl acetate (9-60)

To a stirred mixture of 2-((8-acetamido-2-bromo-6-fluoro-5-methyl-1-oxo-1,2,3,4-tetrahydronaphthalen-2-yl)methoxy)ethyl acetate (9-59) (4.20 g, 9.76 mmol, 1 equiv) in DMSO (84 mL) was added sodium azide (1.27 g, 19.52 mmol, 2 equiv) at 20° C. After stirring at 20° C., for 4 h under nitrogen atmosphere, it was quenched by addition of water (100 mL) at 0° C., adjusted to pH 8 by addition of saturated aqueous sodium bicarbonate solution at 0° C., and extracted with dichloromethane (3×150 mL). The combined organic layers were dried over Na₂SO₄, filtered, concentrated, and the residue purified by silica gel column chromatography (petroleum ether/ethyl acetate) to give 2-((8-acetamido-2-azido-6-fluoro-5-methyl-1-oxo-1,2,3,4-tetrahydronaphthalen-2-yl)methoxy)ethyl acetate (9-60) (1.40 g, 36% yield). ¹H NMR (400 MHz, DMSO-D₆) δ ppm 11.55 (s, 1H), 8.30 (d, J=13.05 Hz, 1H), 4.08-4.14 (m, 2H), 3.87 (s, 2H), 3.66 (t, J=4.58 Hz, 2H), 2.90-2.98 (m, 2H), 2.20-2.26 (m, 1H), 2.19 (s, 3H), 2.12 (s, 3H), 2.05-2.11 (m, 1H), 1.98 (s, 3H). ¹⁹F NMR (376 MHz, DMSO-D₆) δ ppm −102.54. LCMS (ESI+) m/7: [MH]⁺ calcd for C₁₈H₂₂FN₄O₅⁺: 393.1, found: 393.2.

### 8-Amino-2-azido-6-fluoro-2-((2-hydroxyethoxy)methyl)-5-methyl-3,4-dihydronaphthalen-1(2H)-one (9-61)

To a stirred mixture of 2-((8-acetamido-2-azido-6-fluoro-5-methyl-1-oxo-1,2,3,4-tetrahydronaphthalen-2-yl)methoxy)ethyl acetate (9-60) (1.40 g, 3.56 mmol) in methanol (28 mL) was added hydrochloric acid (2 M, 28.00 mL). After stirring at 60° C., for 3 h, it was cooled to room temperature, quenched by addition of water (30 mL) at 20° C., adjusted to pH 7 by addition of saturated aqueous sodium bicarbonate solution at 0° C., and extracted with ethyl acetate (3×40 mL). The combined organic layers were dried over Na₂SO₄, filtered, and concentrated to give 8-amino-2-azido-6-fluoro-2-((2-hydroxyethoxy)methyl)-5-methyl-3,4-dihydronaphthalen-1(2H)-one (9-61) (1.00 g, 90% yield), which was used directly in the next step without further purification. ¹H NMR (400 MHz, DMSO-D₆) 8 ppm 7.48-7.58 (m, 1H), 6.42 (d, J=12.51 Hz, 1H), 4.57-4.64 (m, 1H), 3.72-3.87 (m, 2H), 3.45-3.55 (m, 4H), 2.78 (br t, J=5.75 Hz, 2H), 2.13-2.24 (m, 1H), 1.97 (s, 3H), 1.90-1.96 (m, 1H), ¹⁹F NMR (376 MHz, DMSO-D₆) δ ppm −106.16. LCMS (ESI+) m/z: [MH]⁺ calcd for C₁₄H₁₈FN₄O₃⁺: 309.2, found: 309.2.

### (9S)-1-Azido-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione (9-62)

After the stirred mixture of 8-amino-2-azido-6-fluoro-2-((2-hydroxyethoxy)methyl)-5-methyl-3,4-dihydronaphthalen-1(2H)-one (9-61) (250 mg, 810 μmol, 1 equiv). (S)-4-ethyl-4-hydroxy-7,8-dihydro-1H-pyrano[3,4-f]indolizine-3,6,10(4H)-trione (1-4) (426 mg, 1.62 mmol, 2 equiv) and 4-methylbenzenesulfonic acid (55.8 mg, 324 μmol, 0.4 equiv) in toluene (11.5 mL) was degassed and purged with argon for 3 times, it was stirred at 120° C., for 18 h under argon atmosphere. Then, it was cooled to room temperature, quenched by addition of water (20 mL) at 20° C., and extracted with dichloromethane (3×20 mL). The combined organic layers were dried over Na₂SO₄, filtered, concentrated, and the residue purified by silica gel column chromatography (petroleum ether/ethyl acetate) to give (9S)-1-azido-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione (9-62) (149 mg, 34% yield). LCMS (ESI+) m/z: [MH]⁺ calcd for C₂₇H₂₇O₆N₅F⁺: 536.1, found: 536.4.

### (9S)-1-Amino-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione hydrochloride (9-63)

To a stirred mixture of (9S)-1-azido-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione (9-62) (149 mg, 278 μmol, 1 equiv) in tetrahydrofuran (3 mL) and water (0.75 mL) was added PPh₃ (145 mg, 556 μmol, 2 equiv). After stirring at 50° C., for 12 h, it was cooled to room temperature, quenched by addition of aqueous HCl (0.5 M, 20 mL), and extracted with dichloromethane (3× 20 mL). The aqueous layer was lyophilization to give (9S)-1-amino-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione hydrochloride (9-63) (80.0 mg, 45% yield), which was used directly in the next step without further purification. LCMS (ESI+) m/z: [MH]⁺ calcd for C₂₇H₂₉O₆N₃F⁺: 510.2, found: 510.3. SFC (retention time=1.609 min. 1.941 min).

### (1R,9S)-1-Amino-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione hydrochloride (9-64) and (1S,9S)-1-amino-9-ethyl-5-fluoro-9-hydroxy-1-((2-hydroxyethoxy)methyl)-4-methyl-1,2,3,9,12,15-hexahydro-10H,13H-benzo[de]pyrano [3′,4′:6,7]indolizino[1,2-b]quinoline-10,13-dione hydrochloride (9-65)

A mixture of (1R,9S)-1-amino-9-ethyl-5-fluoro-9-hydroxy-1-((2-hydroxy ethoxy)methyl)-4-methyl-1,2,3,9,12,15-hexahydro-10H,13H-benzo[de]pyrano[3′,4′:6,7]indolizino[1,2-b]quinoline-10,13-dione hydrochloride (9-63) (100 mg, 157 μmol) in DMSO (5 mL) was separated by prep-HPLC to afford (1R,9S)-1-amino-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione hydrochloride (9-64) (compound 9-64 may be the opposite enantiomer of that depicted) (5.20 mg) and (1S,9S)-1-amino-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizino[1,2-b]quinoline-10,13-dione hydrochloride (9-65) (compound 9-65 may be the opposite enantiomer of that depicted) (4.80 mg). Note: *the stereochemistry at this carbon is arbitrarily assigned.

prep-HPLC separation method: Instrument: Gilson 281 Semi-preparative HPLC system Column: Phenomenex Luna C18 100*30 mm*3 um. Mobile phase: A: H₂O (0.04% HCl); B: acetonitrile. Gradient: B from 10.00% to 45.00% in 8.00 min. Flow rate: 25.00 ml/min Monitor wavelength: 220&254 nm, prep-HPLC (9-64, retention time=4.883 min) and SFC (9-65, retention time=5.136 min).

Spectra for 9-64: ¹H NMR (400 MHz, DMSO-D₆) δ ppm 9.21 (br s, 3H), 7.88 (d, J=10.51 Hz, 1H), 7.36 (s, 1H), 6.58 (br s, 1H), 5.59 (s, 2H) 5.45 (s, 2H), 3.88 (s, 2H), 3.40-3.50 (m, 4H), 3.20-3.30 (m, 1H), 3.04-3.18 (m, 1H), 2.62-2.67 (m, 1H), 2.40 (s, 3H), 2.24 (td, J=12.41, 5.32 Hz, 1H), 1.88 (dt, J=14.23, 6.96 Hz, 2H), 0.88 (t, J=7.32 Hz, 3H). ¹⁹F NMR (376 MHz, DMSO-D₆) δ ppm −111.24. LCMS (ESI+) m/z: [MH]⁺ calcd for C₂₇H₂₉O₆N₃F⁺: 510.2, found: 510.3. SFC (retention time=1.929 min).

Spectra for 9-65: ¹H NMR (400 MHz, DMSO-D₆) δ ppm 9.18 (br s, 3H), 7.89 (d, J=10.51 Hz, 1H), 7.36 (s, 1H), 6.55 (br s, 1H), 5.59 (s, 2H), 5.46 (s, 2H), 4.55-4.67 (m, 1H), 3.86 (s, 2H), 3.44-3.54 (m, 4H), 3.26 (br d, J=3.38 Hz, 1H), 3.08-3.20 (m, 1H), 2.67 (br dd, J=8.25, 4.75 Hz, 1H), 2.40 (s, 3H), 2.17-2.29 (m, 1H), 1.80-1.94 (m, 2H), 0.88 (t, J=7.32 Hz, 3H). ¹⁹F NMR (376 MHz, DMSO-D₆) δ ppm −111.21. LCMS (ESI+) m/z: [MH]⁺ calcd for C₂₇H₂₉O₆N₃F⁺: 510.2, found: 510.3. SFC (retention time=1.602 min).

### Example 10

### Synthesis of: N-((1R,9S)-9-Ethyl-5-fluoro-9-hydroxy-1-((2-hydroxyethoxy)methyl)-4-methyl-10,13-dioxo-2,3,9,10,13,15-hexahydro-1H, 12H-benzo[de]pyrano[3′,4′:6,7]indolizino[1,2-b]quinolin-1-yl)acetamide (10-68) and N-((1S,9S)-9-ethyl-5-fluoro-9-hydroxy-1-((2-hydroxyethoxy)methyl)-4-methyl-10,13-dioxo-2,3,9,10,13,15-hexahydro-1H,12H-benzo[de]pyrano[3′,4′:6,7]indolizino[1,2-b]quinolin-1-yl)acetamide (10-69)

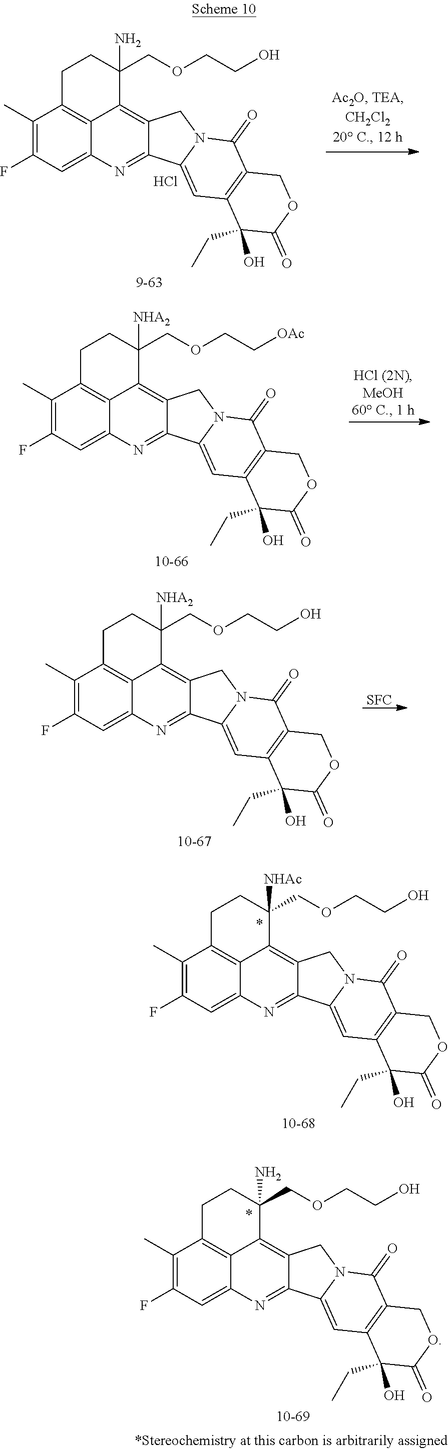

### 2-(((9S)-1-Acetamido-9-ethyl-5-fluoro-9-hydroxy-4-methyl-10,13-dioxo-2,3,9,10,13,15-hexahydro-1H,12H-benzol[de]pyrano[3′,4′:6,7]indolizino[1,2-b]quinolin-1-yl)methoxy)ethyl acetate (10-66)

To a stirred mixture of (9S)-1-amino-9-ethyl-5-fluoro-9-hydroxy-1-((2-hydroxyethoxy)methyl)-4-methyl-1,2,3,9,12,15-hexahydro-10H,13H-benzo[de]pyrano[3′,4′:6,7]indolizin o[1,2-b]quinoline-10,13-dione hydrochloride (9-63) (120 mg, 220 μmol, 1.0 equiv) in dichloromethane (2.4 mL) was added triethylamine (222 mg, 2.20 mmol, 304 μL, 10 equiv), stirred at 20° C., for 15 min, then Ac₂O (112.3 mg, 1.10 mmol, 5.0 equiv) added dropwise at 0° C. After stirring at 20° C., for 12 h, it was quenched by addition of water (10 mL) and extracted with dichloromethane (3×10 mL). The combined organic layers were dried over Na₂SO₄, filtered and concentrated to give 2-(((9S)-1-acetamido-9-ethyl-5-fluoro-9-hydroxy-4-methyl-10,13-dioxo-2,3,9,10,13,15-hexahydro-1H,12H-benzo[de]pyrano[3′,4′:6,7]indolizino [1,2-b]quinolin-1-yl)methoxy)ethyl acetate (10-66) (110 mg, 52% yield), which was used directly in the next step without further purification. LCMS (ESI+) m/z: [MH]⁺ calcd for C₃₁H₃₃FN₃O₈⁺: 594.2, found: 594.3.

### N-((9S)-9-Ethyl-5-fluoro-9-hydroxy-1-((2-hydroxyethoxy)methyl)-4-methyl-10,13-dioxo-2,3,9,10,13,15-hexahydro-1H,12H-benzo[de]pyrano[3′,4′:6,7]indolizino[1,2-b]quinolin-1-yl)acetamide (10-67)

To a stirred mixture of 2-(((9S)-1-acetamido-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)methoxy)ethyl acetate (10-66) (110 mg, 185 μmol, 1.0 equiv, purity 67%) in methanol (4.4 mL) was added hydrochloric acid (2 M, 4.4 mL). After stirring at 60° C. for 1 h, it was cooled to room temperature, quenched by addition of water (8.8 mL) at 20° C., and extracted with dichloromethane (3×10 mL). The combined organic layers were dried over Na₂SO₄, filtered, concentrated, and the residue purified by prep-TLC (ethyl acetate/methanol=4/1) to give N-((9S)-9-ethyl-5-fluoro-9-hydroxy-1-((2-hydroxyethoxy)methyl)-4-methyl-10,13-dioxo-2,3,9,10,13,15-hexahydro-1H, 12H-benzo[de]pyrano [3′,4′:6,7]indolizino[1,2-b]quinolin-1-yl)acetamide (10-67) (28 mg, 40% yield). ¹H NMR (400 MHz, DMSO-D₆) δ ppm 8.57 (d, J=6.13 Hz, 1H), 7.79 (d, J=10.88 Hz, 1H), 7.30 (s, 1H), 6.51 (d, J=11.51 Hz, 1H), 5.39-5.52 (m, 3H), 4.85 (dd, J=19.14, 3.13 Hz, 1H), 4.57-4.67 (m, 1H), 3.69 (br d. J=7.00 Hz, 2H), 3.36-3.52 (m, 4H), 3.17-3.27 (m, 1H), 2.95-3.09 (m, 1H), 2.80-2.88 (m, 1H), 2.38 (s, 3H), 2.18-2.26 (m, 1H), 1.99 (d, J=3.13 Hz, 3H), 1.77-1.92 (m, 2H), 0.81-0.93 (m, 3H). ¹⁹F NMR (376 MHz, DMSO-D₆) δ ppm −111.99. LCMS (ESI+) m/z: [MH]⁺ calcd for C₂₉H₃₁FN₃O₇⁺: 552.2, found: 552.3. SFC (retention time=1.193 min, 1.344 min).

### N-((1R,9S)-9-Ethyl-5-fluoro-9-hydroxy-1-((2-hydroxyethoxy)methyl)-4-methyl-10,13-dioxo-2,3,9,10,13,15-hexahydro-1H,12H-benzo [de]pyrano[3′,4′:6,7]indolizin o[1,2-b]quinolin-1-yl)acetamide (10-68) and N-((1S,9S)-9-ethyl-5-fluoro-9-hydroxy-1-((2-hydroxyethoxy)methyl)-4-methyl-10,13-dioxo-2,3,9,10,13,15-hexahydro-1H,12H-benzo[de]pyrano[3′,4′:6,7]indolizino[1,2-b]quinolin-1-yl)acetamide (10-69)

N-((9S)-9-ethyl-5-fluoro-9-hydroxy-1-((2-hydroxyethoxy)methyl)-4-methyl-10,13-dioxo-2,3,9,10,13,15-hexahydro-1H, 12H-benzo[de]pyrano[3′,4′:6,7]indolizino[1,2-b]quinolin-1-yl)acetamide (10-67) (38 mg, 68 μmol) was separated by SFC (Column: DAICEL CHIRALCEL OD (250 mm*30 mm, 10 um): mobile phase: [CO₂-methanol]: B %: 15%, isocratic elution mode. Flow rate: 70.00 g/min. Monitor wavelength: 220&254 nm, Column temperature: 40° C.) to afford N-((1R,9S)-9-ethyl-5-fluoro-9-hydroxy-1-((2-hydroxyethoxy)methyl)-4-methyl-10,13-dioxo-2,3,9,10,13,15-hexahydro-1H,12H-benzo[de]pyrano[3′,4′:6,7]indolizino[1,2-b]quinolin-1-yl) acetamide (10-68) (compound 10-68 may be the opposite enantiomer of that depicted) (4.01 mg) and N-((1S,95)-9-ethyl-5-fluoro-9-hydroxy-1-((2-hydroxyethoxy)methyl)-4-methyl-10,13-dioxo-2,3,9,10,13,15-hexahydro-1H,12H-benzo[de]pyrano[3′,4′:6,7]indolizino[1,2-b]quinolin-1-yl)acetamide (10-69) (compound 10-69 may be the opposite enantiomer of that depicted) (2.11 mg). Note: the stereochemistry is arbitrarily assigned.

Spectra for 10-68: ¹H NMR (400 MHz, DMSO-D₆) δ ppm 8.58 (s, 1H), 7.79 (d, J=10.63 Hz, 1H), 7.31 (s, 1H), 6.51 (s, 1H), 5.49 (d, J=19.14 Hz, 1H), 5.42 (s, 2H), 4.86 (d, J=19.14 Hz, 1H), 4.58-4.67 (m, 1H), 3.66-3.74 (m, 2H), 3.45-3.50 (m, 3H), 3.38-3.43 (m, 1H), 3.08-3.10 (m, 1H), 2.97-3.06 (m, 1H), 2.81-2.91 (m, 1H), 2.38 (s, 3H), 2.18-2.25 (m, 1H), 1.98 (s, 3H), 1.79-1.92 (m, 2H), 0.87 (t, J=7.38 Hz, 3H), 19F NMR (376 MHz, DMSO-D₆) δ ppm −111.98. LCMS (ESI+) m/z: [MH]⁺ calcd for C₂₉H₃₁O₇N₃F⁺: 552.2, found: 552.4. SFC (retention time=1.189 min).

Spectra for 10-69: ¹H NMR (400 MHz, DMSO-D₆) δ ppm 8.56 (s, 1H), 7.73-7.85 (m, 1H), 7.30 (s, 1H), 6.53 (br s, 1H), 5.43 (d, J=19.2 Hz, 1H), 5.42 (s, 1H), 4.85 (d, J=19.2 Hz, 1H), 4.59-4.69 (m, 1H), 3.68 (s, 2H), 3.43-3.49 (m, 4H), 3.13-3.20 (m, 1H), 2.95-3.11 (m, 1H), 2.82-2.93 (m, 1H), 2.35-2.40 (m, 3H), 2.18-2.27 (m, 1H), 1.99 (s, 3H), 1.78-1.93 (m, 2H), 0.86 (br t, J=7.32 Hz, 3H). ¹⁹F NMR (376 MHz, DMSO-D₆) δ ppm −111.95. LCMS (ESI+) m/z: [MH]⁺ calcd for C₂₉H₃₁O₇N₃F⁺: 552.2, found: 552.4. SFC (retention time=1.327 min).

### Example 11

### Synthesis of: (1S,9S)-1-((S)-1-Amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (11-76), (1S,9S)-1-((R)-1-Amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (11-78), (1R,9S)-1-((S)-1-Amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (11-80), and (1R,9S)-1-((R)-1-Amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (11-82)

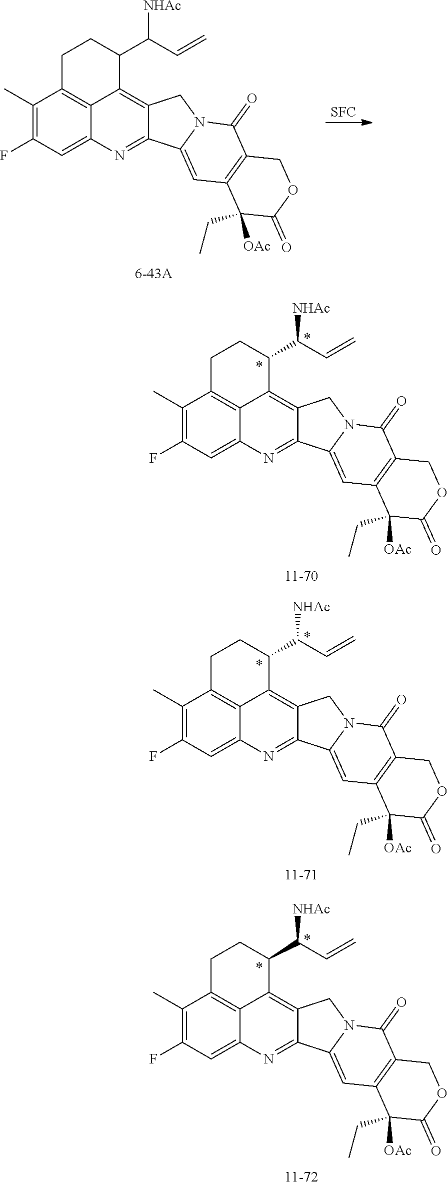 [SMILES: C=CC(NC(C)=O)C1CCc2c(C)c(F)cc3nc4c(c1c23)Cn1c-4cc2c(c1=O)COC(=O)[C@@]2(CC)OC(C)=O ; "SFC" ; C=C[C@H](NC(C)=O)[C@H]1CCc2c(C)c(F)cc3nc4c(c1c23)Cn1c-4cc2c(c1=O)COC(=O)[C@@]2(CC)OC(C)=O ; C=C[C@@H](NC(C)=O)[C@H]1CCc2c(C)c(F)cc3nc4c(c1c23)Cn1c-4cc2c(c1=O)COC(=O)[C@@]2(CC)OC(C)=O ; C=C[C@H](NC(C)=O)[C@@H]1CCc2c(C)c(F)cc3nc4c(c1c23)Cn1c-4cc2c(c1=O)COC(=O)[C@@]2(CC)OC(C)=O]

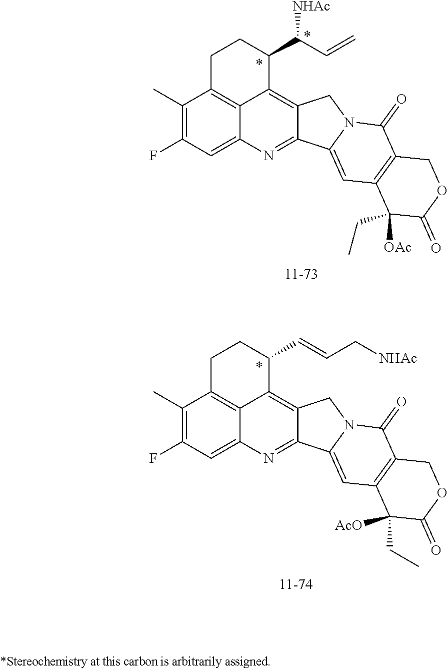 [SMILES: C=C[C@@H](NC(C)=O)[C@@H]1CCc2c(C)c(F)cc3nc4c(c1c23)Cn1c-4cc2c(c1=O)COC(=O)[C@@]2(CC)OC(C)=O ; CC[C@@]1(OC(C)=O)C(=O)OCc2c1cc1n(c2=O)Cc2c-1nc1cc(F)c(C)c3c1c2[C@@H](/C=C/CNC(C)=O)CC3]

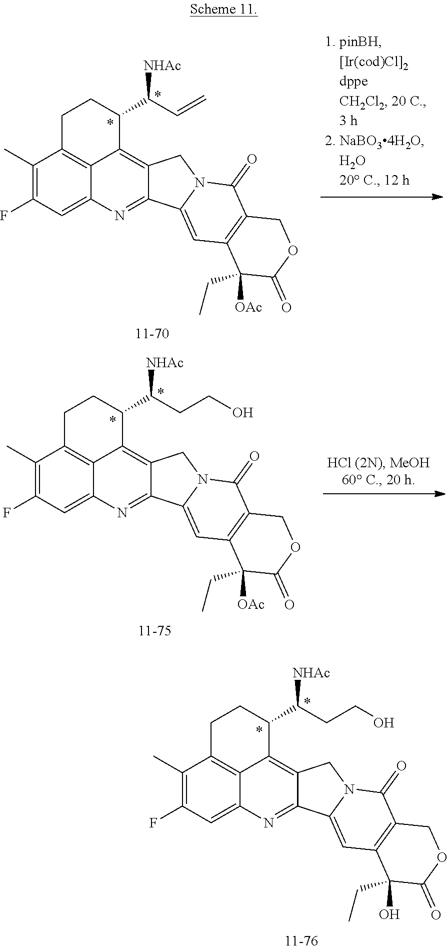 [SMILES: C=C[C@H](NC(C)=O)[C@H]1CCc2c(C)c(F)cc3nc4c(c1c23)Cn1c-4cc2c(c1=O)COC(=O)[C@@]2(CC)OC(C)=O ; CC[C@@]1(OC(C)=O)C(=O)OCc2c1cc1n(c2=O)Cc2c-1nc1cc(F)c(C)c3c1c2[C@@H]([C@H](CCO)NC(C)=O)CC3 ; CC[C@@]1(O)C(=O)OCc2c1cc1n(c2=O)Cc2c-1nc1cc(F)c(C)c3c1c2[C@@H]([C@H](CCO)NC(C)=O)CC3]

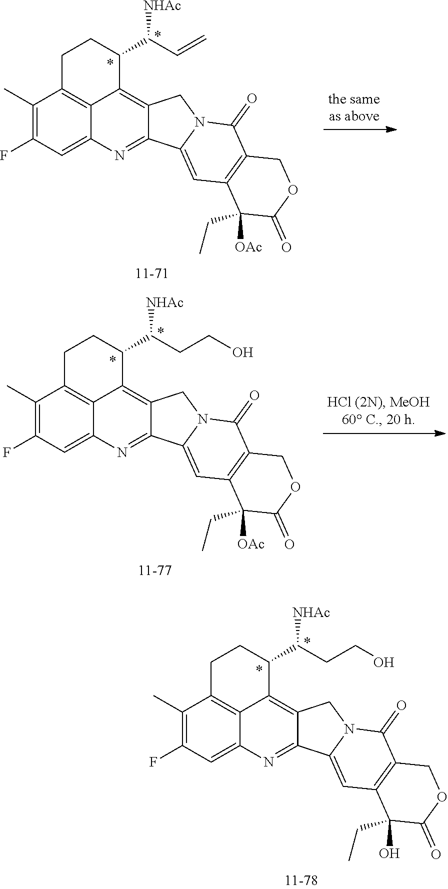 [SMILES: C=C[C@@H](NC(C)=O)[C@H]1CCc2c(C)c(F)cc3nc4c(c1c23)Cn1c-4cc2c(c1=O)COC(=O)[C@@]2(CC)OC(C)=O ; CC[C@@]1(OC(C)=O)C(=O)OCc2c1cc1n(c2=O)Cc2c-1nc1cc(F)c(C)c3c1c2[C@@H]([C@@H](CCO)NC(C)=O)CC3 ; CC[C@@]1(O)C(=O)OCc2c1cc1n(c2=O)Cc2c-1nc1cc(F)c(C)c3c1c2[C@@H]([C@@H](CCO)NC(C)=O)CC3]

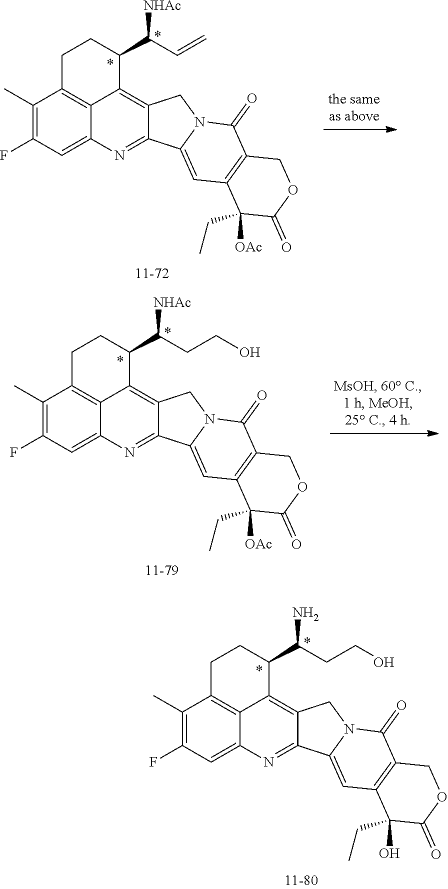 [SMILES: C=C[C@H](NC(C)=O)[C@@H]1CCc2c(C)c(F)cc3nc4c(c1c23)Cn1c-4cc2c(c1=O)COC(=O)[C@@]2(CC)OC(C)=O ; CC[C@@]1(OC(C)=O)C(=O)OCc2c1cc1n(c2=O)Cc2c-1nc1cc(F)c(C)c3c1c2[C@H]([C@H](CCO)NC(C)=O)CC3 ; CC[C@@]1(O)C(=O)OCc2c1cc1n(c2=O)Cc2c-1nc1cc(F)c(C)c3c1c2[C@H]([C@@H](N)CCO)CC3]

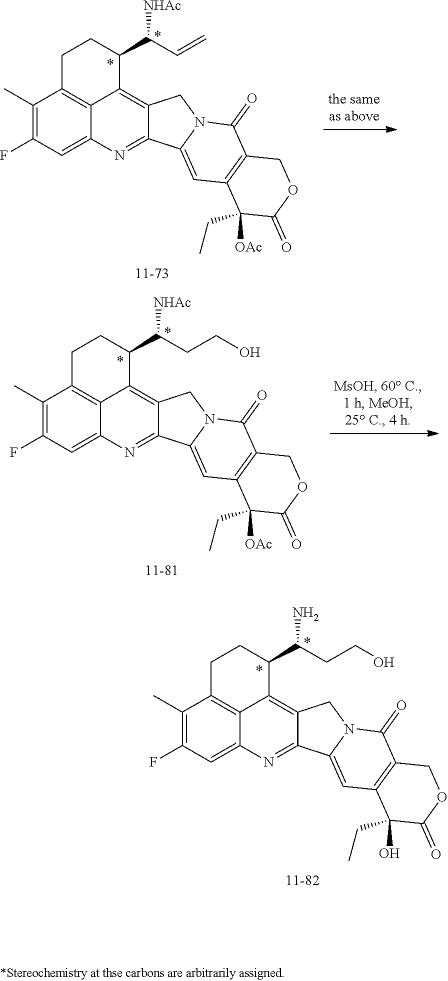 [SMILES: C=C[C@@H](NC(C)=O)[C@@H]1CCc2c(C)c(F)cc3nc4c(c1c23)Cn1c-4cc2c(c1=O)COC(=O)[C@@]2(CC)OC(C)=O ; CC[C@@]1(OC(C)=O)C(=O)OCc2c1cc1n(c2=O)Cc2c-1nc1cc(F)c(C)c3c1c2[C@H]([C@@H](CCO)NC(C)=O)CC3 ; CC[C@@]1(O)C(=O)OCc2c1cc1n(c2=O)Cc2c-1nc1cc(F)c(C)c3c1c2[C@H]([C@H](N)CCO)CC3]

### (1S,9S)-1-((S)-1-Acetamidoallyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-70), (1S,9S)-1-((R)-1-acetamidoallyl)-9-ethyl-5-fluoro-4-methyl-10,13-dioxo-2,3,9,10,13,15-hexahydro-1H,12H-benzol[de]pyrano[3′,4′:6,7]indolizino[1,2-b]quinolin-9-yl acetate (11-71), (1R,9S)-1-((S)-1-acetamidoallyl)-9-ethyl-5-fluoro-4-methyl-10,13-dioxo-2,3,9,10,13,15-hexahydro-1H,12H-benzo[de]pyrano[3′,4′:6,7 [indolizino]1,2-b]quinolin-9-yl acetate (11-72), (1R,9S)-1-((R)-1-acetamidoallyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-73), and (1R,9S)-1-((E)-3-acetamidoprop-1-en-1-yl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-74)

(9S)-1-(1-acetamidoallyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (6-43A) (5.0 g) was separated by SFC (Instrument: Column: REGIS(s,s) WHELK-01 (250) mm*50 mm, 10 um). Mobile phase: A for CO₂ and B for methanol:acetonitrile=1:1. Gradient: B %=60.00% isocratic elution mode. Flow rate: 200.00 g/min. Monitor wavelength: 220&254 nm. Column temperature: 40° C. System back pressure: 100 bar) to give (1S,9S)-1-((S)-1-acetamidoallyl)-9-ethyl-5-fluoro-4-methyl-10,13-dioxo-2,3,9,10,13,15-hexahydro-1H,12H-benzo[de]pyrano[3′,4′:6,7]-indolizino[1,2-b]quinolin-9-yl acetate (11-70) (compound 11-70 may be the opposite enantiomer of that depicted) (300 mg). (1S,9S)-1-((R)-1-acetamidoallyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-71) (compound 11-71 may be the opposite enantiomer of that depicted) (230) mg), a mixture of 11-72 and 11-73, and (1R,9S)-1-((F)-3-acetamidoprop-1-en-1-yl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-74) (1.0 g) (compound 11-74 may be the opposite enantiomer of that depicted). The mixture of 11-72 and 11-73 was further separated by SFC (Instrument: Waters SFC80 Preparative SFC System. Column: DAICEL CHIRALCEL OD (250 mm*30 mm, 10 um). Mobile phase: A for CO₂ and B for methanol. Gradient: B %=38.00% isocratic elution mode. Flow rate: 64.00 g/min. Monitor wavelength: 220&254 nm. Column temperature: 40° C. System back pressure: 100 bar) to give (1R,9S)-1-((S)-1-acetamidoallyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-72) (140 mg) (compound 11-72 may be the opposite enantiomer of that depicted), and (1R,9S)-1-((R)-1-acetamidoallyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-73) (380 mg) (compound 11-73 may be the opposite enantiomer of that depicted). Note: The stereochemistries at these carbons are arbitrarily assigned.

Spectra for 11-70: ¹H NMR (400 MHz, DMSO-D₆) δ ppm 8.21 (d, J=9.13 Hz, 1H) 7.75 (d, J=11.01 Hz, 1H) 7.01 (s, 1H) 5.70 (ddd, J=16.85, 10.47, 6.07 Hz, 1H) 5.48 (s, 2H) 5.36 (d, J=18.64 Hz, 1H) 5.12 (d, J=18.64 Hz, 1H) 4.76-4.93 (m, 2H) 4.49-4.62 (m, 1H) 3.34-3.41 (m, 1H) 3.12-3.17 (m, 1H) 2.98-3.09 (m, 1H) 2.30-2.39 (m, 4H) 2.21 (s, 3H) 2.09-2.17 (m, 2H) 1.81-1.94 (m, 4H) 0.93 (t, J=7.38 Hz, 3H). ¹⁹F NMR (376 MHz, DMSO-D₆) δ ppm −111.55. LCMS (ESI+) m/z: [MH]⁺ calcd for C₃₁H₃₁FN₃O₆⁺: 560.2, found: 560.4. SFC (retention time=0.771 min).

Spectra for 11-71: ¹H NMR (400 MHz, DMSO-D₆) δ ppm 8.26 (d, J=9.13 Hz, 1H) 7.71 (d, J=11.01 Hz, 1H) 7.00 (s, 1H) 5.91 (ddd. J=17.39, 10.01, 7.88 Hz, 1H) 5.30-5.59 (m, 4H) 5.07-5.30 (m, 2H) 4.52-4.65 (m, 1H) 2.95-3.20 (m, 3H) 2.27-2.40 (m, 4H) 2.04-2.25 (m, 5H) 1.89-2.03 (m, 1H) 1.52 (s, 3H) 0.92 (t, J=7.44 Hz, 3H). ¹⁹F NMR (376 MHz, DMSO-D₆) δ ppm −112.05. LCMS (ESI+) m/z: [MH]⁺ calcd for C₃₁H₃₁FN₃O₆⁺: 560.2, found: 560.4. SFC (retention time=1.050 min).

Spectra for 11-72: ¹H NMR (400 MHz, DMSO-D₆) δ ppm 8.24 (d, J=9.16 Hz, 1H) 7.72 (d, J=11.04 Hz, 1H) 7.00 (s, 1H) 5.91 (ddd. J=17.44, 9.91, 7.91 Hz, 1H) 5.31 (s, 4H) 5.25 (d, J=17.07 Hz, 1H) 5.13 (d, J=10.67 Hz, 1H) 4.55-4.64 (m, 1H) 3.31-3.40 (m, 1H) 2.98-3.15 (m, 2H) 2.38 (s, 3H) 2.26-2.31 (m, 1H) 2.22 (s, 3H) 1.82-2.01 (m, 2H) 1.51 (s, 3H) 0.90 (t, J=7.34 Hz, 3H). ¹⁹F NMR (376 MHz, DMSO-D₆) δ ppm −111.98. LCMS (ESI+) m/z: [MH]⁺ calcd for C₃₁H₃₁FN₃O₆⁺: 560.2, found: 560.3.

Spectra for 11-73: ¹H NMR (400 MHz, DMSO-D₆) δ ppm 8.22 (d, J=9.29 Hz, 1H) 7.75 (d, J=10.92 Hz, 1H) 7.01 (s, 1H) 5.70 (ddd, J=16.88, 10.48, 6.15 Hz, 1H) 5.49 (d, J=1.63 Hz, 2H) 5.06-5.40 (m, 2H) 4.74-4.94 (m, 2H) 4.45-4.61 (m, 1H) 3.35-3.39 (m, 1H) 3.12-3.25 (m, 1H) 2.99-3.08 (m, 1H) 2.31-2.40 (m, 4H) 2.22 (s, 3H) 2.06-2.18 (m, 2H) 1.87 (s, 4H) 0.90 (t, J=7.34 Hz, 3H). ¹⁹F NMR (376 MHz, DMSO-D₆) δ ppm −111.48. LCMS (ESI+) m/z: [MH]⁺ calcd for C₃₁H₃₁FN₃O₆⁺: 560.2, found: 560.3.

Spectra for 11-74: ¹H NMR (400 MHz, DMSO-D₆) δ ppm 7.70-7.84 (m, 2H) 7.02 (s, 1H) 5.78 (dd, J=15.45, 6.57 Hz, 1H) 5.42-5.54 (m, 2H) 5.35 (d, J=18.89 Hz, 1H) 5.18 (dt, J=15.45, 5.66 Hz, 1H) 5.04 (d, J=18.76 Hz, 1H) 4.14 (br s, 1H) 3.59 (br s, 1H) 3.08-3.21 (m, 1H) 2.88-3.01 (m, 1H) 2.38 (s, 3H) 2.12-2.24 (m, 7H) 1.74 (s, 6H) 0.91 (t, J=7.38 Hz, 3H). ¹⁹F NMR (376 MHz, DMSO-D₆) δ ppm −111.12. LCMS (ESI+) m/z: [MH]⁺ calcd for C₃₁H₃₁FN₃O₆⁺: 560.2, found: 560.4. SFC (retention time=3.398 min).

### (1S,9S)-1-((S)-1-Acetamido-3-hydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-75)

To a stirred mixture of (1S,9S)-1-((S)-1-acetamidoallyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (11-70) (300 mg, 536 μmol, 1.0 equiv) and 4,4,5,5-tetramethyl-1,3,2-dioxaborolane (411 mg, 3.22 mmol, 466 μL, 6.0 equiv) in dichloromethane (6 mL) were added a solution of chloroiridium; (1Z,5Z)-cycloocta-1,5-diene (36.0 mg, 53.6 μmol, 0.1 equiv) and 2-diphenylphosphanylethyl(diphenyl)phosphane (42.7 mg, 107 μmol, 0.2 equiv) in dichloromethane (1 mL). After stirring at 20° C., for 3 h, a mixture of sodium 3-oxidodioxaborirane tetrahydrate (412 mg, 2.68 mmol, 515 μL, 5.0 equiv) in H₂O (3 mL) was added and stirred at 20° C., for 12 h. The reaction mixture was extracted with dichloromethane (3×10 mL), combined organic layers washed with brine, dried over Na₂SO₄, filtered, and concentrated, and the residue purified by silica gel column chromatography (ethyl acetate/methanol) to give (1S,9S)-1-((S)-1-acetamido-3-hydroxypropyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (11-75) (50 mg, 16% yield). ¹H NMR (400 MHz, DMSO-D₆) δ ppm 7.81 (br d, J=9.3 Hz, 1H) 7.73 (br d. J=11.0 Hz, 1H) 7.02 (s, 1H) 5.49 (s, 2H) 5.43 (br d, J=18.5 Hz, 1H) 5.18 (d, J=18.6 Hz, 1H) 4.07-4.19 (m, 2H) 3.21-3.29 (m, 2H) 3.10-3.17 (m, 1H) 2.95-3.06 (m, 1H) 2.31-2.41 (m, 5H) 2.12-2.21 (m, 5H) 1.80-1.94 (m, 1H) 1.73 (s, 3H) 1.57-1.65 (m, 1H) 1.33-1.43 (m, 1H) 0.92 (t, J=7.38 Hz, 3H). ¹⁹F NMR (376 MHz, DMSO-D₆) δ ppm −111.68. LCMS (ESI+) m/z: [MH]⁺ calcd for C₃₁H₃₃FN₃O₇⁺: 578.2, found: 578.4.

### (1S,9S)-1-((S)-1-Amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (11-76)

To a stirred mixture of (1S,9S)-1-((S)-1-acetamido-3-hydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-75) (50 mg, 86.5 μmol, 1.0 equiv) in methanol (1.0 mL) was added HCl (2 M, 2 mL). After stirring at 60° C. for 20 h, it was cooled to room temperature, filtered and the filtrate purified by prep-HPLC (Instrument: Gilson 281 Semi-preparative HPLC system, Column: Phenomenex luna C18 100*40 mm*5 um, Mobile phase: A: H₂O (0.04% HCl); B: acetonitrile, Gradient: B from 10.00% to 40.00% in 8.00 min, Flow rate: 60.00 ml/min, Monitor wavelength: 220&254 nm) to give (1S,9S)-1-((S)-1-amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (11-76) (10.2 mg, 23% yield). ¹H NMR (400 MHz, DMSO-D₆) δ ppm 8.04 (br d, J=0.88 Hz, 3H) 7.80 (d, J=10.88 Hz, 1H) 7.32 (s, 1H) 6.36-6.68 (m, 1H) 5.44 (s, 2H) 5.39 (br d, J=18.89 Hz, 1H) 5.17-5.26 (m, 1H) 3.54-3.63 (m, 2H) 3.27-3.34 (m, 2H) 3.22 (br d, J=13.76 Hz, 1H) 3.01-3.14 (m, 1H) 2.58 (br s, 1H) 2.39 (s, 3H) 1.94-2.06 (m, 1H) 1.87 (tt, J=13.68, 6.96 Hz, 2H) 1.63-1.79 (m, 1H) 1.22-1.38 (m, 1H) 0.89 (t, J=7.25 Hz, 3H). ¹⁹F NMR (376 MHz, DMSO-D₆) δ ppm −111.64. LCMS (ESI+) m/z: [MH]⁺ calcd for C₂₇H₂₉FN₃O₅⁺: 494.2, found: 494.4. SFC (retention time=3.178 min). Note: *The stereochemistries at these carbons are arbitrarily assigned.

### (1S,9S)-1-((R)-1-Acetamido-3-hydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-77)

11-77 was synthesized in a similar fashion to that of 11-75.

Spectra for 11-77: ¹H NMR (400 MHz, DMSO-D₆) δ ppm 7.93 (d, J=8.88 Hz, 1H) 7.71 (d, J=11.01 Hz, 1H) 7.00 (s, 1H) 5.28-5.59 (m, 4H) 4.34 (t, J=5.00 Hz, 1H) 4.16 (qd, J=8.98, 3.44 Hz, 1H) 3.36-3.45 (m, 1H) 3.32-3.35 (m, 1H) 3.21-3.28 (m, 1H) 3.02-3.09 (m, 2H) 2.31-2.40 (m, 4H) 2.21 (s, 3H) 2.11-2.18 (m, 2H) 1.92-2.02 (m, 1H) 1.68 (br d, J=4.13 Hz, 2H) 1.49 (s, 3H) 0.92 (t, J=7.38 Hz, 3H). ¹⁹F NMR (376 MHz, DMSO-D₆) δ ppm −111.14. LCMS (ESI+) m/z: [MH]⁺ calcd for C₃₁H₃₃FN₃O₇⁺: 578.2, found: 578.4.

### (1S,9S)-1-((R)-1-Amino-3-hydroxypropyl)-9-ethyl-5-fluoro-9-hydroxy-4-methyl-1,2,3,9,12,15-hexahydro-10H,13H-benzo[de]pyrano[3′4′: 6,7]indolizino[1,2-b]quinoline-10,13-dione hydrochloride (11-78)

11-78 was synthesized in a similar fashion to that of 11-76.

Spectra for 11-78: ¹H NMR (400 MHz, DMSO-D₆) δ ppm 7.73-8.02 (m, 4H) 7.34 (s, 1H) 6.14-6.89 (m, 1H) 5.37-5.50 (m, 3H) 5.25-5.34 (m, 1H) 3.42-3.65 (m, 4H) 3.08 (br d. J=6.88 Hz, 2H) 2.51-2.56 (m, 1H) 2.38 (s, 3H) 1.97-2.11 (m, 1H) 1.79-1.91 (m, 3H) 1.65-1.77 (m, 1H) 0.88 (t, J=7.25 Hz, 3H). ¹⁹F NMR (376 MHz, DMSO-D₆) δ ppm −111.91. LCMS (ESI+) m/z: [MH]⁺ calcd for C₂₇H₂₉FN₃O₅⁺: 494.2, found: 494.3. SFC (retention time=2.531 min). Note: *The stereochemistries at these carbons are arbitrarily assigned.

### (1R,9S)-1-((S)-1-Acetamido-3-hydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-79)

11-79 was synthesized in a similar fashion to that of 11-75.

Spectra for 11-79: ¹H NMR (400 MHz, DMSO-D₆) δ ppm 7.90 (br d, J=8.88 Hz, 1H) 7.71 (d, J=11.13 Hz, 1H) 7.00 (s, 1H) 5.19-5.61 (m, 4H) 4.34 (t, J=5.00 Hz, 1H) 4.06-4.25 (m, 1H) 3.38-3.42 (m, 1H) 3.27-3.29 (m, 2H) 3.10 (br d, J=7.38 Hz, 2H) 2.30-2.39 (m, 4H) 2.22 (s, 3H) 2.09-2.13 (m, 2H) 1.86-2.02 (m, 1H) 1.59-1.75 (m, 2H) 1.38-1.53 (m, 3H) 0.90 (br t, J=7.32 Hz, 3H). ¹⁹F NMR (376 MHz, DMSO-D₆) δ ppm −112.07. LCMS (ESI+) m/z: [MH]⁺ calcd for C₃₁H₃₃FN₃O₇⁺: 578.2, found: 578.4.

### (1R,9S)-1-((S)-1-Amino-3-hydroxypropyl)-9-ethyl-5-fluoro-9-hydroxy-4-methyl-1,2,3,9,12,15-hexahydro-10H,13H-benzo[de]pyrano[3′_4′: 6,7]indolizino[1,2-b]quinoline-10,13-dione hydrochloride (11-80)

11-80 was synthesized in a similar fashion to that of 11-76.

Spectra for 11-80: ¹H NMR (400 MHz, DMSO-D₆) δ ppm 7.79-7.87 (m, 3H) 7.34 (s, 1H) 6.56 (s, 1H) 5.37-5.50 (m, 3H) 5.26-5.34 (m, 1H) 4.88 (br s, 1H) 3.45-3.73 (m, 4H) 3.08 (br d, J=7.38 Hz, 2H) 2.50-2.56 (m, 1H) 2.38 (s, 3H) 1.96-2.08 (m, 1H) 1.79-1.95 (m, 3H) 1.65-1.78 (m, 1H) 0.87 (t, J=7.32 Hz, 3H). ¹⁹F NMR (376 MHz, DMSO-D₆) δ ppm −111.91. LCMS (ESI+) m/z: [MH]⁺ calcd for C₂₇H₂₉FN₃O₅⁺: 494.2, found: 494.4. SFC (retention time=4.328 min). Note: *The stereochemistries at these carbons are arbitrarily assigned.

### (1R,9S)-1-((R)-1-acetamido-3-hydroxypropyl)-9-ethyl-5-fluoro-4-methyl-10,13-dioxo-2,3,9,10,13,15-hexahydro-1H,12H-benzo[de]pyrano[3′ 4′: 6 7]indolizino[1,2-b]quinolin-9-yl acetate (11-81)

11-81 was synthesized in a similar fashion to that of 11-75.

Spectra for 11-81: ¹H NMR (400 MHz, DMSO-D₆) δ ppm 7.69-7.85 (m, 2H) 7.02 (s, 1H) 5.35-5.58 (m, 3H) 5.21 (d, J=18.64 Hz, 1H) 4.07-4.17 (m, 2H) 3.35-3.38 (m, 1H) 3.22-3.30 (m, 2H) 3.17 (d, J=5.25 Hz, 1H) 2.95-3.05 (m, 1H) 2.32-2.39 (m, 4H) 2.22 (s, 3H) 2.08-2.19 (m, 2H) 1.80-1.94 (m, 1H) 1.67-1.75 (m, 3H) 1.51-1.65 (m, 1H) 1.31-1.44 (m, 1H) 0.90 (t, J=7.38 Hz, 3H). ¹⁹F NMR (376 MHz, DMSO-D₆) ¿ ppm −111.65. LCMS (ESI+) m/z: [MH]⁺ calcd for C₃₁H₃₃FN₃O₇⁺: 578.2, found: 578.5.

### (1R,9S)-1-((R)-1-Amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (11-82)

11-82 was synthesized in a similar fashion to that of 11-76.

Spectra for 11-82: ¹H NMR (400 MHz, DMSO-D₆) δ ppm 8.17 (br s, 3H) 7.79 (d, J=10.88 Hz, 1H) 7.32 (s, 1H) 6.20-6.80 (m, 1H) 5.44 (s, 2H) 5.38 (d, J=18.89 Hz, 1H) 5.13-5.29 (m, 1H) 3.46-3.57 (m, 2H) 3.38-3.44 (m, 1H) 3.32 (dt. J=11.16, 5.74 Hz, 1H) 3.23 (br d, J=12.88 Hz, 1H) 2.99-3.14 (m, 1H) 2.60 (br d. J=13.38 Hz, 1H) 2.39 (s, 3H) 1.94-2.04 (m, 1H) 1.81-1.93 (m, 2H) 1.69-1.80 (m, 1H) 1.25-1.38 (m, 1H) 0.87 (t, J=7.32 Hz, 3H). ¹H NMR (400 MHz, DMSO-D₆+D₂O) δ ppm 7.75 (d, J=10.88 Hz, 1H) 7.35 (s, 1H) 5.30-5.49 (m, 3H) 5.19 (d, J=18.76 Hz, 1H) 3.49-3.61 (m, 2H) 3.35-3.44 (m, 1H) 3.26-3.35 (m, 1H) 3.12-3.25 (m, 1H) 2.98-3.11 (m, 1H) 2.47 (br s, 1H) 2.36 (s, 3H) 1.95-2.09 (m, 1H) 1.85 (tt, J=14.45, 7.13 Hz, 2H) 1.73 (br dd, J=13.57, 6.57 Hz, 1H) 1.25-1.42 (m, 1H) 0.85 (t, J=7.32 Hz, 3H). ¹⁹F NMR (376 MHz, DMSO-D₆) δ ppm −111.67. LCMS (ESI+) m/z: [MH]⁺ calcd for C₂₇H₂₉FN₃O₅⁺: 494.2, found: 494.4. SFC (retention time=6.619 min). Note: *The stereochemistries at these carbons are arbitrarily assigned.

### Example 12

### Synthesis of: N-((2R,3R)-3-((1S,9S)-9-Ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)-2,3-dihydroxypropyl)acetamide (12-86) and N-((2S,3S)-3-((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)-2,3-dihydroxypropyl)acetamide (12-87)

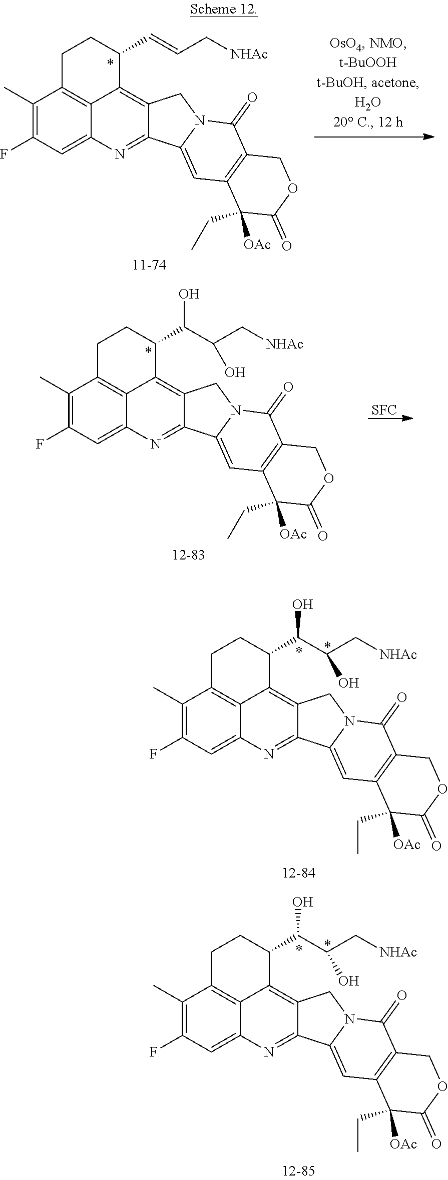

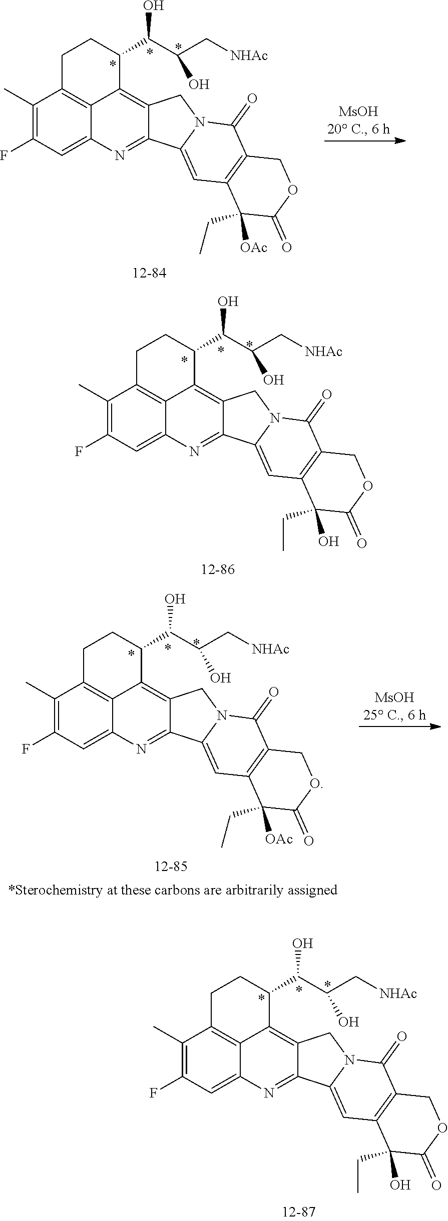 [SMILES: CC[C@@]1(OC(C)=O)C(=O)OCc2c1cc1n(c2=O)Cc2c-1nc1cc(F)c(C)c3c1c2[C@@H]([C@@H](O)[C@H](O)CNC(C)=O)CC3 ; CC[C@@]1(O)C(=O)OCc2c1cc1n(c2=O)Cc2c-1nc1cc(F)c(C)c3c1c2[C@@H]([C@@H](O)[C@H](O)CNC(C)=O)CC3 ; CC[C@@]1(OC(C)=O)C(=O)OCc2c1cc1n(c2=O)Cc2c-1nc1cc(F)c(C)c3c1c2[C@@H]([C@H](O)[C@@H](O)CNC(C)=O)CC3 ; CC[C@@]1(O)C(=O)OCc2c1cc1n(c2=O)Cc2c-1nc1cc(F)c(C)c3c1c2[C@@H]([C@H](O)[C@@H](O)CNC(C)=O)CC3]

### (1S,9S)-1-((1R)-3-Acetamido-1,2-dihydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (12-83)

To a stirred mixture of (1R,9S)-1-((E)-3-acetamidoprop-1-en-1-yl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (11-74) (500 mg, 893 μmol, 1.0 equiv) in acetone (17.5 mL). H₂O (2.5 mL) and tert-butyl alcohol (5 mL), was added 4-methylmorpholine 4-oxide (439 mg, 3.75 mmol, 396 μL, 4.2 equiv), osmium tetraoxide (45.4 mg, 178 μmol, 9.27 μL, 0.2 equiv) and 2-hydroperoxy-2-methyl-propane (8.05 mg, 89.3 μmol, 8.57 μL, 0.1 equiv). After stirring at 20° C., for 12 h, it was quenched by addition of H₂O (20 mL) and extracted with dichloromethane (3×20 mL). The combined organic layers were washed with brine, dried over Na₂SO₄, filtered, and concentrated to give (1S,9S)-1-((1R)-3-acetamido-1,2-dihydroxypropyl)-9-ethyl-5-fluoro-4-methyl-10,13-dioxo-2,3,9,10,13,15-hexahydro-1H, 12H-benzo[de]pyrano [3′,4′:6,7]indolizino[1,2-b]quinolin-9-yl acetate (12-83) (160 mg, 30% yield), which was used directly in the next step without purification. LCMS (ESI+) m/z: [MH]⁺ calcd for C₃₁H₃₃FN₃O₈⁺: 594.2, found: 594.3. SFC (retention time=1.847 min. 2.465 min).

### (1S,9S)-1-((1R,2R)-3-Acetamido-1,2-dihydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (12-84) and (1S,9S)-1-((1S,2S)-3-acetamido-1,2-dihydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (12-85)

### (1S,9S)-1-((1R)-3-acetamido-1,2-dihydroxy propyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (12-83) (190 mg, 320 μmol) was separated by SFC (Instrument: Waters SFC80Q Preparative SFC System: Column: REGIS (S,S) WHELK-01 (250 mm*25 mm, 10 um); Mobile phase: A for CO₂ and B for ethyl alcohol: Gradient: B %=56.00% isocratic elution mode: Flow rate: 80.00 g/min; Monitor wavelength: 220&254 nm: Column temperature: 40° C.: System back pressure: 100 bar) to give (1S,9S)-1-((1R,2R)-3-acetamido-1,2-dihydroxypropyl)-9-ethyl-5-fluoro-4-methyl-10,13-dioxo-2,3,9,10,13,15-hexahydro-1H, 12H-benzo[de]pyrano[3′,4′:6,7]indolizino [1,2-b]quinolin-9-yl acetate (12-84) (34.0 mg) and (1S,95)-1-((1S,2S)-3-acetamido-1,2-dihydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (12-85) (41.0 mg)

Spectra for 12-84: ¹H NMR (400 MHz, DMSO-D₆) δ ppm 7.84 (t, J=5.69 Hz, 1H) 7.74 (d, J=11.01 Hz, 1H) 7.01 (s, 1H) 5.41-5.57 (m, 3H) 5.29 (d, J=19.01 Hz, 1H) 5.01 (d, J=7.13 Hz, 1H) 4.60 (d, J=5.88 Hz, 1H) 3.84 (q. J=6.88 Hz, 1H) 3.48-3.62 (m, 2H) 3.17 (br t, J=6.00 Hz, 2H) 3.03-3.11 (m, 1H) 2.85-3.02 (m, 1H) 2.26-2.36 (m, 4H) 2.21 (s, 3H) 2.14 (dt. J=9.47, 7.08 Hz, 2H) 1.86-1.97 (m, 1H) 1.80 (s, 3H) 0.90 (t, J=7.38 Hz, 3H). ¹⁹F NMR (376 MHz, DMSO-D₆) δ ppm −112.17. LCMS (ESI+) m/z: [MH]⁺ calcd for C₃₁H₃₃FN₃O₈⁺: 594.2, found: 594.4. SFC (retention time=1.212 min).

Spectra for 12-85: ¹H NMR (400 MHz, DMSO-D₆) δ ppm 7.72 (d, J=10.88 Hz, 1H) 7.57 (br t, J=5.32 Hz, 1H) 7.01 (s, 1H) 5.45-5.61 (m, 3H) 5.27 (d, J=19.39 Hz, 1H) 4.94 (d, J=7.50 Hz, 1H) 4.77 (d, J=7.25 Hz, 1H) 3.51-3.57 (m, 1H) 3.43-3.50 (m, 1H) 3.13-3.24 (m, 2H) 2.98-3.04 (m, 2H) 2.92 (dt, J=13.32, 4.72 Hz, 1H) 2.58-2.65 (m, 1H) 2.36 (s, 3H) 2.22 (s, 3H) 2.14 (dt, J=10.69, 7.16 Hz, 2H) 1.80-1.92 (m, 1H) 1.65 (s, 3H) 0.90 (t, J=7.38 Hz, 3H). ¹⁹F NMR (376 MHz, DMSO-D₆) δ ppm −111.92. LCMS (ESI+) m/z: [MH]⁺ calcd for C₃₁H₃₃FN₃O₈⁺: 594.2, found: 594.2. SFC (retention time=1.605 min).

### N-((2R,3R)-3-((1S,9S)-9-Ethyl-5-fluoro-9-hydroxy-4-methyl-10,13-dioxo-2,3,9,10,13,15-hexahydro-1H,12H-benzo [de]pyrano[3′ 4′:6,7]indolizino[1,2-6]quinolin-1-yl)-2,3-dihydroxypropyl)acetamide (12-86)

After a mixture of (1S,9S)-1-((1R,2R)-3-acetamido-1,2-dihydroxypropyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (12-84) (63.0 mg, 106 μmol) in methanesulfonic acid (1.2 mL) was stirred at 25° C., for 6 h, it was filtered and the residue purified by prep-HPLC (Instrument: Gilson 281 Semi-preparative HPLC system; Column: Phenomenex Luna C18 100*30 mm*3 um; Mobile phase: A: H₂O (0.2% formic acid): B: acetonitrile: Gradient: B from 10.00% to 50.00% in 8.00 min; Flow rate: 25.00 mL/min; Monitor wavelength: 220&254 nm) to give N-((2R,3R)-3-((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)-2,3-dihydroxypropyl)acetamide (12-86) (15.2 mg, 25% yield). Note: *the stereochemistries at these carbons are arbitrarily assigned. ¹H NMR (400 MHz, DMSO-D₆) δ ppm 7.86 (br t, J=5.57 Hz, 1H) 7.76 (d, J=11.13 Hz, 1H) 7.31 (s, 1H) 6.47-6.56 (m, 1H) 5.36-5.54 (m, 3H) 5.29 (d, J=19.01 Hz, 1H) 5.03 (br d, J=6.88 Hz, 1H) 4.67 (d, J=5.75 Hz, 1H) 3.84 (q, J=6.50 Hz, 1H) 3.48-3.61 (m, 2H) 3.05-3.22 (m, 3H) 2.84-3.01 (m, 1H) 2.29-2.42 (m, 4H) 1.76-1.97 (m, 6H) 0.87 (t, J=7.25 Hz, 3H). ¹⁹F NMR (376 MHz, DMSO-D₆) δ ppm −112.44. LCMS (ESI+) m/z: [MH]⁺ calcd for C₂₉H₃₁FN₃O₇⁺: 552.2, found: 552.2. SFC (retention time=2.308 min).

### N-((2S,3S)-3-((1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,3-dihydroxypropyl)acetamide (12-87)

12-87 was synthesized in a similar fashion to that of 12-86.

Spectra for 12-87: ¹H NMR (400 MHz, DMSO-D₆) δ ppm 7.73 (d, J=10.88 Hz, 1H) 7.62 (br t, J=5.44 Hz, 1H) 7.31 (s, 1H) 6.51 (s, 1H) 5.52 (d, J=19.51 Hz, 1H) 5.43 (s, 2H) 5.27 (d, J=19.39 Hz, 1H) 4.96 (br d. J=7.00 Hz, 1H) 4.82 (d, J=7.25 Hz, 1H) 3.44-3.58 (m, 2H) 3.12-3.24 (m, 2H) 2.92-3.08 (m, 3H) 2.59-2.66 (m, 1H) 2.36 (s, 3H) 1.78-1.94 (m, 3H) 1.65 (s, 3H) 0.87 (t, J=7.25 Hz, 3H). ¹⁹F NMR (376 MHz, DMSO-D₆) δ ppm −112.19. LCMS (ESI+) m/z: [MH]⁺ calcd for C₂₉H₃₁FN₃O₇⁺: 552.2, found: 552.2. SFC (retention time=2.999 min).

### Example 13

### Synthesis of: N—((S)-1-((1S,9S)-9-Ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)-3-hydroxypropyl)acetamide (13-88). N—((R)-1-((15.95)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)-3-hydroxypropyl)acetamide (13-89), N—((S)-1-((1R,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypropyl)acetamide (13-90) and N—((R)-1-((1R,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypropyl)acetamide (13-91)

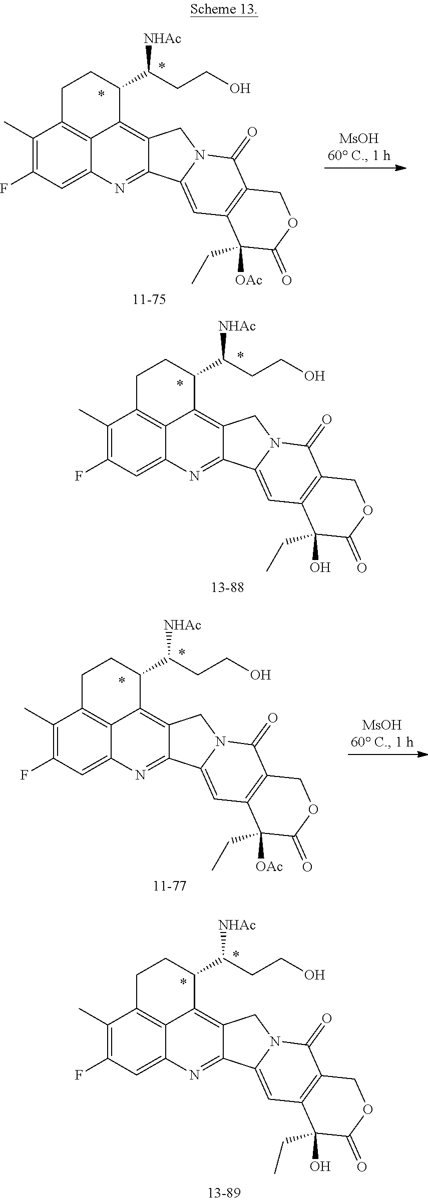 [SMILES: CC[C@@]1(OC(C)=O)C(=O)OCc2c1cc1n(c2=O)Cc2c-1nc1cc(F)c(C)c3c1c2[C@@H]([C@H](CCO)NC(C)=O)CC3 ; CC[C@@]1(O)C(=O)OCc2c1cc1n(c2=O)Cc2c-1nc1cc(F)c(C)c3c1c2[C@@H]([C@H](CCO)NC(C)=O)CC3 ; CC[C@@]1(OC(C)=O)C(=O)OCc2c1cc1n(c2=O)Cc2c-1nc1cc(F)c(C)c3c1c2[C@@H]([C@@H](CCO)NC(C)=O)CC3 ; CC[C@@]1(O)C(=O)OCc2c1cc1n(c2=O)Cc2c-1nc1cc(F)c(C)c3c1c2[C@@H]([C@@H](CCO)NC(C)=O)CC3]

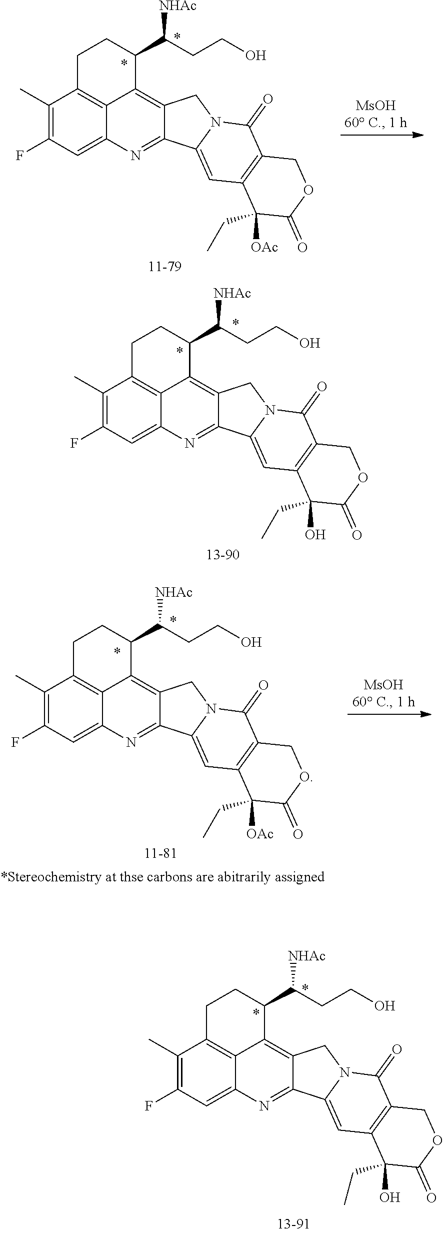

### N—((S)-1-((1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypropyl)acetamide (13-88)

After a mixture of (1S,9S)-1-((S)-1-acetamido-3-hydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-75) (120 mg, 208 μmol) and methanesulfonic acid (1.2 mL) was stirred at 60° C., for 1 h, it was cooled to room temperature, diluted with methanol (0.5 mL), filtered, and the filtrate purified by prep-HPLC (Instrument: Gilson 281 Semi-preparative HPLC system, Column: Phenomenex Luna C18 100*30 mm*3 um, Mobile phase: A: H₂O (0.2% formic acid); B: acetonitrile, Gradient: B from 10.00% to 50.00% in 8.00 min, Flow rate: 25.00 ml/min, Monitor wavelength: 220&254 nm) to give N—((S)-1-((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)-3-hydroxypropyl)acetamide (13-88) (12.3 mg, 11% yield). ¹H NMR (400 MHz, DMSO-D₆) δ ppm 7.81 (d, J=9.26 Hz, 1H) 7.75 (d, J=11.01 Hz, 1H) 7.31 (s, 1H) 6.52 (s, 1H) 5.36-5.49 (m, 3H) 5.19 (d, J=18.64 Hz, 1H) 4.06-4.20 (m, 2H) 3.36 (br s, 1H) 3.29 (br s, 2H) 3.16 (br dd, J=11.44, 4.57 Hz, 1H) 3.00 (br dd, J=17.70, 4.57 Hz, 1H) 2.29-2.39 (m, 4H) 1.79-1.96 (m, 3H) 1.73 (s, 3H) 1.54-1.68 (m, 1H) 1.31-1.43 (m, 1H) 0.89 (t, J=7.32 Hz, 3H). ¹⁹F NMR (376 MHz, DMSO-D₆) δ ppm −111.89. LCMS (ESI+) m/z: [MH]⁺ calcd for C₂₉H₃₁FN₃O₆⁺: 536.2, found: 536.2. SFC (retention time)=1.146 min. Note: *the stereochemistries at these carbons are arbitrarily assigned.

### N—((R)-1-((1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypropyl)acetamide (13-89)

13-89 was synthesized in a similar fashion to that of 13-88.

Spectra for 13-89: ¹H NMR (400 MHz, DMSO-D₆) δ ppm 7.93 (d, J=8.82 Hz, 1H) 7.72 (d, J=10.97 Hz, 1H) 7.30 (s, 1H) 6.51 (s, 1H) 5.39-5.51 (m, 3H) 5.28-5.36 (m, 1H) 4.34 (t, J=5.01 Hz, 1H) 4.11-4.23 (m, 1H) 3.37-3.45 (m, 1H) 3.27 (br d, J=7.63 Hz, 2H) 3.09 (br d, J=6.68 Hz, 2H) 2.35-2.44 (m, 4H) 1.81-2.03 (m, 3H) 1.59-1.79 (m, 2H) 1.48 (s, 3H) 0.88 (t, J=7.33 Hz, 3H) ¹⁹F NMR (376 MHz, DMSO-D₆) δ ppm-112.37. LCMS (ESI+) m/z: [MH]⁺ calcd for C₂₉H₃₁FN₃O₆⁺: 536.2, found: 536.2. SFC (retention time)=1.589 min. Note: *the stereochemistries at these carbons are arbitrarily assigned.

### N—((S)-1-((1R,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypropyl)acetamide (13-90)

13-90 was synthesized in a similar fashion to that of 13-88.

Spectra for 13-90: ¹H NMR (400 MHz, DMSO-D₆) δ ppm 7.90 (br d, J=8.94 Hz, 1H) 7.72 (d, J=10.97 Hz, 1H) 7.30 (s, 1H) 6.50 (s, 1H) 5.26-5.53 (m, 4H) 4.36 (t, J=4.95 Hz, 1H) 4.10-4.24 (m, 1H) 3.35-3.46 (m, 2H) 3.21-3.27 (m, 1H) 3.02-3.15 (m, 2H) 2.35-2.44 (m, 4H) 1.92-2.03 (m, 1H) 1.82-1.89 (m, 2H) 1.61-1.79 (m, 2H) 1.45 (s, 3H) 0.87 (t, J=7.27 Hz, 3H). ¹⁹F NMR (376 MHz, DMSO-D₆) δ ppm −112.38. LCMS (ESI+) m/z: [MH]⁺ calcd for C₂₉H₃₁FN₃O₆⁺: 536.2, found: 536.2. SFC (retention time)=2.448 min. Note: *the stereochemistries at these carbons are arbitrarily assigned.

N—((R)-1-((1R,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypropyl)acetamide (13-91);

13-91 was synthesized in a similar fashion to that of 13-88.

Spectra for 13-91: ¹H NMR (400 MHz, DMSO-D₆) δ ppm 7.80 (d, J=9.13 Hz, 1H) 7.74 (d, J=10.88 Hz, 1H) 7.31 (s, 1H) 6.53 (s, 1H) 5.35-5.49 (m, 3H) 5.20 (d, J=18.64 Hz, 1H) 4.05-4.20 (m, 2H) 3.36 (br d, J=8.88 Hz, 1H) 3.29 (br d, J=4.13 Hz, 2H) 3.12-3.22 (m, 1H) 3.00 (br dd, J=17.76, 4.13 Hz, 1H) 2.28-2.41 (m, 4H) 1.78-1.96 (m, 3H) 1.72 (s, 3H) 1.55-1.67 (m, 1H) 1.30-1.45 (m, 1H) 0.87 (t, J=7.25 Hz, 3H), 19F NMR (376 MHz, DMSO-D₆) δ ppm −111.92. LCMS (ESI+) m/z: [MH]⁺ calcd for C₂₉H₃₁FN₃O₆⁺: 536.2, found: 536.2. SFC (retention time)=2.064 min. Note: *the stereochemistries at these carbons are arbitrarily assigned.

### Example 14

### Synthesis of: (1S,9S)-9-Ethyl-5-fluoro-9-hydroxy-1-((S)-3-hydroxy-1-(isopropylamino)propyl)-4-methyl-1,2,3,9,12,15-hexahydro-10H,13H-benzo[de]pyrano[3′,4′:6,7]indolizino[1,2-b]quinoline-10,13-dione (14-92), (1S,9S)-9-ethyl-5-fluoro-9-hydroxy-1-((R)-3-hydroxy-1-(isopropylamino)propyl)-4-methyl-1,2,3,9,12,15-hexahydro-10H,13H-benzo[de]pyrano[3′,4′:6,7]indolizino[1,2-b]quinoline-10,13-dione (14-93). (1R,9S)-9-ethyl-5-fluoro-9-hydroxy-1-((S)-3-hydroxy-1-(isopropylamino)propyl)-4-methyl-1,2,3,9,12,15-hexahydro-10H,13H-benzo[de]pyrano[3′,4′:6,7]indolizino[1,2-b]quinoline-10,13-dione (14-94), and (1R,9S)-9-ethyl-5-fluoro-9-hydroxy-1-((R)-3-hydroxy-1-(isopropylamino)propyl)-4-methyl-1,2,3,9,12,15-hexahydro-10H,13H-benzo[de]pyrano[3′,4′:6,7]indolizino[1,2-b]quinoline-10,13-dione (14-95)

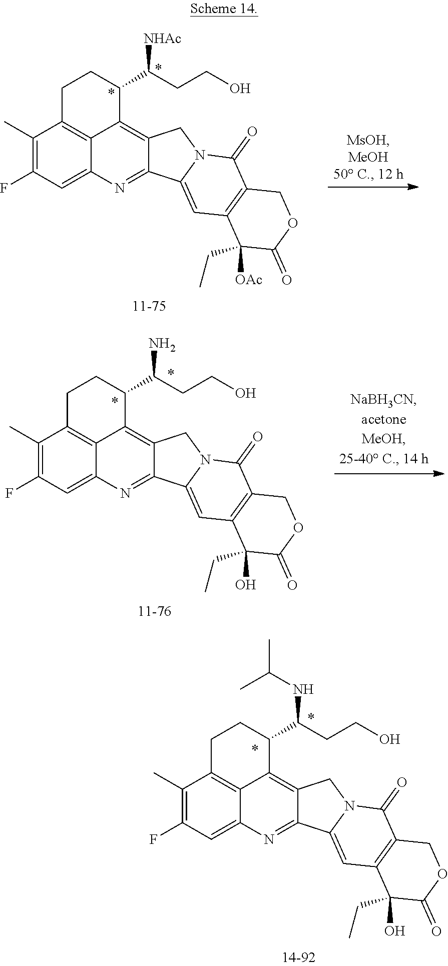 [SMILES: CC[C@@]1(OC(C)=O)C(=O)OCc2c1cc1n(c2=O)Cc2c-1nc1cc(F)c(C)c3c1c2[C@@H]([C@H](CCO)NC(C)=O)CC3 ; CC[C@@]1(O)C(=O)OCc2c1cc1n(c2=O)Cc2c-1nc1cc(F)c(C)c3c1c2[C@@H]([C@@H](N)CCO)CC3 ; CC[C@@]1(O)C(=O)OCc2c1cc1n(c2=O)Cc2c-1nc1cc(F)c(C)c3c1c2[C@@H]([C@H](CCO)NC(C)C)CC3]

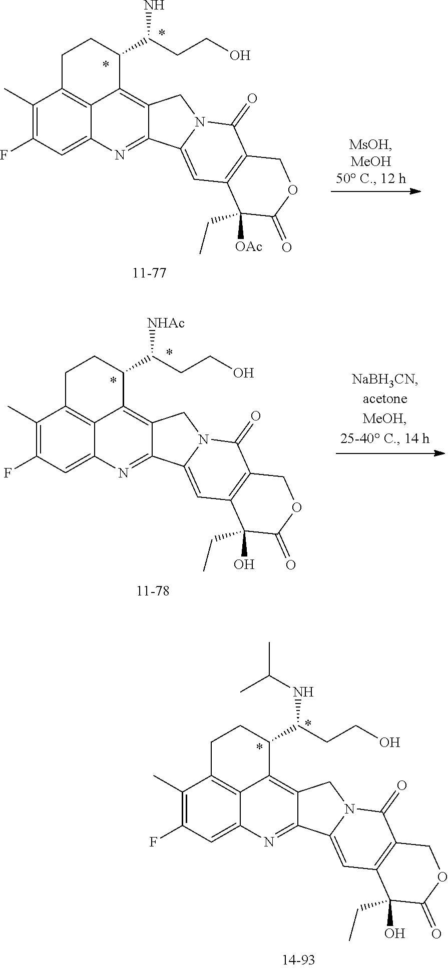 [SMILES: CC[C@@]1(OC(C)=O)C(=O)OCc2c1cc1n(c2=O)Cc2c-1nc1cc(F)c(C)c3c1c2[C@@H]([C@H](N)CCO)CC3 ; CC[C@@]1(O)C(=O)OCc2c1cc1n(c2=O)Cc2c-1nc1cc(F)c(C)c3c1c2[C@@H]([C@@H](CCO)NC(C)=O)CC3 ; CC[C@@]1(O)C(=O)OCc2c1cc1n(c2=O)Cc2c-1nc1cc(F)c(C)c3c1c2[C@@H]([C@@H](CCO)NC(C)C)CC3]

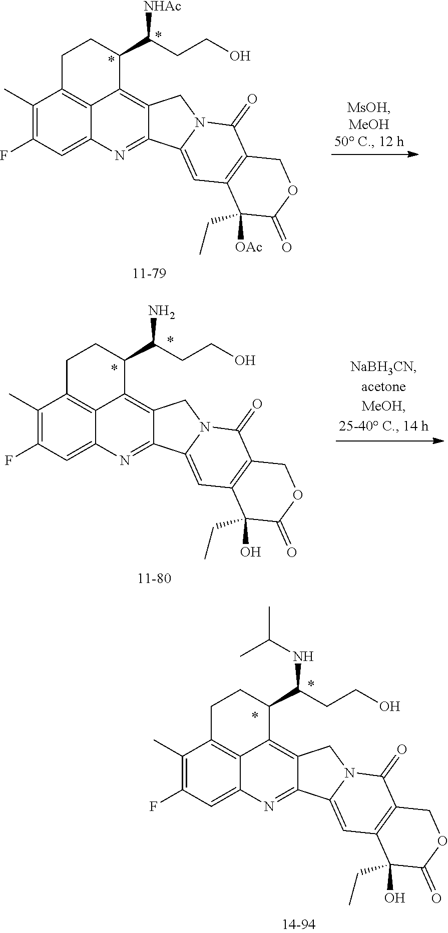 [SMILES: CC[C@@]1(OC(C)=O)C(=O)OCc2c1cc1n(c2=O)Cc2c-1nc1cc(F)c(C)c3c1c2[C@H]([C@H](CCO)NC(C)=O)CC3 ; CC[C@@]1(O)C(=O)OCc2c1cc1n(c2=O)Cc2c-1nc1cc(F)c(C)c3c1c2[C@H]([C@@H](N)CCO)CC3 ; CC[C@@]1(O)C(=O)OCc2c1cc1n(c2=O)Cc2c-1nc1cc(F)c(C)c3c1c2[C@H]([C@H](CCO)NC(C)C)CC3]

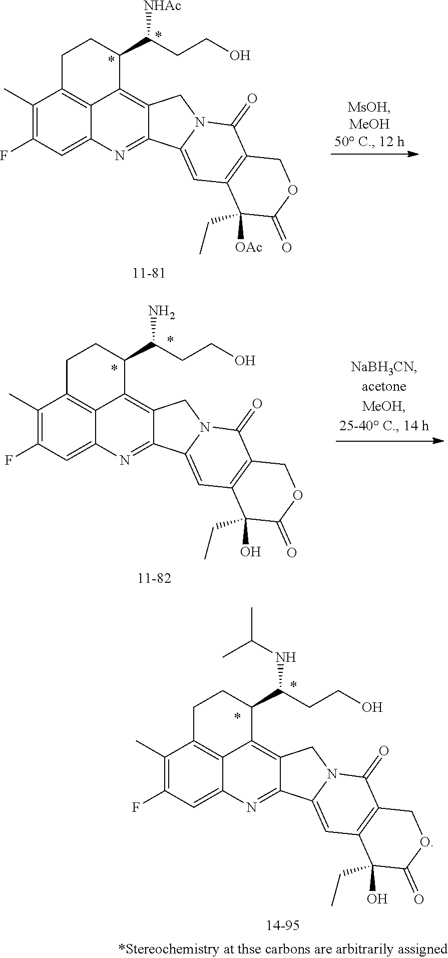 [SMILES: CC[C@@]1(OC(C)=O)C(=O)OCc2c1cc1n(c2=O)Cc2c-1nc1cc(F)c(C)c3c1c2[C@H]([C@@H](CCO)NC(C)=O)CC3 ; CC[C@@]1(O)C(=O)OCc2c1cc1n(c2=O)Cc2c-1nc1cc(F)c(C)c3c1c2[C@H]([C@H](N)CCO)CC3 ; CC[C@@]1(O)C(=O)OCc2c1cc1n(c2=O)Cc2c-1nc1cc(F)c(C)c3c1c2[C@H]([C@@H](CCO)NC(C)C)CC3]

### (1S,9S)-1-((S)-1-Amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (11-76)

To a stirred mixture of (1S,9S)-1-((S)-1-acetamido-3-hydroxypropyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (11-75) (290 mg, 502 μmol, 1.0 equiv) in methanol (2.9 mL) was added methanesulfonic acid (434 mg, 4.52 mmol, 323 μL, 9.0 equiv). After stirring at 50° C., for 12 h, it was cooled to room temperature, adjusted to pH 7 by addition of N,N-diisopropylethylamine. The resulting mixture was used directly in the next step without further purification. LCMS (ESI+) m/z: [MH]⁺ calcd for C₂₇H₂₉FN₃O₅⁺: 494.2, found: 494.3.

### (1S,9S)-9-Ethyl-5-fluoro-9-hydroxy-1-((S)-3-hydroxy-1-(isopropylamino)propyl)-4-methyl-1,2,3,9,12,15-hexahydro-10H,13H-benzo[de]pyrano[3′,4′:6,7]indolizino[1,2-b]quinoline-10,13-dione (14-92)

The above mixture of (1S,9S)-1-((S)-1-amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (11-76) was diluted with methanol (3 mL) and added acetone (353 mg, 6.08 mmol, 446 μL, 20 equiv), and stirred at 40° C., for 1 h. Then sodium cyanoborohydride (95.5 mg, 1.51 mmol, 5.0 eq) was added. After stirring at 25° C., for 13 h, it was filtered and the filtrate purified by prep-HPLC (Instrument: Gilson 281 Semi-preparative HPLC system; Column: Phenomenex Gemini-NX 150*30 mm*5 um: Mobile phase: A: H₂O (0.2% formic acid); B: acetonitrile; Gradient: B from 15.00% to 45.00% in 20.00 min; Flow rate: 25.00 mL/min: Monitor wavelength: 220&254 nm) to give (1S,9S)-9-ethyl-5-fluoro-9-hydroxy-1-((S)-3-hydroxy-1-(isopropylamino)propyl)-4-methyl-1,2,3,9,12,15-he xahydro-10H,13H-benzo[de]pyrano[3′,4′:6,7]indolizino[1,2-b]quinoline-10,13-dione (14-92) (15.8 mg, two steps yield 12%).

¹H NMR (400 MHz, DMSO-D₆) δ ppm 7.73 (br d. J=10.88 Hz, 1H) 7.31 (s, 1H) 6.50 (s, 1H) 5.29-5.53 (m, 4H) 4.20-4.67 (m, 1H) 3.32-3.37 (m, 3H) 3.23-3.28 (m, 1H) 3.02-3.17 (m, 2H) 2.45 (br d, J=3.63 Hz, 1H) 2.36 (s, 3H) 1.70-2.05 (m, 4H) 1.36-1.52 (m, 3H) 0.89 (br t, J=7.25 Hz, 6H) 0.58 (br s, 3H). ¹⁹F NMR (376 MHz, DMSO-D₆) 8 ppm −112.25. LCMS (ESI+) m/z: [MH]⁺ calcd for C₃₀H₃₅FN₃O₅⁺: 536.2, found: 535.8. SFC (retention time)=1.913 min. Note: *the stereochemistries at these carbons are arbitrarily assigned.

### (1S,9S)-9-Ethyl-5-fluoro-9-hydroxy-1-((R)-3-hydroxy-1-(isopropylamino)propyl)-4-methyl-1,2,3,9,12,15-hexahydro-10H,13H-benzo [de]pyrano[3′,4′:6,7]indolizino[ 1,2-b]quinoline-10,13-dione (14-93)

14-93 was synthesized in a similar fashion to that of 14-92.

Spectra for 14-93: ¹H NMR (400 MHz, DMSO-D₆) δ ppm 7.71 (d, J=11.01 Hz, 1H) 7.31 (s, 1H) 6.53 (br s, 1H) 5.61 (br d. J=18.64 Hz, 1H) 5.32-2.43 (m, 3H) 3.25-3.32 (m, 3H) 2.95-3.08 (m, 2H) 2.91 (br t, J=7.88 Hz, 1H) 2.32-2.40 (m, 4H) 2.10-2.21 (m, 1H) 1.74-2.02 (m, 3H) 1.65-1.72 (m, 1H), 1.52-1.65 (m, 1H) 0.87 (br t, J=7.19 Hz, 3H) 0.76 (d, J=6.13 Hz, 3H) 0.19 (br d, J=6.00 Hz, 3H). ¹⁹F NMR (376 MHz, DMSO-D₆) δ ppm −112.22. LCMS (ESI+) m/z: [MH]⁺ calcd for C₃₀H₃₅FN₃O₅⁺: 536.2, found: 536.3. SFC (retention time=2.167 min). Note: *the stereochemistries at these carbons are arbitrarily assigned.

### (1R,9S)-9-Ethyl-5-fluoro-9-hydroxy-1-((S)-3-hydroxy-1-(isopropylamino)propyl)-4-methyl-1,2,3,9,12,15-hexahydro-10H,13H-benzo[de]pyrano[3′,4′:6,7]indolizino[1,2-b]quinoline-10,13-dione (14-94)

14-94 was synthesized in a similar fashion to that of 14-92.

Spectra for 14-94: ¹H NMR (400 MHz, DMSO-D₆) δ ppm 7.73 (d, J=11.01 Hz, 1H) 7.31 (s, 1H) 6.48 (s, 1H) 5.65 (br d, J=18.76 Hz, 1H) 5.43 (s, 2H) 5.35 (br d, J=18.8 Hz, 1H) 3.44-3.62 (m, 2H) 3.31-3.40 (m, 1H), 2.99-3.14 (m, 2H) 2.85-2.92 (m, 1H) 2.34-2.42 (m, 4H) 2.04-2.19 (m, 1H) 1.75-1.96 (m, 3H) 1.65-1.74 (m, 1H) 1.53-1.64 (m, 1H) 0.87 (t, J=7.32 Hz, 3H) 0.76 (br s, 3H) 0.12 (br s, 3H). ¹⁹F NMR (376 MHz, DMSO-D₆) δ ppm −112.34. LCMS (ESI+) m/z: [MH]⁺ calcd for C₃₀H₃₅FN₃O₅⁺: 536.2, found: 536.2. SFC (retention time)=1.372 min. Note: *the stereochemistries at these carbons are arbitrarily assigned.

### (1R,9S)-9-Ethyl-5-fluoro-9-hydroxy-1-((R)-3-hydroxy-1-(isopropylamino)propyl)-4-methyl-1,2,3,9,12,15-hexahydro-10H,13H-benzo[de]pyrano[3′,4′:6,7]indolizino[1,2-b]quinoline-10,13-dione (14-95)

14-95 was synthesized in a similar fashion to that of 14-92.

Spectra for 14-95: ¹H NMR (400 MHz, DMSO-D₆) δ ppm 7.73 (br d, J=11.13 Hz, 1H) 7.31 (s, 1H) 6.43-6.55 (m, 1H) 5.31-5.48 (m, 4H) 4.22-4.66 (m, 1H) 3.33-3.47 (m, 3H) 3.25-3.30 (m, 1H) 3.02-3.18 (m, 2H) 2.42-2.47 (m, 1H) 2.36 (s, 3H) 1.66-2.03 (m, 4H) 1.43 (br s, 3H) 0.87 (br t, J=7.32 Hz, 6H) 0.34-0.71 (m, 3H). ¹⁹F NMR (376 MHz, DMSO-D₆) δ ppm −112.25. LCMS (ESI+) m/z: [MH]⁺ calcd for C₃₀H₃₅FN₃O₅⁺: 536.2, found: 535.8. SFC (retention time)=1.541 min. Note: *the stereochemistries at these carbons are arbitrarily assigned.

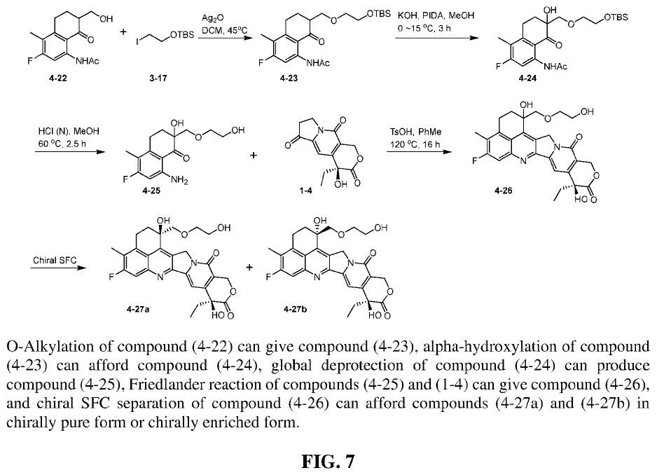

Alternative and/or additional synthesis routes for the compounds described in Example 14 can be found in FIG. 4.

### Example 15

### Synthesis of: (1S,9S)-1-((S)-1-(Dimethylamino)-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (15-96), (1S,9S)-1-((R)-1-(dimethylamino)-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (15-97), (1S,9S)-1-((R)-1-(dimethylamino)-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (15-98) and (1R,9S)-1-((R)-1-(dimethylamino)-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (15-99)

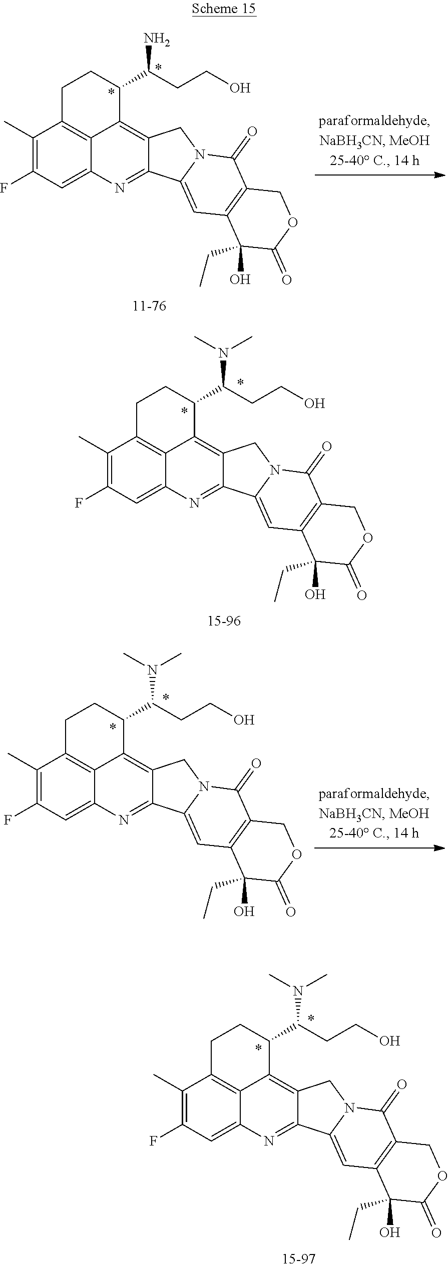 [SMILES: CC[C@@]1(O)C(=O)OCc2c1cc1n(c2=O)Cc2c-1nc1cc(F)c(C)c3c1c2[C@@H]([C@@H](N)CCO)CC3 ; CC[C@@]1(O)C(=O)OCc2c1cc1n(c2=O)Cc2c-1nc1cc(F)c(C)c3c1c2[C@@H]([C@H](CCO)N(C)C)CC3 ; CC[C@@]1(O)C(=O)OCc2c1cc1n(c2=O)Cc2c-1nc1cc(F)c(C)c3c1c2[C@@H]([C@@H](CCO)N(C)C)CC3 ; CC[C@@]1(O)C(=O)OCc2c1cc1n(c2=O)Cc2c-1nc1cc(F)c(C)c3c1c2[C@@H]([C@@H](CCO)N(C)C)CC3]

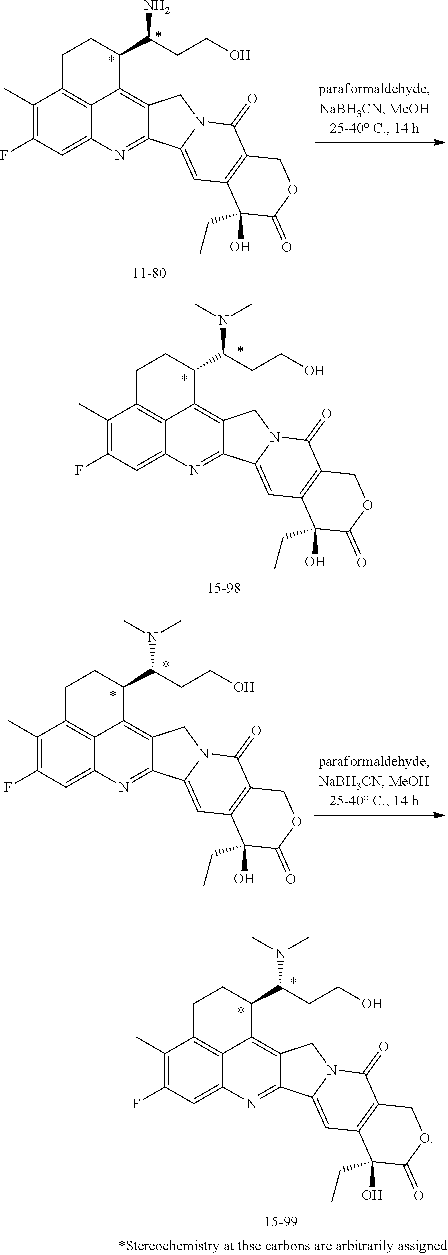 [SMILES: CC[C@@]1(O)C(=O)OCc2c1cc1n(c2=O)Cc2c-1nc1cc(F)c(C)c3c1c2[C@H]([C@@H](N)CCO)CC3 ; CC[C@@]1(O)C(=O)OCc2c1cc1n(c2=O)Cc2c-1nc1cc(F)c(C)c3c1c2[C@@H]([C@H](CCO)N(C)C)CC3 ; CC[C@@]1(O)C(=O)OCc2c1cc1n(c2=O)Cc2c-1nc1cc(F)c(C)c3c1c2[C@H]([C@@H](CCO)N(C)C)CC3 ; CC[C@@]1(O)C(=O)OCc2c1cc1n(c2=O)Cc2c-1nc1cc(F)c(C)c3c1c2[C@H]([C@@H](CCO)N(C)C)CC3]

### (1S,9S)-1-((S)-1-(Dimethylamino)-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (15-96)

After the mixture of (1S,9S)-1-((S)-1-amino-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (11-76) (150 mg, 303 μmol, 1.0 equiv) and paraformaldehyde (155 mg, 4.56 mmol, 15 equiv) in methanol (3 mL) was stirred at 40° C., for 1 h, sodium cyanoborohydride (57.3 mg, 912 μmol, 3.0 equiv) was added. Then the mixture was stirred at 25° C., for 13 h, filtered, and filtrate purified by prep-HPLC (Instrument: Gilson 281 Semi-preparative HPLC system: Column: Phenomenex Gemini-NX 150*30 mm*5 um: Mobile phase: A: H₂O (0.2% formic acid); B: acetonitrile; Gradient: B from 15.00% to 45.00% in 20.00 min: Flow rate: 25.00 mL/min: Monitor wavelength: 220&254 nm) to give (1S,9S)-1-((S)-1-(dimethylamino)-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (15-96) (20.5 mg, 12% yield). ¹H NMR (400 MHz, methanol-D₄) δ ppm 7.54-7.66 (m, 2H) 5.59 (d, J=16.26 Hz, 1H) 5.24-5.46 (m, 3H) 3.45 (br d, J=9.76 Hz, 1H) 3.17-3.28 (m, 1H) 3.03-3.16 (m, 2H) 2.92-3.03 (m, 2H) 2.68 (br d, J=11.51 Hz, 1H) 2.53 (s, 6H) 2.41 (s, 3H) 1.82-2.06 (m, 4H) 1.12-1.23 (m, 1H) 1.01 (t, J=7.38 Hz, 3H), 19F NMR (376 MHz, methanol-D₄) δ ppm −112.83. LCMS (ESI+) m/z: [MH]⁺ calcd for C₂₉H₃₃FN₃O₅⁺: 522.2, found: 522.2. Chiral HPLC (retention time)=6.542 min. Note: *the stereochemistries at these carbons are arbitrarily assigned.

### (1S,9S)-1-((R)-1-(Dimethylamino)-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (15-97)

15-97 was synthesized in a similar fashion to that of 15-96.

Spectra for 15-97: ¹H NMR (400 MHz, methanol-D4) δ ppm 7.53-7.67 (m, 2H), 5.58 (d, J=16.26 Hz, 1H), 5.38 (d, J=16.26 Hz, 1H), 5.32 (s, 2H), 3.60-3.73 (m, 1H), 3.53 (dt, J=10.38, 7.00 Hz, 2H), 3.07-3.24 (m, 2H), 2.99 (br t, J=7.75 Hz, 1H), 2.30-2.56 (m, 10H), 1.74-2.22 (m, 5H), 1.02 (t, J=7.38 Hz, 3H). ¹⁹F NMR (376 MHz, methanol-D4) δ ppm −113.304. LCMS (ESI+) m/z: [MH]⁺ calcd for C₂₉H₃₃FN₃O₅⁺: 522.2, found: 522.2. SFC (retention time=6.693 min). Note: *the stereochemistries at these carbons are arbitrarily assigned.

### (1R,9S)-1-((S)-1-(Dimethylamino)-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (15-98)

15-98 was synthesized in a similar fashion to that of 15-96.

Spectra for 15-98: ¹H NMR (400 MHz, methanol-D₄) δ ppm 7.61-7.70 (m, 2H) 5.61 (d, J=16.26 Hz, 1H) 5.28-5.47 (m, 3H) 3.69 (dt, J=11.57, 5.85 Hz, 1H) 3.46-3.60 (m, 2H) 3.07-3.24 (m, 2H) 2.94-3.05 (m, 1H) 2.31-2.48 (m, 10H) 2.02-2.15 (m, 2H) 1.93-2.02 (m, 2H) 1.81-1.93 (m, 1H) 1.01 (t, J=7.38 Hz, 3H). ¹⁹F NMR (376 MHz, methanol-D₄) δ ppm −113.37. LCMS (ESI+) m/z: [MH]⁺ calcd for C₂₉H₃₃FN₃O₅⁺: 522.2, found: 522.2. Chiral HPLC (retention time)=8.066 min. Note: *the stereochemistries at these carbons are arbitrarily assigned.

### (1R,9S)-1-((R)-1-(Dimethylamino)-3-hydroxypropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (15-99)

15-99 was synthesized in a similar fashion to that of 15-96.

Spectra for 15-99: ¹H NMR (400 MHz, methanol-D₄) δ ppm 7.58-7.71 (m, 2H) 5.62 (d, J=16.26 Hz, 1H) 5.29-5.48 (m, 3H) 3.48 (br d, J=9.76 Hz, 1H) 3.24 (br dd, J=13.45, 4.44 Hz, 1H) 3.09-3.21 (m, 2H) 3.05 (dt, J=10.63, 6.82 Hz, 2H) 2.66-2.74 (m, 1H) 2.57 (s, 6H) 2.45 (s, 3H) 1.87-2.06 (m, 4H) 1.24 (dq, J=11.73, 6.89 Hz, 1H) 1.03 (t, J=7.38 Hz, 3H). ¹⁹F NMR (376 MHz, methanol-D₄) δ ppm −112.89. LCMS (ESI+) m/z: [MH]⁺ calcd for C₂₉H₃₃FN₃O₅⁺: 522.2, found: 522.2. Chiral HPLC (retention time)=7.606 min. Note: *the stereochemistries at these carbons are arbitrarily assigned.

### Example 16

### Synthesis of: (1S,9S)-1-((R)-1-Amino-2-hydroxyethyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (16-102), (1S,9S)-1-((S)-1-amino-2-hydroxyethyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (16-105), (1R,9S)-1-((R)-1-amino-2-hydroxyethyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (16-108) and (1R,9S)-1-((S)-1-amino-2-hydroxyethyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (16-111)

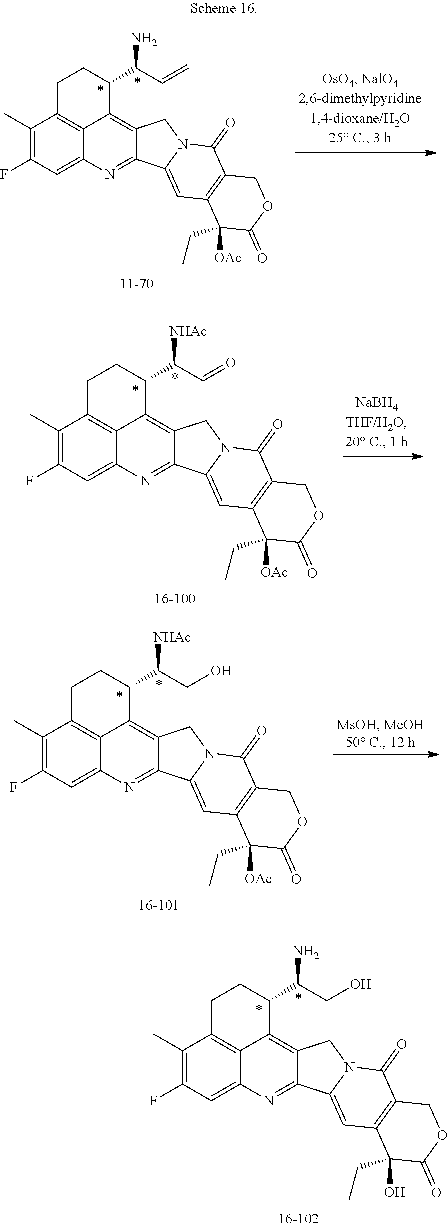 [SMILES: C=C[C@H](N)[C@H]1CCc2c(C)c(F)cc3nc4c(c1c23)Cn1c-4cc2c(c1=O)COC(=O)[C@@]2(CC)OC(C)=O ; CC[C@@]1(OC(C)=O)C(=O)OCc2c1cc1n(c2=O)Cc2c-1nc1cc(F)c(C)c3c1c2[C@@H]([C@H](C=O)NC(C)=O)CC3 ; CC[C@@]1(OC(C)=O)C(=O)OCc2c1cc1n(c2=O)Cc2c-1nc1cc(F)c(C)c3c1c2[C@@H]([C@H](CO)NC(C)=O)CC3 ; CC[C@@]1(O)C(=O)OCc2c1cc1n(c2=O)Cc2c-1nc1cc(F)c(C)c3c1c2[C@@H]([C@@H](N)CO)CC3]

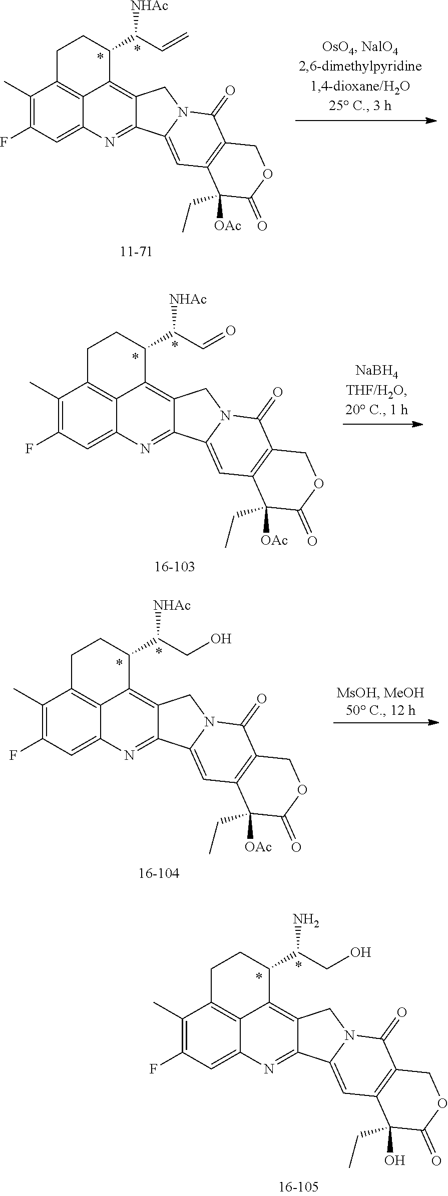 [SMILES: C=C[C@@H](NC(C)=O)[C@H]1CCc2c(C)c(F)cc3nc4c(c1c23)Cn1c-4cc2c(c1=O)COC(=O)[C@@]2(CC)OC(C)=O ; CC[C@@]1(OC(C)=O)C(=O)OCc2c1cc1n(c2=O)Cc2c-1nc1cc(F)c(C)c3c1c2[C@@H]([C@@H](C=O)NC(C)=O)CC3 ; CC[C@@]1(OC(C)=O)C(=O)OCc2c1cc1n(c2=O)Cc2c-1nc1cc(F)c(C)c3c1c2[C@@H]([C@@H](CO)NC(C)=O)CC3 ; CC[C@@]1(O)C(=O)OCc2c1cc1n(c2=O)Cc2c-1nc1cc(F)c(C)c3c1c2[C@@H]([C@H](N)CO)CC3]

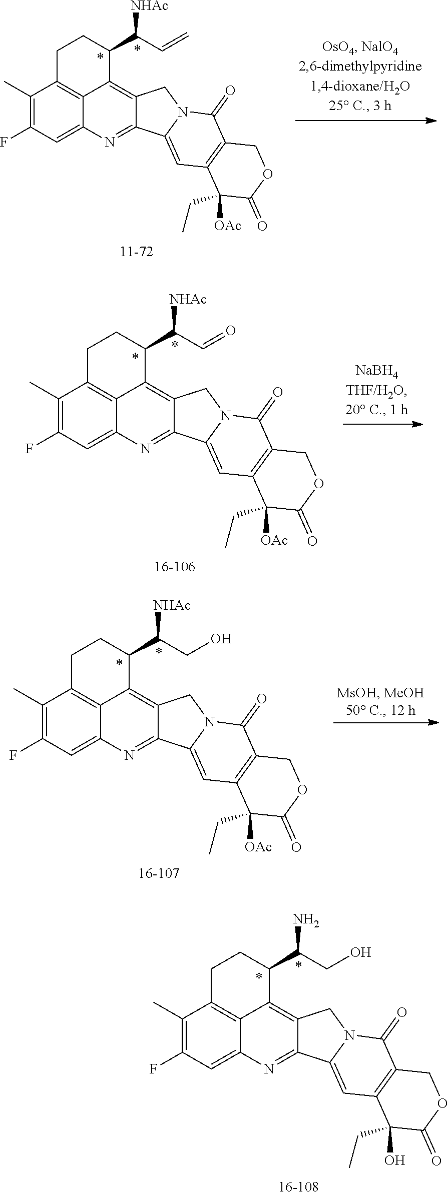 [SMILES: C=C[C@H](NC(C)=O)[C@@H]1CCc2c(C)c(F)cc3nc4c(c1c23)Cn1c-4cc2c(c1=O)COC(=O)[C@@]2(CC)OC(C)=O ; CC[C@@]1(OC(C)=O)C(=O)OCc2c1cc1n(c2=O)Cc2c-1nc1cc(F)c(C)c3c1c2[C@H]([C@H](C=O)NC(C)=O)CC3 ; CC[C@@]1(OC(C)=O)C(=O)OCc2c1cc1n(c2=O)Cc2c-1nc1cc(F)c(C)c3c1c2[C@H]([C@H](CO)NC(C)=O)CC3 ; CC[C@@]1(O)C(=O)OCc2c1cc1n(c2=O)Cc2c-1nc1cc(F)c(C)c3c1c2[C@H]([C@@H](N)CO)CC3]

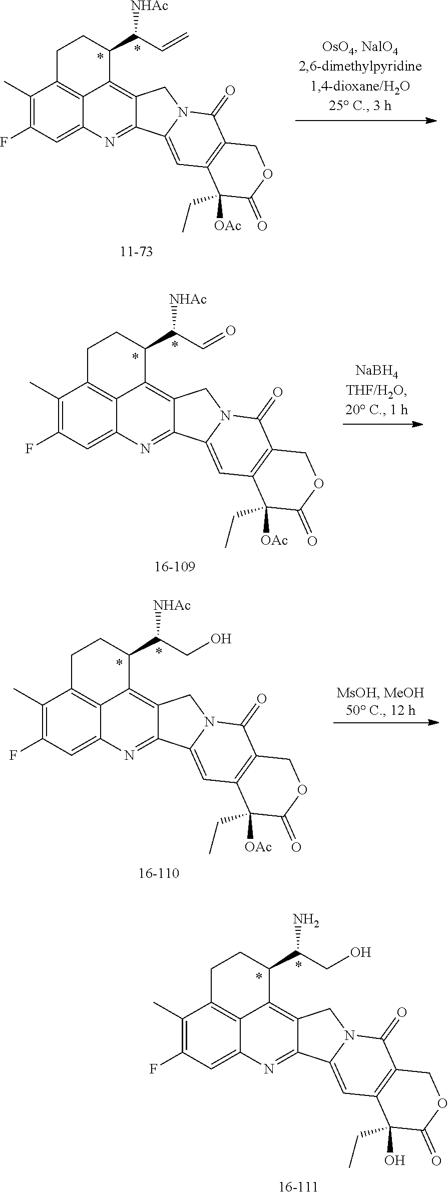 [SMILES: C=C[C@@H](NC(C)=O)[C@@H]1CCc2c(C)c(F)cc3nc4c(c1c23)Cn1c-4cc2c(c1=O)COC(=O)[C@@]2(CC)OC(C)=O ; CC[C@@]1(OC(C)=O)C(=O)OCc2c1cc1n(c2=O)Cc2c-1nc1cc(F)c(C)c3c1c2[C@H]([C@@H](C=O)NC(C)=O)CC3 ; CC[C@@]1(OC(C)=O)C(=O)OCc2c1cc1n(c2=O)Cc2c-1nc1cc(F)c(C)c3c1c2[C@H]([C@@H](CO)NC(C)=O)CC3 ; CC[C@@]1(O)C(=O)OCc2c1cc1n(c2=O)Cc2c-1nc1cc(F)c(C)c3c1c2[C@H]([C@H](N)CO)CC3]

### (1S,9S)-1-((R)-1-acetamido-2-oxoethyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (16-100)

To a stirred mixture of (1S,9S)-1-((S)-1-acetamidoallyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (11-70) (100 mg, 179 μmol, 1.0 equiv) in dioxane (3.0 mL) and water (1.0 mL) were added osmium (VIII) oxide (4.54 mg, 17.9 μmol, 0.1 equiv) and 2,6-dimethylpyridine (38.3 mg, 357 μmol, 41.6 L, 2.0 equiv), sodium periodate (153 mg, 715 μmol, 39.6 μL, 4.0 equiv). After stirring at 25° C., for 3 h, it was quenched by addition of saturated sodium sulfite solution (5 mL) and extracted with dichloromethane (3×5 mL). The combined organic layers were washed with brine, dried over sodium sulfate, filtered and concentrated to give (1S,9S)-1-((R)-1-acetamido-2-oxoethyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (16-100), which was used directly without further purification. LCMS (ESI+) m/z: [MH]⁺ calcd for C₃₀H₂₉FN₃O₇⁺: 562.2, found: 562.2.

### (1S,9S)-1-((R)-1-Acetamido-2-hydroxyethyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (16-101)

To a stirred mixture of (1S,9S)-1-((R)-1-acetamido-2-oxoethyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (16-100) (100 mg, 142 μmol, 1.0 equiv) in tetrahydrofuran (2.0 mL) and water (1.0 mL) was added NaBH₄ (2.69 mg, 71.2 μmol, 0.5 equiv). After stirring at 20° C., for 1 h, it was extracted with dichloromethane (3×5 mL), combined organic layers washed with brine (10 mL), dried over sodium sulfate, filtered and concentrated to give (1S,9S)-1-((R)-1-acetamido-2-hydroxyethyl)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl acetate (16-101) (60 mg, two steps yield 59%), which was used directly in the next step. ¹H NMR (400 MHz, DMSO-D₆) δ ppm 8.45 (s, 1H) 8.01 (d, J=9.41 Hz, 1H) 7.69 (d, J=11.00 Hz, 1H) 7.00 (s, 1H) 5.42-5.59 (m, 3H) 5.27-5.38 (m, 1H) 4.91-5.06 (m, 1H) 4.10 (tt, J=8.85, 4.42 Hz, 1H) 3.74 (br dd, J=11.07, 4.83 Hz, 1H) 3.44 (br d, J=11.62 Hz, 2H) 3.04-3.19 (m, 2H) 2.37 (s, 4H) 2.20 (s, 3H) 2.10-2.18 (m, 2H) 1.92-1.98 (m, 1H) 1.39 (s, 3H) 0.92 (t, J=7.34 Hz, 3H). ¹⁹F NMR (376 MHz, DMSO-D₆) δ ppm −112.30. LCMS (ESI+) m/z: [MH]⁺ calcd for C₃₀H₃₁FN₃O₇⁺: 564.2, found: 564.3.

### (1S,9S)-1-((R)-1-Amino-2-hydroxyethyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (16-102)

To a stirred mixture of (1S,9S)-1-((R)-1-acetamido-2-hydroxyethyl)-9-ethyl-5-fluoro-4-methyl-10,13-dioxo-2,3,9,10,13,15-hexahydro-1H, 12H-benzo[de]pyrano[3′,4′:6,7]indolizino[1,2-b]quinolin-9-yl acetate (16-101) (60 mg, 106 μmol, 1.0 equiv) in methanol (1.2 mL) was added methanesulfonic acid (0.6 mL). After stirring at 50° C., for 12 h, it was cooled to room temperature, filtered, and the filtrate purified by prep-HPLC (Instrument: Gilson 281 Semi-preparative HPLC system, Column: Phenomenex Luna C18 75*30 mm*3 um, Mobile phase: A: water (0.04% HCl); B; acetonitrile, Gradient: B from 20.00% to 53.00% in 8.00 min, Flow rate: 25.00 mL/min, Monitor wavelength: 220&254 nm) to give (1S,9S)-1-((R)-1-amino-2-hydroxyethyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (16-102) (23.1 mg, 45% yield). ¹H NMR (400 MHz, DMSO-D₆) δ ppm 8.29 (br d, J=1.67 Hz, 3H) 7.79 (d, J=10.85 Hz, 1H) 7.31 (s, 1H) 6.53 (br s, 1H) 5.19-5.61 (m, 5H) 3.66 (br d, J=10.13 Hz, 1H) 3.21-3.38 (m, 3H) 3.10-3.17 (m, 1H) 3.04 (br dd, J=17.76, 3.93 Hz, 1H) 2.44-2.48 (m, 1H) 2.38 (s, 3H) 1.96-2.10 (m, 1H) 1.87 (tt, J=14.01, 7.03 Hz, 2H) 0.89 (t, J=7.27 Hz, 3H). ¹⁹F NMR (376 MHz, DMSO-D₆) δ ppm −111.92. LCMS (ESI+) m/z: [MH]⁺ calcd for C₂₆H₂₇FN₃O₅⁺: 480.1, found: 480.2. Chiral HPLC (retention time=6.297 min/12.278 min) showed that only two peaks and the ratio of P1/P2=1.70/98.30, 96.6% de. Note: *the stereochemistries at these carbons are arbitrarily assigned.

### (1S,9S)-1-((S)-1-Amino-2-hydroxyethyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (16-105)

16-105 was synthesized in a similar fashion to that of 16-102.

Spectra for 16-105: ¹H NMR (400 MHz, DMSO-D₆) δ ppm 7.79-7.87 (m, 3H) 7.34 (s, 1H) 6.55 (br s, 1H) 5.23-5.61 (m, 5H) 3.82-3.92 (m, 1H) 3.74 (br d, J=10.76 Hz, 1H) 3.57 (br d. J=10.01 Hz, 1H) 3.39-3.44 (m, 1H) 3.02-3.17 (m, 2H) 2.46 (br s, 1H) 2.37 (s, 3H) 1.95-2.08 (m, 1H) 1.88 (dt, J=13.60, 6.89 Hz, 2H) 0.88 (t, J=7.32 Hz, 3H). ¹H NMR (400 MHz, DMSO-D₆, D₂O) δ ppm 7.76 (d, J=10.88 Hz, 1H) 7.37 (s, 1H) 5.36-5.51 (m, 3H) 5.19-5.32 (m, 1H) 3.74-3.79 (m, 1H) 3.67-3.74 (m, 1H) 3.53 (br d. J=9.88 Hz, 1H) 3.21-3.32 (m, 1H) 2.98-3.10 (m, 2H) 2.42 (br d. J=15.38 Hz, 1H) 2.34 (s, 3H) 1.95-2.08 (m, 1H) 1.85 (tt, J=13.73, 7.10 Hz, 2H) 0.85 (t, J=7.32 Hz, 3H). ¹⁹F NMR (376 MHz, DMSO-D₆) δ ppm −112.13. LCMS (ESI+) m/z: [MH]⁺ calcd for C₂₆H₂₇FN₃O₅⁺: 480.1, found: 480.2. Chiral HPLC (retention time=5.525 min/11.031 min) showed that only two peaks and the ratio of P1/P2=0.93/99.07, 98.14% de. Note: *the stereochemistries at these carbons are arbitrarily assigned.

### (1R,9S)-1-((R)-1-Amino-2-hydroxyethyl)-9-ethyl-5-fluoro-9-hydroxy-4-methyl-1,2,3,9,12,15-hexahydro-10H,13H-benzo[ de]pyrano[3′ 4′:6,7]indolizino[1,2-b]quinoline-10,13-dione hydrochloride (16-108)

16-108 was synthesized in a similar fashion to that of 16-102.

Spectra for 16-108: ¹H NMR (400 MHz, DMSO-D₆) δ ppm 7.45-8.20 (m, 3H) 7.23-7.41 (m, 1H) 6.25-6.79 (m, 1H) 4.88-5.83 (m, 4H) 3.82-3.93 (m, 1H) 3.75 (br d, J=10.76 Hz, 1H) 3.58 (br d, J=9.76 Hz, 1H) 3.05-3.16 (m, 3H) 2.46 (br d, J=2.00 Hz, 1H) 2.38 (s, 3H) 1.95-2.03 (m, 1H) 1.81-1.94 (m, 2H) 0.88 (br t, J=7.25 Hz, 3H), 1H NMR (400 MHz, DMSO-D₆, D₂O) δ ppm 7.79 (d, J=10.76 Hz, 1H) 7.37 (s, 1H) 5.20-5.55 (m, 4H) 3.81-3.87 (m, 1H) 3.73 (br s, 1H) 3.55 (br d. J=10.01 Hz, 1H) 3.32 (dt, J=8.72, 4.33 Hz, 1H) 2.96-3.18 (m, 2H) 2.43 (br d, J=12.63 Hz, 1H) 2.36 (s, 3H) 1.95-2.08 (m, 1H) 1.87 (tt, J=14.63, 7.25 Hz, 2H) 0.86 (t, J=7.25 Hz, 3H). ¹⁹F NMR (376 MHz, DMSO-D₆) δ ppm −112.12. LCMS (ESI+) m/z: [MH]⁺ calcd for C₂₆H₂₇FN₃O₅⁺: 480.1, found: 480.2. Chiral HPLC (retention time=6.277 min/12.288 min) showed that only two peaks and the ratio of P1/P2=98.96/1.04, 97.92% de. Note: *the stereochemistries at these carbons are arbitrarily assigned.

### (1R,9S)-1-((S)-1-Amino-2-hydroxyethyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione hydrochloride (16-111)

16-111 was synthesized in a similar fashion to that of 16-102.

Spectra for 16-111: ¹H NMR (400 MHz, DMSO-D₆) δ ppm 7.60-8.21 (m, 2H) 7.33 (s, 1H) 6.54 (s, 1H) 5.22-5.58 (m, 5H) 3.60-3.65 (m, 1H) 3.40-3.42 (m, 1H) 3.18-3.28 (m, 2H) 3.11-3.17 (m, 1H) 3.02-3.10 (m, 1H) 2.46 (br s, 1H) 2.39 (s, 3H) 1.95-2.06 (m, 1H) 1.81-1.94 (m, 2H) 0.87 (t, J=7.25 Hz, 3H). ¹⁹F NMR (376 MHz, DMSO-D₆) δ ppm −111.91. LCMS (ESI+) m/z: [MH]⁺ calcd for C₂₆H₂₇FN₃O₅⁺: 480.1, found: 480.2. Chiral HPLC (retention time=5.521 min/11.027 min) showed that only two peaks and the ratio of P1/P2=99.43/0.57, 98.86% de. Note: *the stereochemistries at these carbons are arbitrarily assigned.

### Example 17

### Synthesis of: (1S,9S)-9-Ethyl-5-fluoro-9-hydroxy-1-((R)-2-hydroxy-1-(isopropylamino)ethyl)-4-methyl-1,2,3,9,12,15-hexahydro-10H,13H-benzo[de]pyrano[3′,4′:6,7]indolizino[1,2-b]quinoline-10,13-dione (17-112), (1S,9S)-9-ethyl-5-fluoro-9-hydroxy-1-((S)-2-hydroxy-1-(isopropylamino)ethyl)-4-methyl-1,2,3,9,12,15-hexahydro-10H,13H-benzo[de]pyrano[3′,4′:6,7]indolizino[1,2-b]quinoline-10,13-dione (17-114), (1R,9S)-9-ethyl-5-fluoro-9-hydroxy-1-((R)-2-hydroxy-1-(isopropylamino)ethyl)-4-methyl-1,2,3,9,12,15-hexahydro-10H,13H-benzo[de]pyrano[3′,4′:6,7]indolizino[1,2-b]quinoline-10,13-dione (17-113) and (1R,9S)-9-ethyl-5-fluoro-9-hydroxy-1-((S)-2-hydroxy-1-(isopropylamino)ethyl)-4-methyl-1,2,3,9,12,15-hexahydro-10H,13H-benzo[de]pyrano[3′,4′:6,7]indolizino[1,2-b]quinoline-10,13-dione (17-115)

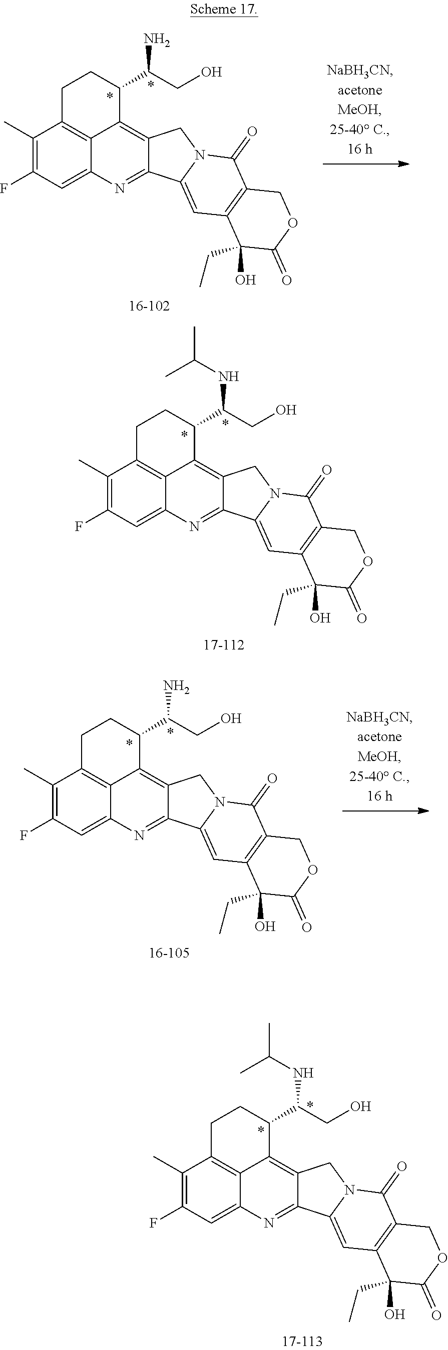

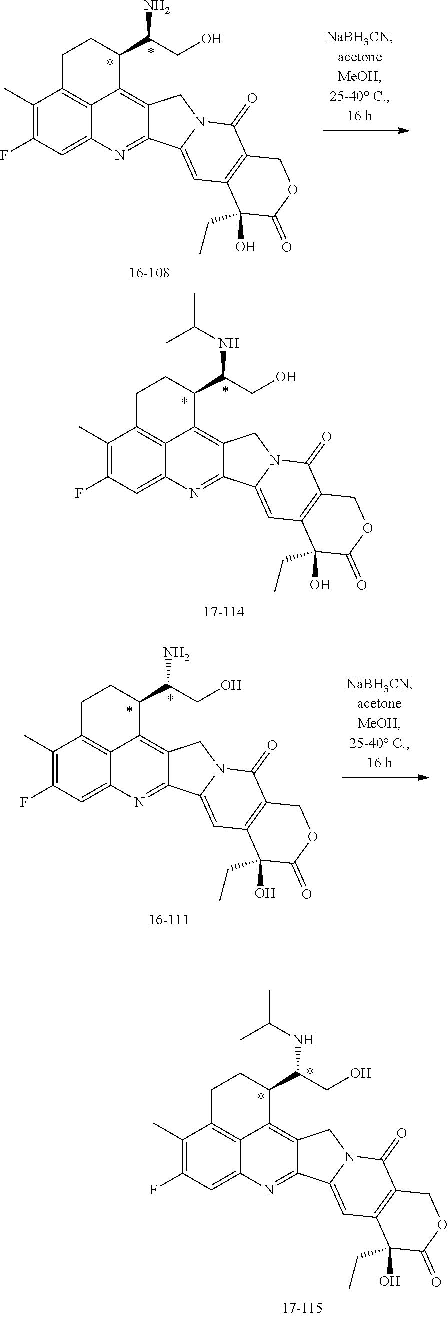

### (1S,9S)-9-Ethyl-5-fluoro-9-hydroxy-1-((R)-2-hydroxy-1-(isopropylamino)ethyl)-4-methyl-1,2,3,9,12,15-hexahydro-10H,13H-benzo[de]pyrano[3′,4′:6,7]indolizino[1,2-b]quinoline-10,13-dione (17-112)

To a stirred mixture of (1S,9S)-1-((R)-1-amino-2-hydroxyethyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (16-102) (100 mg, 209 μmol, 1.0 equiv) in methanol (2.0 mL), was added acetone (242 mg, 4.18 mmol, 20 eq), heated at 40° C., for 2 h, sodium cyanoborohydride (65.5 mg, 1.05 mmol, 5.0 eq) added. After stirring at 25° C., for 14 h, it was filtered and the filtrate purified by prep-HPLC (Instrument: Gilson 281 Semi-preparative HPLC system, Column: Phenomenex Gemini C18 75*40 mm*3 um. Mobile phase: A: water (0.2% formic acid); B: acetonitrile, Gradient: B from 15.00% to 40.00% in 20.00 min, Flow rate: 25.00 mL/min. Monitor wavelength: 220&254 nm) to give (1S,9S)-9-ethyl-5-fluoro-9-hydroxy-1-((R)-2-hydroxy-1-(isopropylamino)ethyl)-4-methyl-1,2,3,9,12,15-hexahydro-10H,13H-benzo[de]pyrano[3′,4′:6,7]indolizino[1,2-b]quinoline-10,13-dione (17-112) (9.1 mg, 8% yield). ¹H NMR (400 MHz, DMSO-D₆) δ ppm 8.39 (s, 0.63H) 7.72 (d, J=11.01 Hz, 1H) 7.30 (s, 1H) 6.50 (br s, 1H) 5.59 (d, J=19.14 Hz, 1H) 5.42 (d, J=2.00 Hz, 2H) 5.24 (d, J=19.26 Hz, 1H) 4.63-5.03 (m, 1H) 3.20 (br s, 2H) 2.99-3.05 (m, 1H) 2.86 (br dd, J=11.19, 2.56 Hz, 1H) 2.63-2.77 (m, 4H) 2.31-2.41 (m, 4H) 1.79-1.93 (m, 3H) 0.94 (d, J=6.13 Hz, 3H) 0.89 (t, J=7.32 Hz, 3H) 0.77 (d, J=6.00 Hz, 3H). ¹H NMR (400 MHz, DMSO-D₆, D₂O) δ ppm 8.33 (s, 0.63H) 7.69 (d, J=10.88 Hz, 1H) 7.32 (s, 1H) 5.56 (d, J=19.14 Hz, 1H) 5.33-5.46 (m, 2H) 5.22 (d, J=19.14 Hz, 1H) 3.41 (br dd, J=11.32, 3.94 Hz, 1H) 3.34 (br s, 1H) 3.10-3.24 (m, 1H) 2.95-3.07 (m, 1H) 2.83 (br dd, J=11.26, 2.75 Hz, 1H) 2.73-2.78 (m, 1H) 2.59-2.71 (m, 3H) 2.33 (s, 3H) 1.78-1.93 (m, 3H) 0.93 (d, J=6.00 Hz, 3H) 0.87 (t, J=7.32 Hz, 3H) 0.76 (d, J=6.13 Hz, 3H). ¹⁹F NMR (376 MHz, DMSO-D₆) δ ppm −112.34. LCMS (ESI+) m/z: [MH]⁺ calcd for C₂₉H₃₃FN₃O₅⁺: 522.2, found: 522.2. SFC (retention time=1.021 min) showed that only one peak, 100% de. Note: *the stereochemistries at these carbons are arbitrarily assigned.

### (1S,9S)-9-Ethyl-5-fluoro-9-hydroxy-1-((S)-2-hydroxy-1-(isopropylamino)ethyl)-4-methyl-1,2,3,9,12,15-hexahydro-10H,13H-benzo[de]pyrano[3′,4′:6,7]indolizino[1,2-b]quinoline-10,13-dione (17-113)

17-113 was synthesized in a similar fashion to that of 17-112.

Spectra for 17-113: ¹H NMR (400 MHz, DMSO-D₆) δ ppm 8.41 (s, 0.50H) 7.72 (d, J=11.01 Hz, 1H) 7.30 (s, 1H) 6.50 (br s, 1H) 5.70 (d, J=18.64 Hz, 1H) 5.37-5.50 (m, 2H) 5.32 (d, J=18.64 Hz, 1H) 4.72-4.97 (m, 1H) 3.56-3.73 (m, 2H) 3.07 (br d. J=7.25 Hz, 2H) 2.62 (br d. J=9.76 Hz, 1H) 2.37 (s, 4H) 2.10-2.19 (m, 1H) 1.88 (dt, J=13.91, 6.86 Hz, 3H) 0.89 (t, J=7.32 Hz, 3H) 0.70 (d, J=6.13 Hz, 3H) −0.02 (d, J=6.00 Hz, 3H). ¹⁹F NMR (376 MHz, DMSO-D₆) δ ppm −112.56. LCMS (ESI+) m/z: [MH]⁺ calcd for C₂₉H₃₃FN₃O₅⁺: 522.2, found: 522.2. SFC (retention time=0.839 min) showed that only one peak, 100% de. Note: *the stereochemistries at these carbons are arbitrarily assigned.

### (1R,9S)-9-Ethyl-5-fluoro-9-hydroxy-1-((R)-2-hydroxy-1-(isopropylamino)ethyl)-4-methyl-1,2,3,9,12,15-hexahydro-10H,13H-benzo[de]pyrano[3′,4′:6,7]indolizino[1,2-b]quinoline-10,13-dione (17-114)

17-114 was synthesized in a similar fashion to that of 17-112.

Spectra for 17-114: ¹H NMR (400 MHz, DMSO-D₆) δ ppm 8.26 (s, 0.54H) 7.72 (d, J=11.01 Hz, 1H) 7.31 (s, 1H) 6.47 (br s, 1H) 5.71 (d, J=18.76 Hz, 1H) 5.43 (d, J=1.63 Hz, 2H) 5.32 (d, J=18.76 Hz, 1H) 4.58-5.09 (m, 1H) 3.67-3.73 (m, 1H) 3.63 (br d, J=2.38 Hz, 1H) 3.28-3.31 (m, 3H) 3.07 (br d, J=7.25 Hz, 2H) 2.60 (br d, J=10.01 Hz, 1H) 2.31-2.41 (m, 4H) 2.10-2.37 (m, 1H) 1.76-1.98 (m, 3H) 0.87 (t, J=7.38 Hz, 3H) 0.69 (d, J=6.13 Hz, 3H) −0.08 (d, J=6.00 Hz, 3H). ¹⁹F NMR (376 MHz, DMSO-D₆) δ ppm −112.56. LCMS (ESI+) m/z: [MH]′ calcd for C₂₉H₃₃FN₃O₅⁺: 522.2, found: 522.2. SFC (retention time=1.031 min/1.779 min) showed that only two peaks and the ratio of P1/P2=1.94/98.06, 96.12% de. Note: *the stereochemistries at these carbons are arbitrarily assigned.

### (1R,9S)-9-Ethyl-5-fluoro-9-hydroxy-1-((S)-2-hydroxy-1-(isopropylamino)ethyl)-4-methyl-1,2,3,9,12,15-hexahydro-10H,13H-benzo[de]pyrano[3′,4′:6,7]indolizino[1,2-b]quinoline-10,13-dione (17-115)

17-115 was synthesized in a similar fashion to that of 17-112.

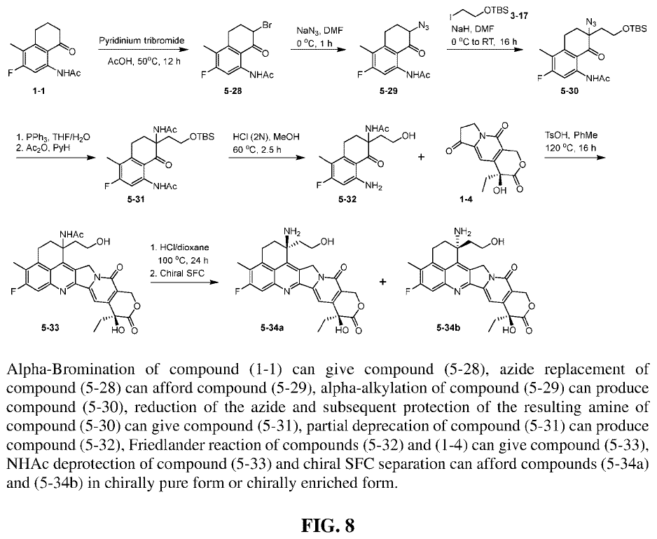

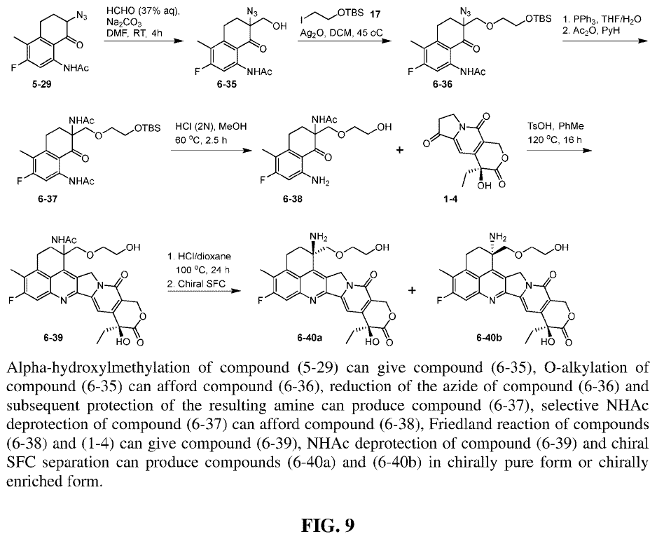

Spectra for 17-115: ¹H NMR (400 MHz, DMSO-D₆) δ ppm 8.35 (s, 0.35H) 7.79 (d, J=11.01 Hz, 1H) 7.38 (s, 1H) 6.45-6.69 (m, 1H) 5.66 (d, J=19.39 Hz, 1H) 5.50 (s, 2H) 5.32 (d, J=19.26 Hz, 1H) 4.92 (br s, 1H) 3.25 (br d, J=12.76 Hz, 2H) 3.09 (br dd, J=17.26, 4.38 Hz, 2H) 2.92-2.97 (m, 1H) 2.72-2.83 (m, 4H) 2.43 (s, 3H) 1.84-2.02 (m, 3H) 1.01 (d, J=6.13 Hz, 3H) 0.94 (br t, J=7.25 Hz, 3H) 0.82 (d, J=6.13 Hz, 3H), 1H NMR (400 MHz, DMSO-D₆, D₂O) δ ppm 8.34 (s, 0.35H) 7.69 (d, J=10.88 Hz, 1H) 7.33 (s, 1H) 5.55 (br d, J=19.26 Hz, 1H) 5.33-5.47 (m, 2H) 5.16-5.29 (m, 1H) 3.41 (br dd, J=11.07, 3.94 Hz, 1H) 3.35 (br d, J=2.25 Hz, 1H) 3.12-3.20 (m, 1H) 3.00 (br dd, J=17.39, 3.75 Hz, 1H) 2.84 (br dd, J=11.13, 3.00 Hz, 1H) 2.73-2.77 (m, 1H) 2.67 (br s, 1H) 2.64-2.69 (m, 2H) 2.33 (s, 3H) 1.86 (dq, J=14.43, 7.07 Hz, 3H) 0.92 (d, J=6.13 Hz, 3H) 0.83-0.88 (m, 3H) 0.74 (d, J=6.00 Hz, 3H). ¹⁹F NMR (376 MHz, DMSO-D₆) δ ppm −112.32. LCMS (ESI+) m/z: [MH]⁺ calcd for C₂₉H₃₃FN₃O₅⁺: 522.2, found: 522.2. SFC (retention time=0.839 min) showed that three peaks. P1/P2/P3=1.03/2.21/96.76, 93.52% de. Note: *the stereochemistries at these carbons are arbitrarily assigned. Alternative and/or additional synthesis routes for the compounds described in Example 17 can be found in FIG. 5.

### Example A

### CTG Assay of Payloads (Jeko-1 and MDA-MB-468)

The CTG assay is a method of determining the number of viable cells in culture based on quantitation of the ATP present, an indicator of metabolically active cells. The cell assay requires the addition of a single reagent, Cell Titer Glo, in which cells are lysed and generation of a luminescent signal is produced. The luminescent signal is proportional to the amount of ATP present. The amount of ATP is directly proportional to the number of cells present in culture. For our assay, cells are ensured to be in log-phase for either Jeko-1 or MDA-MB-468. Cells are transferred to 96 wells and treated with compounds in three-fold serial dilution starting from 1 mM to 0.0000508 mM (10 points dilution), for 72 h. Cell viability is analyzed with CellTiter-Glo® Luminescent Cell Viability Assay (Promega) following manufactures' instruction. Percentage of viable cells in each compound concentration is determined by normalizing with the luminescence of vehicle control and plotted into percentage of viability versus dose response curve by nonlinear fit in GraphPad Prism software. Compound IC₅₀ is calculated as the concentration of compound killing 50% of cells. Results for Jeko-1 are summarized in TABLE 7.

### Example B

### Human Hepatocyte Clearance (HHEP CL)

Suspensions of human hepatocytes (from 10 mixed gender human donors, final concentration 0.5×10⁶ cell/mL) in Williams' E medium are incubated for 90 min with a test compound (0.90% acetonitrile and 0.10% DMSO, final concentration 1 mM) and positive controls (7-Ethoxycoumarin, 7-Hydroxycoumarin, 0.90% acetonitrile and 0.10% DMSO, final concentration 3 mM), with constant shaking at about 600 rpm at 37° C., in an incubator at 5% CO₂ and 95% humidity. The total volume of incubation was 200 μL. A sample (25 mL) is taken out at TO, 15, 30, 60 and 90 min, which is added intermediately to the ice-cold stop solution (acetonitrile with 200 ng/ml of tobutamide and labetalol as internal standard) (125 μl), and vortexed at 500 rpm for 10 min, centrifuged at 3220×g for 20 min at 4° C. Analytical plates are sealed and stored at 4° C., until LCMS analysis. Viability of hepatocytes at pre-incubation is determined to be 84.5%. Results for HHEP CL are summarized in TABLE 7.

### Example C

### Human Liver Microsome Clearance (HLM CL)

Working solution is prepared by adding 5 μL of compound and control stock solution (10 mM in dimethyl sulfoxide, DMSO) to 495 μL of acetonitrile (ACN) (intermediate solution concentration: 100 μM, 99% CAN and 1% DMSO. The appropriate concentrations of microsome working solutions are prepared in 100 mM potassium phosphate buffer. After reaction plates containing mixtures of compound and microsomes are pre-incubated at 37° C., for 10 min. 98 mL of 2 mM of NADPH and 2 mM of MgCl₂ solution is added to start the reaction. The final concentrations of incubation medium are as follows: microsome-0.5 mg protein/mL, test compound/control compound-1 mM, NADPH—1 mM, MgCl₂—1 mM, acetonitrile 0.99%, DMSO 0.01%. Incubations are performed at 37° C., for 60 min. Samples are taken out at T0, T5, T15, T30, T45 and T60, which is added intermediately to the ice-cold stop solution (acetonitrile with 200) ng/ml of tobutamide and labetalol as internal standard) (125 μl), shaken for 10 min, centrifuged at 4000 rpm for 20 min at 4° C. Analytical plates are analyzed by LCMS.

Human liver microsome clearance assay assess metabolism by the cytochrome P450 system (phase I enzymes). These enzymes oxidize substrates by incorporating oxygen atoms into hydrocarbons, thus causing the introduction of hydroxyl groups, or N- O- and S-dealkylation of substrates and forming more polar products easier to be cleared. Human hepatocyte clearance assay measures more broadly the overall cellular metabolism of the test compound (phase I and phase II enzyme pathways). Phase II enzymes catalyze the conjugation reaction of xenobiotic metabolites and charged species, such as glutathione, sulfate, glycine, or glucuronic acid to form even more polar compounds for easier clearance.

The payloads with higher intrinsic clearance may provide better therapeutic index due to their potential lower systemic plasma exposure. (Maderna, A.; Doroski, M; Subramanyam, C.; Porte, A.; Leverett, C. A.; Vetelino, B. C.; Chen, Z.; Risley, H.; Parris, K.; Pandit, J.; Varghese. A. H.; Shanker. S.; Song. C.; Sukuru, S. C. K.; Farley. K. A.; Wagenaar, M. M.; Shapiro, M. J.; Musto, S.; Lam, M-H.; Loganzo, F.; O'Donnell, C. J. “Discovery of cytotoxic dolastatin 10 analogues with N-terminal modifications” Journal Medicinal Chemistry, 2014, 57, 10527-10543). The payloads with higher intrinsic clearance likely have an improved safety profile because the payload, which is potentially toxic to healthy cells, is quickly removed from the plasma, decreasing its chance of interacting with healthy cells. Results for HLM CL are summarized in TABLE 7.

### Example D

### PAMPA (Parallel Artificial Membrane Permeability Assay)

PAMPA is a method which determines the permeability of substances from a donor compartment, through a lipid-infused artificial membrane into an acceptor compartment. See Ottaviani. G.; Martel. S.; Carrupt. P-A. “Parallel Artificial Membrane Permeability Assay; A New Membrane for the Fast Prediction of Passive Human Skin Permeability”. Journal of Medicinal Chemistry, 2006, 49(13), 3948-3954). A multi-well microtitre plate is used for the donor and a membrane/acceptor compartment is placed on top: the whole assembly is commonly referred to as a “sandwich”. At the beginning of the test, the drug is added to the donor compartment, and the acceptor compartment is drug-free. After an incubation period which may include stirring, the sandwich is separated, and the amount of drug is measured in each compartment. Mass balance allows calculation of drug that remains in the membrane.

The PAMPA was performed by Pion Inc using the GIT-0 lipid and 5 mM donor solution in pH 5.0 and pH 7.4 PRISMA buffer (containing 0.05% DMSO). The higher PAMPA data has been associated with better bystander killing. (Ogitani Y.; Hagihara K.; Oitate, M.; Naito, H.; Agatsuma T. “Bystander killing effect of DS-8201a, a novel anti-human epidermal growth factor receptor 2 antibody-drug conjugate, in tumors with human epidermal growth factor receptor 2 heterogeneity” Cancer Science. 2016, 107 (7), 1039-1046).

Higher permeability is important because it implies greater potential for “bystander killing”. That is, once the payload has neutralized a tumor cell, a more permeable payload is more likely to escape the neutralized tumor cell, then imbed in a neighboring tumor cell. Once there, it can neutralize the tumor cell, escape, embed in another neighboring tumor cell, and repeat the process. Results for PAMPA are summarized in TABLE 7.

### Example E

### Development of Anti-ROR-1-Specific Monoclonal Antibodies

Novel and diversified anti-ROR-1 specific monoclonal antibodies were developed to bind to multiple regions of the ROR-1 extracellular domain (ECD) by employing an antibody development campaign using three strategies: (1) mice of cohort 1 were immunized using full length ROR-1 ECD: (2) mice of cohorts 2 and 3 were immunized with the ROR-1 IgG-like domain; and (3) mice of cohort 4 were immunized with a short region of the human IgG-like sequence of ROR-1. After immunization of the mice, monoclonal antibodies were generated using conventional approaches. Briefly, unique variable heavy and light chain pairs from hybridoma and phage display campaigns were cloned into vectors designed to express full length antibodies as IgGs in HEK293 cells under the control of a CMV promoter. Antibody expression vectors were complexed with polyethylenimine and transfected into HEK293 cultures. After 5 days of shaking at 37° C. in 293 cell culture media, antibodies were captured on agarose-based protein A resin. After several stringent washes, antibodies were eluted in glycine solution, pH 3, neutralized with Hepes, pH 9, and buffer exchanged into PBS.

Several monoclonal antibodies were developed using these approaches and the generated antibodies were subjected to additional screening to assess specific characteristics of the antibodies. To fully evaluate the characteristics of the novel antibodies several assays were performed. First, confirmation of antibody binding to the ROR-1 epitope was confirmed both biochemically, as well as, in ROR-1 positive cell lines. The specificity of binding was assessed biochemically by screening binding to human ROR-2 protein, rodent ROR-1 protein, as well as, in cell-based assays. Further screening parameters included analyses of antibody internalization, epitope binning against known anti-ROR-1 antibodies (UC961 and 4a5), binding to human ROR-1 Ig-like domain, thermal shift, and assessment for self-interaction with Affinity-Capture Self-Interaction Nanoparticle Spectroscopy (AC-SINS).

### Example F

### Assay Evaluating Saturation Concentrations and Human ROR-1 Binding Affinities of Anti-ROR-1 Specific Monoclonal Antibodies

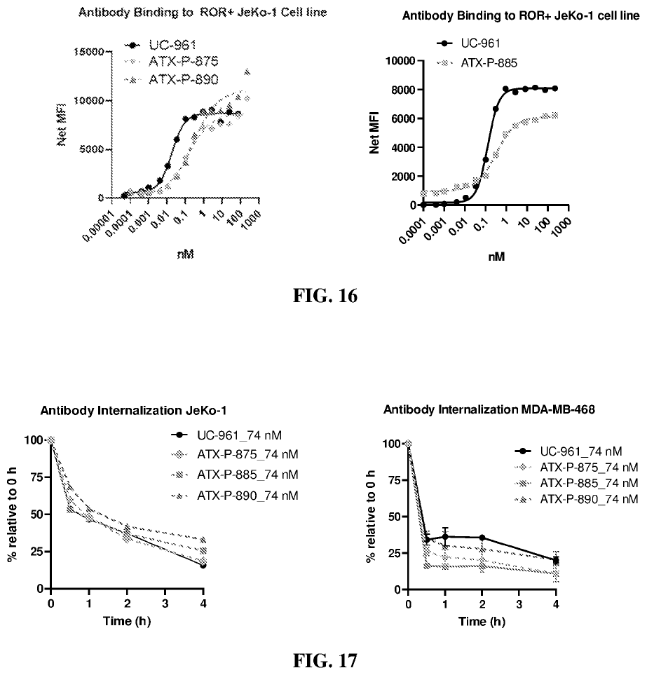

A cell binding saturation assay was developed to evaluate how well the anti-ROR-1 antibodies developed in Example 16 bound to endogenously expressed extracellular ROR-1 protein on cell lines. More specifically, the anti-ROR-1 monoclonal antibodies developed in Example 16, e.g., ATX-P-875. ATX-P-885, and ATX-P-890, were analyzed in a cellular binding assay. Briefly, two ROR-1 positive cell lines. JeKo-1 and MDA-MB-468, were incubated in a titration series concentration of each antibody construct. Cells were then washed and subjected to secondary antibody staining and detection by flow cytometry. Mean fluorescence (MFI) was determined by analysis on cytometer software. The binding of ATX-P-875, ATX-P-885, and ATX-P-890 was compared to cell binding saturation data for the monoclonal anti-ROR-1 antibody UC961. (See FIG. 16). As shown in FIG. 16, the cell binding saturation for antibodies ATX-P-875, ATX-P-885, and ATX-P-890 were comparable to the cell binding saturation for UC961 though a greater concentration of ATX-P-875 was needed to achieve saturation, as compared to UC961. ATX-P-890 and ATX-P-885 were as good or improved, respectively, compared to UC-961 in concentrations needed to achieve binding saturation. Comparable saturation to UC961 demonstrates that the anti-ROR-1 antibodies ATX-P-875, ATX-P-885, and ATX-P-890 have a similar affinity to the human ROR-1 target as a clinically approved antibody UC-961.

### Example G

### Assay Evaluating Capacity of Anti-ROR-1 Specific Monoclonal Antibodies to Internalize ROR-1 on Human ROR-1-Positive Tumor Cells

After a saturating concentration (74 nM) was determined in the binding assay, the anti-ROR-1 antibodies developed herein (ATX-P-875, P-885, P-890) were evaluated for their capacity to internalize the ROR-1 receptor on human ROR-1 positive cells (JeKo-1 and MDA-MB-468). Briefly, the ROR positive cell lines were incubated with antibody at super saturating conditions so as to bind all available ROR-1 receptors. Excess antibody was washed off and the cells were incubated at 37° C., for a designated amount of time over a four-hour time course. At the end of each time point, internalization was stopped by placing an aliquot of cells on ice. The antibody remaining on the surface was detected using a labeled secondary antibody and flow cytometry. Percent internalization was calculated based on time zero, and time zero was assumed that 100% of available receptors are on the cell surface. The results in FIG. 17 demonstrate that all antibodies internalize ROR-1 receptor on JeKo-1 and MDA-MB-468 cells by a reduction of at least 75% over 4 hours. Unexpectedly, in MDA-MB-468, internalization of two of the anti-ROR-1 antibodies (ATX-P-875 and ATX-P-890) was improved over the clinically used UC961 anti-ROR-1 antibody providing evidence that the ATX-P-875 and ATX-P-890 antibodies have an improved ability to internalize the ROR-1 receptor from the surface of solid tumors.

### Example H

### Epitope Binding Studies of Anti-ROR-1 Specific Monoclonal Antibodies

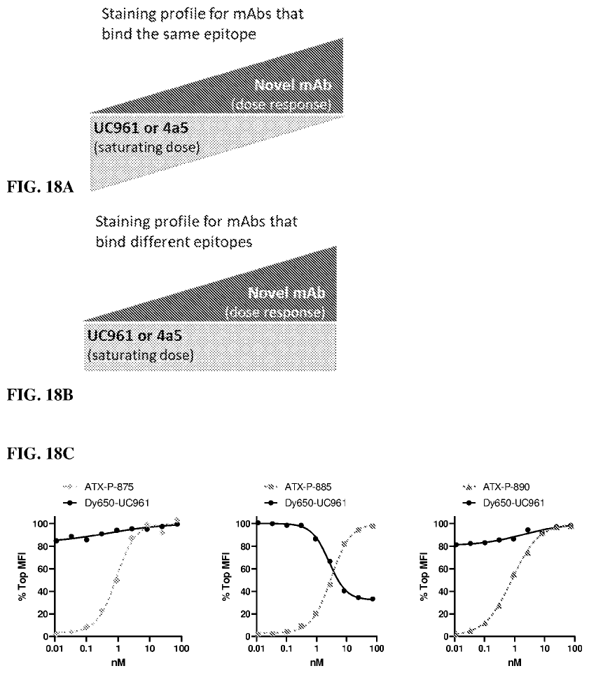

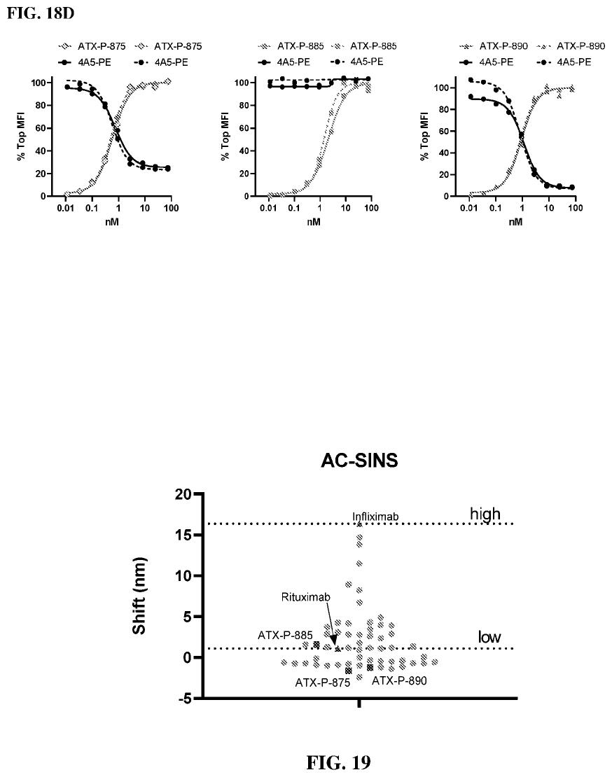

Cellular binning was also employed to determine if monoclonal antibodies ATX-P-875. ATX-P-885, and ATX-P-890 bound to the same epitopes as conventionally known anti-ROR-1 binding monoclonal antibodies UC961 and 4A5 (controls). In Step 1 of the cellular binning experiments, ATX-P-875, ATX-P-885, and ATX-P-890 monoclonal antibodies were separately incubated with ROR-1 expressing cells (MDA-MB-468) at various amounts. In Step 2, a fluorescently labeled secondary antibody recognizing the novel antibodies was incubated with the samples. And finally in Step 3, the ROR-1 expressing cells coated with ATX-P-875. ATX-P-885, and ATX-P-890 were incubated with a saturating dose of labeled UC961 (Dy650-UC 961) or 4A5 antibody (PE 4A5) and analyzed by flow cytometry. The UC961 and 4A5 staining signal was then compared to the novel antibody staining signal to determine if the ATX-P-875. ATX-P-885, and ATX-P-890 antibodies bound the same epitope as the known ROR-1 binding antibodies UC961 and 4A5. FIG. 18A below shows the staining profile expected if the ATX-P-875, ATX-P-885, and ATX-P-890 antibodies bound the same epitope as the UC961 and 4A5 antibodies. FIG. 18B, shows the expected profile if the ATX-P-875. ATX-P-885, and ATX-P-890 antibodies bound to a separate epitope on ROR-1 than the UC961 or 4A5 antibodies. Briefly, if binding the same epitope, increased novel antibody concentration would block the binding of prelabeled competitor antibody, thereby reducing the signal of the competitor at higher concentrations. In the case antibodies bound separate epitopes, each antibody, novel and competitor, would have increased staining with increased dose as there would be no competition for binding to the receptor. The cellular binning data obtained in MDA-MB-468 cells indicated that ATX-P-885 appreciably bound the same epitope as UC961 and both ATX-P-875 and ATX-P-890 appreciably bound the same epitope as 4A5. (See FIG. 18C, 18D and FIG. 19). The ability of the antibodies developed herein to bind distinct ROR-1 epitopes provides the opportunity to regulate the target in a variety of ways.

### Example I

### Biochemical Binning Studies of Anti-ROR-1 Specific Monoclonal Antibodies

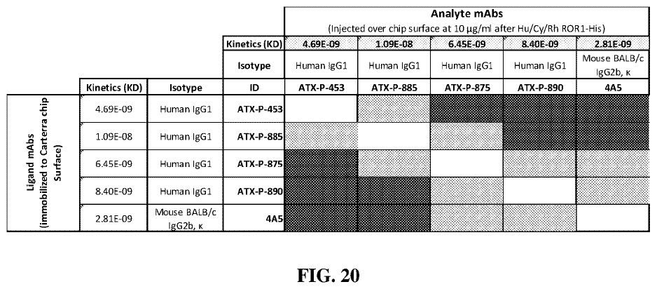

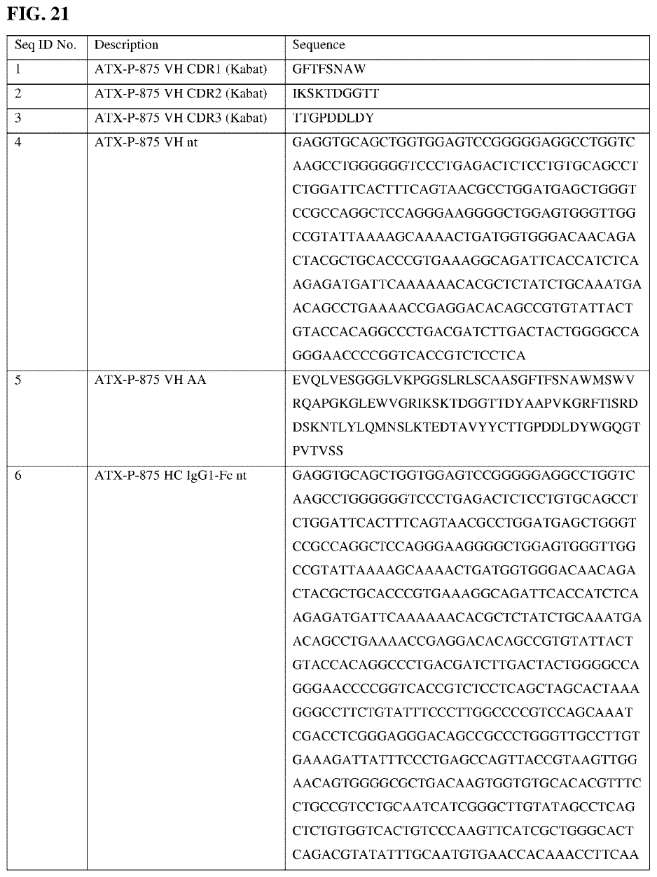

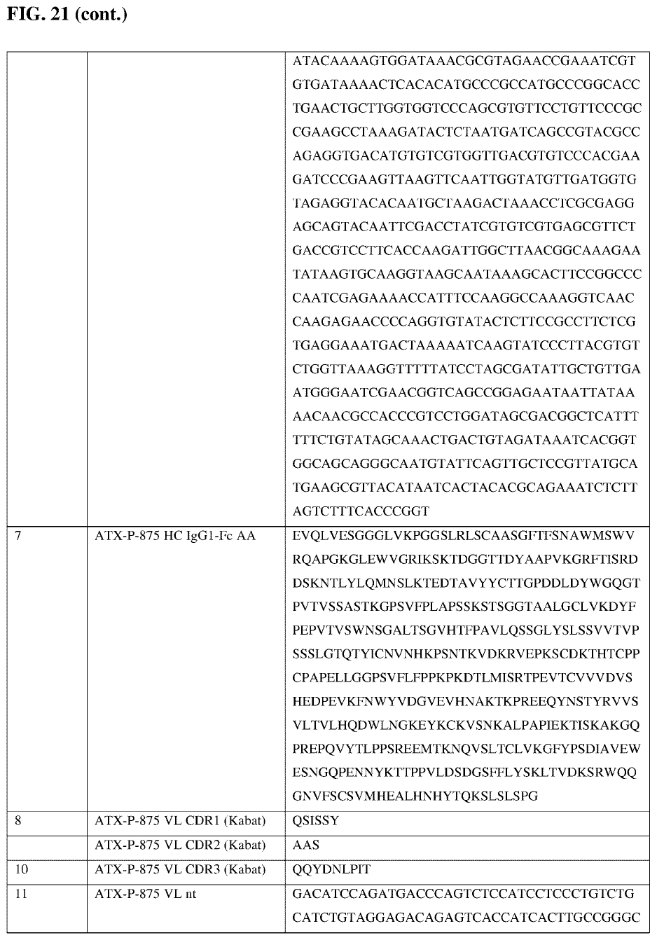

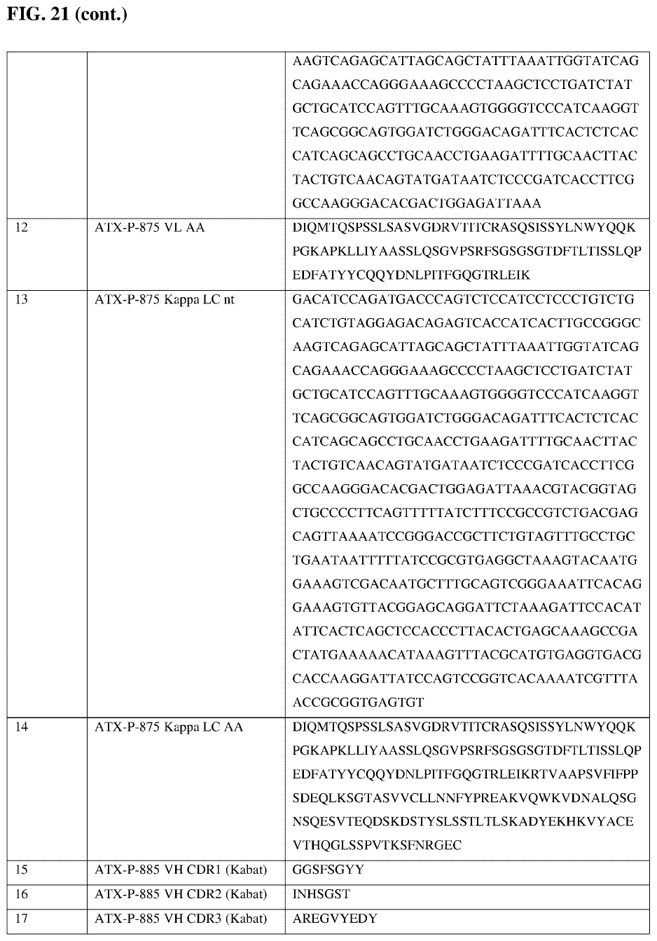

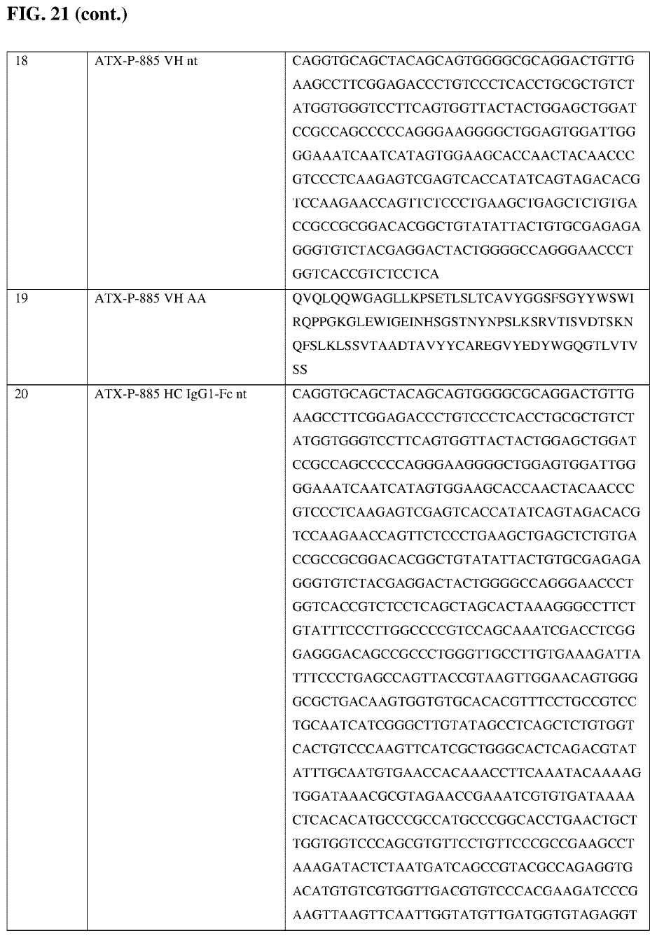

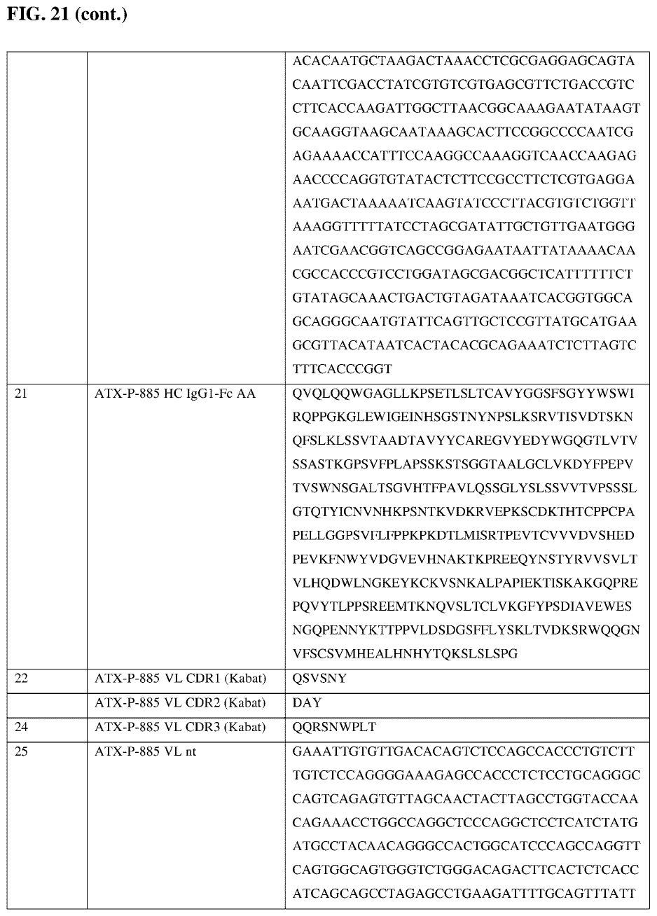

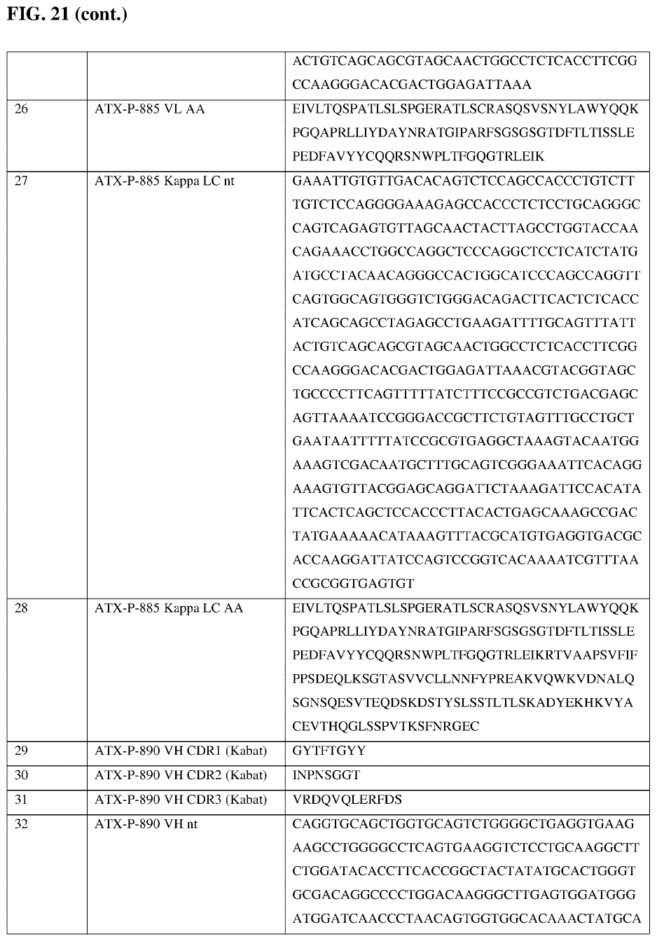

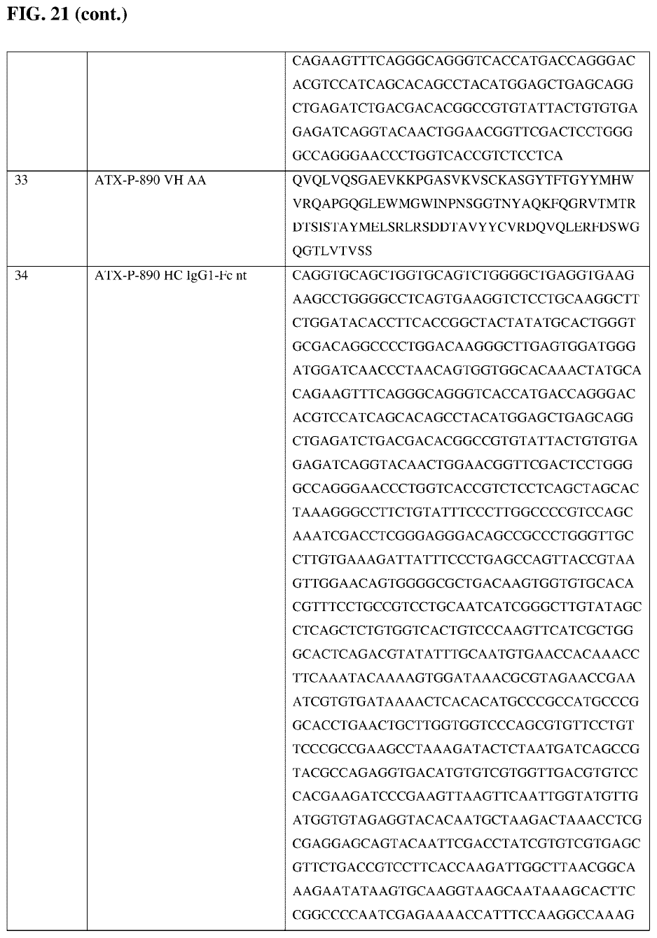

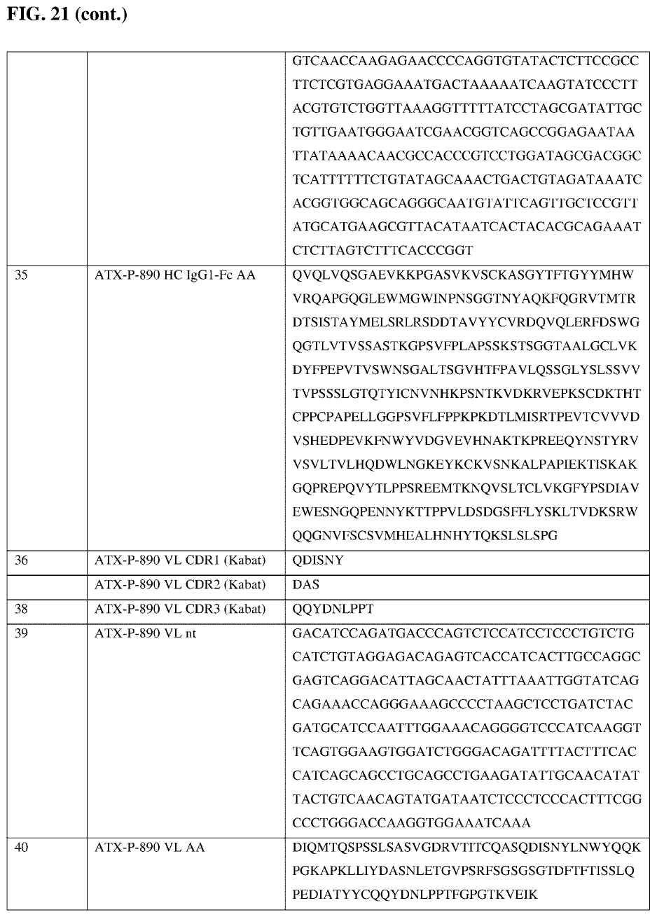

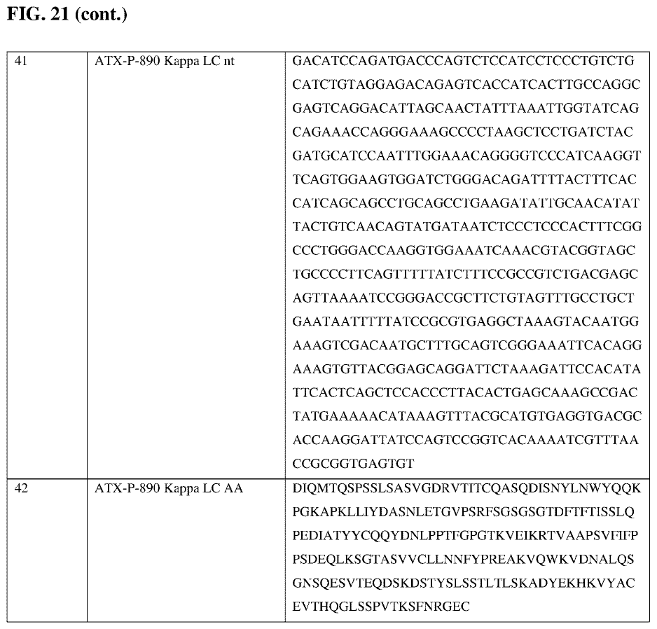

Biochemical binning by SPR was also evaluated for the anti-ROR1 antibodies (ATX-P-875, P-885, P-890) as compared against control anti-ROR-1 antibodies UC961 and 4a5. In these experiments 10 ug/ml of purified clonal protein of Hu/Cy/Rh ROR1-His was covalently coupled to the HC30M chip. Individual dilutions of each antibody at 10 μg/mL were injected over the chip and binding was evaluated by Carterra SPR. Unexpectedly the data demonstrate that there are 3 distinct binding epitopes between ATX-P-875, ATX-P-885, and ATX-P-890 with ATX-885 being the only antibody to impart partial blocking to the UC961 antibody (See FIG. 20). Cellular binning only evaluated the antibodies' ability to block either UC961 or 4a5, two clinically used ROR-1 antibodies. Biochemical SPR evaluation also tested the antibodies' ability to block each other and found that ATX-P-875 was able to block the binding of 4a5 as well as ATX-P-885, while still not being able to block UC961.

### Example J

### Antibody Characterization

Antibody characterization of ATX-P-875, ATX-P-885, and ATX-P-890, as compared to UC961, are summarized in FIG. 19 and TABLES 1-6. An initial assessment of antibody developability was performed by AC-SINS to evaluate the potential for self-interaction (FIG. 19). Control antibody Adalimumab shows an expected low shift and Infliximab shows an expected high shift. The anti-ROR-1 antibodies developed herein, ATX-P-875, ATX-P-885, and ATX-P-890, are in line with control antibodies that do not show significant self-interaction and are not likely to pose a significant developability risk. Additionally, the binding characteristics of monoclonal antibodies ATX-P-875, ATX-P-885, and ATX-P-890 were compared to the binding characteristics for UC961 in additional experiments. TABLES 1-4 provide this antibody characterization data in comparison to the known ROR-1 binding antibody UC961 including tabled results for biochemical binding to purified proteins and measured by SPR (TABLES 1-4), cellular binding to ROR-1 positive cell lines JeKo-1 and MDA-MB-468 (EC50) (TABLE 5), and cellular internalization (% internalized) (TABLE 6). Of particular note, it is believed that the reduced affinity of ATX-P-885 (KD: 1.09E-08) compared to UC961 and other anti-ROR-1 antibodies (ATX-P-875 and ATX-P-890) can provide an unexpected therapeutic benefit. It is contemplated that by binding less tightly to the ROR-1 epitope, the ATX-P-885 antibody can penetrate further into the tumor to reach more distant cells expressing ROR-1 target.

TABLE 1
HUMAN/CYNO/RHESUS ROR-1 BINDING
Hu/Cy/Rh ROR1 - His

| | kₐ | k_{d} | K_{D} | Rmax | Res | % |
|---|---|---|---|---|---|---|
| Name | (M−1 s−1) | (s−1) | (M) | (RU) | SD | Rmax |
| ATX-P-453 | 1.26E+06 | 5.91E−03 | 4.69E−09 | 529.0 | 25.6 | 4.85% |
| (UC961) | | | | | | |
| ATX-P-875 | 4.93E+05 | 3.16E−03 | 6.45E−09 | 593.6 | 22.3 | 3.76% |
| ATX-P-885 | 2.70E+05 | 2.95E−03 | 1.09E−08 | 341.6 | 10.2 | 2.99% |
| ATX-P-890 | 3.92E+05 | 3.14E−03 | 8.40E−09 | 504.2 | 15.0 | 2.97% |

TABLE 2
MOUSE ROR-1 BINDING
Mouse ROR1 - His

| | kₐ | k_{d} | K_{D} | Rmax | Res | % |
|---|---|---|---|---|---|---|
| Name | (M−1 s−1) | (s−1) | (M) | (RU) | SD | Rmax |
| ATX-P-453 | N/A | N/A | N/A | N/A | N/A | N/A |
| (UC961) | | | | | | |
| ATX-P-875 | N/A | N/A | N/A | N/A | N/A | N/A |
| ATX-P-885 | 2.92E+04 | 1.32E−03 | 4.53E−08 | 73.5 | 8.9 | 12.14% |
| ATX-P-890 | N/A | N/A | N/A | N/A | N/A | N/A |

TABLE 3
RAT ROR-1 BINDING
Rat ROR1 - His

| kₐ | kₐ | k_{d} | K_{D} | Rmax | Res | % |
|---|---|---|---|---|---|---|
| (M−1 s−1) | (M−1 s−1) | (s−1) | (M) | (RU) | SD | Rmax |
| N/A | N/A | N/A | N/A | N/A | N/A | N/A |
| N/A | N/A | N/A | N/A | N/A | N/A | N/A |
| N/A | N/A | N/A | N/A | N/A | N/A | N/A |
| N/A | N/A | N/A | N/A | N/A | N/A | N/A |

TABLE 4
HUMAN ROR-2 BINDING
Human ROR2 / NTRKR2-His

| | kₐ | k_{d} | K_{D} | Rmax | Res | % |
|---|---|---|---|---|---|---|
| Name | (M−1 s−1) | (s−1) | (M) | (RU) | SD | Rmax |
| ATX-P-453 | N/A | N/A | N/A | N/A | N/A | N/A |
| (UC961) | | | | | | |
| ATX-P-875 | N/A | N/A | N/A | N/A | N/A | N/A |
| ATX-P-885 | N/A | N/A | N/A | N/A | N/A | N/A |
| ATX-P-890 | N/A | N/A | N/A | N/A | N/A | N/A |

TABLE 5
CELLULAR BINDING SUMMARY
Cellular Binding Summary

| | JeKo-1 | MDA-MB-468 |
|---|---|---|
| Name | (Avg EC50) | (Avg EC50) |
| ATX-P-453 (UC961) | 0.073 | 0.176 |
| ATX-P-875 | 0.145 | 0.708 |
| ATX-P-885 | 1.075 | 2.508 |
| ATX-P-890 | 0.174 | 1.016 |

TABLE 6
CELLULAR INTERNALIZATION SUMMARY
Cellular Internalization Summary

| Name | JeKo-1 (%) | MDA-MB-468(%) |
|---|---|---|
| ATX-P-453 (UC961) | 75 | 80 |
| ATX-P-875 | 75 | 89 |
| ATX-P-885 | 75 | 89 |
| ATX-P-890 | 70 | 80 |

TABLE 7
BIOLOGICAL DATA

| | PAMPA Pe | HHEP Cl | HLM Cl | JeKo-1 |
|---|---|---|---|---|
| | (10⁻⁶ cm/s) | T_{1/2}, | T_{1/2}, | IC₅₀ |
| Compound | pH 5.0/7.4 | min | min | (nM) |
| Dxd | >66.5/5.7 | >217 | 100 | 0.95 |
| Exatecan | >107/16.3 | 143 | 75 | 0.40 |
| SN-38 | 16.9/8.1 | 77 | 81 | 0.92 |
| 14-92 | 17.7/8.4 | >217 | 57 | 1.7 |
| 14-93 | 20.6/9.2 | 185 | 46 | 2.6 |
| 14-94 | 16.5/7.5 | 68 | 6.7 | 1.6 |
| 14-95 | 12.7/6.6 | 85 | 29 | 1.7 |
| 15-96 | 16.7/15.6 | >217 | 96 | 0.52 |
| 15-97 | 36.6/15.7 | 113 | 36 | 0.30 |
| 15-98 | 27.0/11.3 | 78 | 6.7 | 0.25 |
| 15-99 | 14.9/8.5 | 208 | 58 | 0.32 |
| 16-102 | <0.03/0.02 | 105 | 142 | 27.7 |
| 16-105 | 0.07/0.06 | 112 | >145 | 10.2 |
| 16-108 | 0.001/0.002 | 126 | 110 | 8.3 |
| 16-111 | 0.03/0.04 | 133 | 103 | 17.6 |
| 17-112 | 14.4/6.3 | >217 | 62 | 17.0 |
| 17-113 | 26.9/9.7 | 145 | 32 | 4.2 |
| 17-114 | 18.3/8.0 | 48 | 2.1 | 1.9 |
| 17-115 | 10.5/4.8 | 90 | 6.0 | 16.0 |

HHEP Cl: human hepatocytes intrinsic clearance, t_{1/2}, min.
HLM: human liver microsome clearance, t_{1/2}, min.
ND: Not determined.

### Example K

### Preparation of Antibody-Drug Conjugates

The synthesis of the immunoconjugates is accomplished as set forth in this example. The antibodies are produced as described in Example E and are suspended in PBS pH 7.2 with protein concentrations ranging between 10-20 mg/ml. For the reduction and conjugation calculations, a molecular weight of 150000 Da for all antibodies was used.

Each antibody is prepared for reduction by the addition of 5% v/v of 500 mM Tris, 25 mM EDTA, pH 8.5, followed by the addition of TCEP (6 equivalent, 10 mM stock of TCEP in water) and the mixture is maintained at 20° C., for 2 h. This reduction step forms the cysteine residues Cys-SH on the antibodies to facilitate bioconjugation with the toxin-linkers, i.e., compounds of Formula (III) as described herein.

After DMA is added and gently mixed with the above reduced protein solution to achieve a final 10% v/v during conjugation, a toxin-linker stock solution (12 equivalent, 50 mM in DMA) is added and gently mixed. The bioconjugation is allowed to proceed for approximately 16-20 h overnight at 20° C.: it is complete within 2 h with the extended time allowed for maleimide ring opening.

The crude conjugate is buffer exchanged to PBS pH 7.4 using a gravity fed NAP 25 (small scale) or a flow HiPrep G25 (large scale) with the columns prepared and operated according to manufacturer's (Cytivia) instructions. To remove residual toxin, a 100 mg/ml slurry of activated carbon (Sigma/C9157) in PBS pH 7.4 is prepared and added to achieve 1 mg carbon to 1 mg starting antibody mass. It is mixed gently for 2 h, sufficiently to maintain the carbon in suspension. Then, the carbon is removed by centrifugation at 4000 g. Polysorbate 20 (PS20) is added from a 10% w/v stock solution in PBS pH7.4 to achieve a final 0.02% PS20 w/v in the product. The antibody-drug conjugate (ADC) product is terminally filtered through a suitably sized 0.2 μm PES filter (chromatography direct/FIL-S-PES-022-13-100-S) under grade A laminar flow. The final product is analyzed as follows: monomer and [ADC] mg/ml by SEC HPLC, average DAR by PLRP, residual toxin by RP-HPLC, and endotoxin by Endosafe kinetic chromogenic.

Analytical processes are carried out on HPLC instruments Agilent 1100 or 1260.

### Example L

### CTG Assays of Antibody-Drug Conjugates

Novel ROR-1 antibody-drug conjugates (ADCs) are evaluated by CTG assays in a similar manner as was described in Example A and TABLE 7 for screening of payloads. By no means of limiting the scope of the antibody-drug conjugates of the present disclosure and by way of example only, a total of 3 unique antibodies ATX-P-875, ATX-P-885, and ATX-P-890 are conjugated to novel linker/payloads or compounds of Formula (III), including but not limited to compounds 14-92, 14-93, 14-94, 14-95, 15-96, 15-97, 15-98, 15-99 as well as any of the exemplary compounds of Formula (III) described herein Briefly, ROR-positive (JeKo-1/MDA-MB-468) or ROR-negative (Ramos) cells are transferred to 96 wells and treated with the test ADCs in three-fold serial dilution starting from 1 mM to 0.0000508 mM (10 points dilution), for 72 h. Cell viability is analyzed with CellTiter-Glo® Luminescent Cell Viability Assay (Promega) following the manufacturer's instructions. The percentage of viable cells at each ADC concentration is determined by normalizing with the luminescence of vehicle control and plotted into percentage of viability versus dose response curve by nonlinear fit in GraphPad Prism software. The IC₅₀ for each test ADC is calculated as the concentration of compound killing 50% of cells and is benchmarked against UC-961. The ADCs described herein, including those prepared by conjugating antibodies ATX-P-875, ATX-P-885, and ATX-P-890 to compounds 14-92, 14-93, 14-94, 14-95, 15-96, 15-97, 15-98, 15-99 and any of the exemplary compounds of Formula (III), have an IC₅₀ value below 500 nM (for example, below 300 nM, below 100 nM, below 50 nM or below 30 nM) based on CTG assays with Jeko-1 or MDA-MB-468 cells.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

SEQUENCE LISTING
ATX-P-875 VH CDR1 (Kabat)
SEQ ID NO: 1
GFTESNAW
ATX-P-875 VH CDR2 (Kabat)
SEQ ID NO: 2
IKSKTDGGTT
ATX-P-875 VH CDR3 (Kabat)
SEQ ID NO: 3
TTGPDDLDY
ATX-P-875 VH nt
SEQ ID NO: 4
GAGGTGCAGCTGGTGGAGTCCGGGGGAGGCCTGGTCAAGCCTGGGGGGTCCCTGAGACTC
TCCTGTGCAGCCTCTGGATTCACTTTCAGTAACGCCTGGATGAGCTGGGTCCGCCAGGCT
CCAGGGAAGGGGCTGGAGTGGGTTGGCCGTATTAAAAGCAAAACTGATGGTGGGACAACA
GACTACGCTGCACCCGTGAAAGGCAGATTCACCATCTCAAGAGATGATTCAAAAAACACG
CTCTATCTGCAAATGAACAGCCTGAAAACCGAGGACACAGCCGTGTATTACTGTACCACA
GGCCCTGACGATCTTGACTACTGGGGCCAGGGAACCCCGGTCACCGTCTCCTCA
ATX-P-875 VH AA
SEQ ID NO: 5
EVQLVESGGGLVKPGGSLRLSCAASGFTESNAWMSWVRQAPGKGLEWVGRIKSKTDGGTT
DYAAPVKGRFTISRDDSKNTLYLQMNSLKTEDTAVYYCTTGPDDLDYWGQGTPVTVSS
ATX-P-875 HC IgG1-Fc nt
SEQ ID NO: 6
GAGGTGCAGCTGGTGGAGTCCGGGGGAGGCCTGGTCAAGCCTGGGGGGTCCCTGAGACTC
TCCTGTGCAGCCTCTGGATTCACTTTCAGTAACGCCTGGATGAGCTGGGTCCGCCAGGCT
CCAGGGAAGGGGCTGGAGTGGGTTGGCCGTATTAAAAGCAAAACTGATGGTGGGACAACA
GACTACGCTGCACCCGTGAAAGGCAGATTCACCATCTCAAGAGATGATTCAAAAAACACG
CTCTATCTGCAAATGAACAGCCTGAAAACCGAGGACACAGCCGTGTATTACTGTACCACA
GGCCCTGACGATCTTGACTACTGGGGCCAGGGAACCCCGGTCACCGTCTCCTCAGCTAGC
ACTAAAGGGCCTTCTGTATTTCCCTTGGCCCCGTCCAGCAAATCGACCTCGGGAGGGACA
GCCGCCCTGGGTTGCCTTGTGAAAGATTATTTCCCTGAGCCAGTTACCGTAAGTTGGAAC
AGTGGGGCGCTGACAAGTGGTGTGCACACGTTTCCTGCCGTCCTGCAATCATCGGGCTTG
TATAGCCTCAGCTCTGTGGTCACTGTCCCAAGTTCATCGCTGGGCACTCAGACGTATATT
TGCAATGTGAACCACAAACCTTCAAATACAAAAGTGGATAAACGCGTAGAACCGAAATCG
TGTGATAAAACTCACACATGCCCGCCATGCCCGGCACCTGAACTGCTTGGTGGTCCCAGC
GTGTTCCTGTTCCCGCCGAAGCCTAAAGATACTCTAATGATCAGCCGTACGCCAGAGGTG
ACATGTGTCGTGGTTGACGTGTCCCACGAAGATCCCGAAGTTAAGTTCAATTGGTATGTT
GATGGTGTAGAGGTACACAATGCTAAGACTAAACCTCGCGAGGAGCAGTACAATTCGACC
TATCGTGTCGTGAGCGTTCTGACCGTCCTTCACCAAGATTGGCTTAACGGCAAAGAATAT
AAGTGCAAGGTAAGCAATAAAGCACTTCCGGCCCCAATCGAGAAAACCATTTCCAAGGCC
AAAGGTCAACCAAGAGAACCCCAGGTGTATACTCTTCCGCCTTCTCGTGAGGAAATGACT
AAAAATCAAGTATCCCTTACGTGTCTGGTTAAAGGTTTTTATCCTAGCGATATTGCTGTT
GAATGGGAATCGAACGGTCAGCCGGAGAATAATTATAAAACAACGCCACCCGTCCTGGAT
AGCGACGGCTCATTTTTTCTGTATAGCAAACTGACTGTAGATAAATCACGGTGGCAGCAG
GGCAATGTATTCAGTTGCTCCGTTATGCATGAAGCGTTACATAATCACTACACGCAGAAA
TCTCTTAGTCTTTCACCCGGT
ATX-P-875 HC IgG1-Fc AA
SEQ ID NO: 7
EVQLVESGGGLVKPGGSLRLSCAASGFTESNAWMSWVRQAPGKGLEWVGRIKSKTDGGTT
DYAAPVKGRFTISRDDSKNTLYLQMNSLKTEDTAVYYCTTGPDDLDYWGQGTPVTVSSAS
TKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPS
VFLFPPKPKDTLMISRTPEVTCVVVDVSHEDPEVKENWYVDGVEVHNAKTKPREEQYNST
YRVVSVLTVLHQDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYSKLTVDKSRWQQ
GNVFSCSVMHEALHNHYTQKSLSLSPG
ATX-P-875 VL CDR1 (Kabat)
SEQ ID NO: 8
QSISSY
ATX-P-875 VL CDR2 (Kabat)
AAS
ATX-P-875 VL CDR3 (Kabat)
SEQ ID NO: 10
QQYDNLPIT
ATX-P-875 VL nt
SEQ ID NO: 11
GACATCCAGATGACCCAGTCTCCATCCTCCCTGTCTGCATCTGTAGGAGACAGAGTCACC
ATCACTTGCCGGGCAAGTCAGAGCATTAGCAGCTATTTAAATTGGTATCAGCAGAAACCA
GGGAAAGCCCCTAAGCTCCTGATCTATGCTGCATCCAGTTTGCAAAGTGGGGTCCCATCA
AGGTTCAGCGGCAGTGGATCTGGGACAGATTTCACTCTCACCATCAGCAGCCTGCAACCT
GAAGATTTTGCAACTTACTACTGTCAACAGTATGATAATCTCCCGATCACCTTCGGCCAA
GGGACACGACTGGAGATTAAA
ATX-P-875 VL AA
SEQ ID NO: 12
DIQMTQSPSSLSASVGDRVTITCRASQSISSYLNWYQQKPGKAPKLLIYAASSLQSGVPS
RFSGSGSGTDFTLTISSLQPEDFATYYCQQYDNLPITFGQGTRLEIK
ATX-P-875 Kappa LC nt
SEQ ID NO: 13
GACATCCAGATGACCCAGTCTCCATCCTCCCTGTCTGCATCTGTAGGAGACAGAGTCACC
ATCACTTGCCGGGCAAGTCAGAGCATTAGCAGCTATTTAAATTGGTATCAGCAGAAACCA
GGGAAAGCCCCTAAGCTCCTGATCTATGCTGCATCCAGTTTGCAAAGTGGGGTCCCATCA
AGGTTCAGCGGCAGTGGATCTGGGACAGATTTCACTCTCACCATCAGCAGCCTGCAACCT
GAAGATTTTGCAACTTACTACTGTCAACAGTATGATAATCTCCCGATCACCTTCGGCCAA
GGGACACGACTGGAGATTAAACGTACGGTAGCTGCCCCTTCAGTTTTTATCTTTCCGCCG
TCTGACGAGCAGTTAAAATCCGGGACCGCTTCTGTAGTTTGCCTGCTGAATAATTTTTAT
CCGCGTGAGGCTAAAGTACAATGGAAAGTCGACAATGCTTTGCAGTCGGGAAATTCACAG
GAAAGTGTTACGGAGCAGGATTCTAAAGATTCCACATATTCACTCAGCTCCACCCTTACA
CTGAGCAAAGCCGACTATGAAAAACATAAAGTTTACGCATGTGAGGTGACGCACCAAGGA
TTATCCAGTCCGGTCACAAAATCGTTTAACCGCGGTGAGTGT
ATX-P-875 Kappa LC AA
SEQ ID NO: 14
DIQMTQSPSSLSASVGDRVTITCRASQSISSYLNWYQQKPGKAPKLLIYAASSLQSGVPS
RFSGSGSGTDFTLTISSLQPEDFATYYCQQYDNLPITFGQGTRLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSENRGEC
ATX-P-885 VH CDR1 (Kabat)
SEQ ID NO: 15
GGSFSGYY
ATX-P-885 VH CDR2 (Kabat)
SEQ ID NO: 16
INHSGST
ATX-P-885 VH CDR3 (Kabat)
SEQ ID NO: 17
AREGVYEDY
ATX-P-885 VH nt
SEQ ID NO: 18
CAGGTGCAGCTACAGCAGTGGGGCGCAGGACTGTTGAAGCCTTCGGAGACCCTGTCCCTC
ACCTGCGCTGTCTATGGTGGGTCCTTCAGTGGTTACTACTGGAGCTGGATCCGCCAGCCC
CCAGGGAAGGGGCTGGAGTGGATTGGGGAAATCAATCATAGTGGAAGCACCAACTACAAC
CCGTCCCTCAAGAGTCGAGTCACCATATCAGTAGACACGTCCAAGAACCAGTTCTCCCTG
AAGCTGAGCTCTGTGACCGCCGCGGACACGGCTGTATATTACTGTGCGAGAGAGGGTGTC
TACGAGGACTACTGGGGCCAGGGAACCCTGGTCACCGTCTCCTCA
ATX-P-885 VH AA
SEQ ID NO: 19
QVQLQQWGAGLLKPSETLSLTCAVYGGSFSGYYWSWIRQPPGKGLEWIGEINHSGSTNYN
PSLKSRVTISVDTSKNQFSLKLSSVTAADTAVYYCAREGVYEDYWGQGTLVTVSS
ATX-P-885 HC IgG1-Fc nt
SEQ ID NO: 20
CAGGTGCAGCTACAGCAGTGGGGCGCAGGACTGTTGAAGCCTTCGGAGACCCTGTCCCTC
ACCTGCGCTGTCTATGGTGGGTCCTTCAGTGGTTACTACTGGAGCTGGATCCGCCAGCCC
CCAGGGAAGGGGCTGGAGTGGATTGGGGAAATCAATCATAGTGGAAGCACCAACTACAAC
CCGTCCCTCAAGAGTCGAGTCACCATATCAGTAGACACGTCCAAGAACCAGTTCTCCCTG
AAGCTGAGCTCTGTGACCGCCGCGGACACGGCTGTATATTACTGTGCGAGAGAGGGTGTC
TACGAGGACTACTGGGGCCAGGGAACCCTGGTCACCGTCTCCTCAGCTAGCACTAAAGGG
CCTTCTGTATTTCCCTTGGCCCCGTCCAGCAAATCGACCTCGGGAGGGACAGCCGCCCTG
GGTTGCCTTGTGAAAGATTATTTCCCTGAGCCAGTTACCGTAAGTTGGAACAGTGGGGCG
CTGACAAGTGGTGTGCACACGTTTCCTGCCGTCCTGCAATCATCGGGCTTGTATAGCCTC
AGCTCTGTGGTCACTGTCCCAAGTTCATCGCTGGGCACTCAGACGTATATTTGCAATGTG
AACCACAAACCTTCAAATACAAAAGTGGATAAACGCGTAGAACCGAAATCGTGTGATAAA
ACTCACACATGCCCGCCATGCCCGGCACCTGAACTGCTTGGTGGTCCCAGCGTGTTCCTG
TTCCCGCCGAAGCCTAAAGATACTCTAATGATCAGCCGTACGCCAGAGGTGACATGTGTC
GTGGTTGACGTGTCCCACGAAGATCCCGAAGTTAAGTTCAATTGGTATGTTGATGGTGTA
GAGGTACACAATGCTAAGACTAAACCTCGCGAGGAGCAGTACAATTCGACCTATCGTGTC
GTGAGCGTTCTGACCGTCCTTCACCAAGATTGGCTTAACGGCAAAGAATATAAGTGCAAG
GTAAGCAATAAAGCACTTCCGGCCCCAATCGAGAAAACCATTTCCAAGGCCAAAGGTCAA
CCAAGAGAACCCCAGGTGTATACTCTTCCGCCTTCTCGTGAGGAAATGACTAAAAATCAA
GTATCCCTTACGTGTCTGGTTAAAGGTTTTTATCCTAGCGATATTGCTGTTGAATGGGAA
TCGAACGGTCAGCCGGAGAATAATTATAAAACAACGCCACCCGTCCTGGATAGCGACGGC
TCATTTTTTCT
ATX-P-885 HC IgG1-Fc AA
SEQ ID NO: 21
QVQLQQWGAGLLKPSETLSLTCAVYGGSFSGYYWSWIRQPPGKGLEWIGEINHSGSTNYN
PSLKSRVTISVDTSKNQFSLKLSSVTAADTAVYYCAREGVYEDYWGQGTLVTVSSASTKG
PSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPKSCDKTHTCPPCPAPELLGGPSVEL
FPPKPKDTLMISRTPEVTCVVVDVSHEDPEVKENWYVDGVEVHNAKTKPREEQYNSTYRV
VSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNV
FSCSVMHEALHNHYTQKSLSLSPG
ATX-P-885 VL CDR1 (Kabat)
SEQ ID NO: 22
QSVSNY
ATX-P-885 VL CDR2 (Kabat)
DAY
ATX-P-885 VL CDR3 (Kabat)
SEQ ID NO: 24
QQRSNWPLT
ATX-P-885 VL nt
SEQ ID NO: 25
GAAATTGTGTTGACACAGTCTCCAGCCACCCTGTCTTTGTCTCCAGGGGAAAGAGCCACC
CTCTCCTGCAGGGCCAGTCAGAGTGTTAGCAACTACTTAGCCTGGTACCAACAGAAACCT
GGCCAGGCTCCCAGGCTCCTCATCTATGATGCCTACAACAGGGCCACTGGCATCCCAGCC
AGGTTCAGTGGCAGTGGGTCTGGGACAGACTTCACTCTCACCATCAGCAGCCTAGAGCCT
GAAGATTTTGCAGTTTATTACTGTCAGCAGCGTAGCAACTGGCCTCTCACCTTCGGCCAA
GGGACACGACTGGAGATTAAA
ATX-P-885 VL AA
SEQ ID NO: 26
EIVLTQSPATLSLSPGERATLSCRASQSVSNYLAWYQQKPGQAPRLLIYDAYNRATGIPA
RFSGSGSGTDFTLTISSLEPEDFAVYYCQQRSNWPLTFGQGTRLEIK
ATX-P-885 Kappa LC nt
SEQ ID NO: 27
GAAATTGTGTTGACACAGTCTCCAGCCACCCTGTCTTTGTCTCCAGGGGAAAGAGCCACC
CTCTCCTGCAGGGCCAGTCAGAGTGTTAGCAACTACTTAGCCTGGTACCAACAGAAACCT
GGCCAGGCTCCCAGGCTCCTCATCTATGATGCCTACAACAGGGCCACTGGCATCCCAGCC
AGGTTCAGTGGCAGTGGGTCTGGGACAGACTTCACTCTCACCATCAGCAGCCTAGAGCCT
GAAGATTTTGCAGTTTATTACTGTCAGCAGCGTAGCAACTGGCCTCTCACCTTCGGCCAA
GGGACACGACTGGAGATTAAACGTACGGTAGCTGCCCCTTCAGTTTTTATCTTTCCGCCG
TCTGACGAGCAGTTAAAATCCGGGACCGCTTCTGTAGTTTGCCTGCTGAATAATTTTTAT
CCGCGTGAGGCTAAAGTACAATGGAAAGTCGACAATGCTTTGCAGTCGGGAAATTCACAG
GAAAGTGTTACGGAGCAGGATTCTAAAGATTCCACATATTCACTCAGCTCCACCCTTACA
CTGAGCAAAGCCGACTATGAAAAACATAAAGTTTACGCATGTGAGGTGACGCACCAAGGA
TTATCCAGTCCGGTCACAAAATCGTTTAACCGCGGTGAGTGT
ATX-P-885 Kappa LC AA
SEQ ID NO: 28
EIVLTQSPATLSLSPGERATLSCRASQSVSNYLAWYQQKPGQAPRLLIYDAYNRATGIPA
RFSGSGSGTDFTLTISSLEPEDFAVYYCQQRSNWPLTFGQGTRLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSENRGEC
ATX-P-890 VH CDR1 (Kabat)
SEQ ID NO: 29
GYTFTGYY
ATX-P-890 VH CDR2 (Kabat)
SEQ ID NO: 30
INPNSGGT
ATX-P-890 VH CDR3 (Kabat)
SEQ ID NO: 31
VRDQVQLERFDS
ATX-P-890 VH nt
SEQ ID NO: 32
CAGGTGCAGCTGGTGCAGTCTGGGGCTGAGGTGAAGAAGCCTGGGGCCTCAGTGAAGGTC
TCCTGCAAGGCTTCTGGATACACCTTCACCGGCTACTATATGCACTGGGTGCGACAGGCC
CCTGGACAAGGGCTTGAGTGGATGGGATGGATCAACCCTAACAGTGGTGGCACAAACTAT
GCACAGAAGTTTCAGGGCAGGGTCACCATGACCAGGGACACGTCCATCAGCACAGCCTAC
ATGGAGCTGAGCAGGCTGAGATCTGACGACACGGCCGTGTATTACTGTGTGAGAGATCAG
GTACAACTGGAACGGTTCGACTCCTGGGGCCAGGGAACCCTGGTCACCGTCTCCTCA
ATX-P-890 VH AA
SEQ ID NO: 33
QVQLVQSGAEVKKPGASVKVSCKASGYTFTGYYMHWVRQAPGQGLEWMGWINPNSGGTNY
AQKFQGRVTMTRDTSISTAYMELSRLRSDDTAVYYCVRDQVQLERFDSWGQGTLVTVSS
ATX-P-890 HC IgG1-Fc nt
SEQ ID NO: 34
CAGGTGCAGCTGGTGCAGTCTGGGGCTGAGGTGAAGAAGCCTGGGGCCTCAGTGAAGGTC
TCCTGCAAGGCTTCTGGATACACCTTCACCGGCTACTATATGCACTGGGTGCGACAGGCC
CCTGGACAAGGGCTTGAGTGGATGGGATGGATCAACCCTAACAGTGGTGGCACAAACTAT
GCACAGAAGTTTCAGGGCAGGGTCACCATGACCAGGGACACGTCCATCAGCACAGCCTAC
ATGGAGCTGAGCAGGCTGAGATCTGACGACACGGCCGTGTATTACTGTGTGAGAGATCAG
GTACAACTGGAACGGTTCGACTCCTGGGGCCAGGGAACCCTGGTCACCGTCTCCTCAGCT
AGCACTAAAGGGCCTTCTGTATTTCCCTTGGCCCCGTCCAGCAAATCGACCTCGGGAGGG
ACAGCCGCCCTGGGTTGCCTTGTGAAAGATTATTTCCCTGAGCCAGTTACCGTAAGTTGG
AACAGTGGGGCGCTGACAAGTGGTGTGCACACGTTTCCTGCCGTCCTGCAATCATCGGGC
TTGTATAGCCTCAGCTCTGTGGTCACTGTCCCAAGTTCATCGCTGGGCACTCAGACGTAT
ATTTGCAATGTGAACCACAAACCTTCAAATACAAAAGTGGATAAACGCGTAGAACCGAAA
TCGTGTGATAAAACTCACACATGCCCGCCATGCCCGGCACCTGAACTGCTTGGTGGTCCC
AGCGTGTTCCTGTTCCCGCCGAAGCCTAAAGATACTCTAATGATCAGCCGTACGCCAGAG
GTGACATGTGTCGTGGTTGACGTGTCCCACGAAGATCCCGAAGTTAAGTTCAATTGGTAT
GTTGATGGTGTAGAGGTACACAATGCTAAGACTAAACCTCGCGAGGAGCAGTACAATTCG
ACCTATCGTGTCGTGAGCGTTCTGACCGTCCTTCACCAAGATTGGCTTAACGGCAAAGAA
TATAAGTGCAAGGTAAGCAATAAAGCACTTCCGGCCCCAATCGAGAAAACCATTTCCAAG
GCCAAAGGTCAACCAAGAGAACCCCAGGTGTATACTCTTCCGCCTTCTCGTGAGGAAATG
ACTAAAAATCAAGTATCCCTTACGTGTCTGGTTAAAGGTTTTTATCCTAGCGATATTGCT
GTTGAATGGGAATCGAACGGTCAGCCGGAGAATAATTATAAAACAACGCCACCCGTCCTG
GATAGCGACGGCTCATTTTTTCTGTATAGCAAACTGACTGTAGATAAATCACGGTGGCAG
CAGGGCAATGTATTCAGTTGCTCCGTTATGCATGAAGCGTTACATAATCACTACACGCAG
AAATCTCTTAGTCTTTCACCCGGT
ATX-P-890 HC IgG1-Fc AA
SEQ ID NO: 35
QVQLVQSGAEVKKPGASVKVSCKASGYTFTGYYMHWVRQAPGQGLEWMGWINPNSGGTNY
AQKFQGRVTMTRDTSISTAYMELSRLRSDDTAVYYCVRDQVQLERFDSWGQGTLVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGP
SVFLFPPKPKDTLMISRTPEVTCVVVDVSHEDPEVKENWYVDGVEVHNAKTKPREEQYNS
TYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQ
QGNVFSCSVMHEALHNHYTQKSLSLSPG
ATX-P-890 VL CDR1 (Kabat)
SEQ ID NO: 36
QDISNY
ATX-P-890 VL CDR2 (Kabat)
DAS
ATX-P-890 VL CDR3 (Kabat)
SEQ ID NO: 38
QQYDNLPPT
ATX-P-890 VL nt
SEQ ID NO: 39
GACATCCAGATGACCCAGTCTCCATCCTCCCTGTCTGCATCTGTAGGAGACAGAGTCACC
ATCACTTGCCAGGCGAGTCAGGACATTAGCAACTATTTAAATTGGTATCAGCAGAAACCA
GGGAAAGCCCCTAAGCTCCTGATCTACGATGCATCCAATTTGGAAACAGGGGTCCCATCA
AGGTTCAGTGGAAGTGGATCTGGGACAGATTTTACTTTCACCATCAGCAGCCTGCAGCCT
GAAGATATTGCAACATATTACTGTCAACAGTATGATAATCTCCCTCCCACTTTCGGCCCT
GGGACCAAGGTGGAAATCAAA
ATX-P-890 VL AA
SEQ ID NO: 40
DIQMTQSPSSLSASVGDRVTITCQASQDISNYLNWYQQKPGKAPKLLIYDASNLETGVPS
RFSGSGSGTDFTFTISSLQPEDIATYYCQQYDNLPPTFGPGTKVEIK
ATX-P-890 Kappa LC nt
SEQ ID NO: 41
GACATCCAGATGACCCAGTCTCCATCCTCCCTGTCTGCATCTGTAGGAGACAGAGTCACC
ATCACTTGCCAGGCGAGTCAGGACATTAGCAACTATTTAAATTGGTATCAGCAGAAACCA
GGGAAAGCCCCTAAGCTCCTGATCTACGATGCATCCAATTTGGAAACAGGGGTCCCATCA
AGGTTCAGTGGAAGTGGATCTGGGACAGATTTTACTTTCACCATCAGCAGCCTGCAGCCT
GAAGATATTGCAACATATTACTGTCAACAGTATGATAATCTCCCTCCCACTTTCGGCCCT
GGGACCAAGGTGGAAATCAAACGTACGGTAGCTGCCCCTTCAGTTTTTATCTTTCCGCCG
TCTGACGAGCAGTTAAAATCCGGGACCGCTTCTGTAGTTTGCCTGCTGAATAATTTTTAT
CCGCGTGAGGCTAAAGTACAATGGAAAGTCGACAATGCTTTGCAGTCGGGAAATTCACAG
GAAAGTGTTACGGAGCAGGATTCTAAAGATTCCACATATTCACTCAGCTCCACCCTTACA
CTGAGCAAAGCCGACTATGAAAAACATAAAGTTTACGCATGTGAGGTGACGCACCAAGGA
TTATCCAGTCCGGTCACAAAATCGTTTAACCGCGGTGAGTGT
ATX-P-890 Kappa LC AA
SEQ ID NO: 42
DIQMTQSPSSLSASVGDRVTITCQASQDISNYLNWYQQKPGKAPKLLIYDASNLETGVPS
RFSGSGSGTDFTFTISSLQPEDIATYYCQQYDNLPPTFGPGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSENRGEC

1. An immunoconjugate having Formula (I), 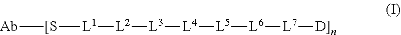
or a pharmaceutically acceptable salt thereof, wherein;
Ab is an antibody or an antigen-binding fragment;
L¹ is 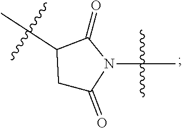 [SMILES: *C1CC(=O)N(*)C1=O]
L² is absent, 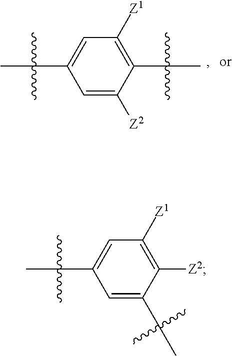
Z¹ and Z² are each individually hydrogen, halogen, —NO₂, —O—(C₁-C₆ alkyl), or C₁-C₆ alkyl;
L³ is —(CH₂)n¹-C(═O)— or —(CH₂CH₂O)n¹-(CH₂)n¹C(═O)—;
n¹ are independently integers of 0 to 12;
L⁴ is a tetrapeptide residue;
L⁵ is absent or —[NH(CH₂)n²]n³-;
n² is an integer of 0 to 6;
n³ is an integer of 0 to 2;
L⁶ is absent or 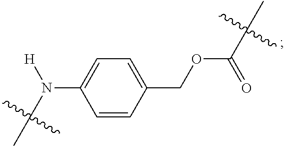 [SMILES: *Nc1ccc(COC(*)=O)cc1]
L⁷ is absent, 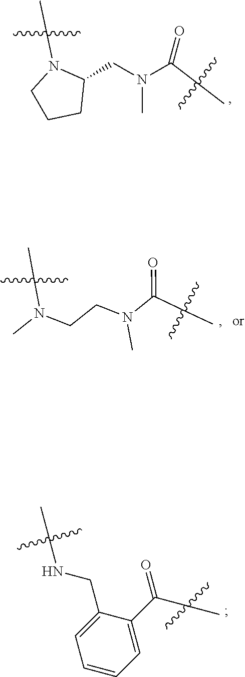
D is a drug moiety; and
n is an integer from 1 to 10;
wherein D is a drug moiety of Formula (II) having the structure: 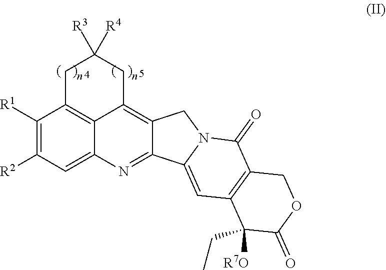
wherein:
R¹ and R² are each individually selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)]ₜCY₃, or a substituted or an unsubstituted —O—(CR⁵R⁶)ₘ—O— such that R¹ and R² taken together form a ring;
R³ is selected from the group consisting of hydrogen, —OH, —N₃, —NH₂, —NH(C═O)—CH₂—R^{3D}, a substituted or an unsubstituted C₁-C₆ alkyl, a substituted or an unsubstituted C₂-C₆ alkenyl and [(CY₂)ₚO(CY₂)_{q}]ₜOH;
R⁴ is selected from —(C₁-C₆ alkyl)-X² and —(C₂-C₆ alkenyl)-X², wherein each of —(C₁-C₆ alkyl)-X² and —(C₂-C₆ alkenyl)-X² is substituted with one or more R^{3A} groups selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl);
R^{3D} is selected from the group consisting of H, —CH₃, —OH and —CH₂Y¹, wherein Y¹ is halogen;
X² is —OR⁹, —SR⁹, or —NHR⁹;
R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl;
n⁴ and n⁵ are each individually 0, 1 or 2, with the proviso that n⁴ and n⁵ are not both 0;
each Y is individually H or halogen;
each m is individually 1 or 2;
each p is individually 1, 2, 3, 4, 5, or 6;
each q is individually 0, 1, 2, 3, 4, 5, or 6;
each t is individually 1, 2, 3, 4, 5, or 6;
R⁷ is H, —COR⁸, —CO₂R⁸, —(CO)—NHR⁸, L⁴, L⁵, L⁶, or L⁷;
R⁸ is a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³;
R⁹ is H, —COR⁸, —CO₂R⁸, —(CO)—NHR⁸, L⁴, L⁵, L⁶, or L⁷, with the proviso that exactly one of R⁷ and R⁹ is L⁴, L⁵, L⁶, or L⁷; and
each X³ is individually —H, —OH, —SH, or —NH₂.

2-11. (canceled)

12. The immunoconjugate of claim 1, or a pharmaceutically acceptable salt thereof, wherein L⁴ is gly-gly-phe-gly (GGFG).

13-21. (canceled)

22. The immunoconjugate of claim 21, or a pharmaceutically acceptable salt thereof, wherein R¹ is C₁-C₃ alkyl and R² is a halogen.

23. The immunoconjugate of claim 21, or a pharmaceutically acceptable salt thereof, wherein R¹ is methyl and R² is F.

24-34. (canceled)

35. The immunoconjugate of claim 1, or a pharmaceutically acceptable salt thereof, wherein R^{3B} and R^{3C} are each a substituted or an unsubstituted C₁-C₆ alkyl.

36-55. (canceled)

56. The immunoconjugate of claim 1, wherein Formula (I) is selected from the group consisting of: 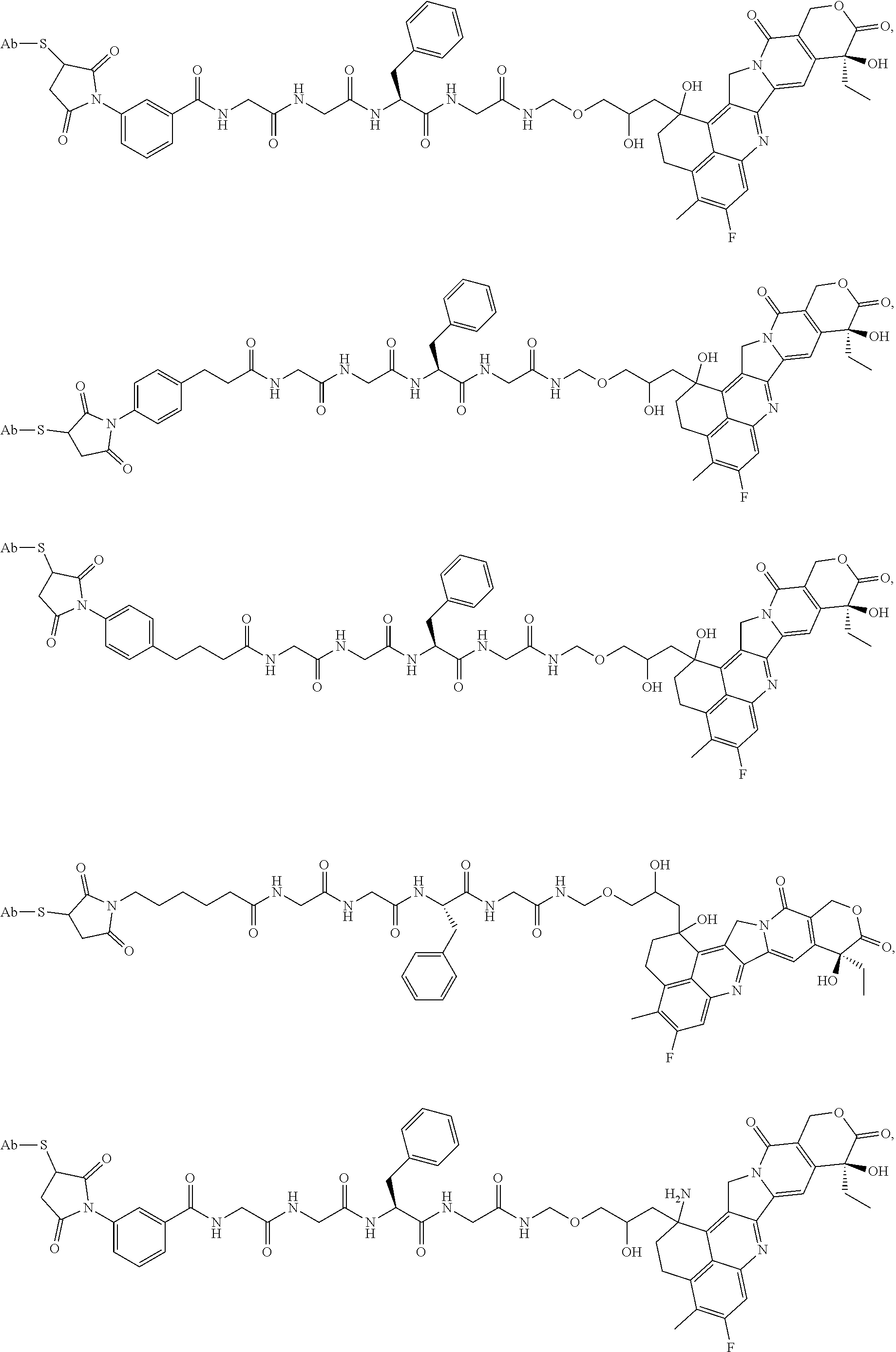 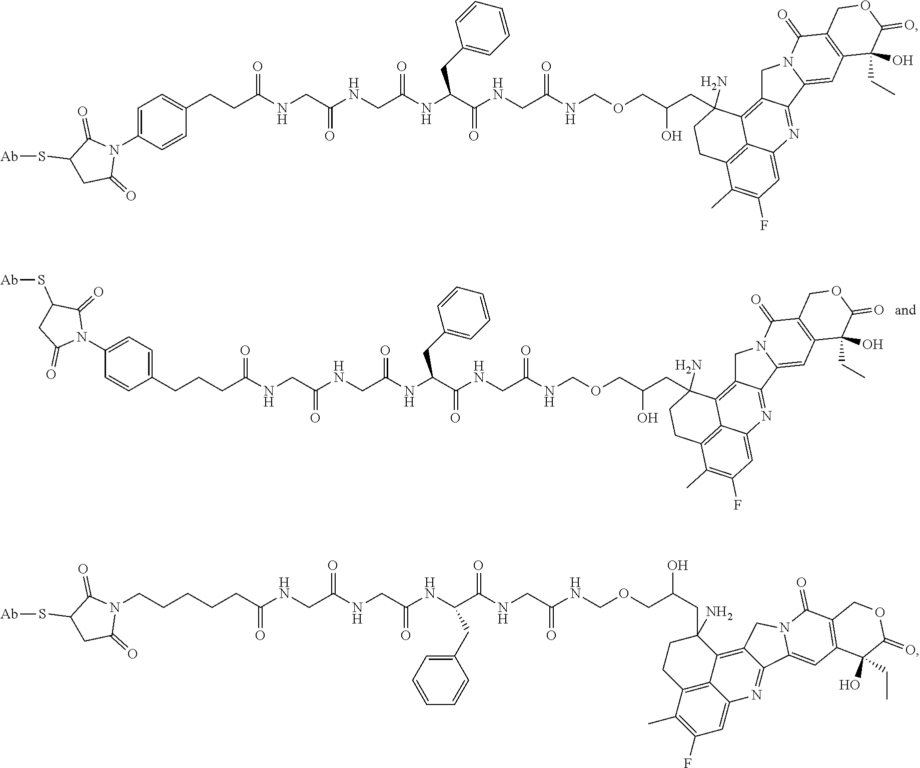
or a pharmaceutically acceptable salt of any of the foregoing.

57. The immunoconjugate of claim 1, wherein Formula (I) is selected from the group consisting of: 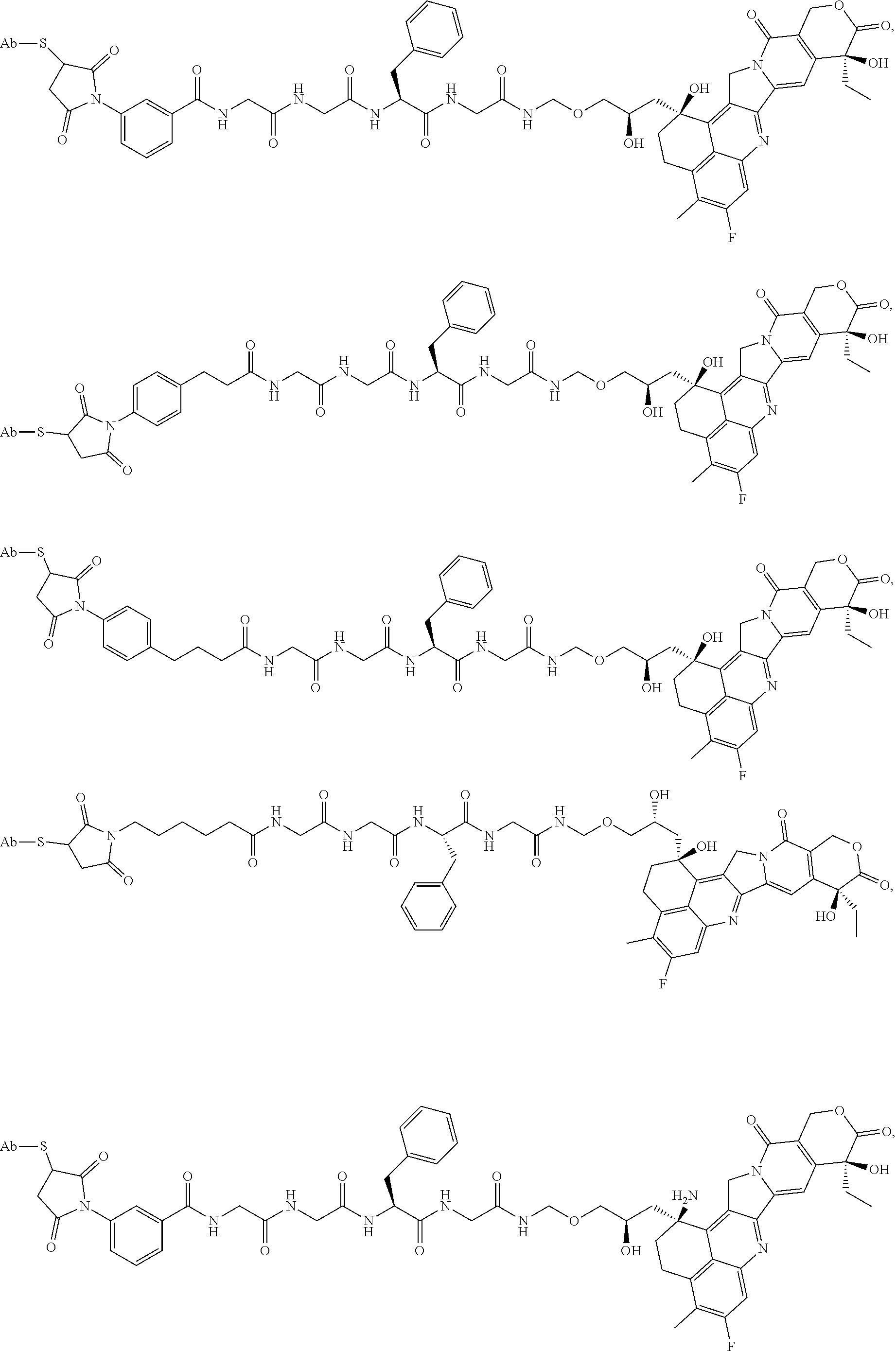 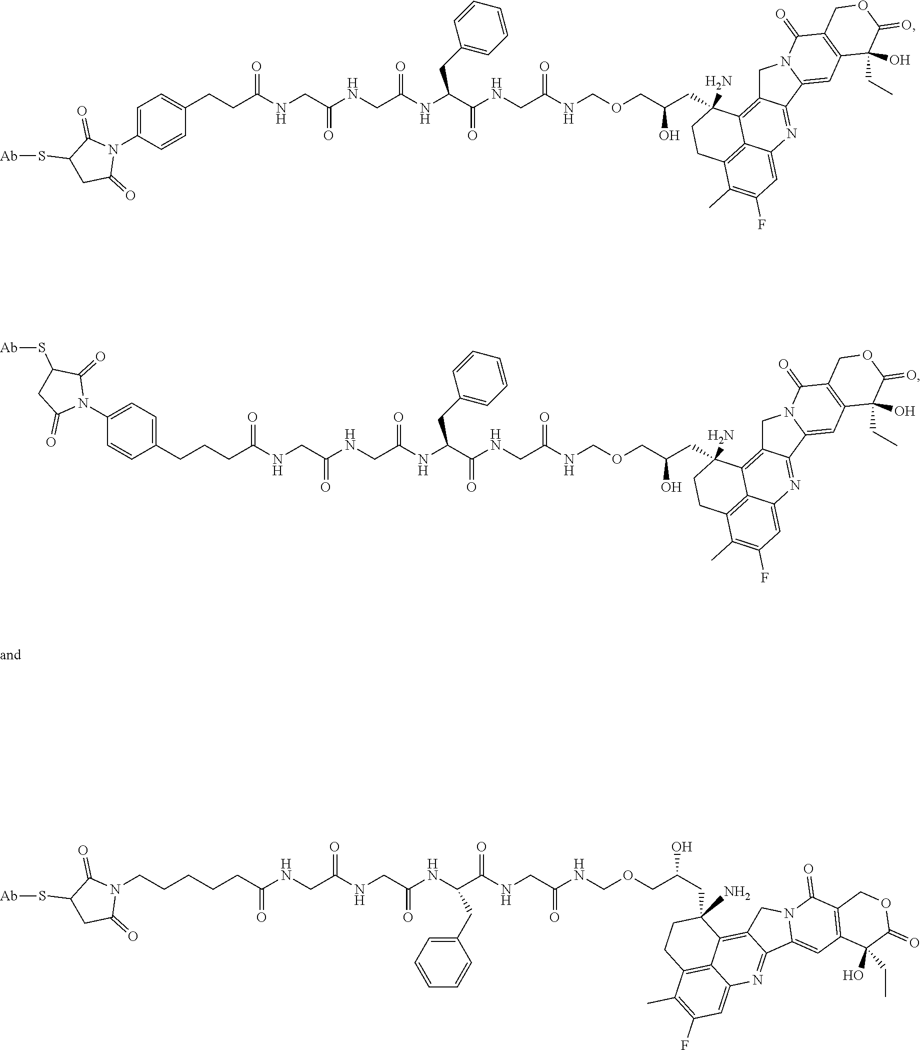
or a pharmaceutically acceptable salt of any of the foregoing.

58. The immunoconjugate of claim 1, wherein Formula (I) is selected from the group consisting of: 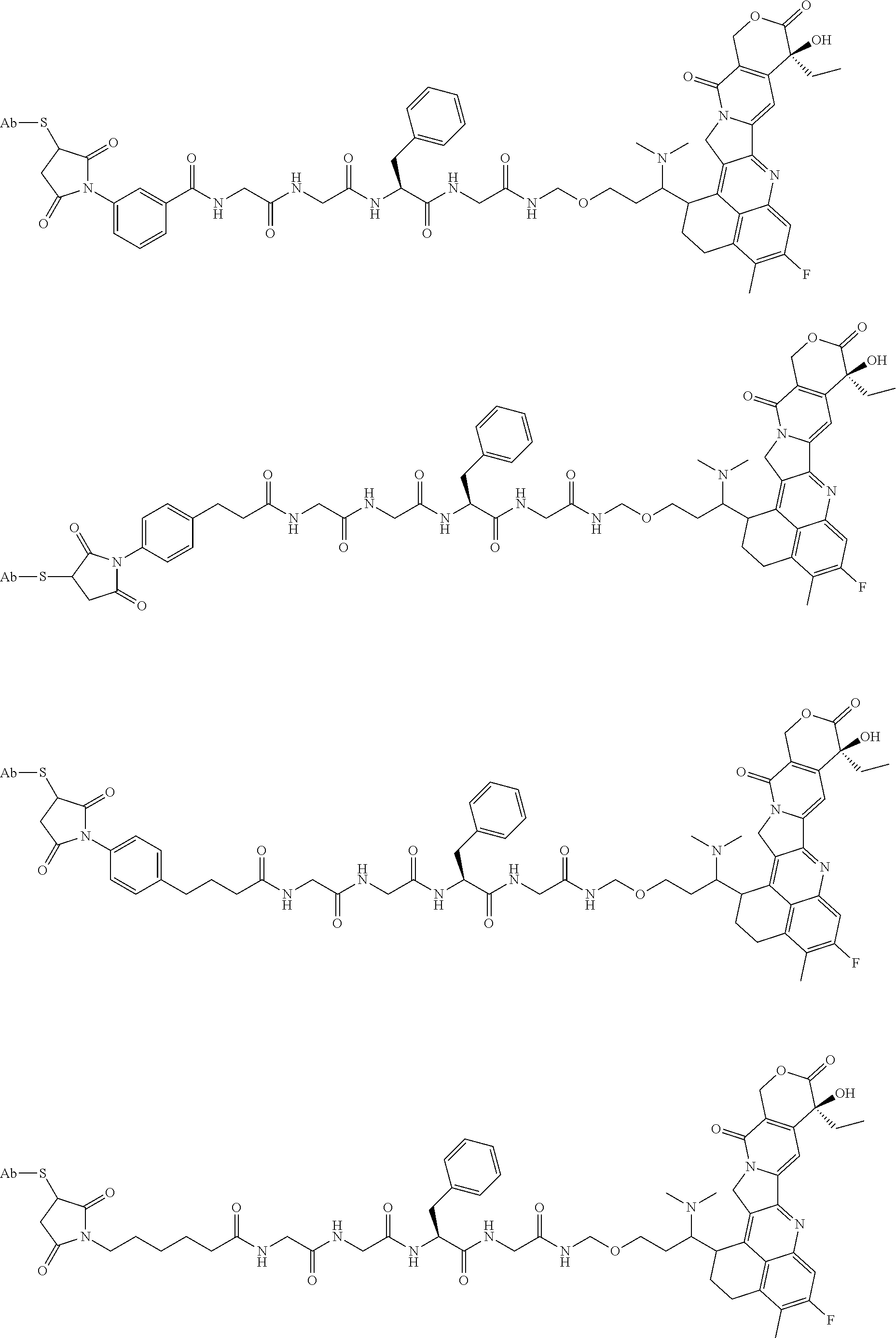 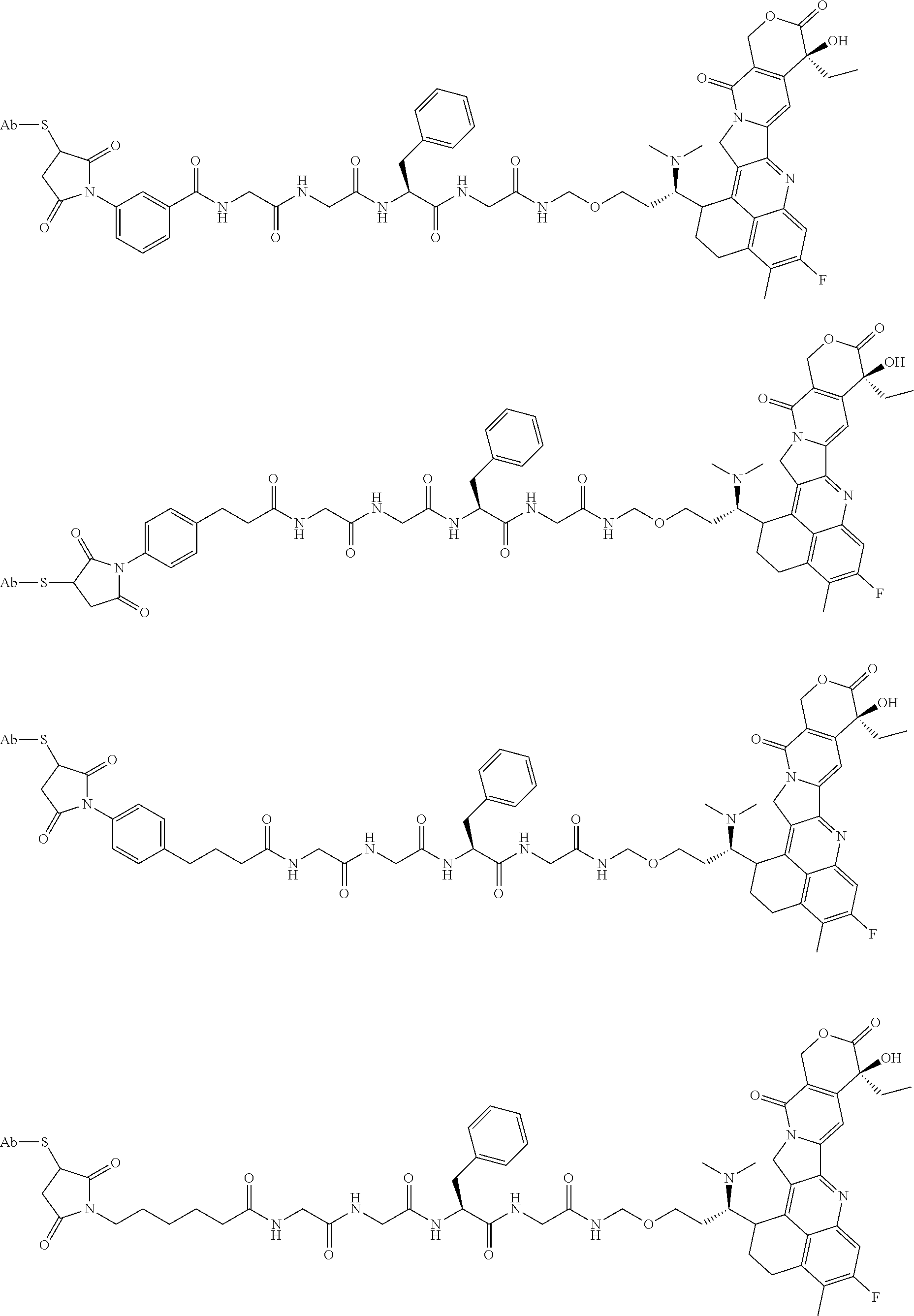 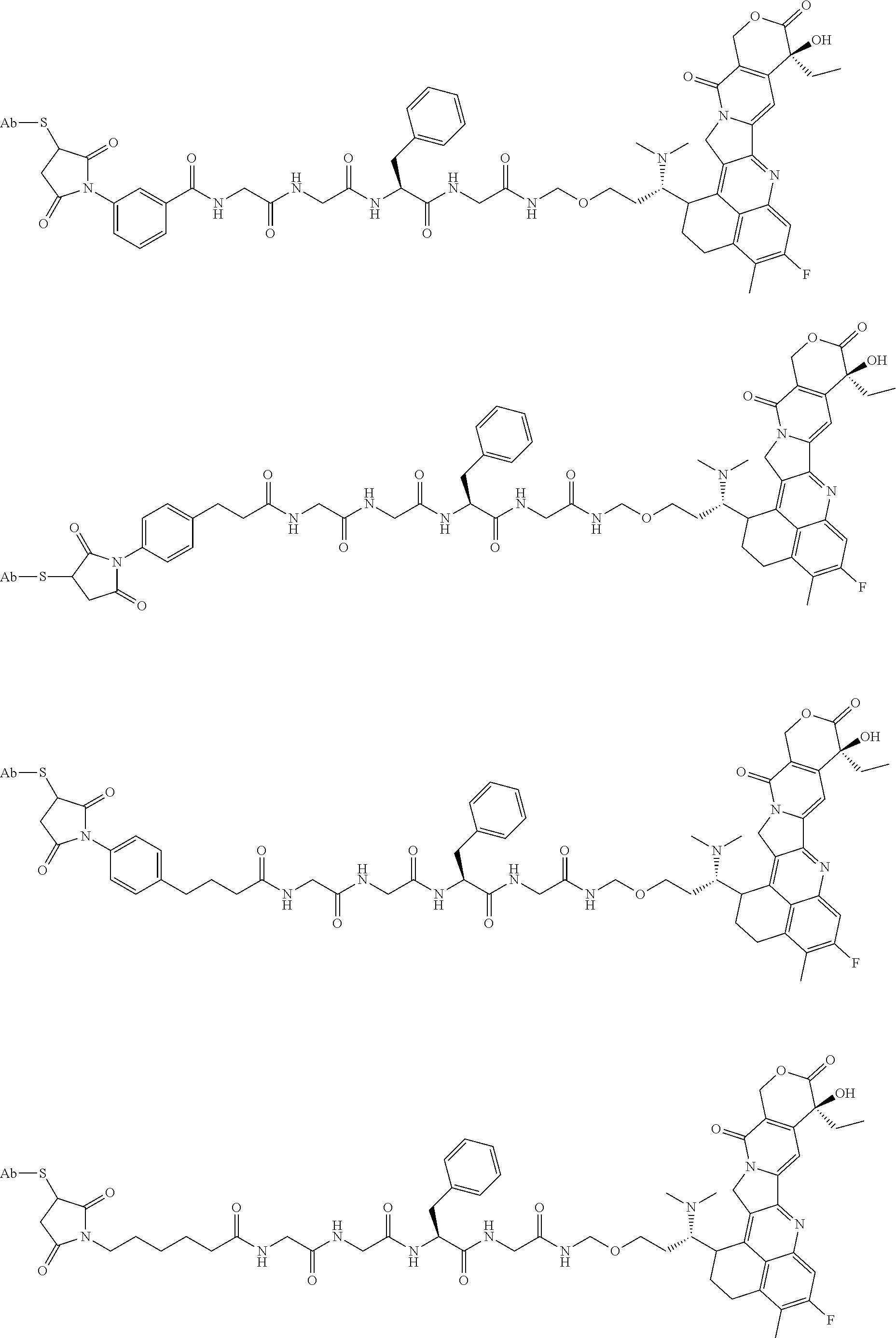 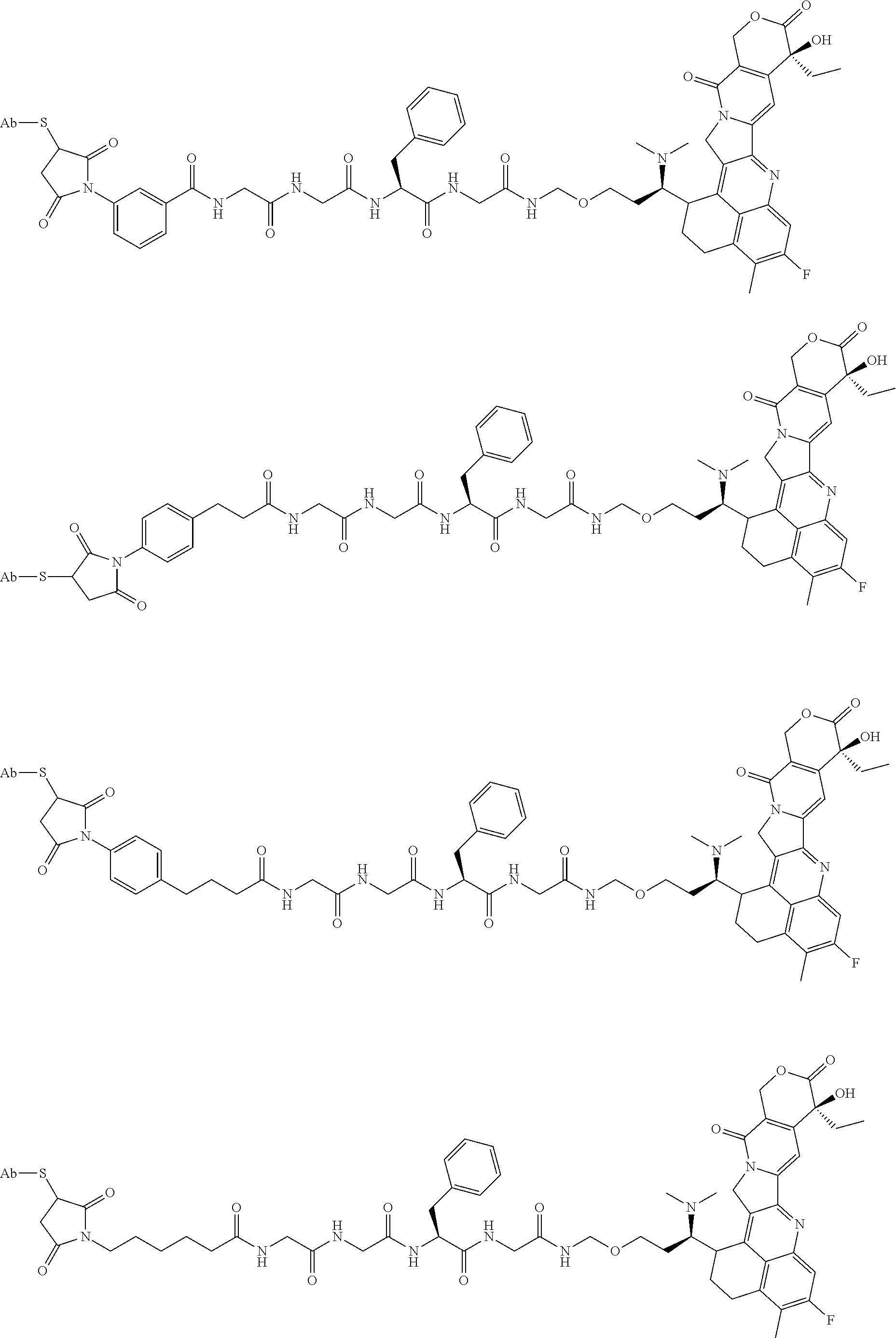 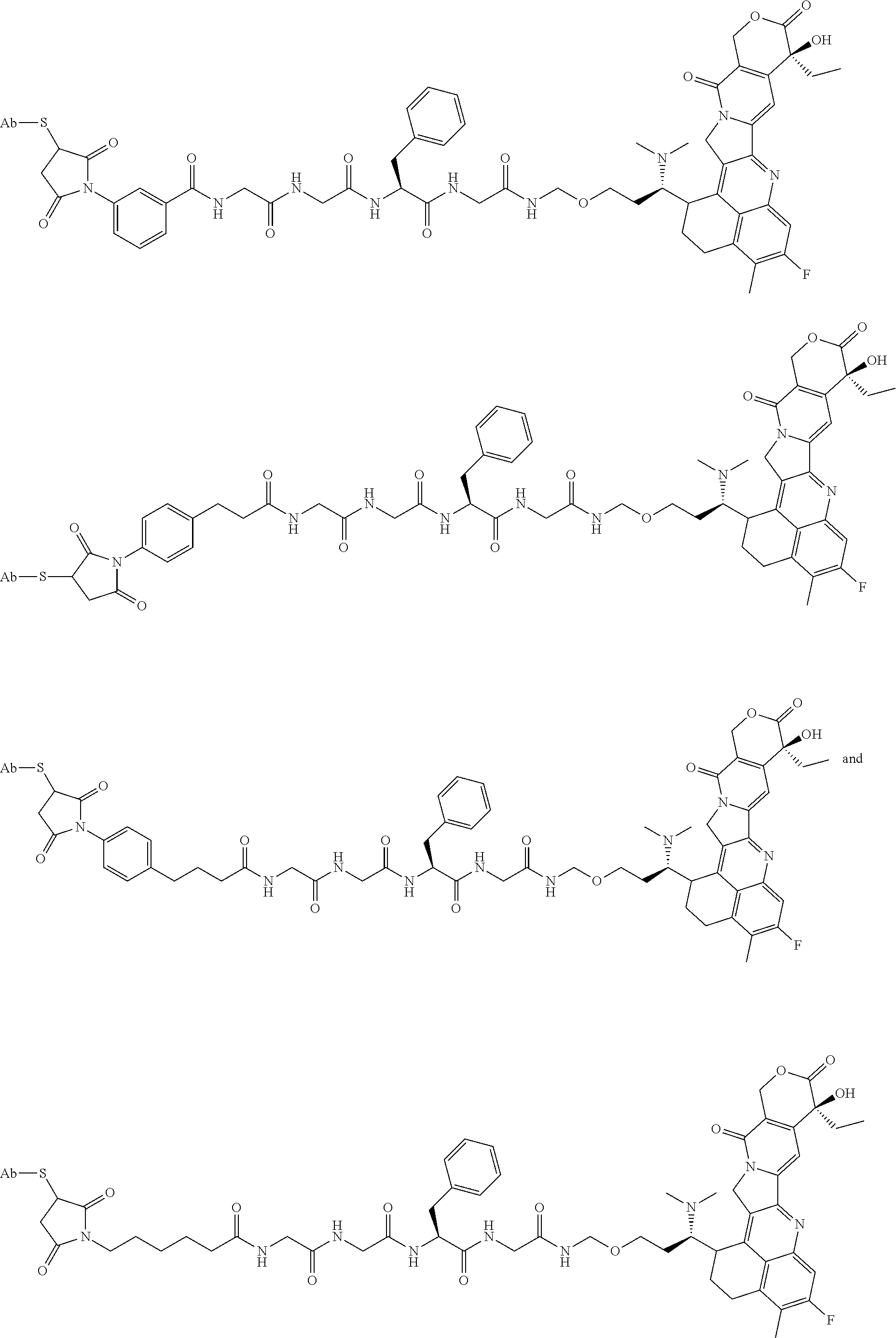
or a pharmaceutically acceptable salt of any of the foregoing.

59. A compound of Formula (IV), or a pharmaceutically acceptable salt thereof, having the structure: 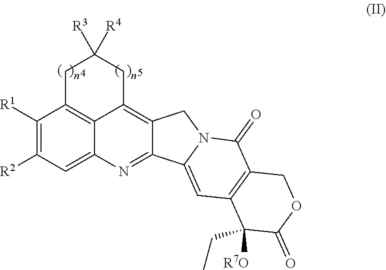
wherein:
R¹ and R² are each individually selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, or a substituted or an unsubstituted —O—(CR⁵R⁶)ₘ—O— such that R¹ and R² taken together form a ring;
R³ is selected from the group consisting of hydrogen, —OH, —N₃, —NH₂, —NH(C═O)—CH₂—R^{3D}, a substituted or an unsubstituted C₁-C₆ alkyl, a substituted or an unsubstituted C₂-C₆ alkenyl and [(CY₂)ₚO(CY₂)_{q}]ₜH;
R⁴ is selected from C₁-C₆ alkyl and C₂-C₆ alkenyl, wherein each of C₁-C₆ alkyl and C₂-C₆ alkenyl is substituted with one or more R^{3A} groups selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl);
R^{3D} is selected from the group consisting of H, —CH₃, —OH and —CH₂Y¹, wherein Y¹ is halogen;
R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl;
n⁴ and n⁵ are each individually 0, 1 or 2, with the proviso that n⁴ and n⁵ are not both 0;
each Y is individually H or halogen;
each m is individually 1 or 2;
each p is individually 1, 2, 3, 4, 5, or 6;
each q is individually 0, 1, 2, 3, 4, 5, or 6; and
each t is individually 1, 2, 3, 4, 5, or 6;
R⁷ is H, —COR⁸, —CO₂R⁸, or —(CO)—NHR⁸; and
R⁸ is a substituted or an unsubstituted C₁-C₆ alkyl, a substituted or an unsubstituted C₁-C₆ haloalkyl, or —[(CY₂)ₚO(CY₂)_{q}]ₜCY₃.

60. The compound of claim 59, or a pharmaceutically acceptable salt thereof, wherein R¹ is C₁-C₃ alkyl and R² is a halogen.

61. The compound of claim 59, or a pharmaceutically acceptable salt thereof, wherein R¹ is methyl and R² is F.

62-72. (canceled)

73. The compound of claim 69, or a pharmaceutically acceptable salt thereof, wherein R^{3B} and R^{3C} are each a substituted or an unsubstituted C₁-C₆ alkyl.

74-82. (canceled)

83. The compound of claim 59, or a pharmaceutically acceptable salt thereof, wherein Formula (IV) has the structure of Formula (IV-a) having the structure: 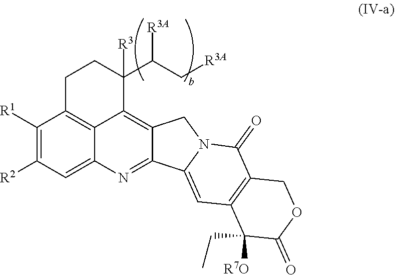
wherein b is 1, 2 or 3.

84. The compound of claim 83, or a pharmaceutically acceptable salt thereof, wherein each R^{3A} is independently-OH.

85. (canceled)

86. The compound of claim 83, or a pharmaceutically acceptable salt thereof, wherein each R^{3A} is independently-NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl).

87-91. (canceled)

92. The compound of claim 59, or a pharmaceutically acceptable salt thereof, wherein the compound of Formula (IV) is selected from the group consisting of:

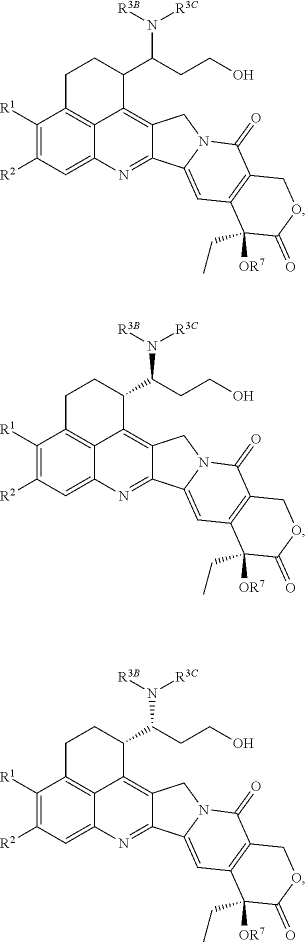

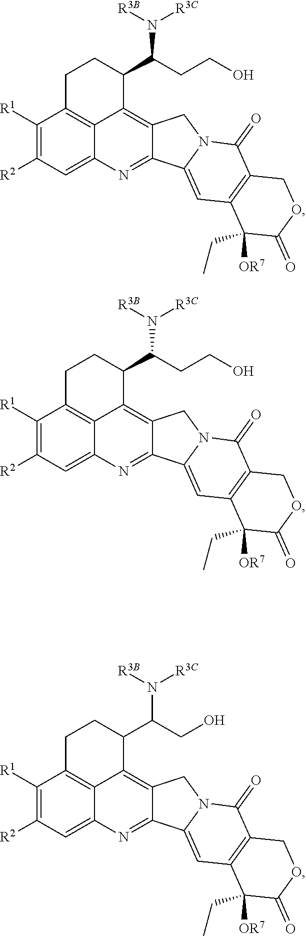

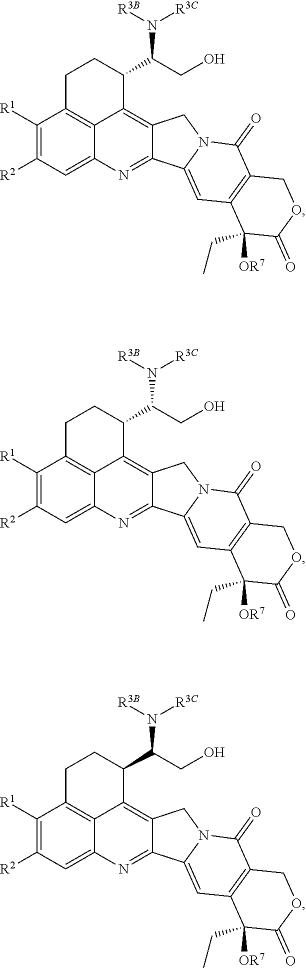

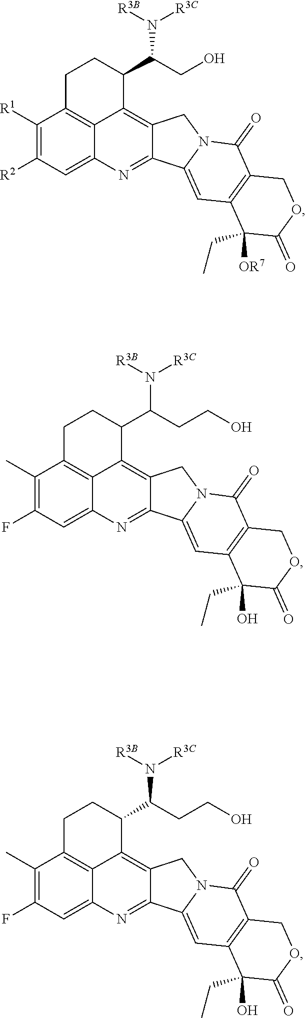

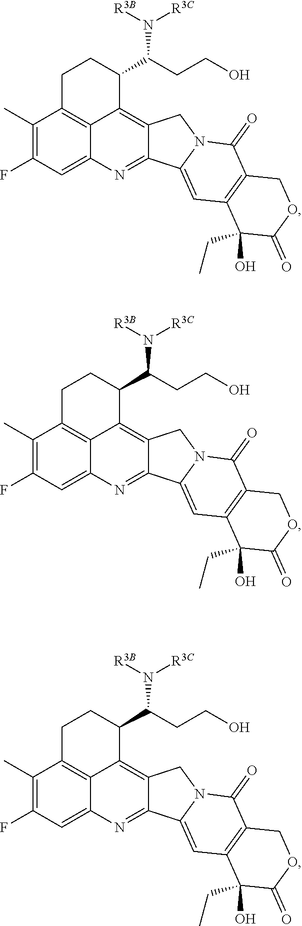

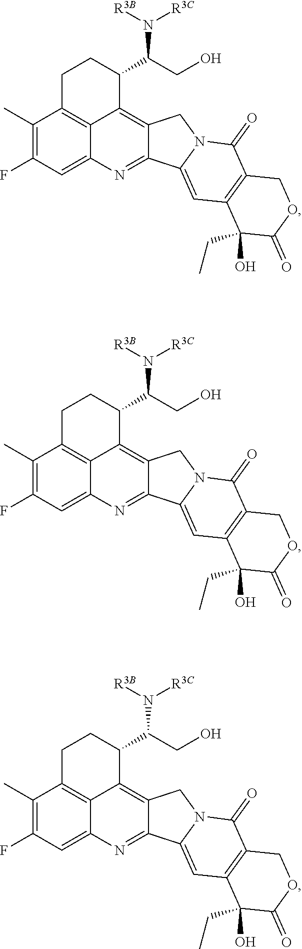

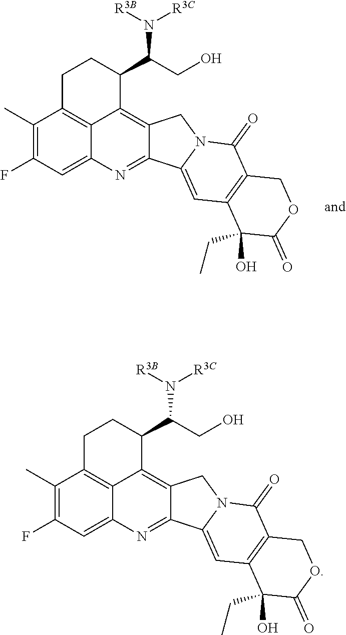

93. The compound of claim 59, or a pharmaceutically acceptable salt thereof, wherein the compound of Formula (IV) is selected from the group consisting of:

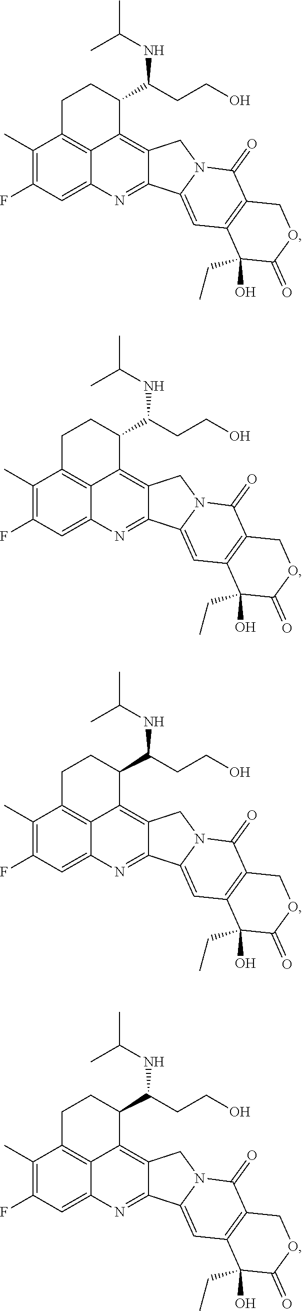 [SMILES: CC[C@@]1(O)C(=O)OCc2c1cc1n(c2=O)Cc2c-1nc1cc(F)c(C)c3c1c2[C@@H]([C@H](CCO)NC(C)C)CC3 ; CC[C@@]1(O)C(=O)OCc2c1cc1n(c2=O)Cc2c-1nc1cc(F)c(C)c3c1c2[C@@H]([C@@H](CCO)NC(C)C)CC3 ; CC[C@@]1(O)C(=O)OCc2c1cc1n(c2=O)Cc2c-1nc1cc(F)c(C)c3c1c2[C@H]([C@H](CCO)NC(C)C)CC3 ; CC[C@@]1(O)C(=O)OCc2c1cc1n(c2=O)Cc2c-1nc1cc(F)c(C)c3c1c2[C@H]([C@@H](CCO)NC(C)C)CC3]

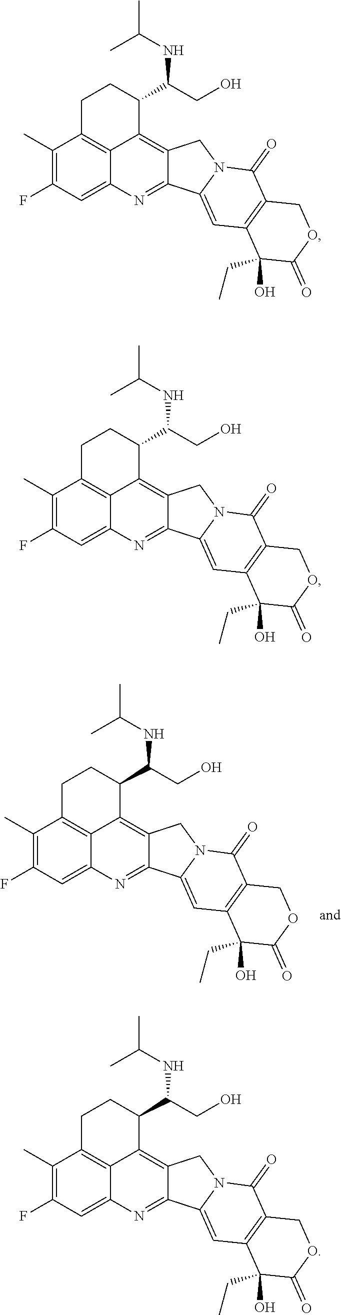 [SMILES: CC[C@@]1(O)C(=O)OCc2c1cc1n(c2=O)Cc2c-1nc1cc(F)c(C)c3c1c2[C@@H]([C@H](CO)NC(C)C)CC3 ; CC[C@@]1(O)C(=O)OCc2c1cc1n(c2=O)Cc2c-1nc1cc(F)c(C)c3c1c2[C@@H]([C@@H](CO)NC(C)C)CC3 ; CC[C@@]1(O)C(=O)OCc2c1cc1n(c2=O)Cc2c-1nc1cc(F)c(C)c3c1c2[C@H]([C@H](CO)NC(C)C)CC3 ; CC[C@@]1(O)C(=O)OCc2c1cc1n(c2=O)Cc2c-1nc1cc(F)c(C)c3c1c2[C@H]([C@@H](CO)NC(C)C)CC3]

94. The compound of claim 59, or a pharmaceutically acceptable salt thereof, wherein the compound of Formula (IV) is selected from the group consisting of;

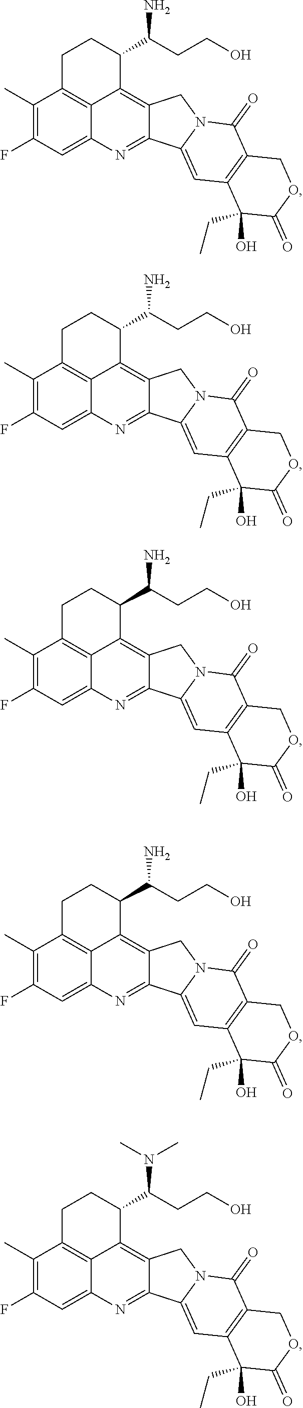 [SMILES: CC[C@@]1(O)C(=O)OCc2c1cc1n(c2=O)Cc2c-1nc1cc(F)c(C)c3c1c2[C@@H]([C@@H](N)CCO)CC3 ; CC[C@@]1(O)C(=O)OCc2c1cc1n(c2=O)Cc2c-1nc1cc(F)c(C)c3c1c2[C@@H]([C@H](N)CCO)CC3 ; CC[C@@]1(O)C(=O)OCc2c1cc1n(c2=O)Cc2c-1nc1cc(F)c(C)c3c1c2[C@H]([C@@H](N)CCO)CC3 ; CC[C@@]1(O)C(=O)OCc2c1cc1n(c2=O)Cc2c-1nc1cc(F)c(C)c3c1c2[C@H]([C@H](N)CCO)CC3 ; CC[C@@]1(O)C(=O)OCc2c1cc1n(c2=O)Cc2c-1nc1cc(F)c(C)c3c1c2[C@@H]([C@H](CCO)N(C)C)CC3]

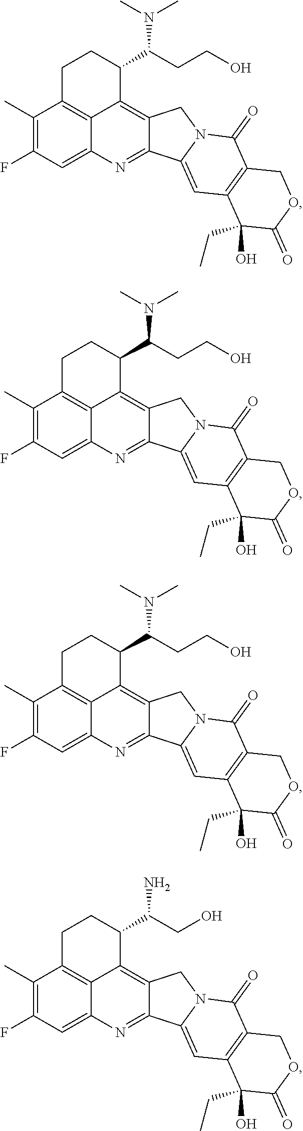

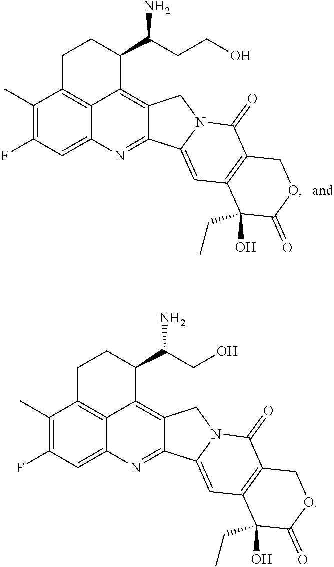 [SMILES: CC[C@@]1(O)C(=O)OCc2c1cc1n(c2=O)Cc2c-1nc1cc(F)c(C)c3c1c2[C@H]([C@@H](N)CCO)CC3 ; "and" ; CC[C@@]1(O)C(=O)OCc2c1cc1n(c2=O)Cc2c-1nc1cc(F)c(C)c3c1c2[C@H]([C@H](N)CO)CC3]

95. The compound of claim 59, or a pharmaceutically acceptable salt thereof, wherein the compound of Formula (IV) is selected from the group consisting of:

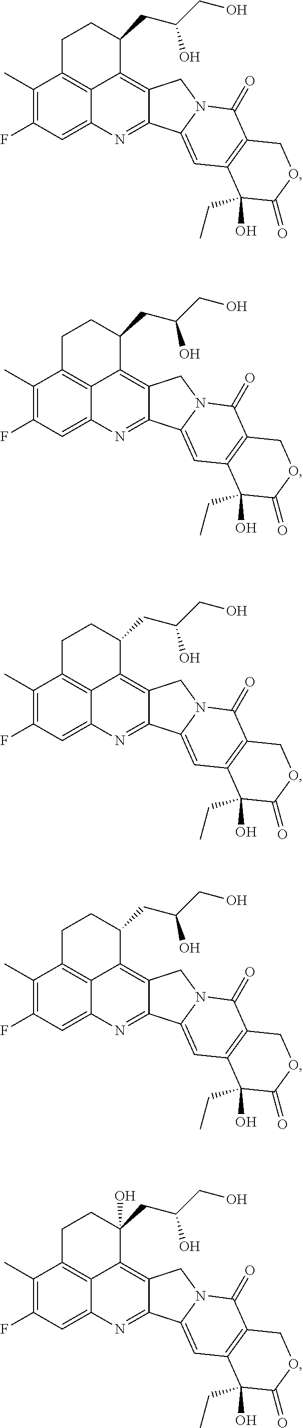 [SMILES: CC[C@@]1(O)C(=O)OCc2c1cc1n(c2=O)Cc2c-1nc1cc(F)c(C)c3c1c2[C@H](C[C@@H](O)CO)CC3 ; CC[C@@]1(O)C(=O)OCc2c1cc1n(c2=O)Cc2c-1nc1cc(F)c(C)c3c1c2[C@H](C[C@H](O)CO)CC3 ; CC[C@@]1(O)C(=O)OCc2c1cc1n(c2=O)Cc2c-1nc1cc(F)c(C)c3c1c2[C@@H](C[C@@H](O)CO)CC3 ; CC[C@@]1(O)C(=O)OCc2c1cc1n(c2=O)Cc2c-1nc1cc(F)c(C)c3c1c2[C@@H](C[C@H](O)CO)CC3 ; CC[C@@]1(O)C(=O)OCc2c1cc1n(c2=O)Cc2c-1nc1cc(F)c(C)c3c1c2[C@](O)(C[C@@H](O)CO)CC3]

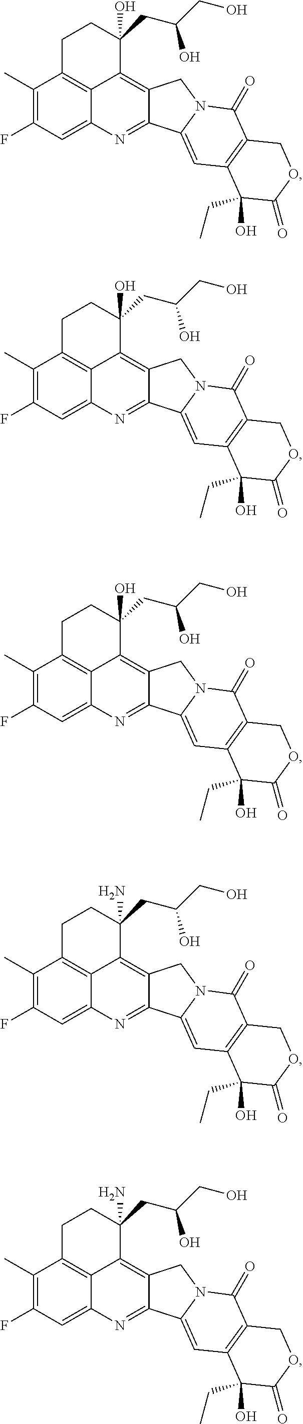

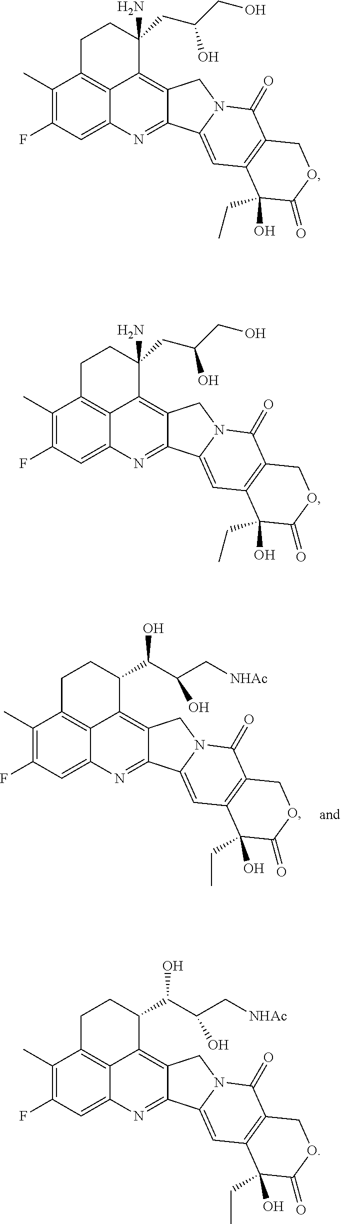 [SMILES: CC[C@@]1(O)C(=O)OCc2c1cc1n(c2=O)Cc2c-1nc1cc(F)c(C)c3c1c2[C@@](N)(C[C@@H](O)CO)CC3 ; CC[C@@]1(O)C(=O)OCc2c1cc1n(c2=O)Cc2c-1nc1cc(F)c(C)c3c1c2[C@@](N)(C[C@H](O)CO)CC3 ; CC[C@@]1(O)C(=O)OCc2c1cc1n(c2=O)Cc2c-1nc1cc(F)c(C)c3c1c2[C@@H]([C@@H](O)[C@H](O)CNC(C)=O)CC3 ; CC[C@@]1(O)C(=O)OCc2c1cc1n(c2=O)Cc2c-1nc1cc(F)c(C)c3c1c2[C@@H]([C@H](O)[C@@H](O)CNC(C)=O)CC3]

96. The compound of claim 59, or a pharmaceutically acceptable salt thereof, wherein the compound of Formula (IV) is selected from the group consisting of;

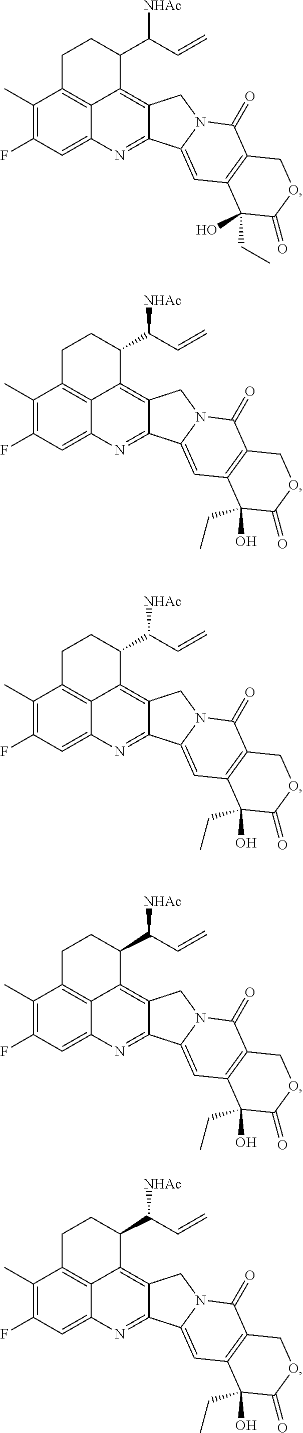 [SMILES: C=CC(NC(C)=O)C1CCc2c(C)c(F)cc3nc4c(c1c23)Cn1c-4cc2c(c1=O)COC(=O)[C@]2(O)CC ; C=C[C@H](NC(C)=O)[C@H]1CCc2c(C)c(F)cc3nc4c(c1c23)Cn1c-4cc2c(c1=O)COC(=O)[C@]2(O)CC ; C=C[C@@H](NC(C)=O)[C@H]1CCc2c(C)c(F)cc3nc4c(c1c23)Cn1c-4cc2c(c1=O)COC(=O)[C@]2(O)CC ; C=C[C@H](NC(C)=O)[C@@H]1CCc2c(C)c(F)cc3nc4c(c1c23)Cn1c-4cc2c(c1=O)COC(=O)[C@]2(O)CC ; C=C[C@@H](NC(C)=O)[C@@H]1CCc2c(C)c(F)cc3nc4c(c1c23)Cn1c-4cc2c(c1=O)COC(=O)[C@]2(O)CC]

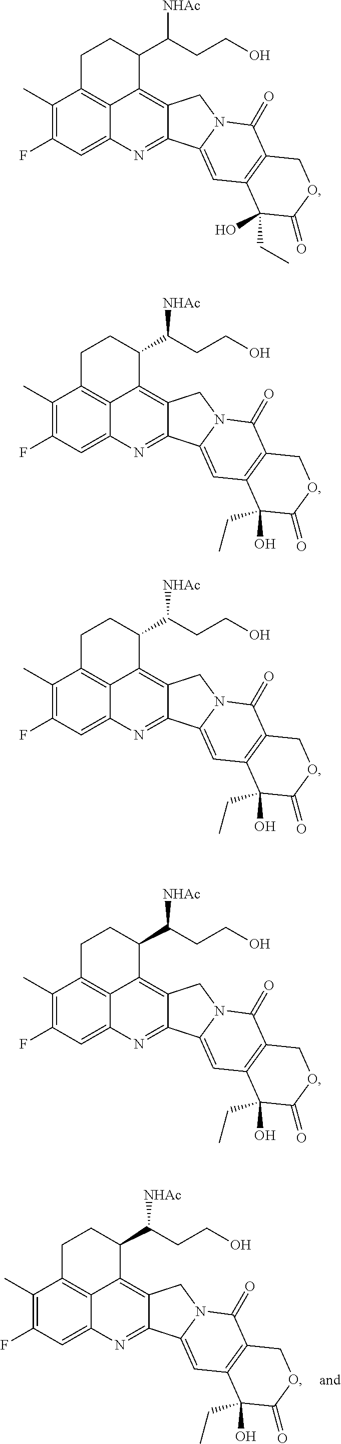 [SMILES: CC[C@@]1(O)C(=O)OCc2c1cc1n(c2=O)Cc2c-1nc1cc(F)c(C)c3c1c2C(C(CCO)NC(C)=O)CC3 ; CC[C@@]1(O)C(=O)OCc2c1cc1n(c2=O)Cc2c-1nc1cc(F)c(C)c3c1c2[C@@H]([C@H](CCO)NC(C)=O)CC3 ; CC[C@@]1(O)C(=O)OCc2c1cc1n(c2=O)Cc2c-1nc1cc(F)c(C)c3c1c2[C@@H]([C@@H](CCO)NC(C)=O)CC3 ; CC[C@@]1(O)C(=O)OCc2c1cc1n(c2=O)Cc2c-1nc1cc(F)c(C)c3c1c2[C@H]([C@H](CCO)NC(C)=O)CC3 ; CC[C@@]1(O)C(=O)OCc2c1cc1n(c2=O)Cc2c-1nc1cc(F)c(C)c3c1c2[C@H]([C@@H](CCO)NC(C)=O)CC3]

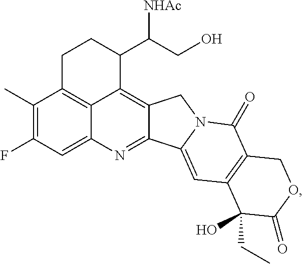 [SMILES: CC[C@@]1(O)C(=O)OCc2c1cc1n(c2=O)Cc2c-1nc1cc(F)c(C)c3c1c2C(C(CO)NC(C)=O)CC3]

97-98. (canceled)

99. A pharmaceutical composition comprising an immunoconjugate of claim 1, or a pharmaceutically active salt thereof, and a pharmaceutically acceptable carrier, diluent, excipient or combination thereof.

100. A method for treating, inhibiting, or ameliorating a cancer or a tumor comprising administering an effective amount of an immunoconjugate of claim 1, to a subject having the cancer or the tumor.

101. The method of claim 100, wherein the cancer or the tumor is selected from lung cancer, urothelial cancer, colorectal cancer, prostate cancer, ovarian cancer, pancreatic cancer, breast cancer, bladder cancer, gastric cancer, gastrointestinal stromal tumor, uterine cervix cancer, esophageal cancer, squamous cell carcinoma, peritoneal cancer, liver cancer, hepatocellular cancer, colon cancer, rectal cancer, colorectal cancer, endometrial cancer, uterine cancer, salivary gland cancer, kidney cancer, vulval cancer, thyroid cancer, penis cancer, leukemia, malignant lymphoma, plasmacytoma, myeloma, or sarcoma.

102-103. (canceled)

104. A conjugate having Formula (III), 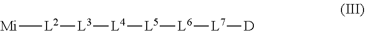
or a pharmaceutically acceptable salt thereof, wherein:
Mi is 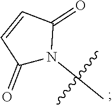 [SMILES: *N1C(=O)C=CC1=O]
L² is absent, 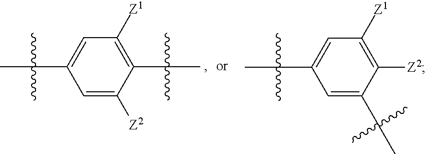
Z¹ and Z² are each individually hydrogen, halogen, —NO₂, —O—(C₁-C₆ alkyl), or C₁-C₆ alkyl;
L³ is —(CH₂)n¹-C(═O)— or —(CH₂CH₂O) n¹-(CH₂)n¹C(═O)—;
n¹ are independently integers of 0 to 12;
L⁴ is a tetrapeptide residue;
L⁵ is absent or —[NH(CH₂)n²]n³-;
n² is an integer of 0 to 6;
n³ is an integer of 0 to 2;
L⁶ is absent or 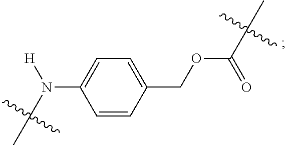 [SMILES: *Nc1ccc(COC(*)=O)cc1]
L⁷ is absent, 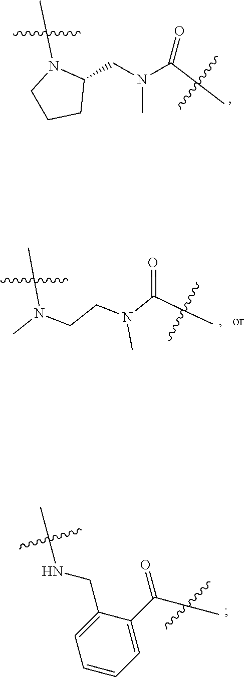
and
D is a drug moiety of Formula (II) having the structure: 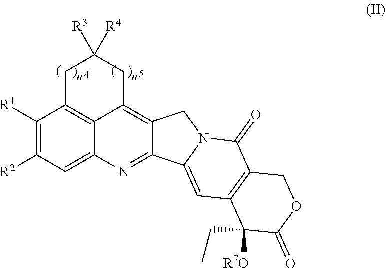
wherein:
R¹ and R² are each individually selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, or a substituted or an unsubstituted —O—(CR⁵R⁶)ₘ—O— such that R¹ and R² taken together form a ring;
R³ is selected from the group consisting of hydrogen, —OH, —N₃, —NH₂, —NH(C═O)—CH₂—R^{3D}, a substituted or an unsubstituted C₁-C₆ alkyl, a substituted or an unsubstituted C₂-C₆ alkenyl and [(CY₂)ₚO(CY₂)_{q}]ₜOH;
R⁴ is selected from —(C₁-C₆ alkyl)-X² and —(C₂-C₆ alkenyl)-X², wherein each of —(C₁-C₆ alkyl)-X² and (C₂-C₆ alkenyl)-X² is substituted with one or more R^{3A} groups selected from —OH and —NR^{3B}R^{3C}, wherein R^{3B} and R^{3C} are each individually hydrogen, a substituted or an unsubstituted C₁-C₆ alkyl or —C(═O) (an unsubstituted C₁-C₆ alkyl);
R^{3D} is selected from the group consisting of H, —CH₃, —OH and —CH₂Y¹, wherein Y¹ is halogen;
X² is —OR⁹, —SR⁹, or —NHR⁹;
R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl;
n⁴ and n⁵ are each individually 0, 1 or 2, with the proviso that n⁴ and n⁵ are not both 0;
each Y is individually H or halogen;
each m is individually 1 or 2;
each p is individually 1, 2, 3, 4, 5, or 6;
each q is individually 0, 1, 2, 3, 4, 5, or 6;
each t is individually 1, 2, 3, 4, 5, or 6;
R⁷ is H, —COR⁸, —CO₂R⁸, (CO)—NHR⁸, L⁴, L⁵, L⁶, or L⁷;
R⁸ is a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³;
R⁹ is H, —COR⁸, —CO₂R⁸, —(CO)—NHR⁸, L⁴, L⁵, L⁶, or L⁷, with the proviso that exactly one of R⁷ and R⁹ is L⁴, L⁵, L⁶, or L⁷; and
each X³ is individually —H, —OH, —SH, or —NH₂.

105-124. (canceled)

125. The conjugate of claim 124, or a pharmaceutically acceptable salt thereof, wherein R¹ is C₁-C₃ alkyl and R² is a halogen.

126. The conjugate of claim 124 or 125, or a pharmaceutically acceptable salt thereof, wherein R¹ is methyl and R² is F.

127-151. (canceled)

152. The conjugate of claim 104, or a pharmaceutically acceptable salt thereof, wherein the conjugate having Formula (III) is selected from the group consisting of: 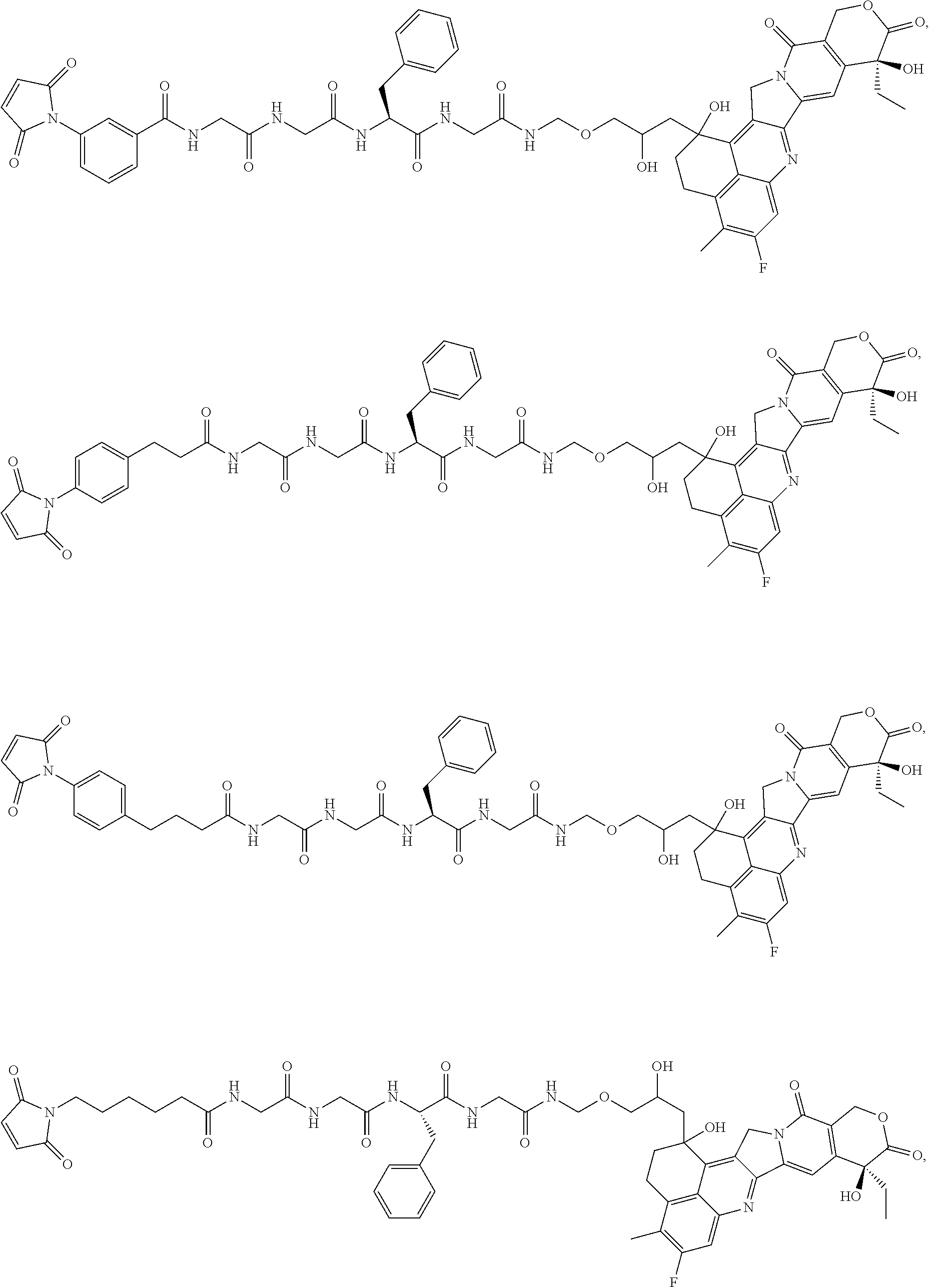 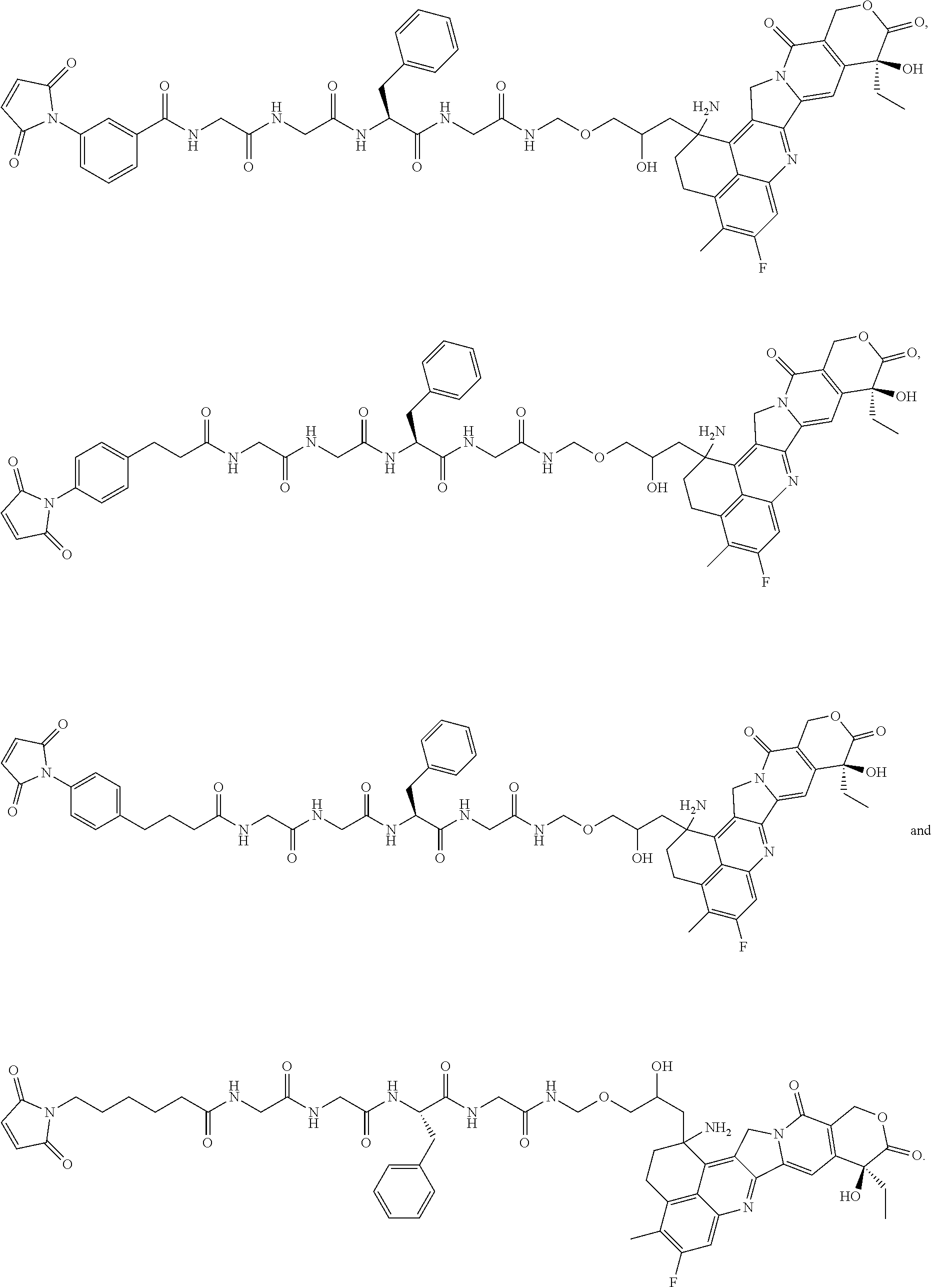
or a pharmaceutically acceptable salt of any of the foregoing.

153. The conjugate of claim 152, or a pharmaceutically acceptable salt thereof, wherein the conjugate having Formula (III) is selected from the group consisting of: 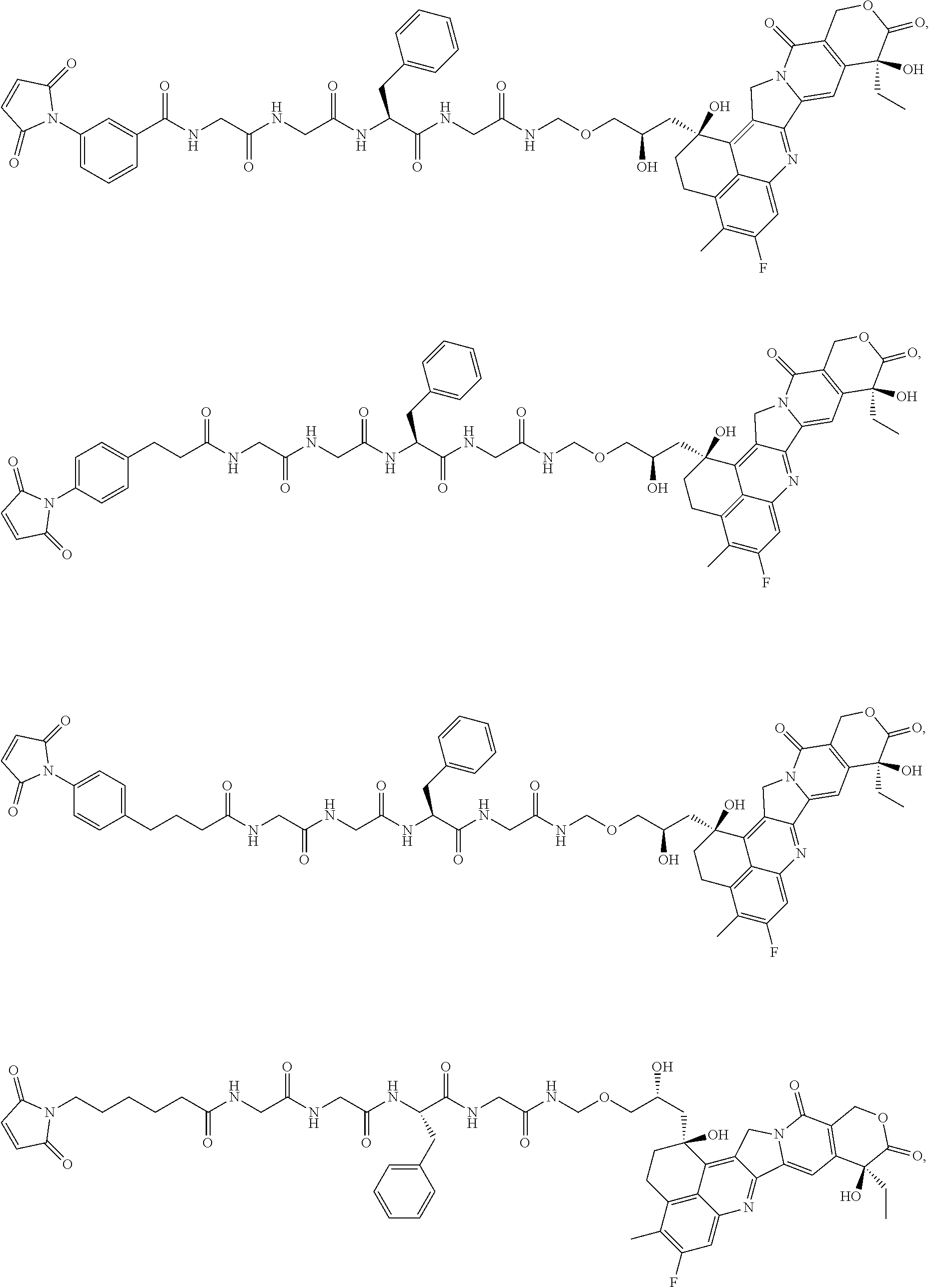 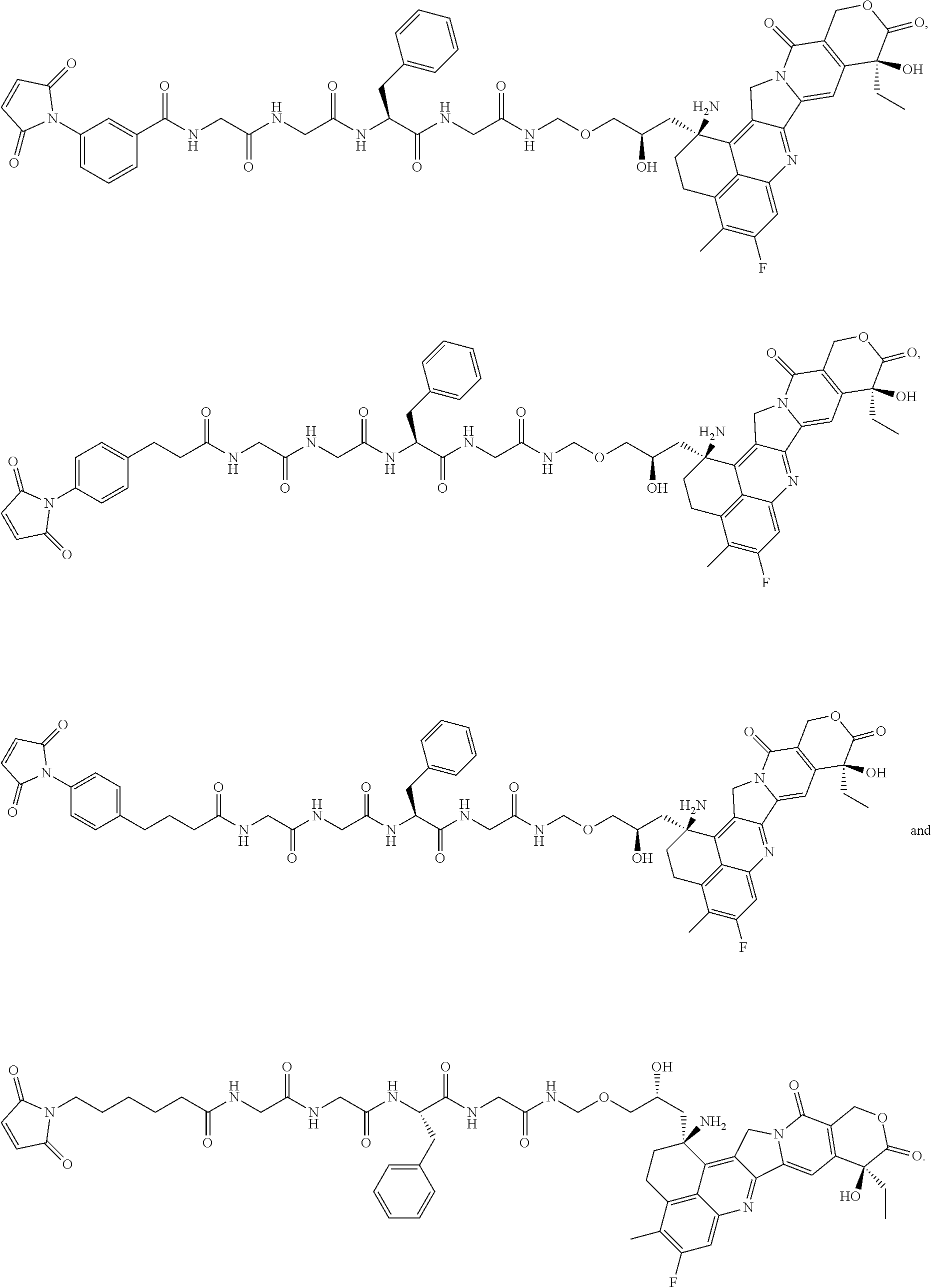
or a pharmaceutically acceptable salt of any of the foregoing.

154. The conjugate of claim 104, wherein Formula (III) is selected from the group consisting of: 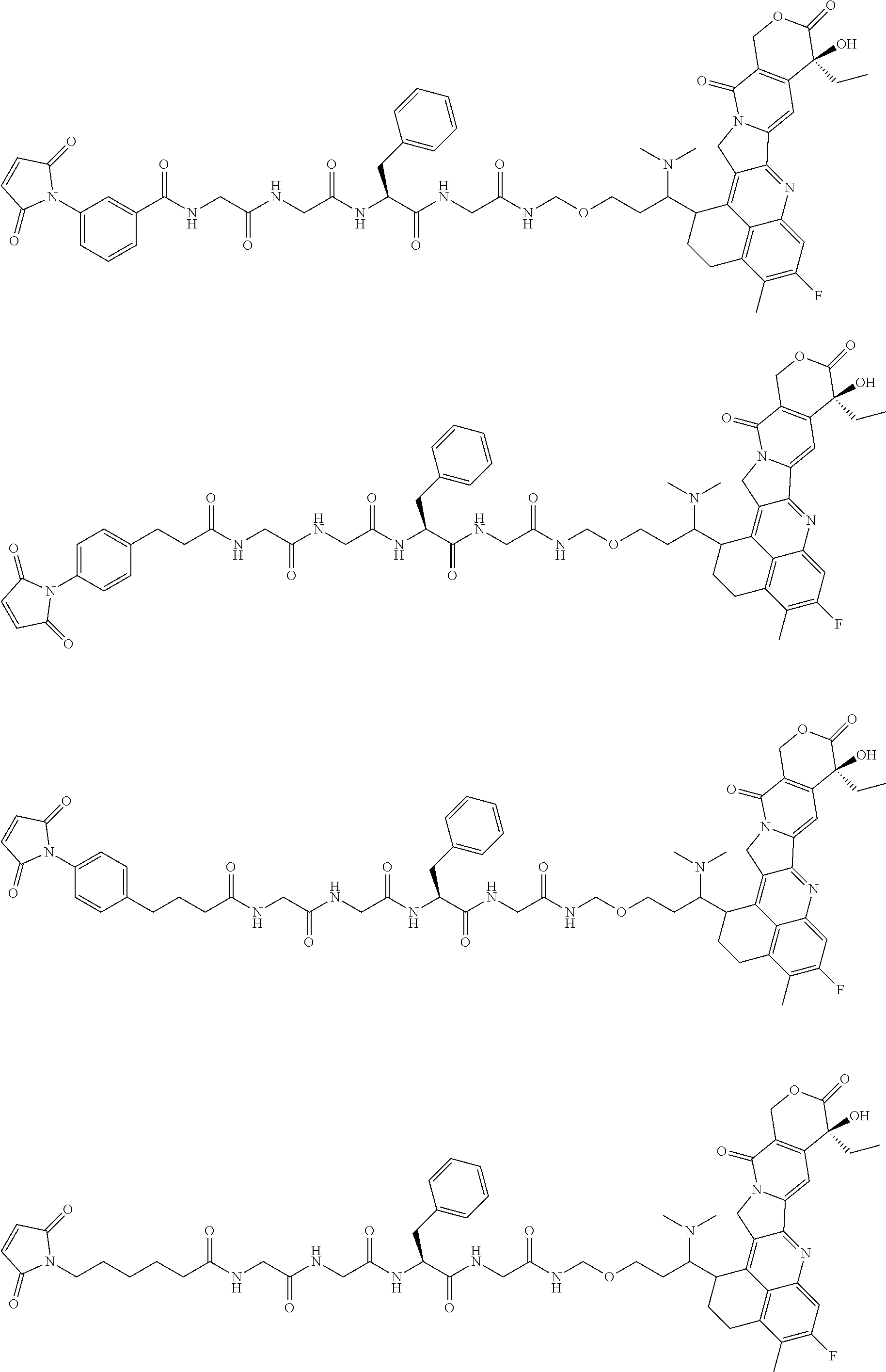 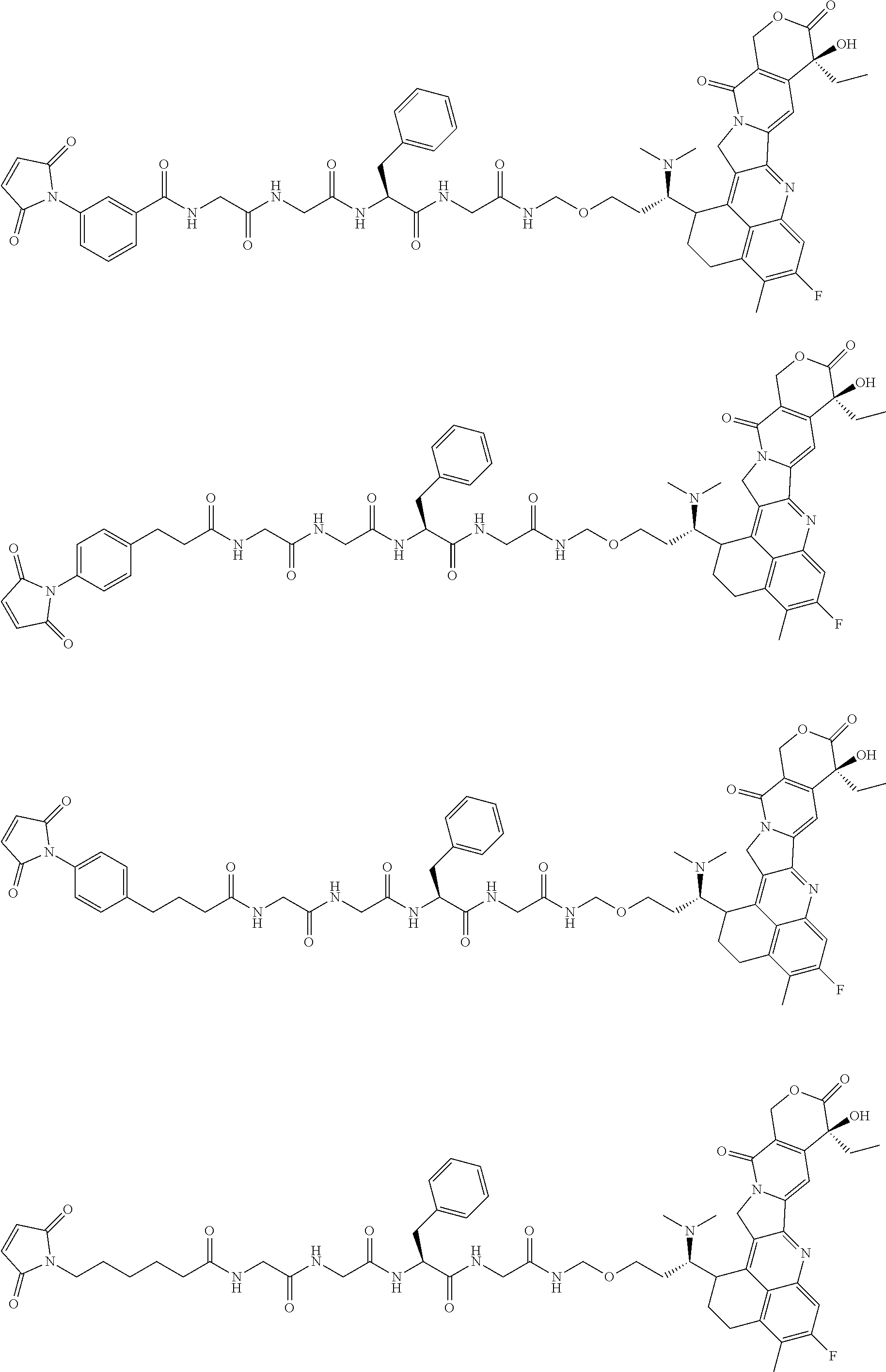 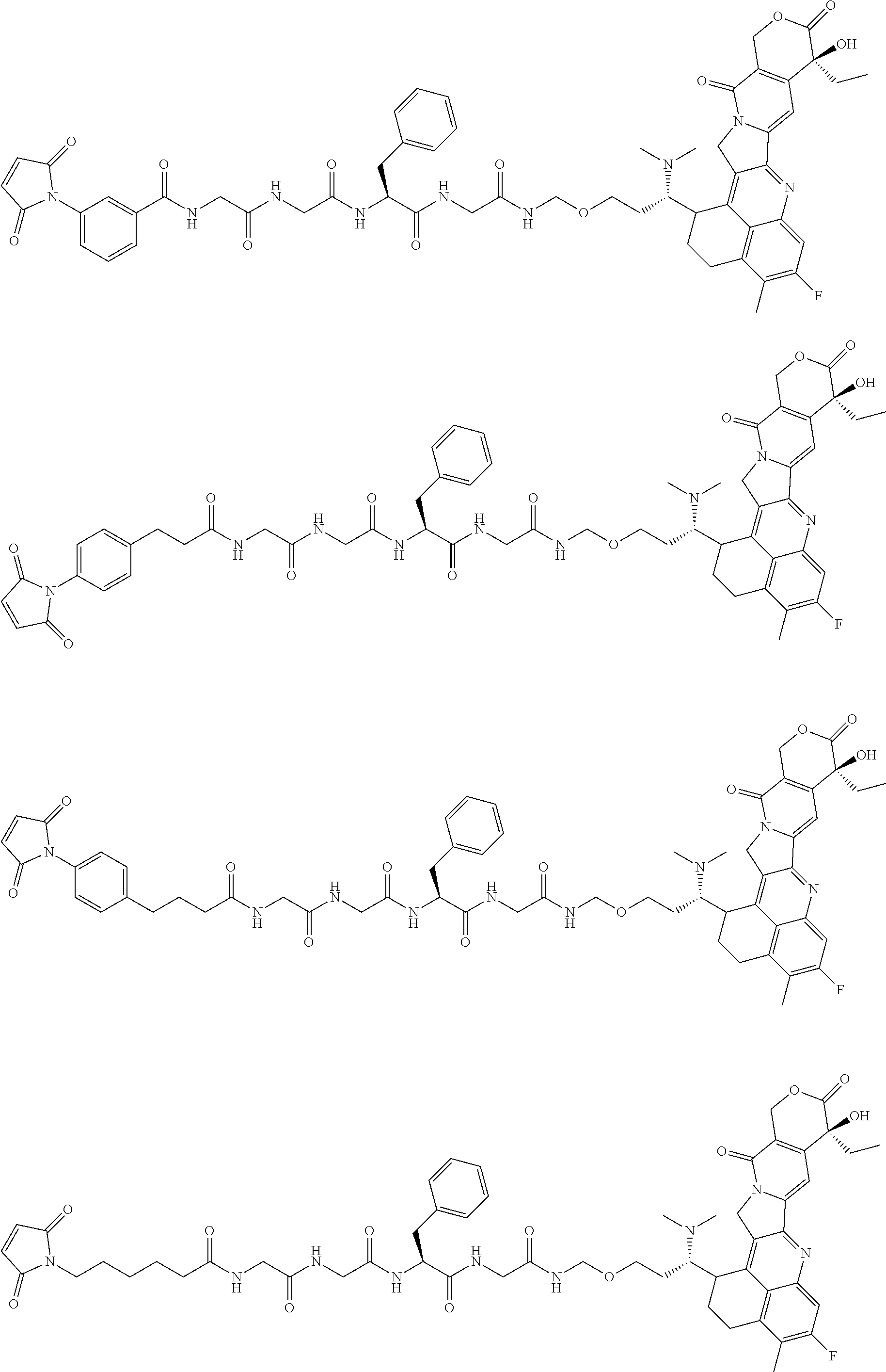 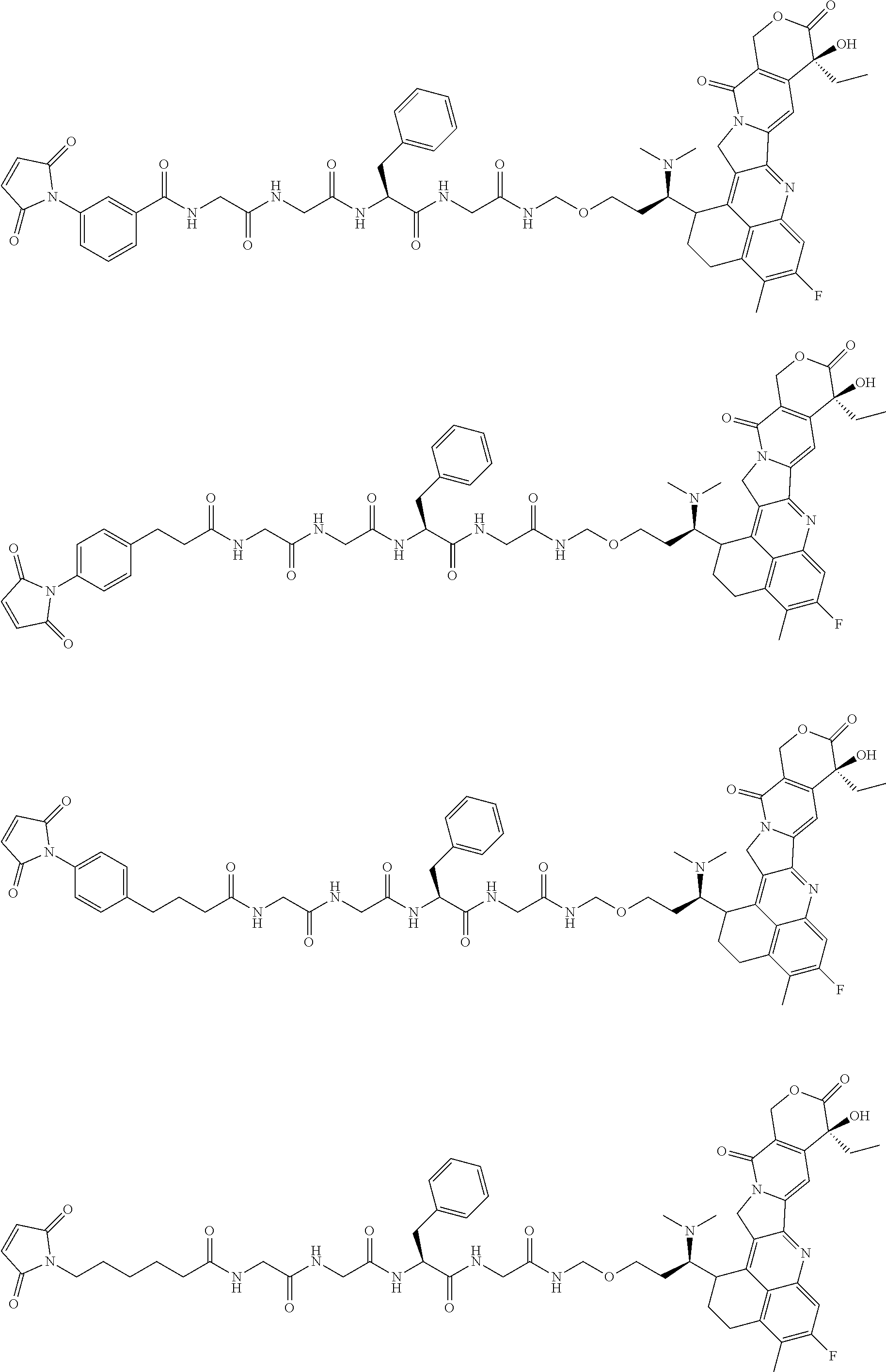 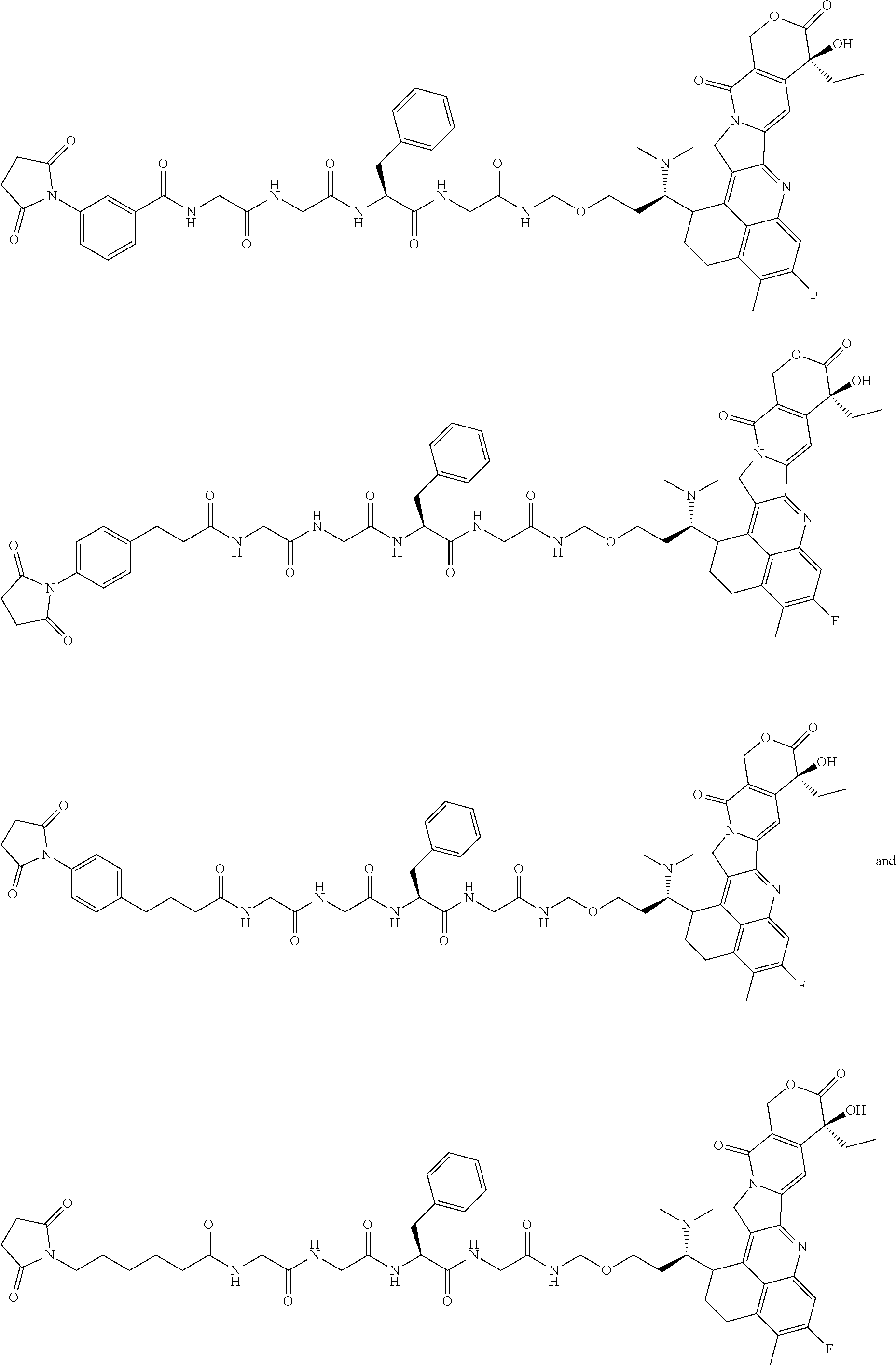
or a pharmaceutically acceptable salt of any of the foregoing.

155-162. (canceled)


SEQUENCE LISTING (46 sequences)

SEQ ID NO: 1 - protein, 8 aa, synthetic construct
GFTFSNAW

SEQ ID NO: 2 - protein, 10 aa, synthetic construct
IKSKTDGGTT

SEQ ID NO: 3 - protein, 9 aa, synthetic construct
TTGPDDLDY

SEQ ID NO: 4 - protein, 354 aa, synthetic construct
GAGGTGCAGCTGGTGGAGTCCGGGGGAGGCCTGGTCAAGCCTGGGGGGTCCCTGAGACTCTCCTGTGCAGCCTCTGGATTCACTTTCAGTAACGCCTGGATGAGCTGGGTCCGCCAGGCTCCAGGGAAGGGGCTGGAGTGGGTTGGCCGTATTAAAAGCAAAACTGATGGTGGGACAACAGACTACGCTGCACCCGTGAAAGGCAGATTCACCATCTCAAGAGATGATTCAAAAAACACGCTCTATCTGCAAATGAACAGCCTGAAAACCGAGGACACAGCCGTGTATTACTGTACCACAGGCCCTGACGATCTTGACTACTGGGGCCAGGGAACCCCGGTCACCGTCTCCTCA

SEQ ID NO: 5 - protein, 118 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTGPDDLDYWGQGTPVTVSS

SEQ ID NO: 6 - protein, 1341 aa, synthetic construct
GAGGTGCAGCTGGTGGAGTCCGGGGGAGGCCTGGTCAAGCCTGGGGGGTCCCTGAGACTCTCCTGTGCAGCCTCTGGATTCACTTTCAGTAACGCCTGGATGAGCTGGGTCCGCCAGGCTCCAGGGAAGGGGCTGGAGTGGGTTGGCCGTATTAAAAGCAAAACTGATGGTGGGACAACAGACTACGCTGCACCCGTGAAAGGCAGATTCACCATCTCAAGAGATGATTCAAAAAACACGCTCTATCTGCAAATGAACAGCCTGAAAACCGAGGACACAGCCGTGTATTACTGTACCACAGGCCCTGACGATCTTGACTACTGGGGCCAGGGAACCCCGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 7 - protein, 447 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTGPDDLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 6 aa, synthetic construct
QSISSY

SEQ ID NO: 9 - skipped

SEQ ID NO: 10 - protein, 9 aa, synthetic construct
QQYDNLPIT

SEQ ID NO: 11 - protein, 321 aa, synthetic construct
GACATCCAGATGACCCAGTCTCCATCCTCCCTGTCTGCATCTGTAGGAGACAGAGTCACCATCACTTGCCGGGCAAGTCAGAGCATTAGCAGCTATTTAAATTGGTATCAGCAGAAACCAGGGAAAGCCCCTAAGCTCCTGATCTATGCTGCATCCAGTTTGCAAAGTGGGGTCCCATCAAGGTTCAGCGGCAGTGGATCTGGGACAGATTTCACTCTCACCATCAGCAGCCTGCAACCTGAAGATTTTGCAACTTACTACTGTCAACAGTATGATAATCTCCCGATCACCTTCGGCCAAGGGACACGACTGGAGATTAAA

SEQ ID NO: 12 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQYDNLPITFGQGTRLEIK

SEQ ID NO: 13 - protein, 642 aa, synthetic construct
GACATCCAGATGACCCAGTCTCCATCCTCCCTGTCTGCATCTGTAGGAGACAGAGTCACCATCACTTGCCGGGCAAGTCAGAGCATTAGCAGCTATTTAAATTGGTATCAGCAGAAACCAGGGAAAGCCCCTAAGCTCCTGATCTATGCTGCATCCAGTTTGCAAAGTGGGGTCCCATCAAGGTTCAGCGGCAGTGGATCTGGGACAGATTTCACTCTCACCATCAGCAGCCTGCAACCTGAAGATTTTGCAACTTACTACTGTCAACAGTATGATAATCTCCCGATCACCTTCGGCCAAGGGACACGACTGGAGATTAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 14 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQYDNLPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 8 aa, synthetic construct
GGSFSGYY

SEQ ID NO: 16 - protein, 7 aa, synthetic construct
INHSGST

SEQ ID NO: 17 - protein, 9 aa, synthetic construct
AREGVYEDY

SEQ ID NO: 18 - protein, 345 aa, synthetic construct
CAGGTGCAGCTACAGCAGTGGGGCGCAGGACTGTTGAAGCCTTCGGAGACCCTGTCCCTCACCTGCGCTGTCTATGGTGGGTCCTTCAGTGGTTACTACTGGAGCTGGATCCGCCAGCCCCCAGGGAAGGGGCTGGAGTGGATTGGGGAAATCAATCATAGTGGAAGCACCAACTACAACCCGTCCCTCAAGAGTCGAGTCACCATATCAGTAGACACGTCCAAGAACCAGTTCTCCCTGAAGCTGAGCTCTGTGACCGCCGCGGACACGGCTGTATATTACTGTGCGAGAGAGGGTGTCTACGAGGACTACTGGGGCCAGGGAACCCTGGTCACCGTCTCCTCA

SEQ ID NO: 19 - protein, 115 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAREGVYEDYWGQGTLVTVSS

SEQ ID NO: 20 - protein, 1332 aa, synthetic construct
CAGGTGCAGCTACAGCAGTGGGGCGCAGGACTGTTGAAGCCTTCGGAGACCCTGTCCCTCACCTGCGCTGTCTATGGTGGGTCCTTCAGTGGTTACTACTGGAGCTGGATCCGCCAGCCCCCAGGGAAGGGGCTGGAGTGGATTGGGGAAATCAATCATAGTGGAAGCACCAACTACAACCCGTCCCTCAAGAGTCGAGTCACCATATCAGTAGACACGTCCAAGAACCAGTTCTCCCTGAAGCTGAGCTCTGTGACCGCCGCGGACACGGCTGTATATTACTGTGCGAGAGAGGGTGTCTACGAGGACTACTGGGGCCAGGGAACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 21 - protein, 444 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAREGVYEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 6 aa, synthetic construct
QSVSNY

SEQ ID NO: 23 - skipped

SEQ ID NO: 24 - protein, 9 aa, synthetic construct
QQRSNWPLT

SEQ ID NO: 25 - protein, 321 aa, synthetic construct
GAAATTGTGTTGACACAGTCTCCAGCCACCCTGTCTTTGTCTCCAGGGGAAAGAGCCACCCTCTCCTGCAGGGCCAGTCAGAGTGTTAGCAACTACTTAGCCTGGTACCAACAGAAACCTGGCCAGGCTCCCAGGCTCCTCATCTATGATGCCTACAACAGGGCCACTGGCATCCCAGCCAGGTTCAGTGGCAGTGGGTCTGGGACAGACTTCACTCTCACCATCAGCAGCCTAGAGCCTGAAGATTTTGCAGTTTATTACTGTCAGCAGCGTAGCAACTGGCCTCTCACCTTCGGCCAAGGGACACGACTGGAGATTAAA

SEQ ID NO: 26 - protein, 107 aa, synthetic construct
EIVLTQSPATLSLSPGERATLSCRASQSVSNYLAWYQQKPGQAPRLLIYDAYNRATGIPARFSGSGSGTDFTLTISSLEPEDFAVYYCQQRSNWPLTFGQGTRLEIK

SEQ ID NO: 27 - protein, 642 aa, synthetic construct
GAAATTGTGTTGACACAGTCTCCAGCCACCCTGTCTTTGTCTCCAGGGGAAAGAGCCACCCTCTCCTGCAGGGCCAGTCAGAGTGTTAGCAACTACTTAGCCTGGTACCAACAGAAACCTGGCCAGGCTCCCAGGCTCCTCATCTATGATGCCTACAACAGGGCCACTGGCATCCCAGCCAGGTTCAGTGGCAGTGGGTCTGGGACAGACTTCACTCTCACCATCAGCAGCCTAGAGCCTGAAGATTTTGCAGTTTATTACTGTCAGCAGCGTAGCAACTGGCCTCTCACCTTCGGCCAAGGGACACGACTGGAGATTAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 28 - protein, 214 aa, synthetic construct
EIVLTQSPATLSLSPGERATLSCRASQSVSNYLAWYQQKPGQAPRLLIYDAYNRATGIPARFSGSGSGTDFTLTISSLEPEDFAVYYCQQRSNWPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 8 aa, synthetic construct
GYTFTGYY

SEQ ID NO: 30 - protein, 8 aa, synthetic construct
INPNSGGT

SEQ ID NO: 31 - protein, 12 aa, synthetic construct
VRDQVQLERFDS

SEQ ID NO: 32 - protein, 357 aa, synthetic construct
CAGGTGCAGCTGGTGCAGTCTGGGGCTGAGGTGAAGAAGCCTGGGGCCTCAGTGAAGGTCTCCTGCAAGGCTTCTGGATACACCTTCACCGGCTACTATATGCACTGGGTGCGACAGGCCCCTGGACAAGGGCTTGAGTGGATGGGATGGATCAACCCTAACAGTGGTGGCACAAACTATGCACAGAAGTTTCAGGGCAGGGTCACCATGACCAGGGACACGTCCATCAGCACAGCCTACATGGAGCTGAGCAGGCTGAGATCTGACGACACGGCCGTGTATTACTGTGTGAGAGATCAGGTACAACTGGAACGGTTCGACTCCTGGGGCCAGGGAACCCTGGTCACCGTCTCCTCA

SEQ ID NO: 33 - protein, 119 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCVRDQVQLERFDSWGQGTLVTVSS

SEQ ID NO: 34 - protein, 1344 aa, synthetic construct
CAGGTGCAGCTGGTGCAGTCTGGGGCTGAGGTGAAGAAGCCTGGGGCCTCAGTGAAGGTCTCCTGCAAGGCTTCTGGATACACCTTCACCGGCTACTATATGCACTGGGTGCGACAGGCCCCTGGACAAGGGCTTGAGTGGATGGGATGGATCAACCCTAACAGTGGTGGCACAAACTATGCACAGAAGTTTCAGGGCAGGGTCACCATGACCAGGGACACGTCCATCAGCACAGCCTACATGGAGCTGAGCAGGCTGAGATCTGACGACACGGCCGTGTATTACTGTGTGAGAGATCAGGTACAACTGGAACGGTTCGACTCCTGGGGCCAGGGAACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 35 - protein, 448 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCVRDQVQLERFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 6 aa, synthetic construct
QDISNY

SEQ ID NO: 37 - skipped

SEQ ID NO: 38 - protein, 9 aa, synthetic construct
QQYDNLPPT

SEQ ID NO: 39 - protein, 321 aa, synthetic construct
GACATCCAGATGACCCAGTCTCCATCCTCCCTGTCTGCATCTGTAGGAGACAGAGTCACCATCACTTGCCAGGCGAGTCAGGACATTAGCAACTATTTAAATTGGTATCAGCAGAAACCAGGGAAAGCCCCTAAGCTCCTGATCTACGATGCATCCAATTTGGAAACAGGGGTCCCATCAAGGTTCAGTGGAAGTGGATCTGGGACAGATTTTACTTTCACCATCAGCAGCCTGCAGCCTGAAGATATTGCAACATATTACTGTCAACAGTATGATAATCTCCCTCCCACTTTCGGCCCTGGGACCAAGGTGGAAATCAAA

SEQ ID NO: 40 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPPTFGPGTKVEIK

SEQ ID NO: 41 - protein, 642 aa, synthetic construct
GACATCCAGATGACCCAGTCTCCATCCTCCCTGTCTGCATCTGTAGGAGACAGAGTCACCATCACTTGCCAGGCGAGTCAGGACATTAGCAACTATTTAAATTGGTATCAGCAGAAACCAGGGAAAGCCCCTAAGCTCCTGATCTACGATGCATCCAATTTGGAAACAGGGGTCCCATCAAGGTTCAGTGGAAGTGGATCTGGGACAGATTTTACTTTCACCATCAGCAGCCTGCAGCCTGAAGATATTGCAACATATTACTGTCAACAGTATGATAATCTCCCTCCCACTTTCGGCCCTGGGACCAAGGTGGAAATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 42 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPPTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 4 aa, synthetic construct
GGFG

SEQ ID NO: 44 - protein, 4 aa, synthetic construct
EGGF

SEQ ID NO: 45 - protein, 4 aa, synthetic construct
SGGF

SEQ ID NO: 46 - protein, 4 aa, synthetic construct
KGGF